(19) United States
(12) Patent Application Publication
(10) Pub. No.: US 2024/0228470 A1
(43) Pub. Date: Jul. 11, 2024

(54) Organic Compound And Organic Light Emitting Device Including Same

(71) Applicant: P&H TECH Co., Ltd, Yongin-si, Gyeonggi-do (KR)

(72) Inventors: Seok-Keun YOON, Gyeonggi-do (KR); Seo-Yong HYUN, Gyeonggi-do (KR); In-Ho LEE, Gyeonggi-do (KR)

(73) Assignee: P&H TECH Co., Ltd, Yongin-si, Gyeonggi-do (KR)

(21) Appl. No.: 18/555,493

(22) PCT Filed: Sep. 27, 2021
(86) PCT No.: PCT/KR2021/013093

§ 371 Date: Oct. 13, 2023

(30) Foreign Application Priority Data
Apr. 14, 2021 (KR) 10-2021-0048347
Oct. 12, 2022 (KR) 10-2022-0130748

(51) Int. Cl. C07D 405/10; C07D 401/10; C07D 405/14; C07D 409/04; C07D 409/10; C07D 409/14; C07D 413/14; C07D 417/14; C07D 491/048; H10K 50/11; H10K 50/15; H10K 50/16; H10K 50/17; H10K 50/18; H10K 85/60

(52) U.S. Cl. CPC C07D 405/10; C07D 401/10; C07D 405/14; C07D 409/04; C07D 409/10; C07D 409/14; C07D 413/14; C07D 417/14; C07D 491/048; H10K 50/11; H10K 50/15; H10K 50/16; H10K 50/171; H10K 50/181; H10K 85/654; H10K 85/6574; H10K 85/6576

(57) ABSTRACT

The present invention provides an organic compound that is employed in an organic layer, such as an electron transport layer, in an organic light-emitting device to realize the characteristics thereof, such as significantly improved excellence in luminosity efficiency, alongside low voltage driving characteristics of the organic light-emitting device. The organic compound and the organic light-emitting device comprising same, according to the present invention, can be industrially utilized in various lighting, display devices and the like.

### TECHNICAL FIELD

The present invention relates to an organic compound, and more particularly, to an organic compound which is employed as a material for an organic layer in an organic light emitting device, and an organic light emitting device which employs the same, thus achieving greatly improved luminescent properties such as low-voltage driving of the device and excellent luminous efficiency.

### BACKGROUND ART

The organic light emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and read, and thus has recently become a subject of intense interest as a next-generation display device.

However, in order for such an organic light emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light emitting device has not yet been sufficiently made.

Thus, further improvements in terms of efficiency and life characteristics are required for good stability, high efficiency, long lifetime, and large size of organic light emitting devices. Particularly, there is a strong need to develop materials constituting each organic layer of organic light emitting devices.

### DISCLOSURE

### Technical Problem

Accordingly, an aspect of the present invention intends to provide a novel organic compound which is employed in an organic layer in an organic light emitting device, achieving excellent luminescent properties such as low-voltage driving of the device and improved luminous efficiency, and an organic light emitting device including the same.

### Technical Solution

An aspect of the present invention provides an organic compound represented by Formula I below and an organic light emitting device including the same in an organic layer in the device.

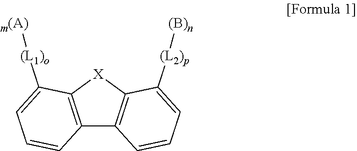

Characteristic structures of Formula I above and structures and definitions of Compounds L₁, L₂, A, and B implemented thereby will be described below.

### Advantageous Effects

When an organic compound according to an embodiment of the present invention is employed as a material for an organic layer such as an electron transport layer in the organic light emitting device, it is possible to achieve luminescent properties such as low-voltage driving of the device and excellent luminous efficiency, and thus can be usefully used in various display devices.

### BEST MODE

Hereinafter, the present invention will be described in more detail.

The present invention relates to an organic compound represented by Formula I below which achieves luminescent properties such as low-voltage driving of a device and excellent luminous efficiency of the organic light emitting device and is characterized by introducing A and B substituents into a characteristic position of a skeletal structure structurally represented by Formula I below, that is, fluorene (dimenzofuran, dibenzothiophene) derivative structures, thus improving low-voltage driving characteristic and luminous efficiency characteristic of the organic light emitting device by means of the structural characteristic.

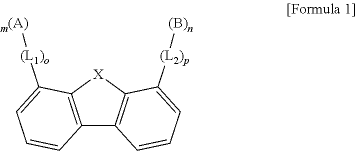

In Formula I above, X is O, S, or CR₁R₂.

R₁ and R₂ are each independently selected from hydrogen, deuterium, a substituted or unsubstituted C1 to C20 alkyl group, a substituted or unsubstituted C3 to C20 cycloalkyl group, a substituted or unsubstituted C6 to C30 aryl group, and a substituted or unsubstituted C2 to C30 heteroaryl group.

The R₁ and R₂ may be bonded to each other to form a substituted ring or an aromatic ring.

L₁ and L₂ may be single bonds or selected from a substituted or unsubstituted C6 to C30 arylene group, and a substituted or unsubstituted C3 to C30 heteroarylene group, o and p are each independently an integer of 0 to 4. When o and p are 2 or larger, the plurality of L₁ and L₂ are the same or different from each other.

A and B are the same or different from each other and are each independently selected from a halogen group, a cyano group, a substituted or unsubstituted C1 to C20 alkyl group, a substituted or unsubstituted C3 to C20 cycloalkyl group, a substituted or unsubstituted C1 to C20 heterocycloalkyl group, a substituted or unsubstituted C6 to C30 aryl group, and a substituted or unsubstituted C2 to C30 heteroaryl group as a characteristic substituent, other than hydrogen. n and m are integers of 1 or 2 and when n and m are 2 or larger, the plurality of A and B are the same or different from each other.

In Formula I according to an embodiment of the present invention, at least one or more of A and B are represented by Structural Formula 1 below. That is, A and B are each defined by any one substituent selected from the substituent definitions and at least one or more of A and B are a substituted or unsubstituted pyridine derivative structure represented by Structural Formula 1 below.

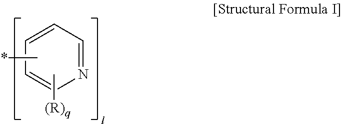

In Structural Formula 1, R is selected from hydrogen, deuterium, halogen, a cyano group, a substituted or unsubstituted C1 to C20 alkyl group, a substituted or unsubstituted C3 to C20 cycloalkyl group, a substituted or unsubstituted C1 to C20 heterocyclo alkyl group, a substituted or unsubstituted C6 to C30 aryl group, a substituted or unsubstituted C2 to C30 heteroaryl group, a substituted or unsubstituted C1 to C20 alkyl amine group, a substituted or unsubstituted C6 to C30 aryl amine group, a substituted or unsubstituted C1 to C20 alkyl silyl group, a substituted or unsubstituted C6 to C30 aryl silyl group, and a substituted or unsubstituted C1 to C20 alkoxy group. q is an integer of 0 to 4 and when q is 2 or larger, the plurality of R is the same or different from each other.

l is an integer of 1 to 4 and when 1 is 2 or larger, the plurality of structures in the bracket ([ ]) is the same or different from each other.

In the meantime, the term “substituted” or “unsubstituted” in the definitions of R, R₁, R₂, L₁, L₂, A, and B indicates substitution with one or more substituents selected from the group consisting of deuterium, a halogen group, a cyano group, a nitro group, a hydroxyl group, an alkyl group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkyl silyl group, and an aryl silyl group, substitution with a substituent to which two or more of the substituents are linked, or having no substituent.

For specific examples, the substituted aryl group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. The number of carbon atoms in the alkoxy groups is not particularly limited but is preferably from 1 to 20 as long as steric hindrance is avoided. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

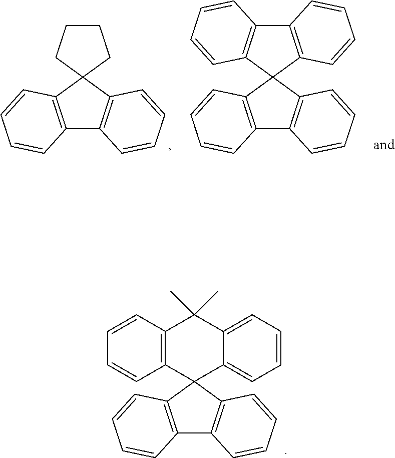

In an embodiment of the present invention, the fluorenyl groups include open structures in which one of the two cyclic organic compounds linked through one atom is cleaved, and examples thereof include

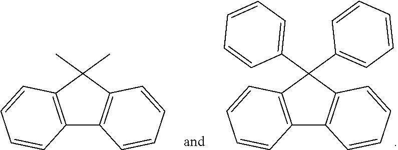

In addition, carbon atoms of the ring may be substituted with any one or more heteroatoms selected from among N, S and O, and examples thereof include

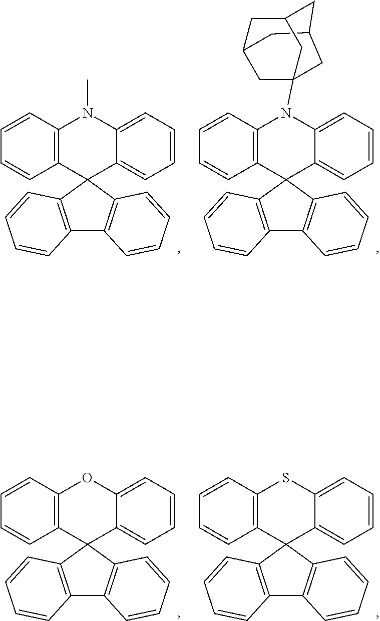

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (Cl), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

The organic compound according to an embodiment of the present invention represented by Formula I above may be used as various organic layers including an electron transport layer in the organic light emitting device due to its structural features.

Preferred and specific examples of the organic compound represented by Formula 1 according to an embodiment of the present invention include the following compounds, but are not limited thereto.

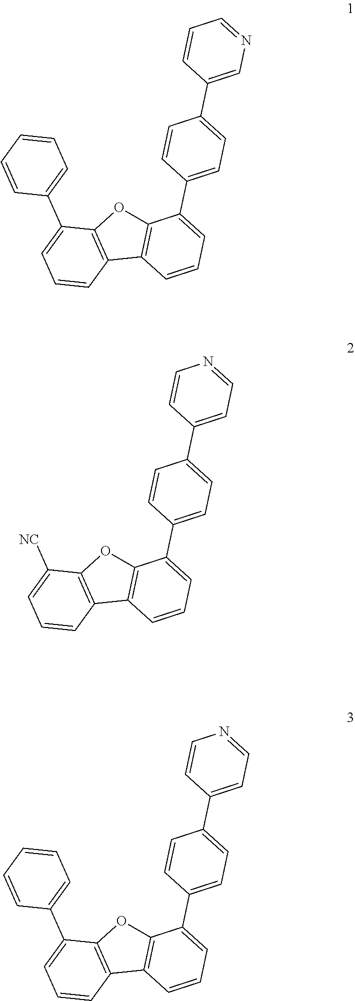 [SMILES: c1ccc(-c2cccc3c2oc2c(-c4ccc(-c5cccnc5)cc4)cccc23)cc1 ; N#Cc1cccc2c1oc1c(-c3ccc(-c4ccncc4)cc3)cccc12 ; c1ccc(-c2cccc3c2oc2c(-c4ccc(-c5ccncc5)cc4)cccc23)cc1]

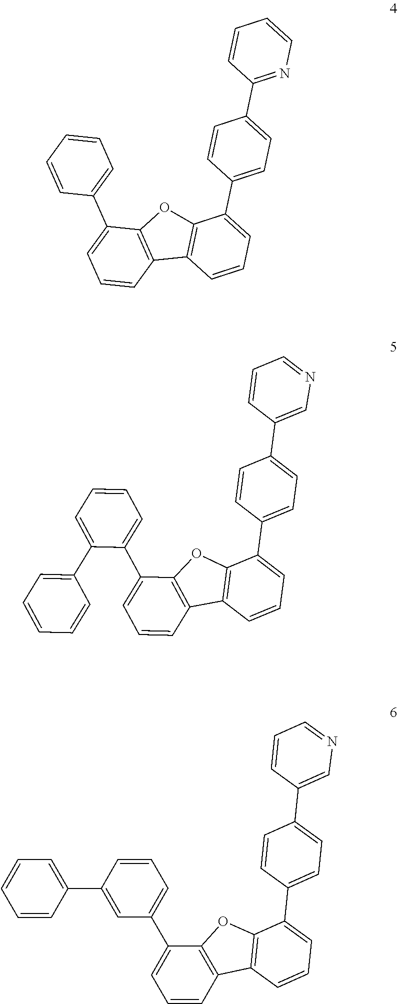 [SMILES: c1ccc(-c2cccc3c2oc2c(-c4ccc(-c5ccccn5)cc4)cccc23)cc1 ; c1ccc(-c2ccccc2-c2cccc3c2oc2c(-c4ccc(-c5cccnc5)cc4)cccc23)cc1 ; c1ccc(-c2cccc(-c3cccc4c3oc3c(-c5ccc(-c6cccnc6)cc5)cccc34)c2)cc1]

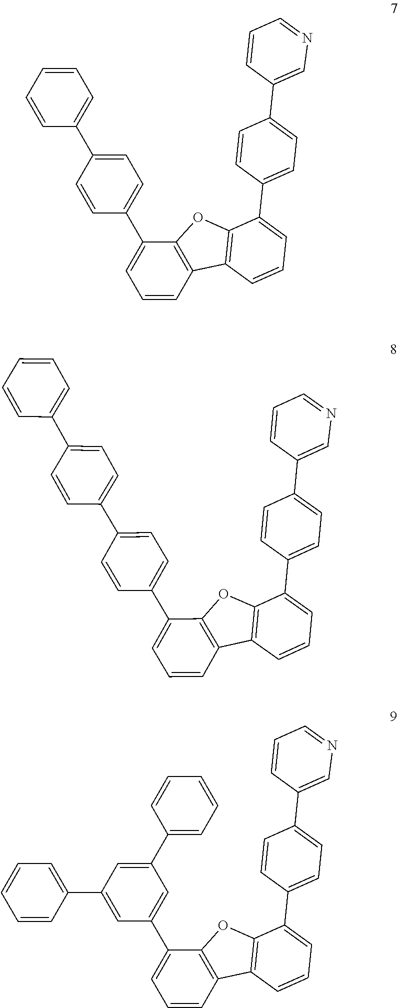 [SMILES: c1ccc(-c2ccc(-c3cccc4c3oc3c(-c5ccc(-c6cccnc6)cc5)cccc34)cc2)cc1 ; c1ccc(-c2ccc(-c3ccc(-c4cccc5c4oc4c(-c6ccc(-c7cccnc7)cc6)cccc45)cc3)cc2)cc1 ; c1ccc(-c2cc(-c3ccccc3)cc(-c3cccc4c3oc3c(-c5ccc(-c6cccnc6)cc5)cccc34)c2)cc1]

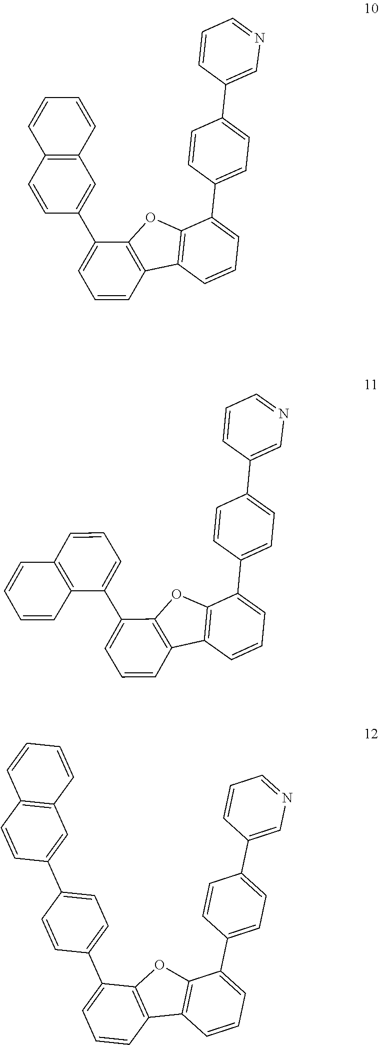 [SMILES: c1cncc(-c2ccc(-c3cccc4c3oc3c(-c5ccc6ccccc6c5)cccc34)cc2)c1 ; c1cncc(-c2ccc(-c3cccc4c3oc3c(-c5cccc6ccccc56)cccc34)cc2)c1 ; c1cncc(-c2ccc(-c3cccc4c3oc3c(-c5ccc(-c6ccc7ccccc7c6)cc5)cccc34)cc2)c1]

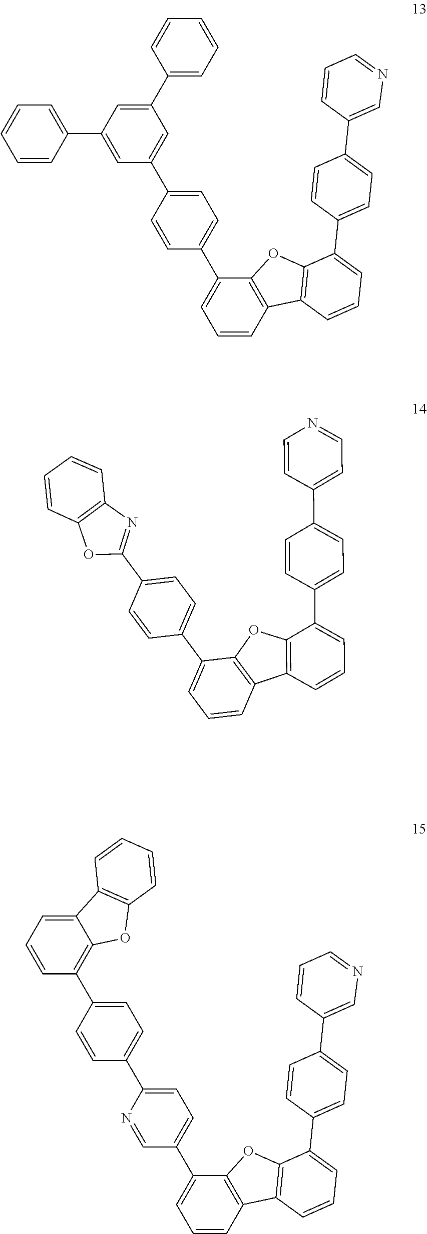 [SMILES: c1ccc(-c2cc(-c3ccccc3)cc(-c3ccc(-c4cccc5c4oc4c(-c6ccc(-c7cccnc7)cc6)cccc45)cc3)c2)cc1 ; c1ccc2oc(-c3ccc(-c4cccc5c4oc4c(-c6ccc(-c7ccncc7)cc6)cccc45)cc3)nc2c1 ; c1cncc(-c2ccc(-c3cccc4c3oc3c(-c5ccc(-c6ccc(-c7cccc8c7oc7ccccc78)cc6)nc5)cccc34)cc2)c1]

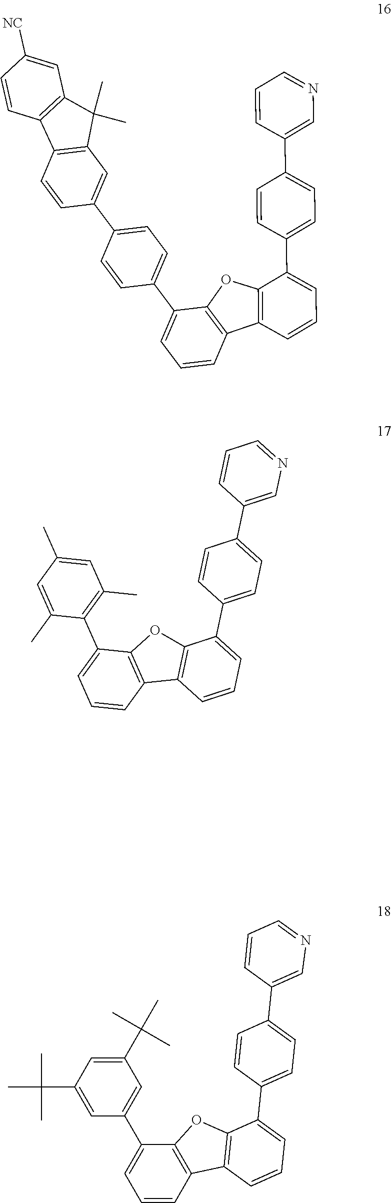 [SMILES: CC1(C)c2cc(C#N)ccc2-c2ccc(-c3ccc(-c4cccc5c4oc4c(-c6ccc(-c7cccnc7)cc6)cccc45)cc3)cc21 ; Cc1cc(C)c(-c2cccc3c2oc2c(-c4ccc(-c5cccnc5)cc4)cccc23)c(C)c1 ; CC(C)(C)c1cc(-c2cccc3c2oc2c(-c4ccc(-c5cccnc5)cc4)cccc23)cc(C(C)(C)C)c1]

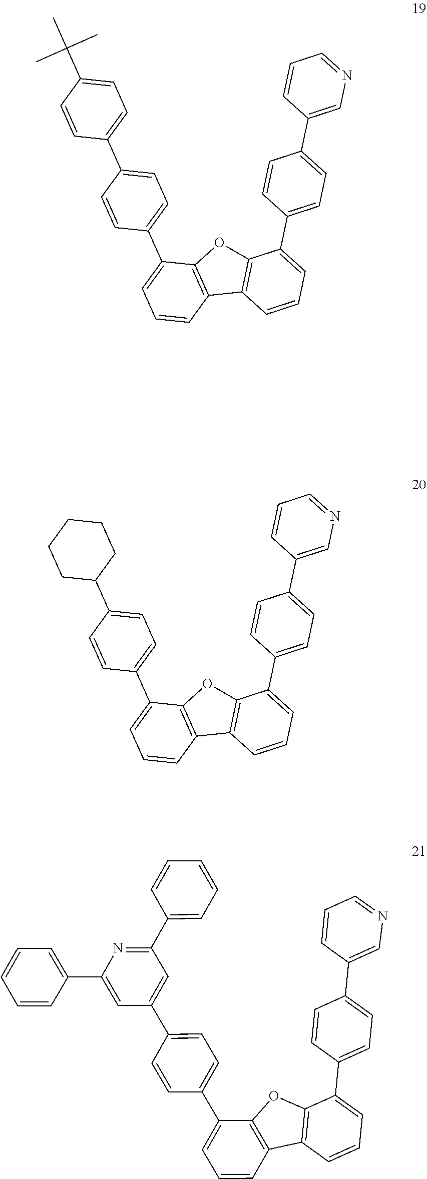 [SMILES: CC(C)(C)c1ccc(-c2ccc(-c3cccc4c3oc3c(-c5ccc(-c6cccnc6)cc5)cccc34)cc2)cc1 ; c1cncc(-c2ccc(-c3cccc4c3oc3c(-c5ccc(C6CCCCC6)cc5)cccc34)cc2)c1 ; c1ccc(-c2cc(-c3ccc(-c4cccc5c4oc4c(-c6ccc(-c7cccnc7)cc6)cccc45)cc3)cc(-c3ccccc3)n2)cc1]

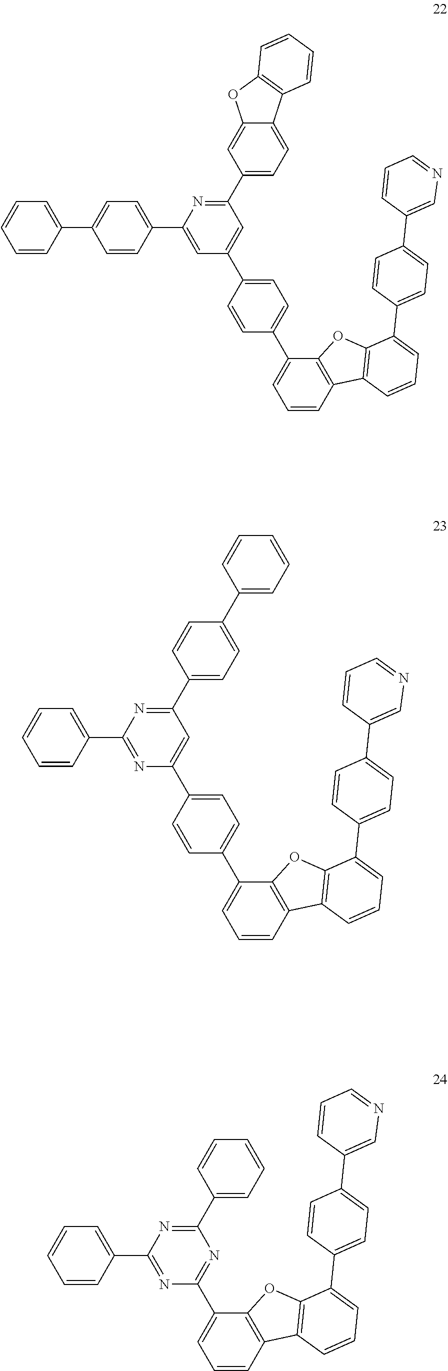 [SMILES: c1ccc(-c2ccc(-c3cc(-c4ccc(-c5cccc6c5oc5c(-c7ccc(-c8cccnc8)cc7)cccc56)cc4)cc(-c4ccc5c(c4)oc4ccccc45)n3)cc2)cc1 ; c1ccc(-c2ccc(-c3cc(-c4ccc(-c5cccc6c5oc5c(-c7ccc(-c8cccnc8)cc7)cccc56)cc4)nc(-c4ccccc4)n3)cc2)cc1 ; c1ccc(-c2nc(-c3ccccc3)nc(-c3cccc4c3oc3c(-c5ccc(-c6cccnc6)cc5)cccc34)n2)cc1]

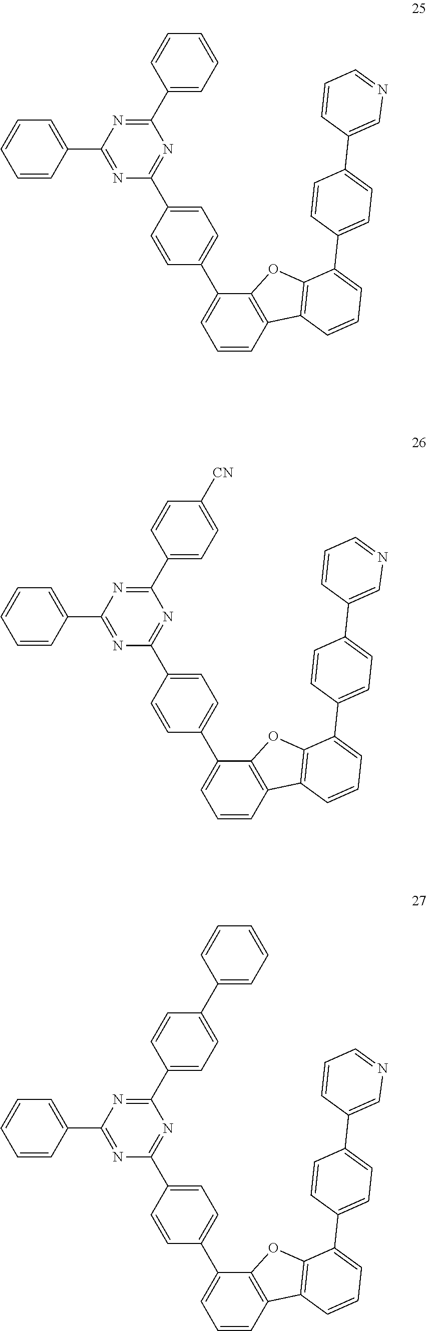 [SMILES: c1ccc(-c2nc(-c3ccccc3)nc(-c3ccc(-c4cccc5c4oc4c(-c6ccc(-c7cccnc7)cc6)cccc45)cc3)n2)cc1 ; N#Cc1ccc(-c2nc(-c3ccccc3)nc(-c3ccc(-c4cccc5c4oc4c(-c6ccc(-c7cccnc7)cc6)cccc45)cc3)n2)cc1 ; c1ccc(-c2ccc(-c3nc(-c4ccccc4)nc(-c4ccc(-c5cccc6c5oc5c(-c7ccc(-c8cccnc8)cc7)cccc56)cc4)n3)cc2)cc1]

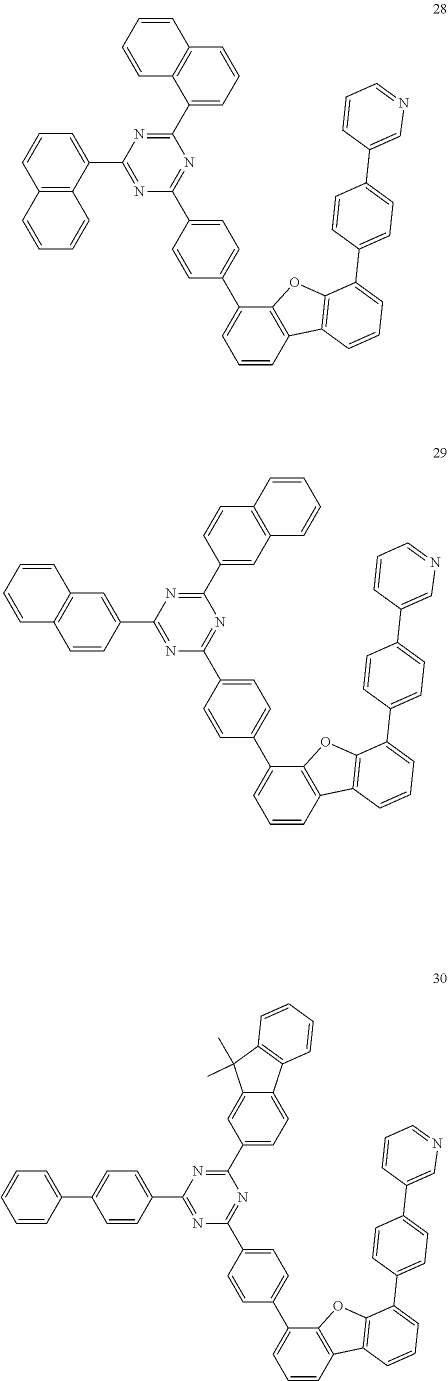 [SMILES: c1cncc(-c2ccc(-c3cccc4c3oc3c(-c5ccc(-c6nc(-c7cccc8ccccc78)nc(-c7cccc8ccccc78)n6)cc5)cccc34)cc2)c1 ; c1cncc(-c2ccc(-c3cccc4c3oc3c(-c5ccc(-c6nc(-c7ccc8ccccc8c7)nc(-c7ccc8ccccc8c7)n6)cc5)cccc34)cc2)c1 ; CC1(C)c2ccccc2-c2ccc(-c3nc(-c4ccc(-c5ccccc5)cc4)nc(-c4ccc(-c5cccc6c5oc5c(-c7ccc(-c8cccnc8)cc7)cccc56)cc4)n3)cc21]

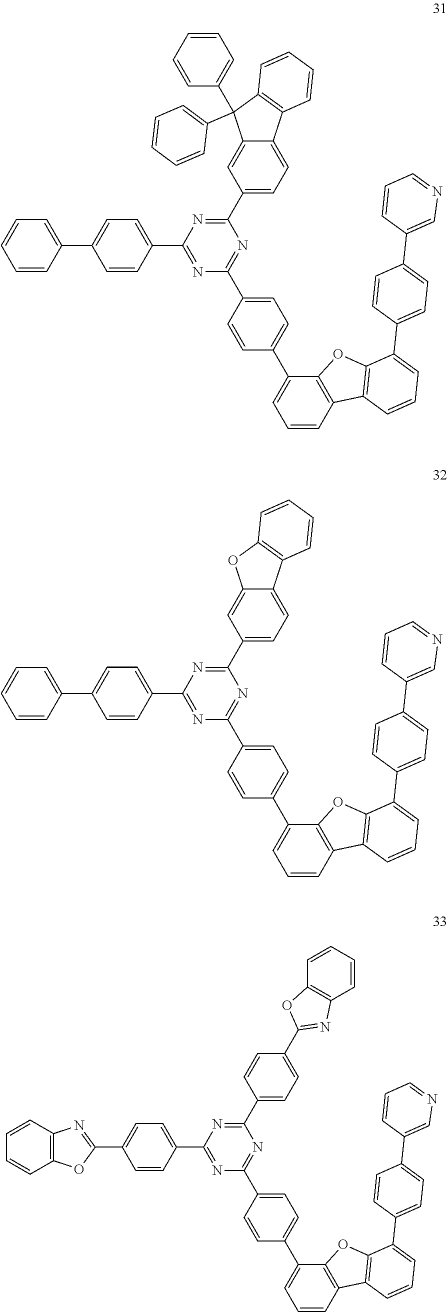 [SMILES: c1ccc(-c2ccc(-c3nc(-c4ccc(-c5cccc6c5oc5c(-c7ccc(-c8cccnc8)cc7)cccc56)cc4)nc(-c4ccc5c(c4)C(c4ccccc4)(c4ccccc4)c4ccccc4-5)n3)cc2)cc1 ; c1ccc(-c2ccc(-c3nc(-c4ccc(-c5cccc6c5oc5c(-c7ccc(-c8cccnc8)cc7)cccc56)cc4)nc(-c4ccc5c(c4)oc4ccccc45)n3)cc2)cc1 ; c1cncc(-c2ccc(-c3cccc4c3oc3c(-c5ccc(-c6nc(-c7ccc(-c8nc9ccccc9o8)cc7)nc(-c7ccc(-c8nc9ccccc9o8)cc7)n6)cc5)cccc34)cc2)c1]

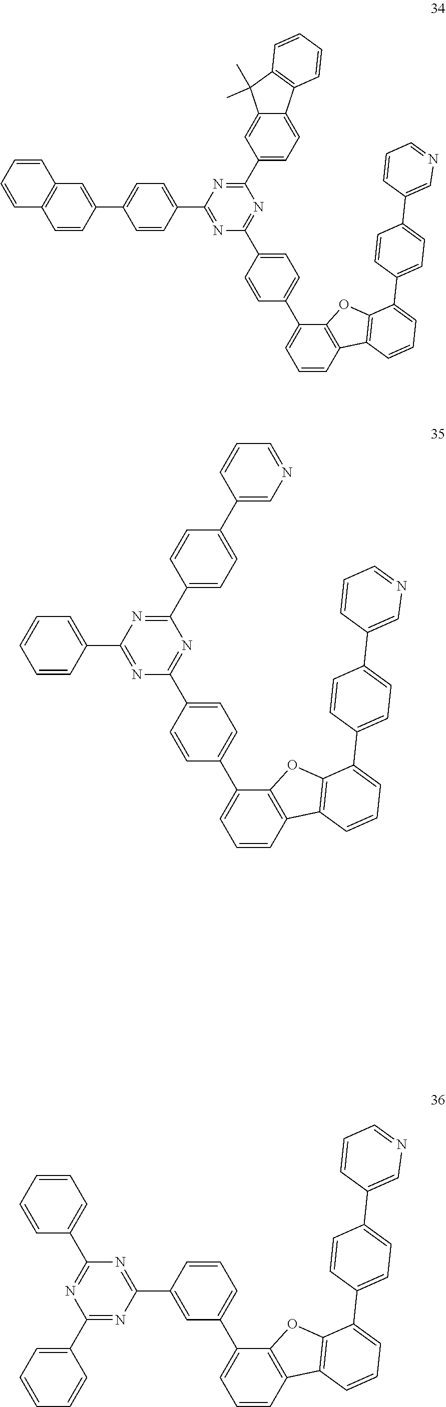 [SMILES: CC1(C)c2ccccc2-c2ccc(-c3nc(-c4ccc(-c5ccc6ccccc6c5)cc4)nc(-c4ccc(-c5cccc6c5oc5c(-c7ccc(-c8cccnc8)cc7)cccc56)cc4)n3)cc21 ; c1ccc(-c2nc(-c3ccc(-c4cccnc4)cc3)nc(-c3ccc(-c4cccc5c4oc4c(-c6ccc(-c7cccnc7)cc6)cccc45)cc3)n2)cc1 ; c1ccc(-c2nc(-c3ccccc3)nc(-c3cccc(-c4cccc5c4oc4c(-c6ccc(-c7cccnc7)cc6)cccc45)c3)n2)cc1]

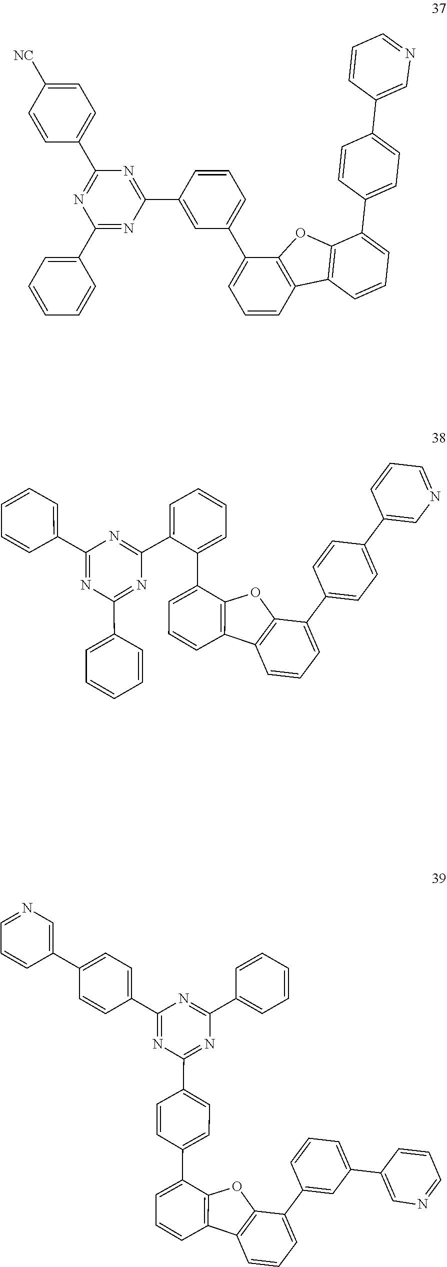 [SMILES: N#Cc1ccc(-c2nc(-c3ccccc3)nc(-c3cccc(-c4cccc5c4oc4c(-c6ccc(-c7cccnc7)cc6)cccc45)c3)n2)cc1 ; c1ccc(-c2nc(-c3ccccc3)nc(-c3ccccc3-c3cccc4c3oc3c(-c5ccc(-c6cccnc6)cc5)cccc34)n2)cc1 ; c1ccc(-c2nc(-c3ccc(-c4cccnc4)cc3)nc(-c3ccc(-c4cccc5c4oc4c(-c6cccc(-c7cccnc7)c6)cccc45)cc3)n2)cc1]

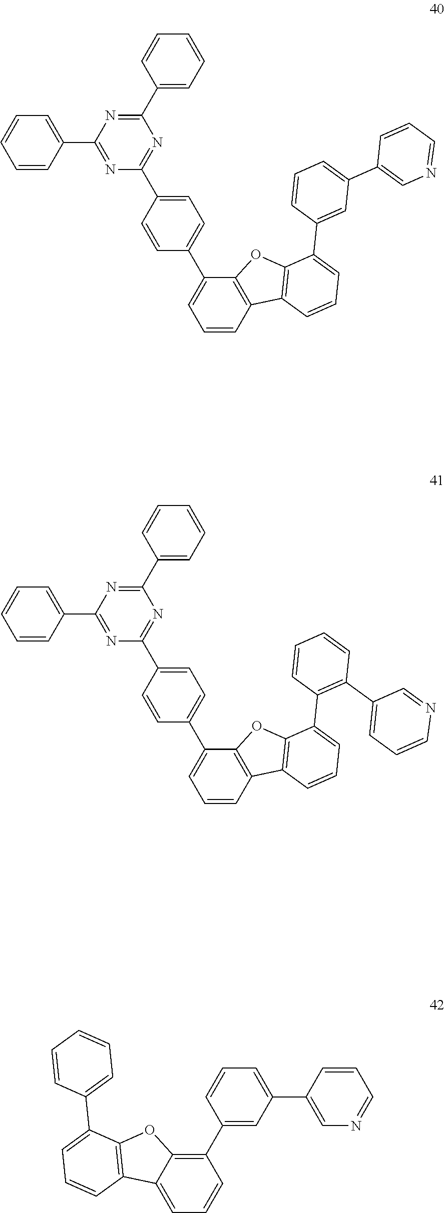 [SMILES: c1ccc(-c2nc(-c3ccccc3)nc(-c3ccc(-c4cccc5c4oc4c(-c6cccc(-c7cccnc7)c6)cccc45)cc3)n2)cc1 ; c1ccc(-c2nc(-c3ccccc3)nc(-c3ccc(-c4cccc5c4oc4c(-c6ccccc6-c6cccnc6)cccc45)cc3)n2)cc1 ; c1ccc(-c2cccc3c2oc2c(-c4cccc(-c5cccnc5)c4)cccc23)cc1]

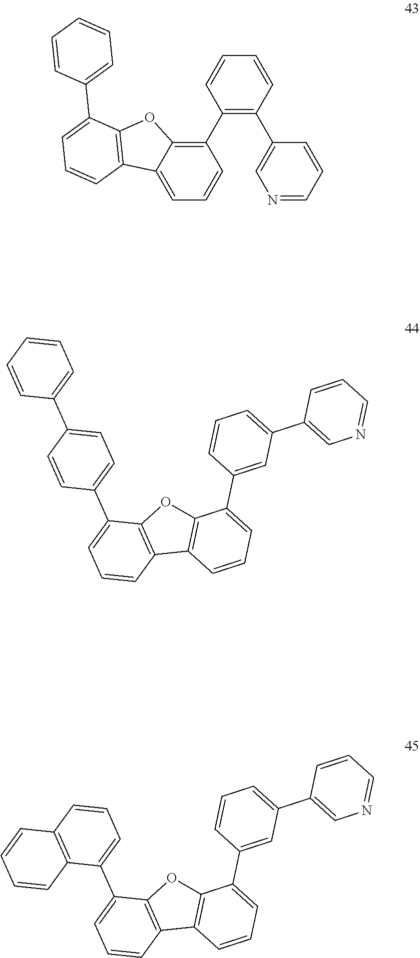 [SMILES: c1ccc(-c2cccc3c2oc2c(-c4ccccc4-c4cccnc4)cccc23)cc1 ; c1ccc(-c2ccc(-c3cccc4c3oc3c(-c5cccc(-c6cccnc6)c5)cccc34)cc2)cc1 ; c1cncc(-c2cccc(-c3cccc4c3oc3c(-c5cccc6ccccc56)cccc34)c2)c1]

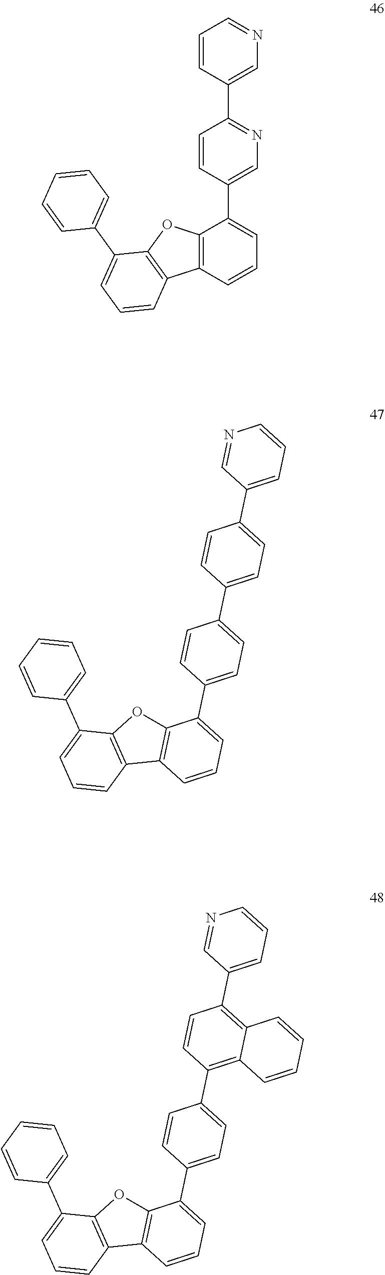 [SMILES: c1ccc(-c2cccc3c2oc2c(-c4ccc(-c5cccnc5)nc4)cccc23)cc1 ; c1ccc(-c2cccc3c2oc2c(-c4ccc(-c5ccc(-c6cccnc6)cc5)cc4)cccc23)cc1 ; c1ccc(-c2cccc3c2oc2c(-c4ccc(-c5ccc(-c6cccnc6)c6ccccc56)cc4)cccc23)cc1]

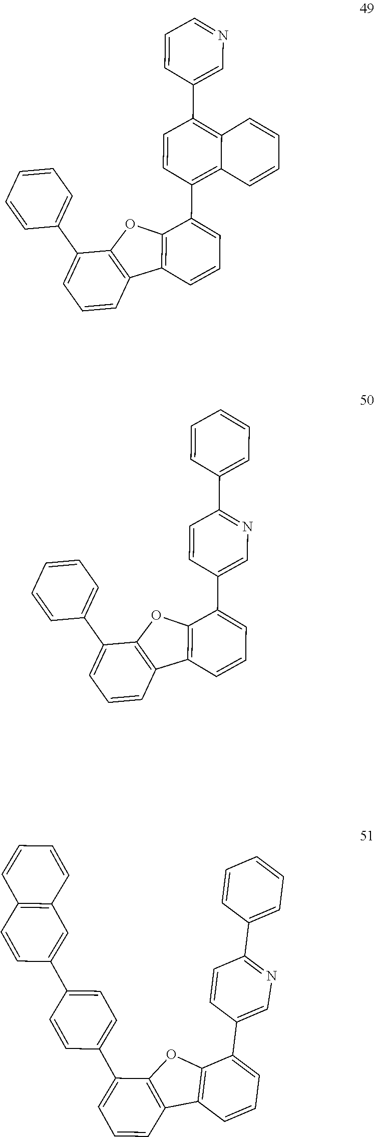 [SMILES: c1ccc(-c2cccc3c2oc2c(-c4ccc(-c5cccnc5)c5ccccc45)cccc23)cc1 ; c1ccc(-c2ccc(-c3cccc4c3oc3c(-c5ccccc5)cccc34)cn2)cc1 ; c1ccc(-c2ccc(-c3cccc4c3oc3c(-c5ccc(-c6ccc7ccccc7c6)cc5)cccc34)cn2)cc1]

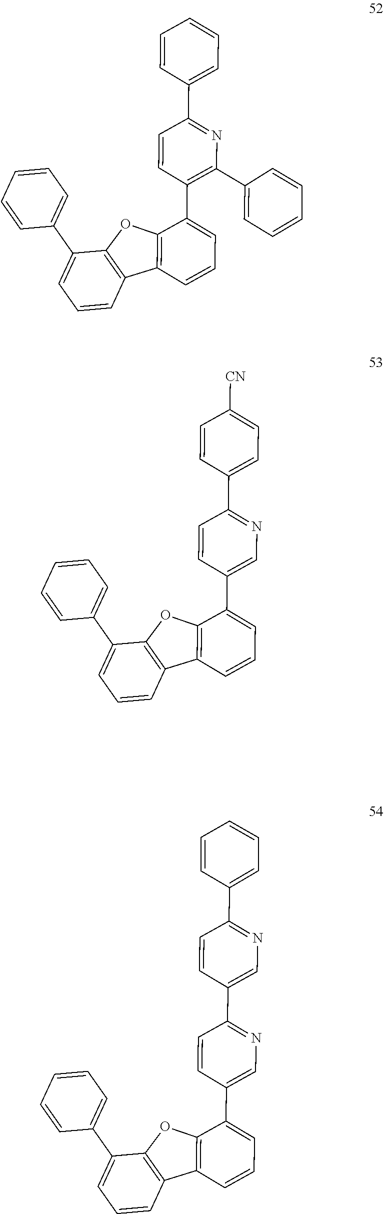 [SMILES: c1ccc(-c2ccc(-c3cccc4c3oc3c(-c5ccccc5)cccc34)c(-c3ccccc3)n2)cc1 ; N#Cc1ccc(-c2ccc(-c3cccc4c3oc3c(-c5ccccc5)cccc34)cn2)cc1 ; c1ccc(-c2ccc(-c3ccc(-c4cccc5c4oc4c(-c6ccccc6)cccc45)cn3)cn2)cc1]

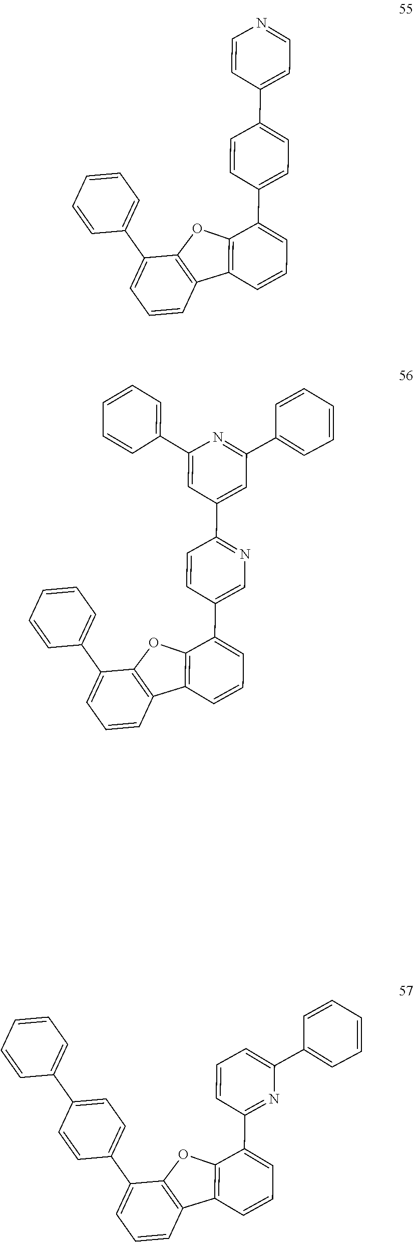 [SMILES: c1ccc(-c2cccc3c2oc2c(-c4ccc(-c5ccncc5)cc4)cccc23)cc1 ; c1ccc(-c2cc(-c3ccc(-c4cccc5c4oc4c(-c6ccccc6)cccc45)cn3)cc(-c3ccccc3)n2)cc1 ; c1ccc(-c2ccc(-c3cccc4c3oc3c(-c5cccc(-c6ccccc6)n5)cccc34)cc2)cc1]

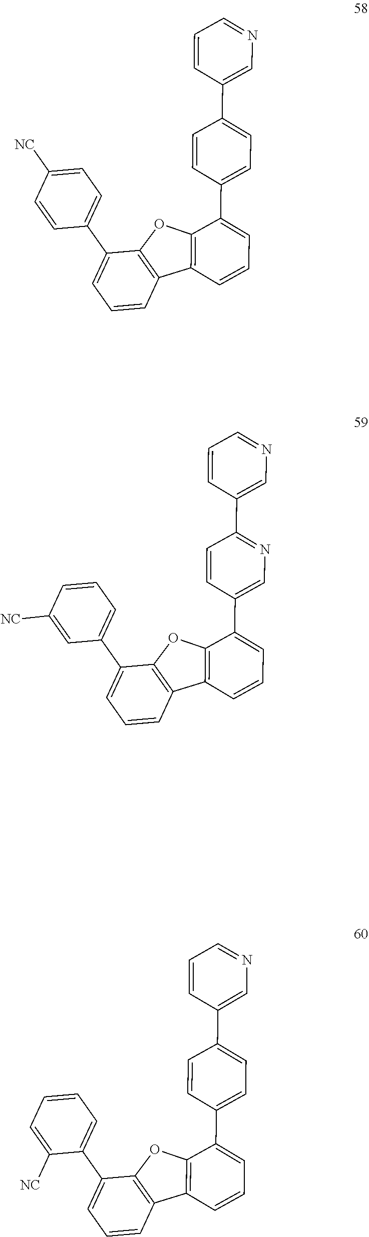 [SMILES: N#Cc1ccc(-c2cccc3c2oc2c(-c4ccc(-c5cccnc5)cc4)cccc23)cc1 ; N#Cc1cccc(-c2cccc3c2oc2c(-c4ccc(-c5cccnc5)nc4)cccc23)c1 ; N#Cc1ccccc1-c1cccc2c1oc1c(-c3ccc(-c4cccnc4)cc3)cccc12]

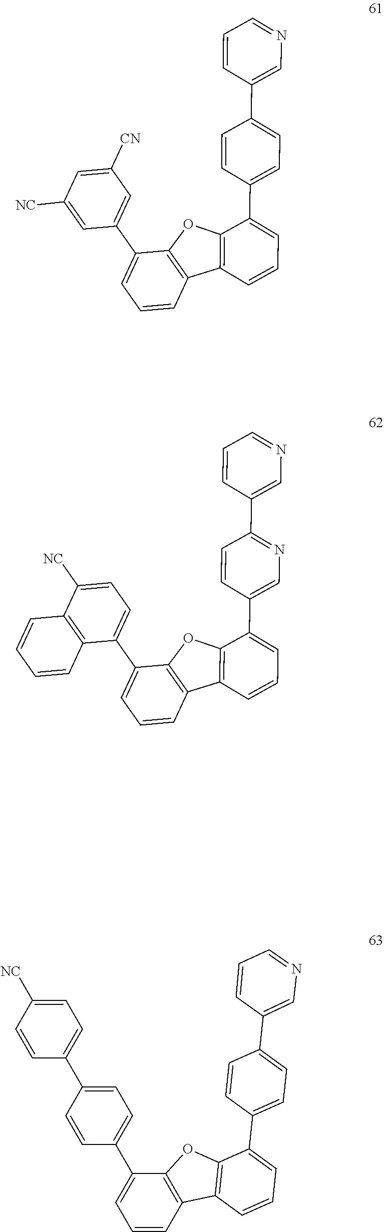 [SMILES: N#Cc1cc(C#N)cc(-c2cccc3c2oc2c(-c4ccc(-c5cccnc5)cc4)cccc23)c1 ; N#Cc1ccc(-c2cccc3c2oc2c(-c4ccc(-c5cccnc5)nc4)cccc23)c2ccccc12 ; N#Cc1ccc(-c2ccc(-c3cccc4c3oc3c(-c5ccc(-c6cccnc6)cc5)cccc34)cc2)cc1]

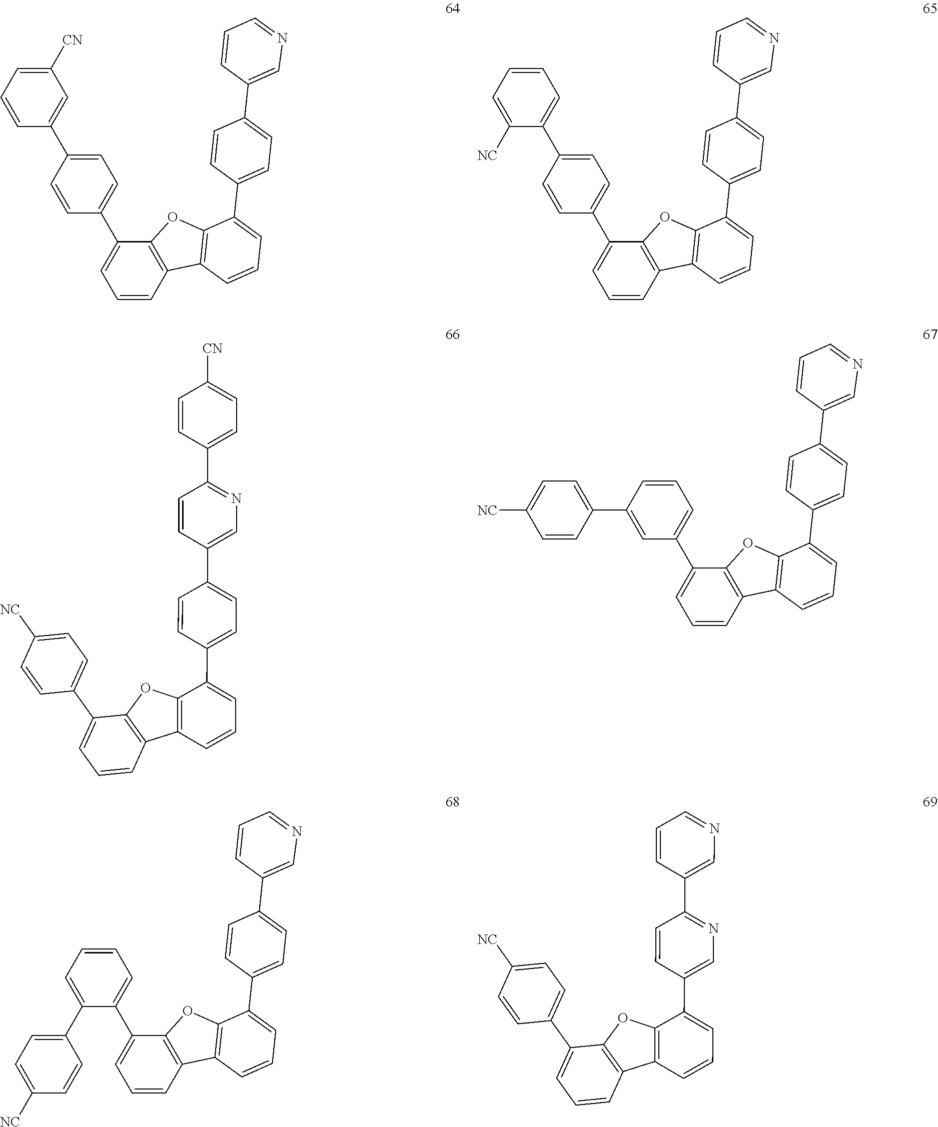 [SMILES: N#Cc1cccc(-c2ccc(-c3cccc4c3oc3c(-c5ccc(-c6cccnc6)cc5)cccc34)cc2)c1 ; N#Cc1ccccc1-c1ccc(-c2cccc3c2oc2c(-c4ccc(-c5cccnc5)cc4)cccc23)cc1 ; N#Cc1ccc(-c2ccc(-c3ccc(-c4cccc5c4oc4c(-c6ccc(C#N)cc6)cccc45)cc3)cn2)cc1 ; N#Cc1ccc(-c2cccc(-c3cccc4c3oc3c(-c5ccc(-c6cccnc6)cc5)cccc34)c2)cc1 ; N#Cc1ccc(-c2ccccc2-c2cccc3c2oc2c(-c4ccc(-c5cccnc5)cc4)cccc23)cc1 ; N#Cc1ccc(-c2cccc3c2oc2c(-c4ccc(-c5cccnc5)nc4)cccc23)cc1]

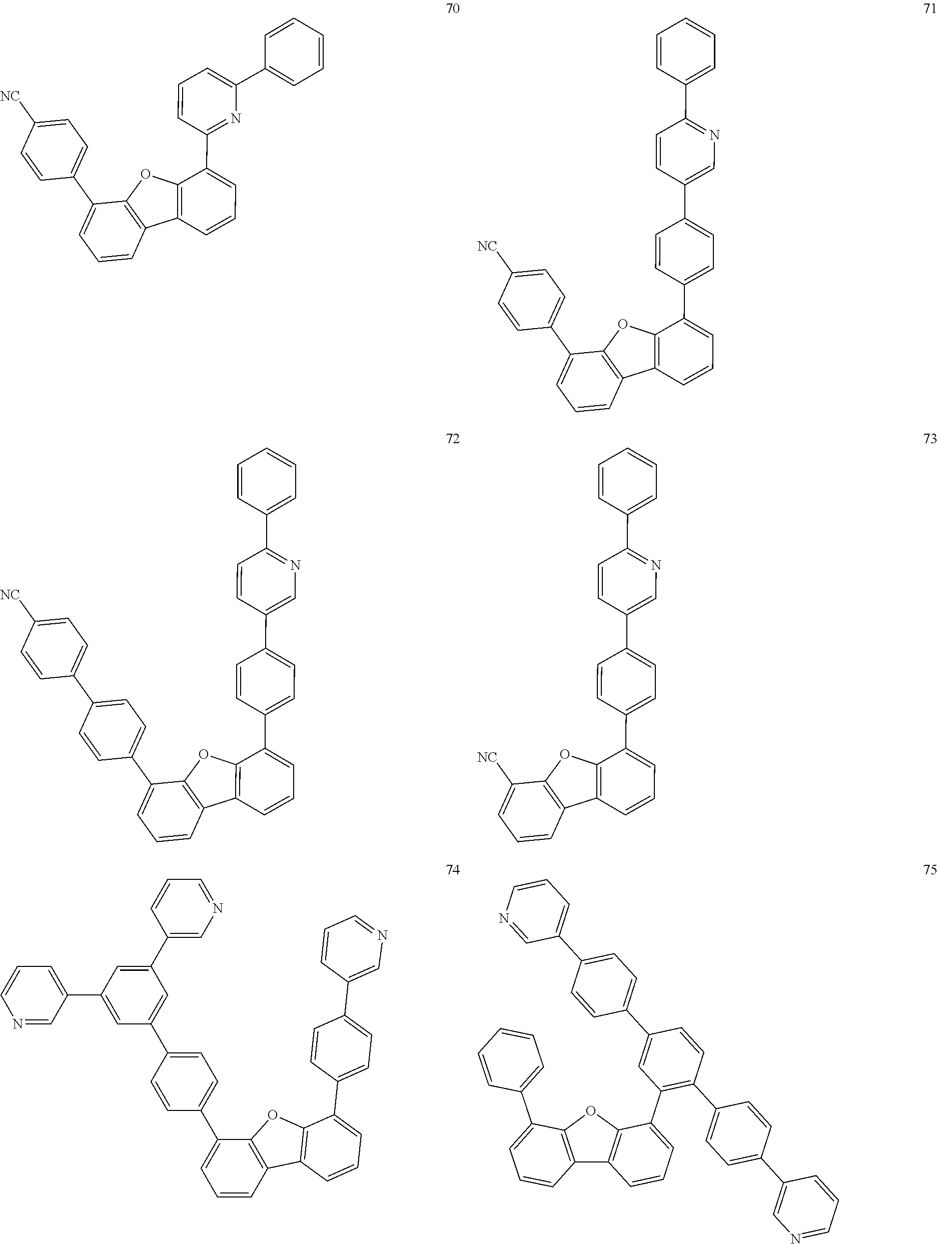

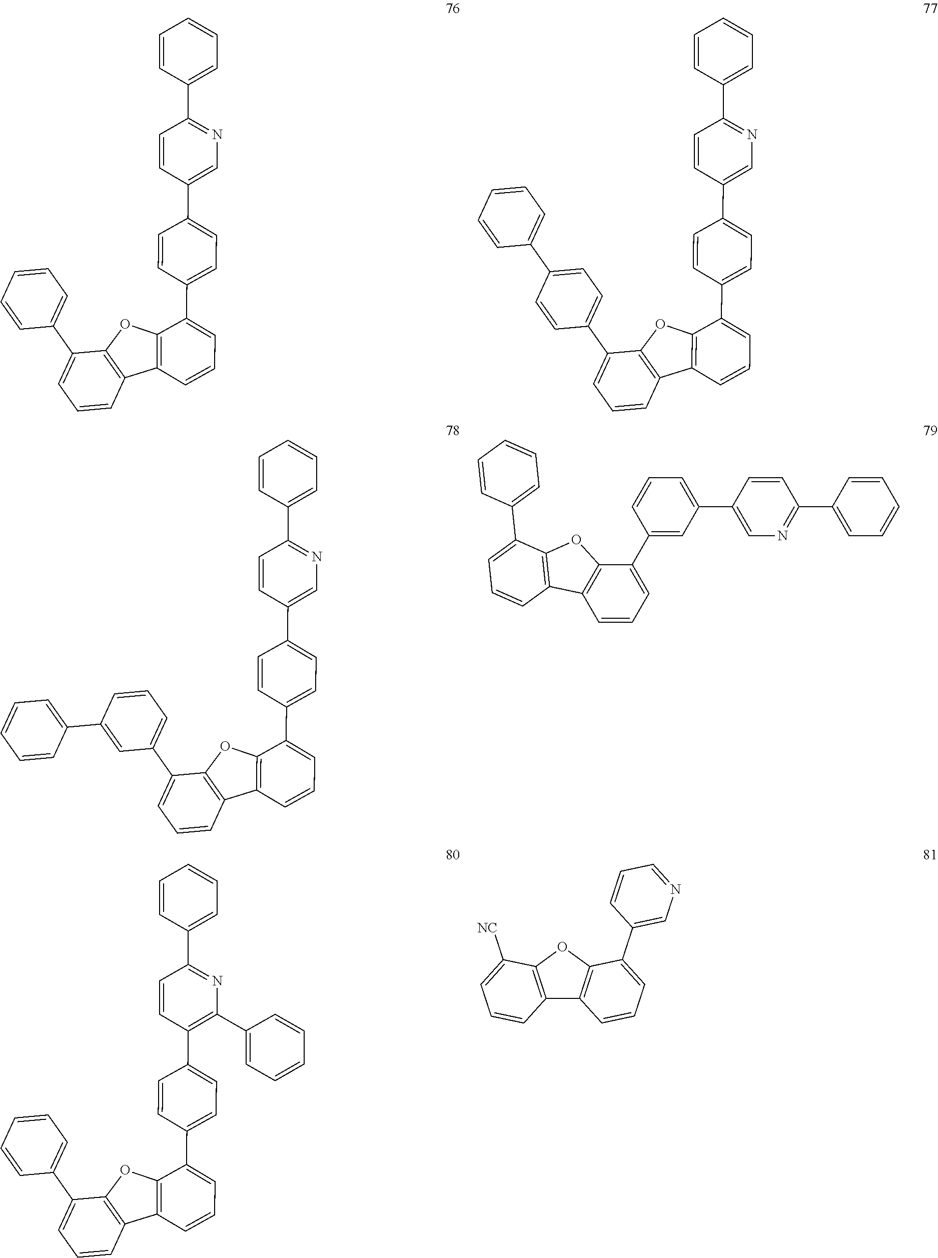

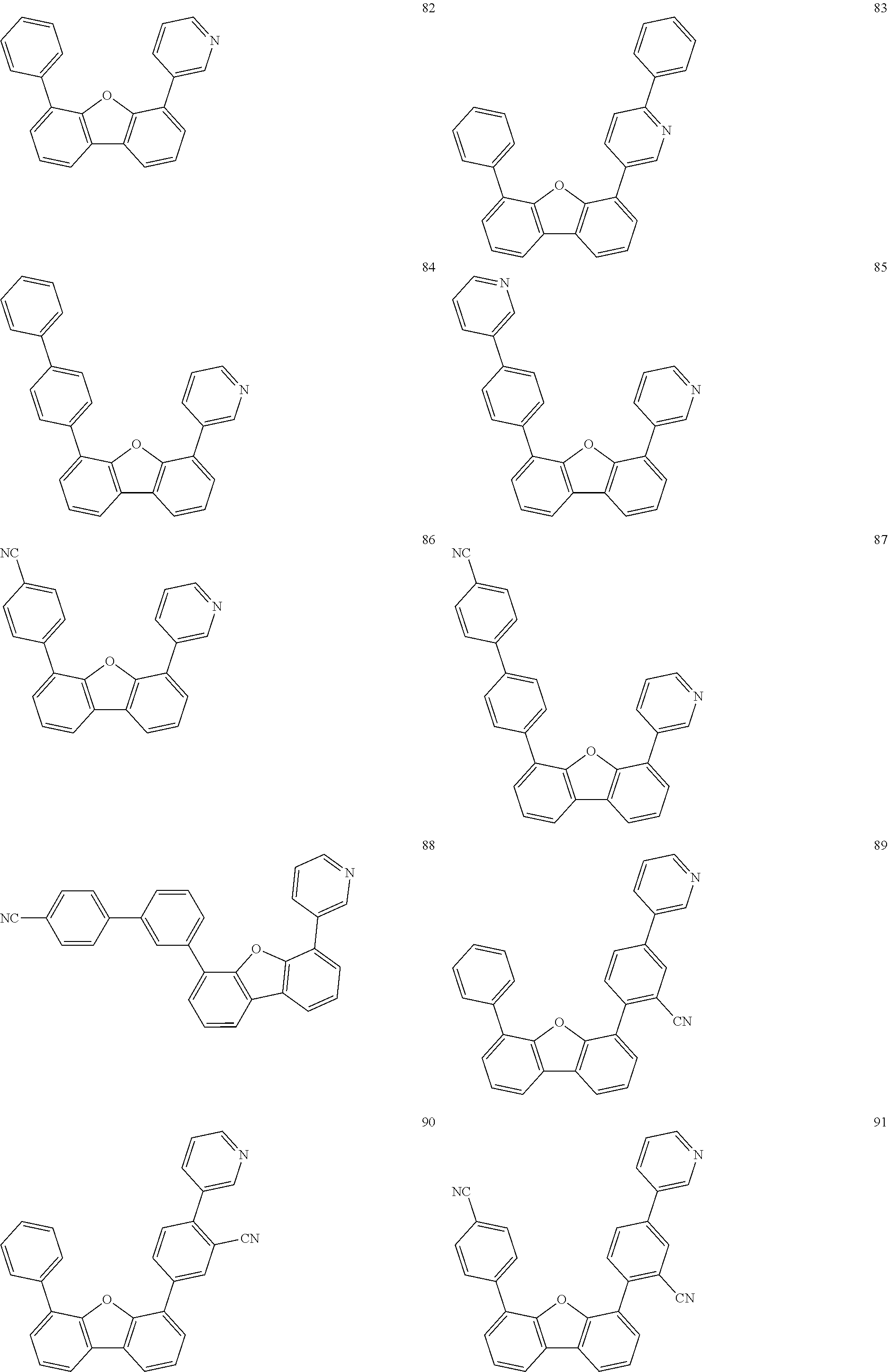

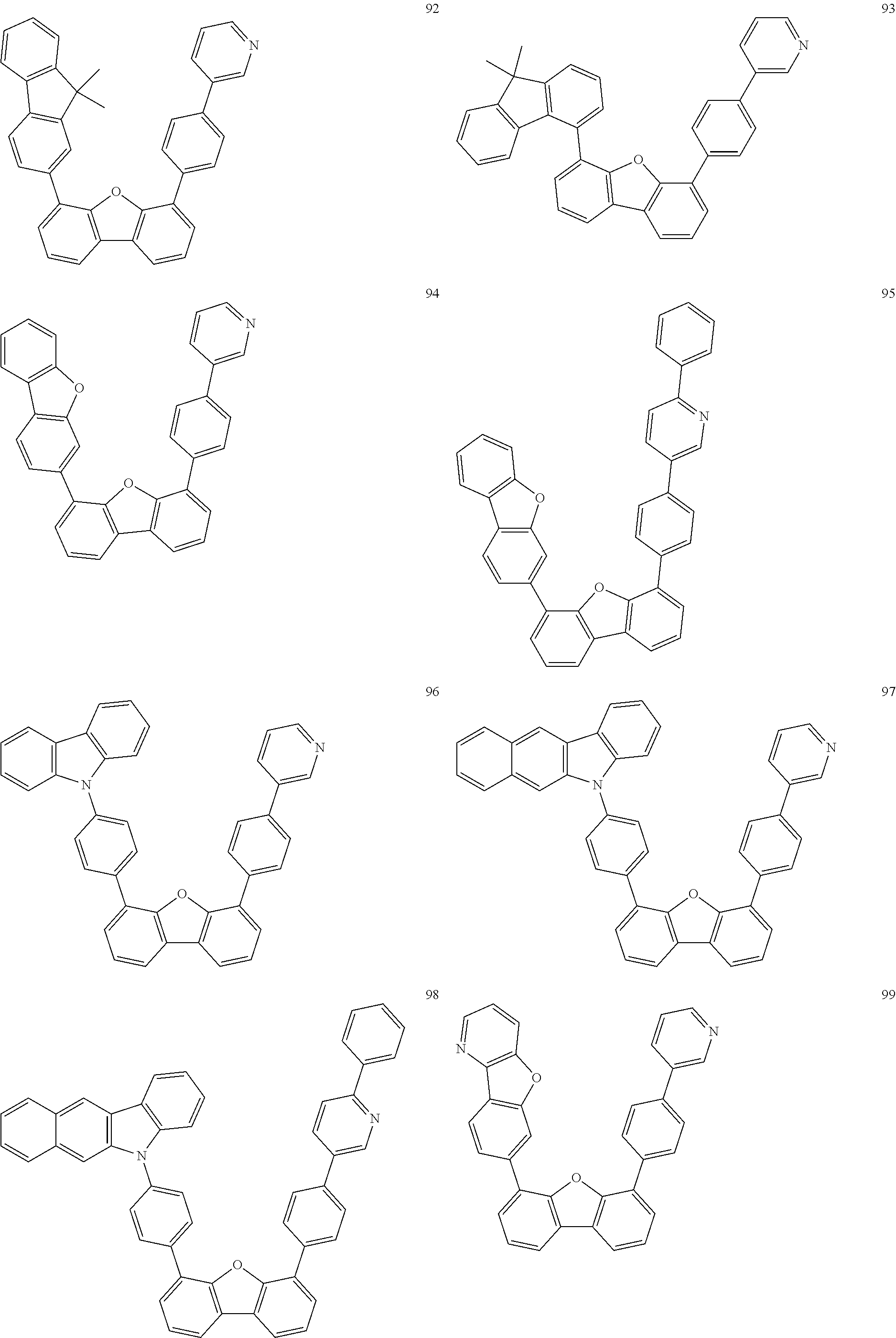

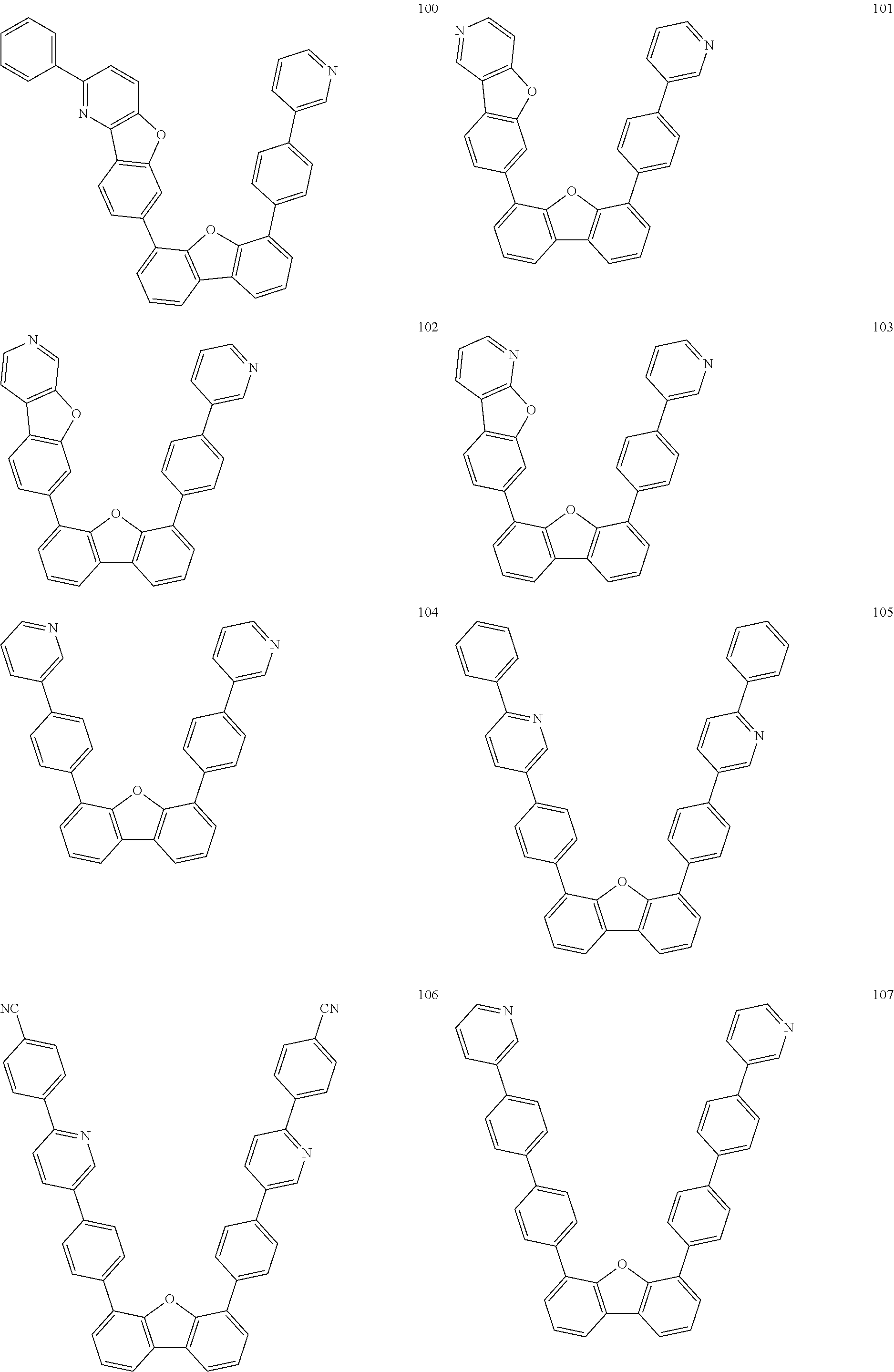

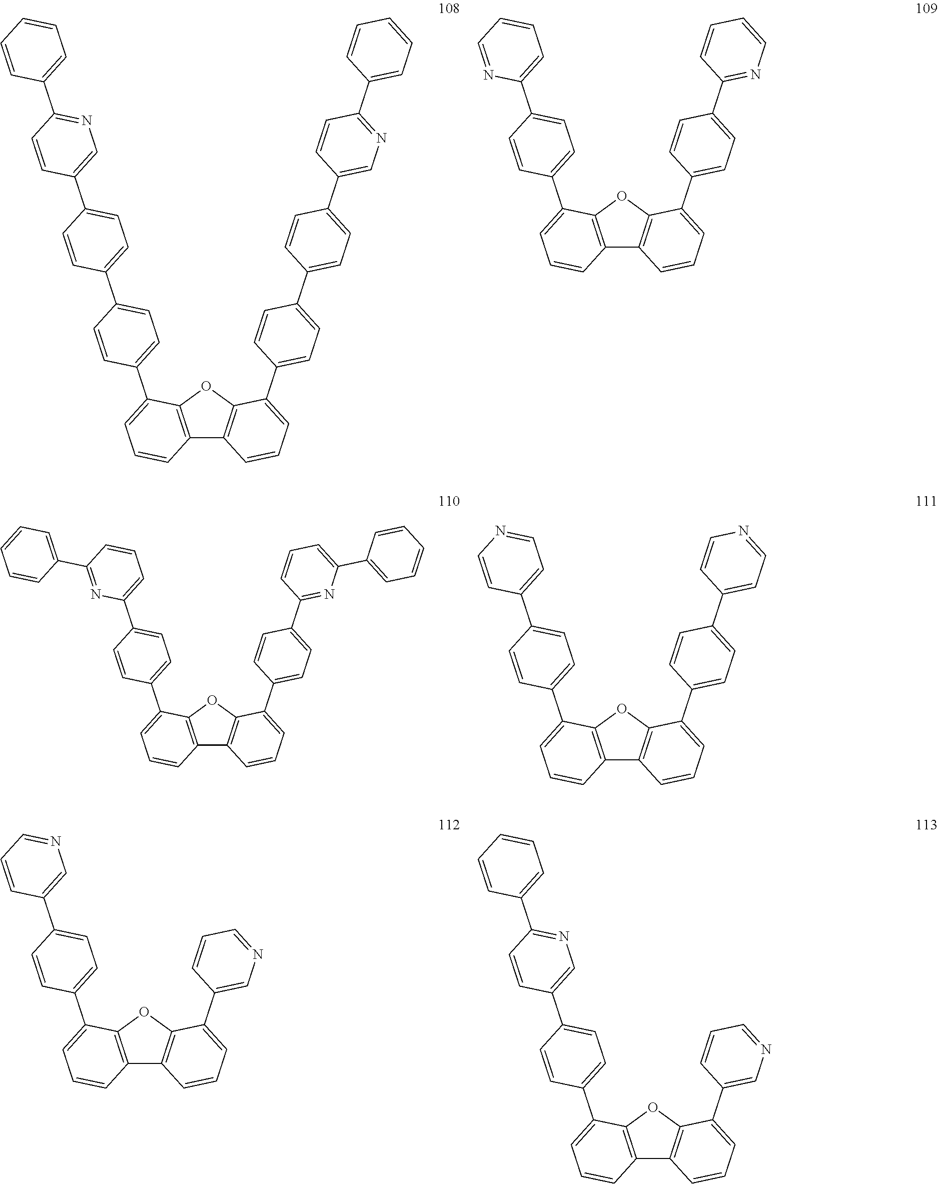 [SMILES: c1ccc(-c2ccc(-c3ccc(-c4ccc(-c5cccc6c5oc5c(-c7ccc(-c8ccc(-c9ccc(-c%10ccccc%10)nc9)cc8)cc7)cccc56)cc4)cc3)cn2)cc1 ; c1ccc(-c2ccc(-c3cccc4c3oc3c(-c5ccc(-c6ccccn6)cc5)cccc34)cc2)nc1 ; c1ccc(-c2cccc(-c3ccc(-c4cccc5c4oc4c(-c6ccc(-c7cccc(-c8ccccc8)n7)cc6)cccc45)cc3)n2)cc1 ; c1cc(-c2ccc(-c3ccncc3)cc2)c2oc3c(-c4ccc(-c5ccncc5)cc4)cccc3c2c1 ; c1cncc(-c2ccc(-c3cccc4c3oc3c(-c5cccnc5)cccc34)cc2)c1 ; c1ccc(-c2ccc(-c3ccc(-c4cccc5c4oc4c(-c6cccnc6)cccc45)cc3)cn2)cc1]

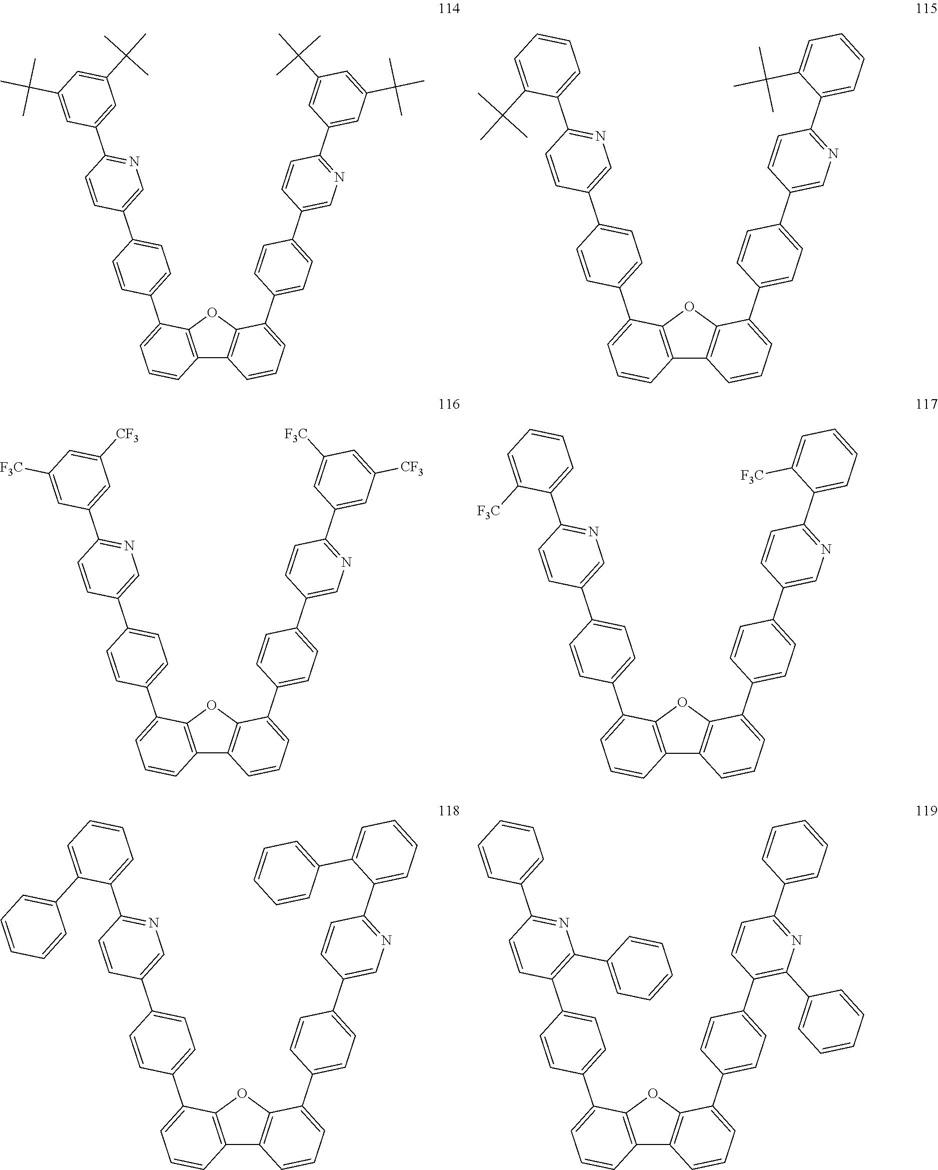

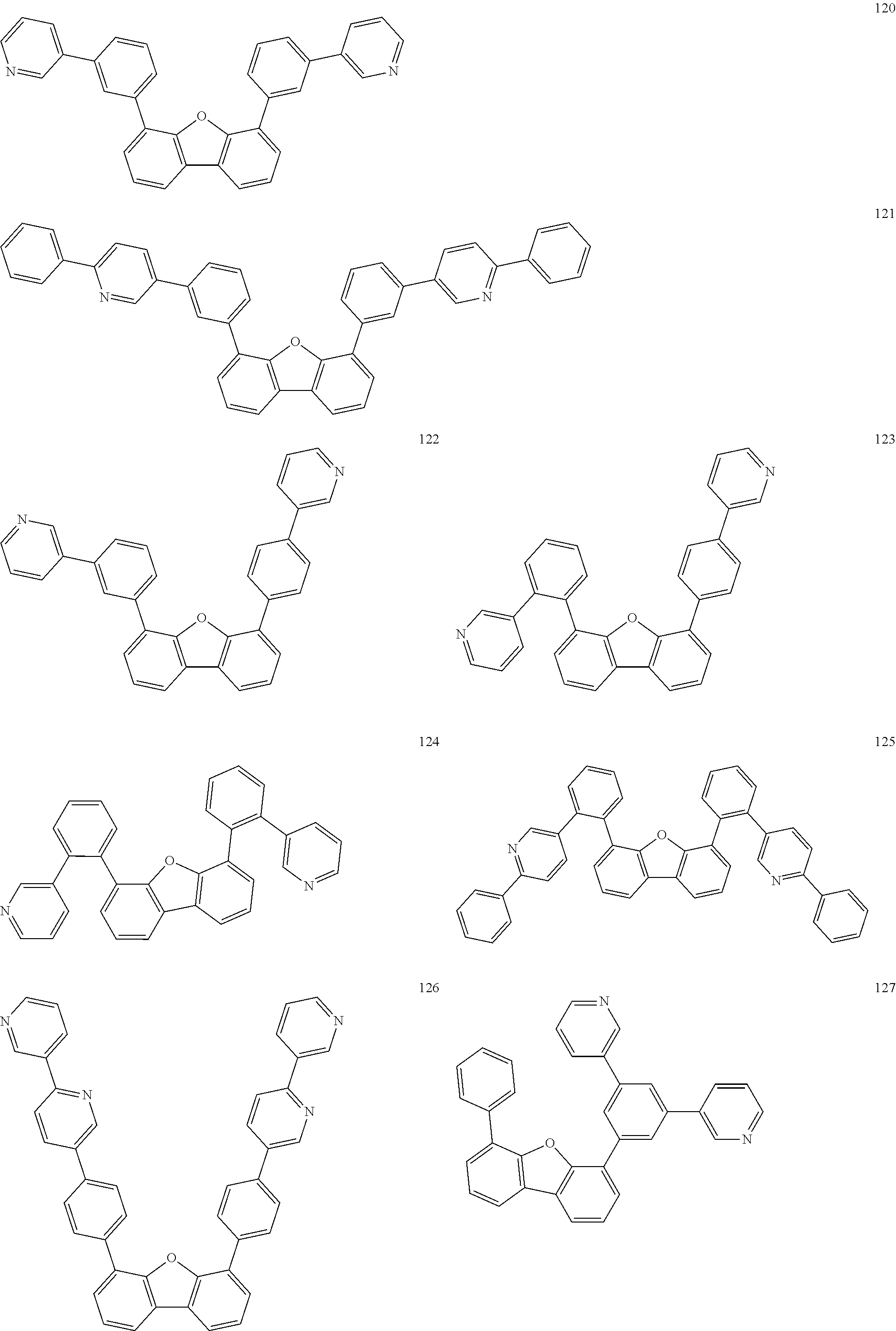

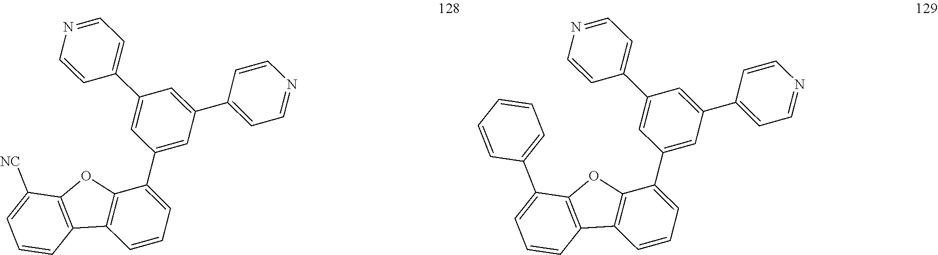 [SMILES: N#Cc1cccc2c1oc1c(-c3cc(-c4ccncc4)cc(-c4ccncc4)c3)cccc12 ; c1ccc(-c2cccc3c2oc2c(-c4cc(-c5ccncc5)cc(-c5ccncc5)c4)cccc23)cc1]

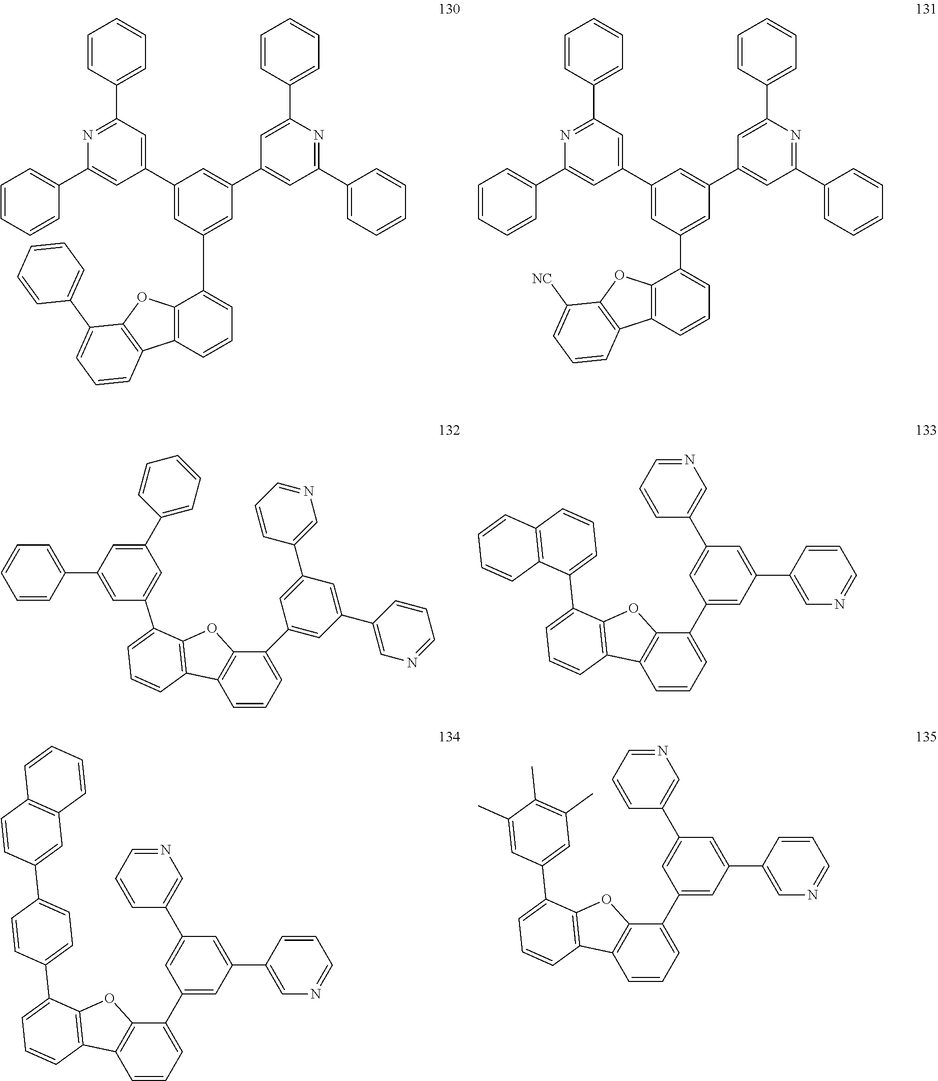

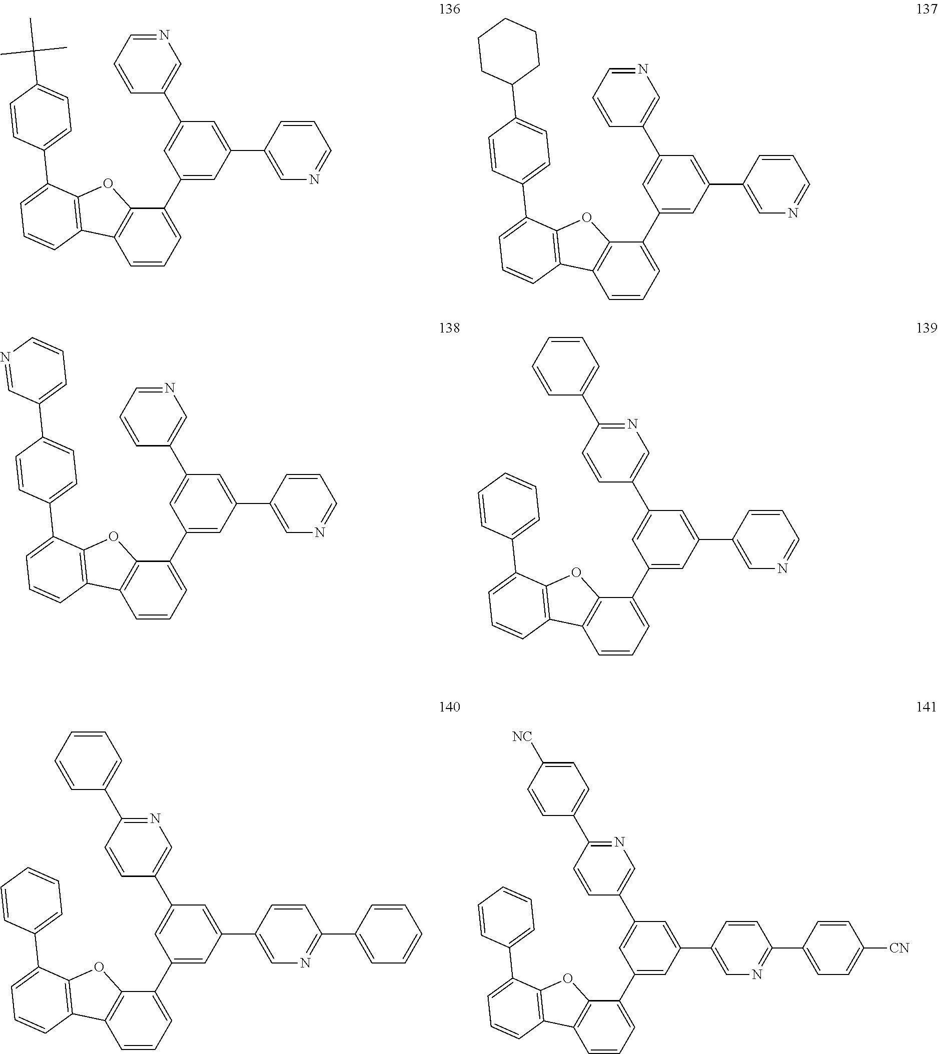

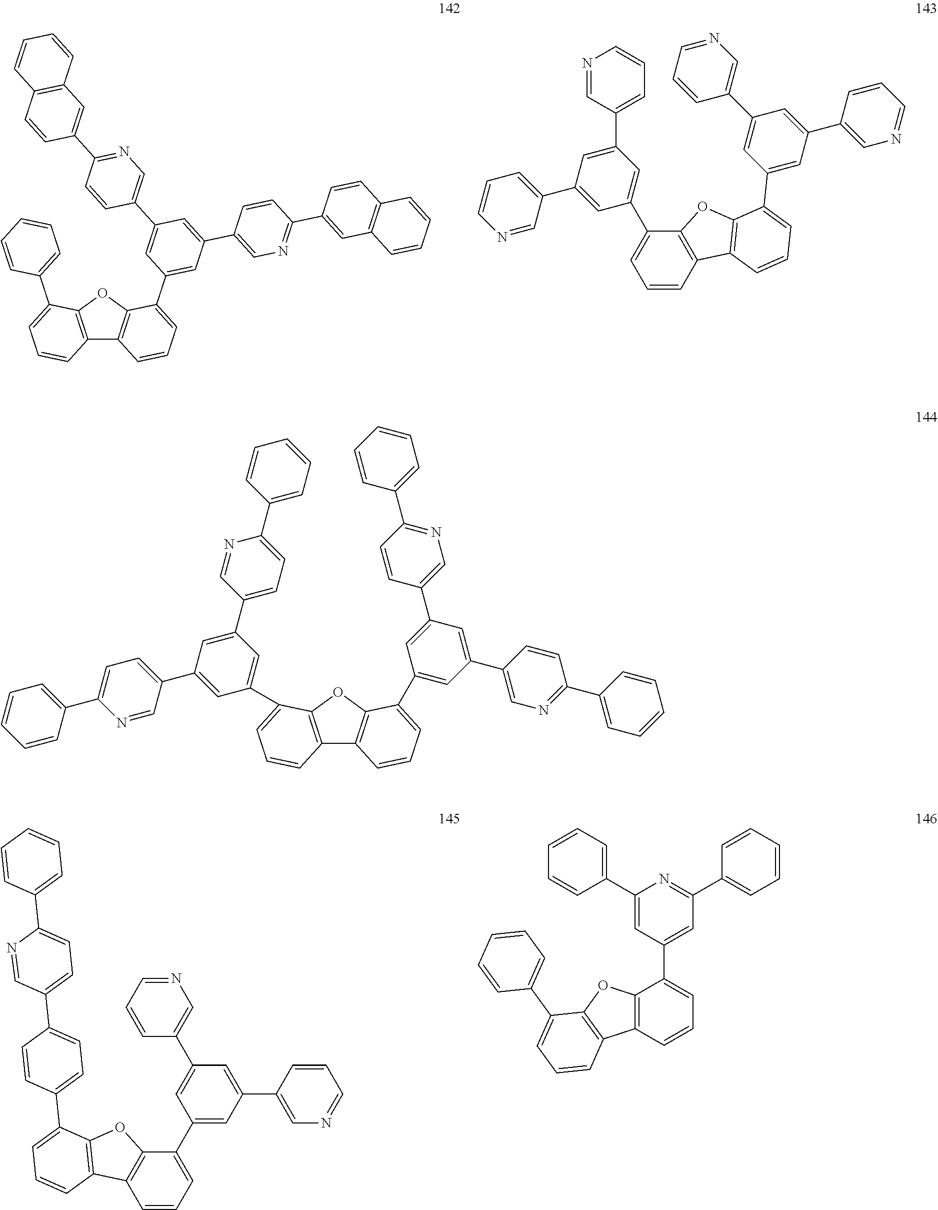 [SMILES: c1ccc(-c2cccc3c2oc2c(-c4cc(-c5ccc(-c6ccc7ccccc7c6)nc5)cc(-c5ccc(-c6ccc7ccccc7c6)nc5)c4)cccc23)cc1 ; c1cncc(-c2cc(-c3cccnc3)cc(-c3cccc4c3oc3c(-c5cc(-c6cccnc6)cc(-c6cccnc6)c5)cccc34)c2)c1 ; c1ccc(-c2ccc(-c3cc(-c4ccc(-c5ccccc5)nc4)cc(-c4cccc5c4oc4c(-c6cc(-c7ccc(-c8ccccc8)nc7)cc(-c7ccc(-c8ccccc8)nc7)c6)cccc45)c3)cn2)cc1 ; c1ccc(-c2ccc(-c3ccc(-c4cccc5c4oc4c(-c6cc(-c7cccnc7)cc(-c7cccnc7)c6)cccc45)cc3)cn2)cc1 ; c1ccc(-c2cc(-c3cccc4c3oc3c(-c5ccccc5)cccc34)cc(-c3ccccc3)n2)cc1]

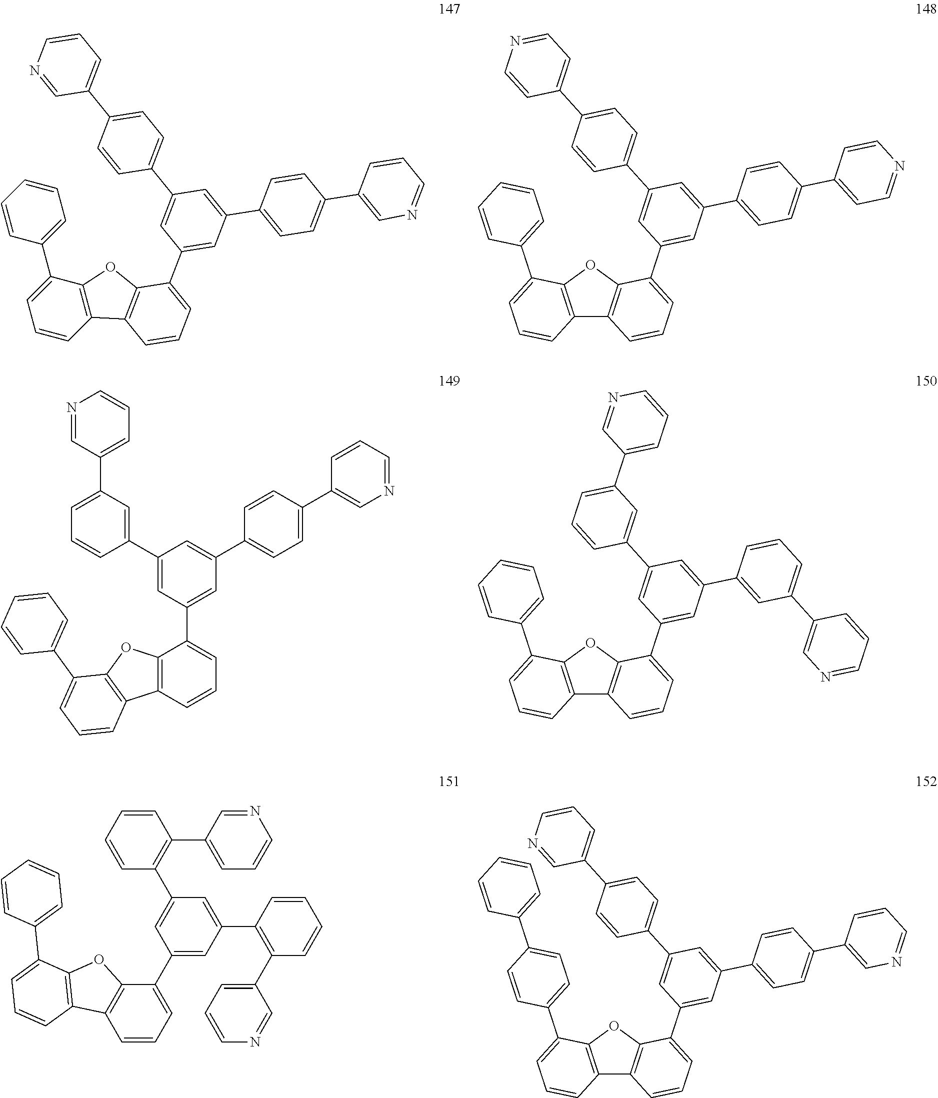

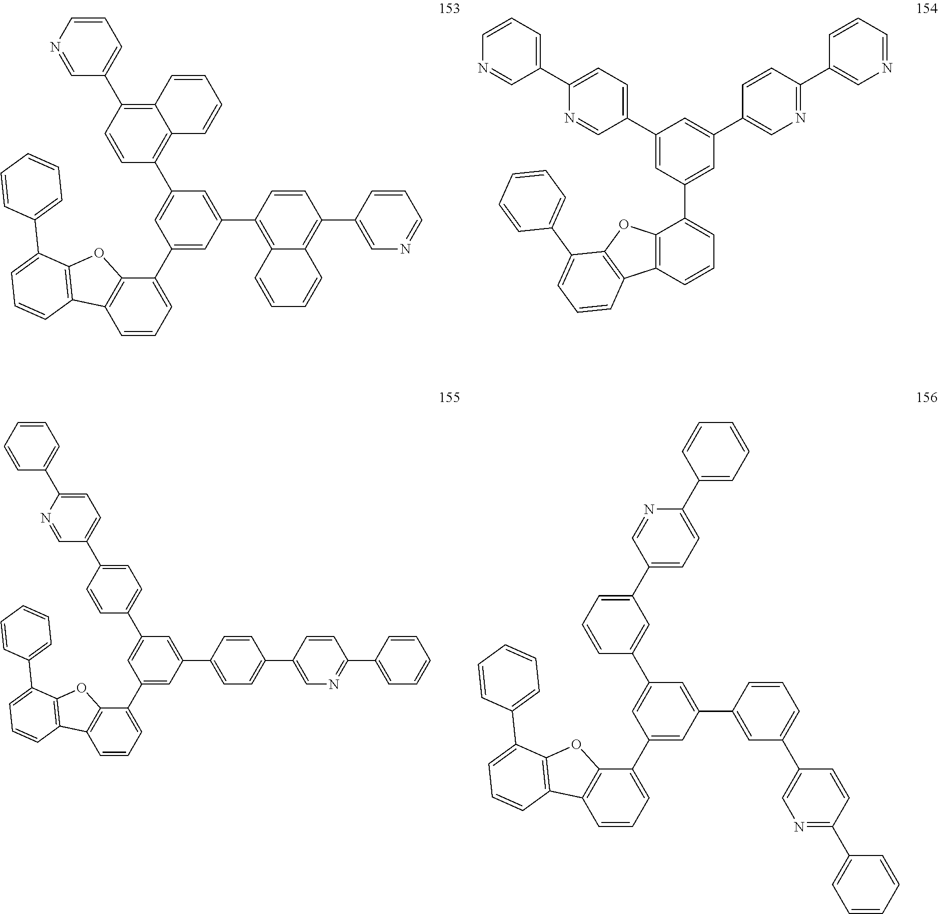 [SMILES: c1ccc(-c2cccc3c2oc2c(-c4cc(-c5ccc(-c6cccnc6)c6ccccc56)cc(-c5ccc(-c6cccnc6)c6ccccc56)c4)cccc23)cc1 ; c1ccc(-c2cccc3c2oc2c(-c4cc(-c5ccc(-c6cccnc6)nc5)cc(-c5ccc(-c6cccnc6)nc5)c4)cccc23)cc1 ; c1ccc(-c2ccc(-c3ccc(-c4cc(-c5ccc(-c6ccc(-c7ccccc7)nc6)cc5)cc(-c5cccc6c5oc5c(-c7ccccc7)cccc56)c4)cc3)cn2)cc1 ; c1ccc(-c2ccc(-c3cccc(-c4cc(-c5cccc(-c6ccc(-c7ccccc7)nc6)c5)cc(-c5cccc6c5oc5c(-c7ccccc7)cccc56)c4)c3)cn2)cc1]

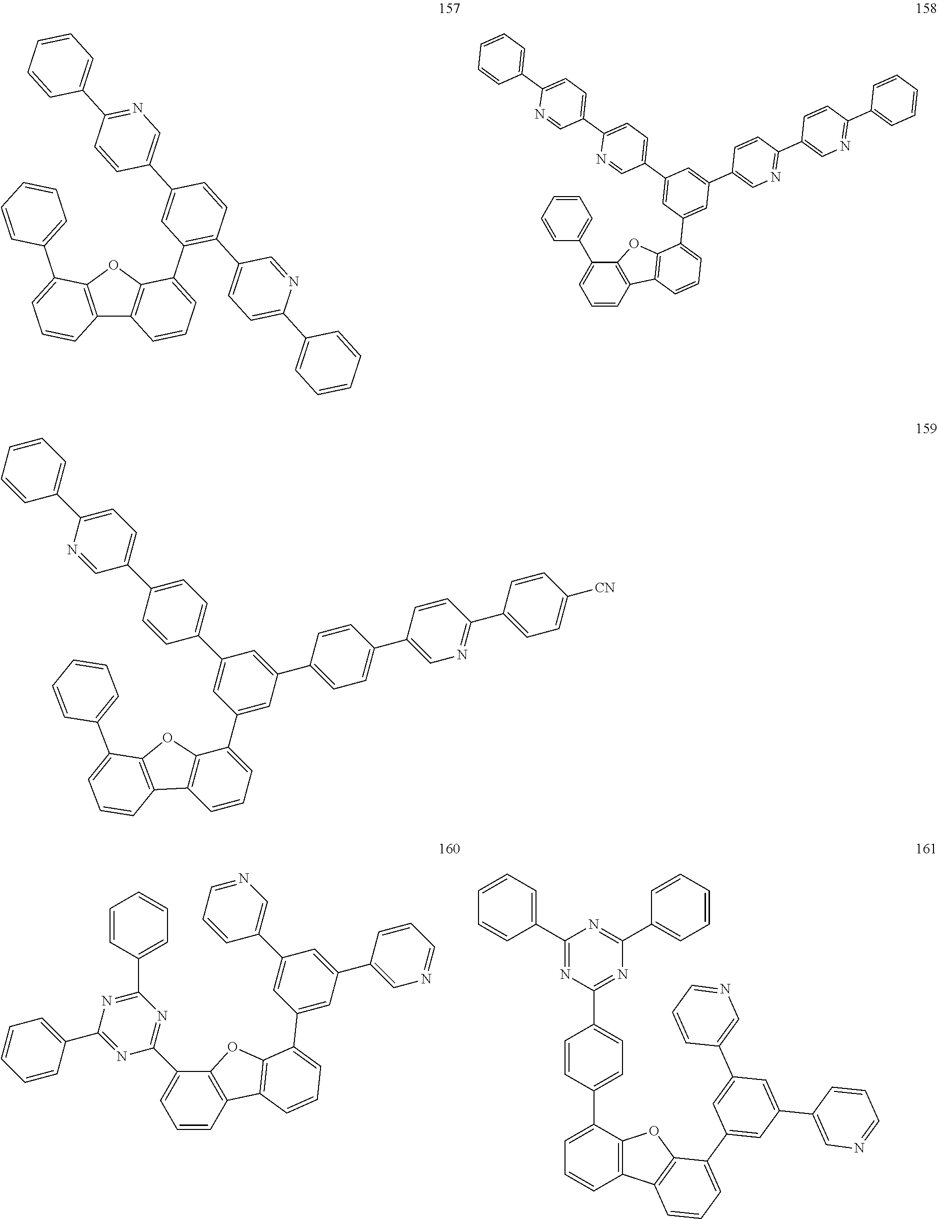 [SMILES: c1ccc(-c2ccc(-c3ccc(-c4ccc(-c5ccccc5)nc4)c(-c4cccc5c4oc4c(-c6ccccc6)cccc45)c3)cn2)cc1 ; c1ccc(-c2ccc(-c3ccc(-c4cc(-c5ccc(-c6ccc(-c7ccccc7)nc6)nc5)cc(-c5cccc6c5oc5c(-c7ccccc7)cccc56)c4)cn3)cn2)cc1 ; N#Cc1ccc(-c2ccc(-c3ccc(-c4cc(-c5ccc(-c6ccc(-c7ccccc7)nc6)cc5)cc(-c5cccc6c5oc5c(-c7ccccc7)cccc56)c4)cc3)cn2)cc1 ; c1ccc(-c2nc(-c3ccccc3)nc(-c3cccc4c3oc3c(-c5cc(-c6cccnc6)cc(-c6cccnc6)c5)cccc34)n2)cc1 ; c1ccc(-c2nc(-c3ccccc3)nc(-c3ccc(-c4cccc5c4oc4c(-c6cc(-c7cccnc7)cc(-c7cccnc7)c6)cccc45)cc3)n2)cc1]

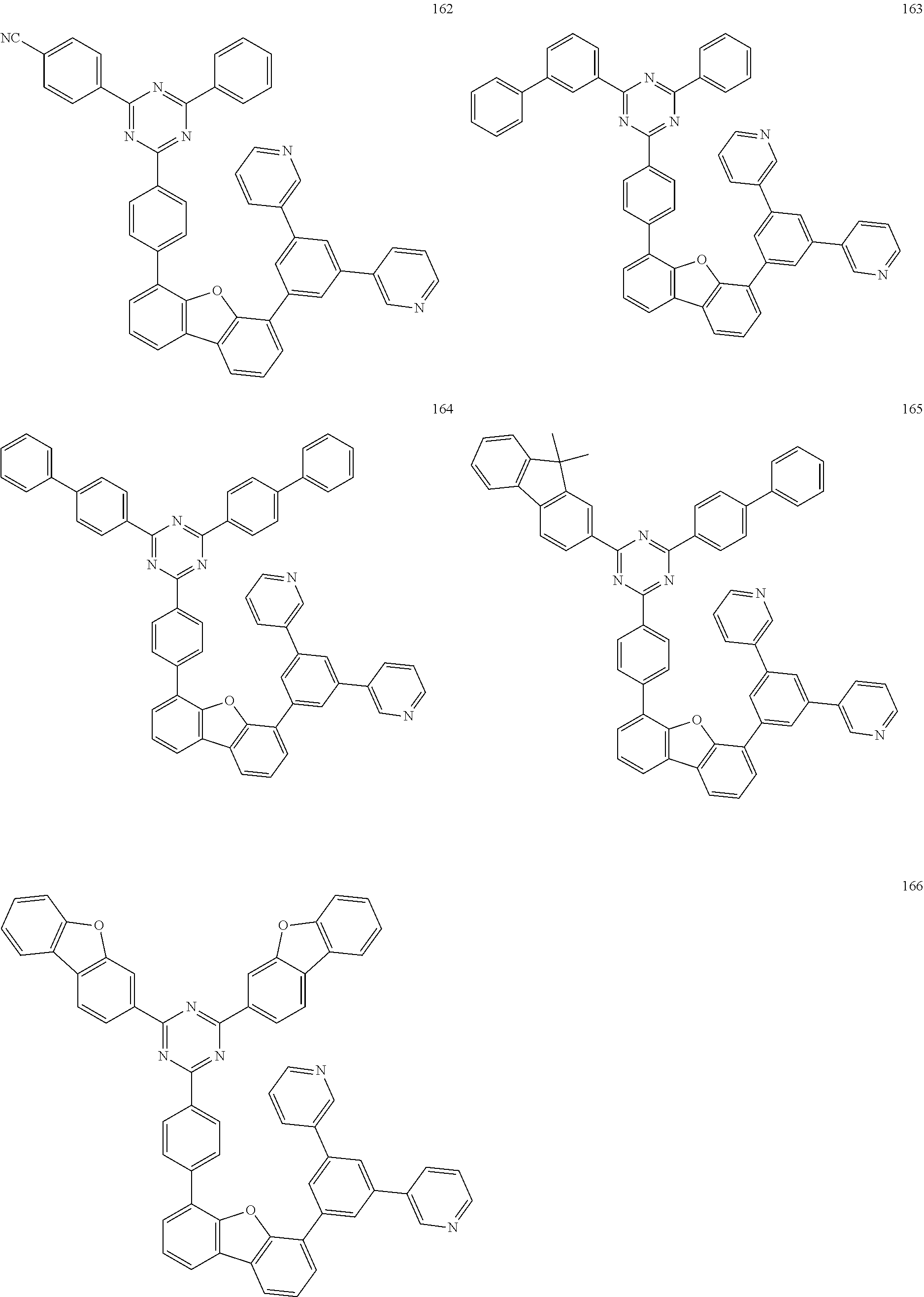

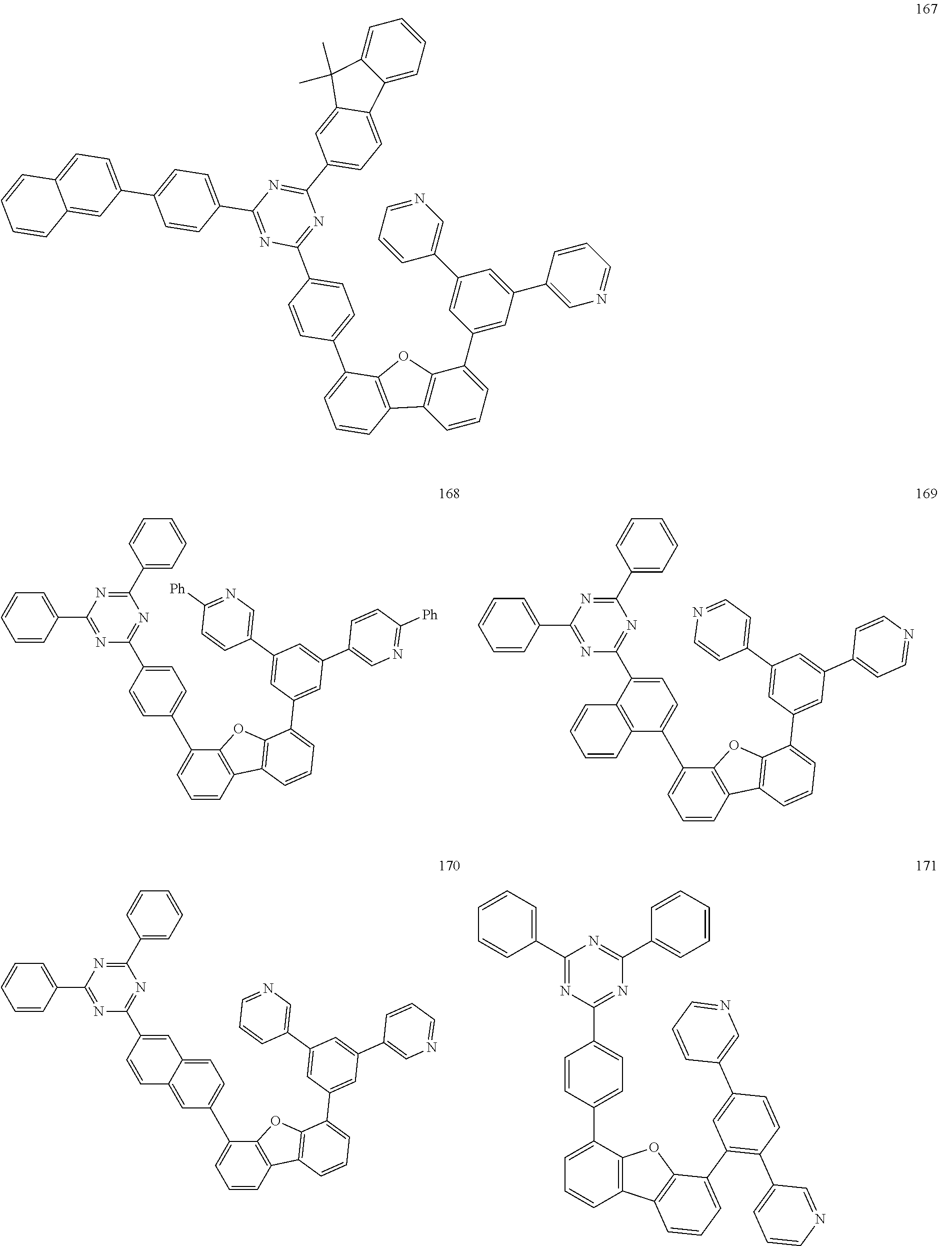

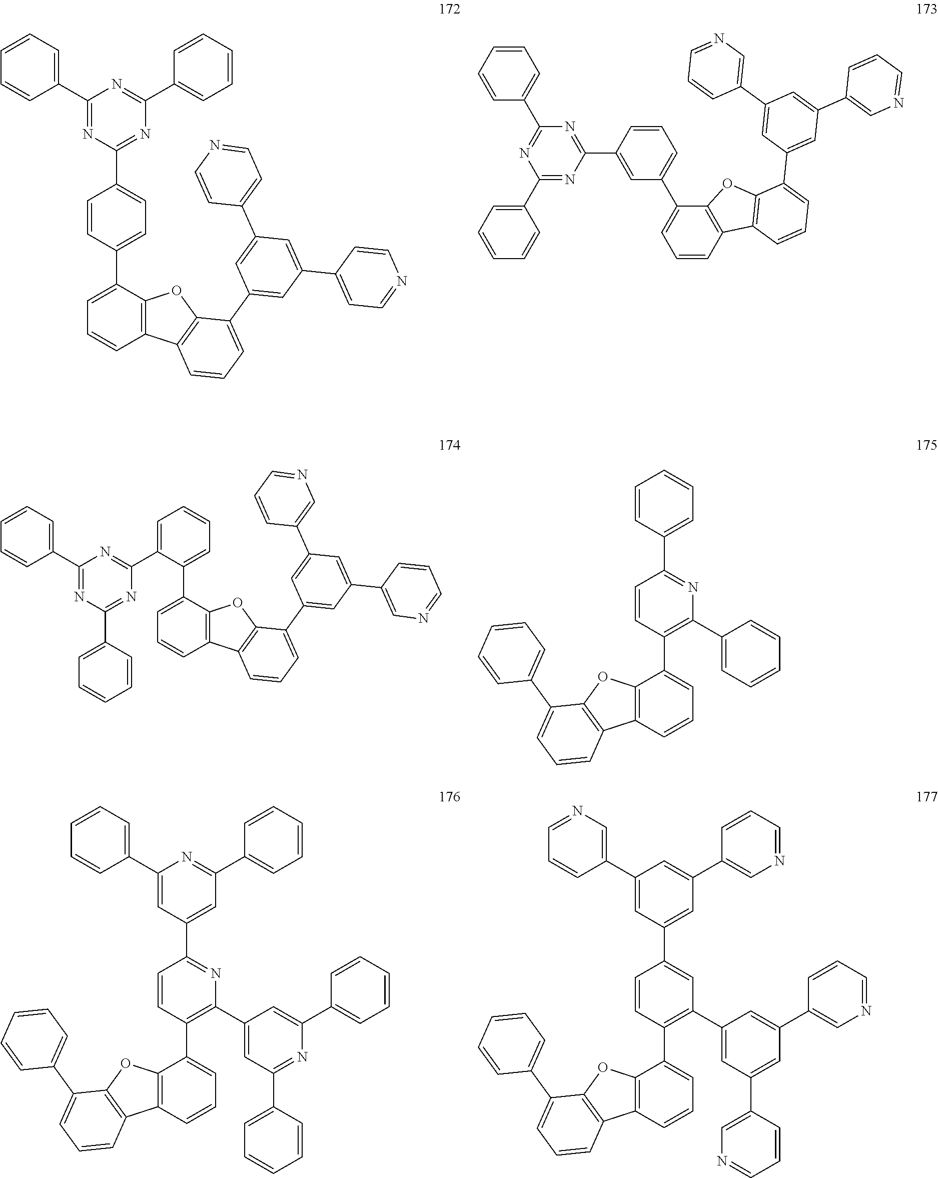

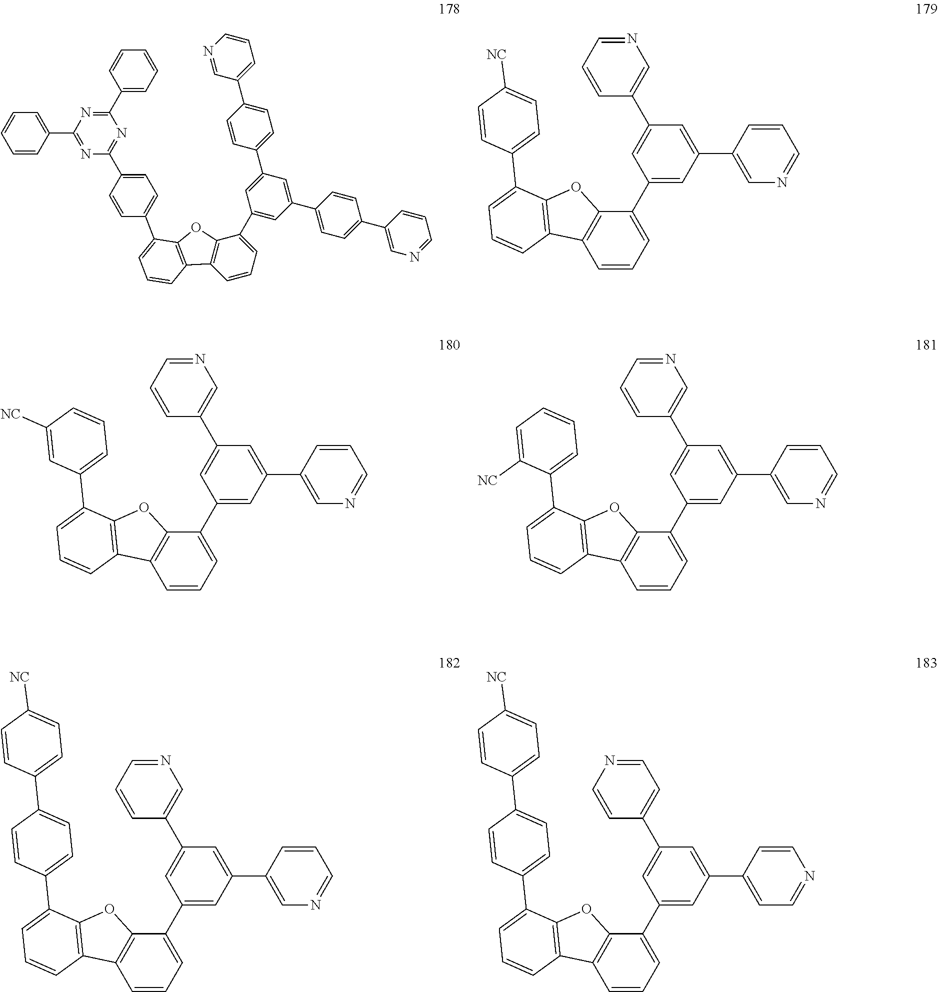 [SMILES: c1ccc(-c2nc(-c3ccccc3)nc(-c3ccc(-c4cccc5c4oc4c(-c6cc(-c7ccc(-c8cccnc8)cc7)cc(-c7ccc(-c8cccnc8)cc7)c6)cccc45)cc3)n2)cc1 ; N#Cc1ccc(-c2cccc3c2oc2c(-c4cc(-c5cccnc5)cc(-c5cccnc5)c4)cccc23)cc1 ; N#Cc1cccc(-c2cccc3c2oc2c(-c4cc(-c5cccnc5)cc(-c5cccnc5)c4)cccc23)c1 ; N#Cc1ccccc1-c1cccc2c1oc1c(-c3cc(-c4cccnc4)cc(-c4cccnc4)c3)cccc12 ; N#Cc1ccc(-c2ccc(-c3cccc4c3oc3c(-c5cc(-c6cccnc6)cc(-c6cccnc6)c5)cccc34)cc2)cc1 ; N#Cc1ccc(-c2ccc(-c3cccc4c3oc3c(-c5cc(-c6ccncc6)cc(-c6ccncc6)c5)cccc34)cc2)cc1]

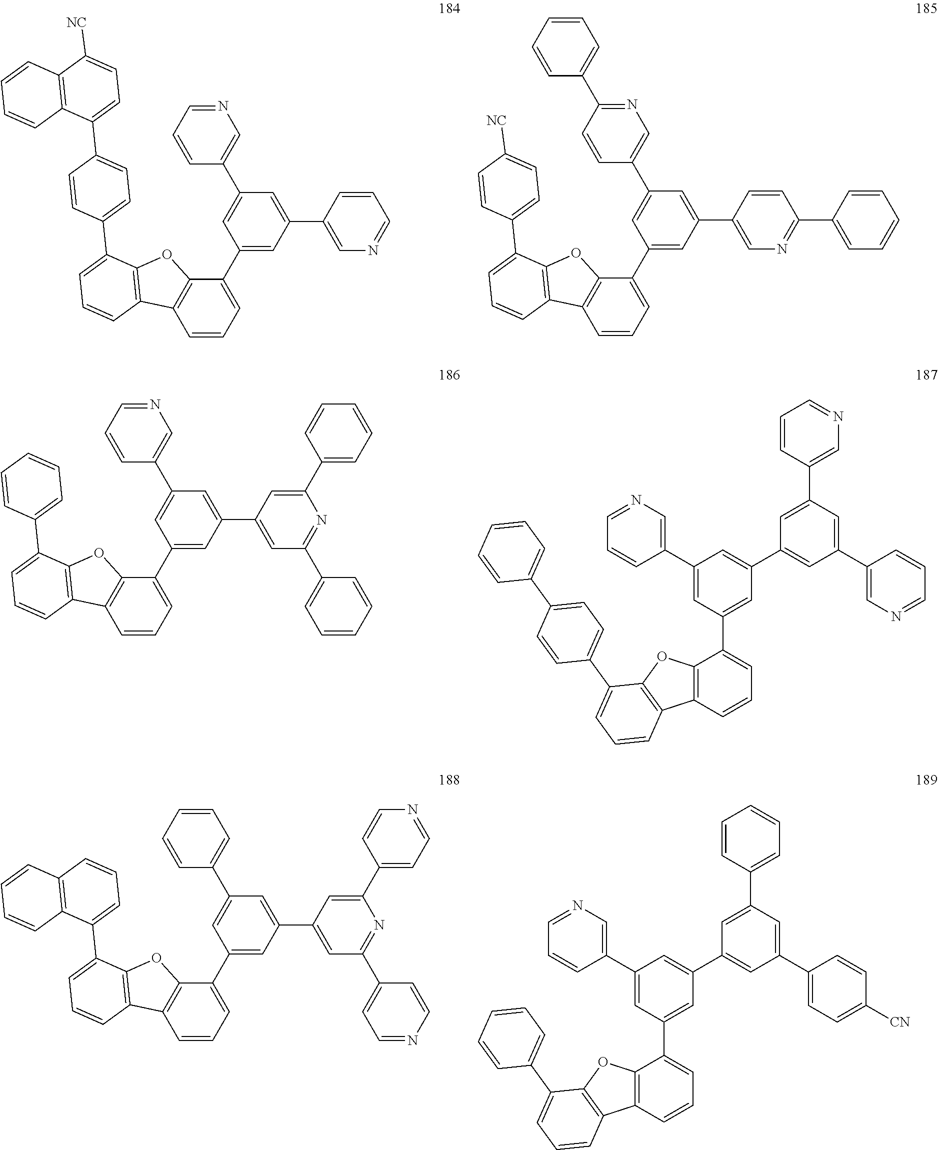

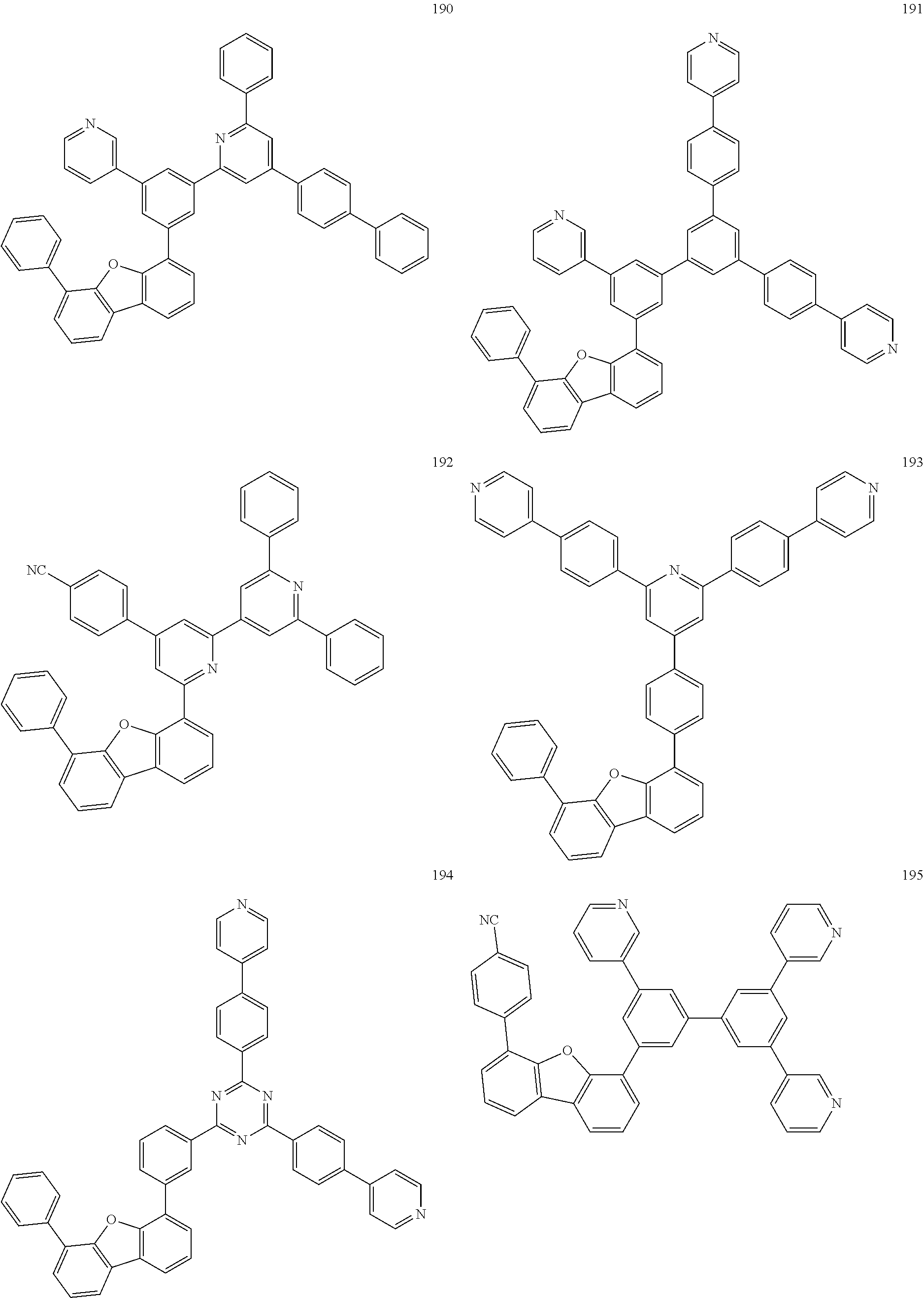

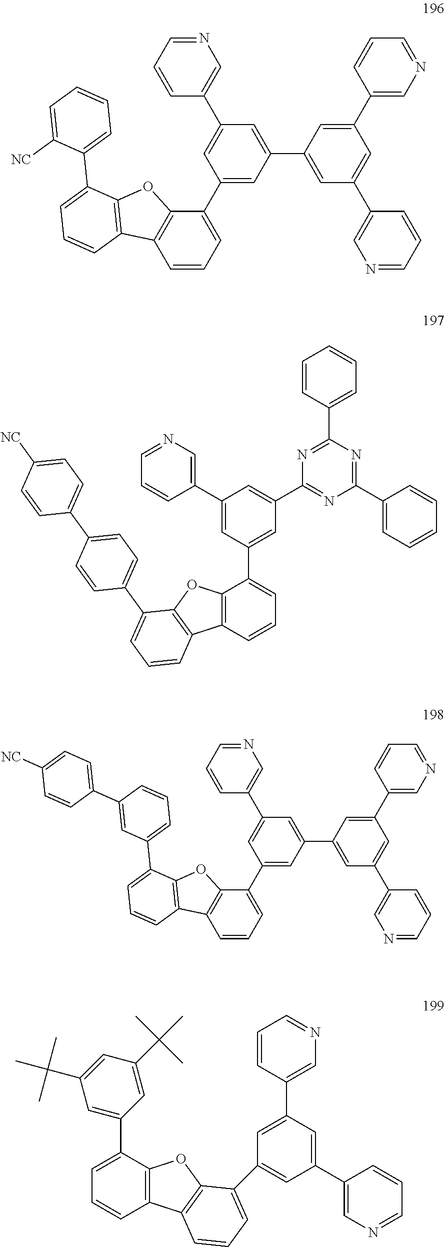 [SMILES: N#Cc1ccccc1-c1cccc2c1oc1c(-c3cc(-c4cccnc4)cc(-c4cc(-c5cccnc5)cc(-c5cccnc5)c4)c3)cccc12 ; N#Cc1ccc(-c2ccc(-c3cccc4c3oc3c(-c5cc(-c6cccnc6)cc(-c6nc(-c7ccccc7)nc(-c7ccccc7)n6)c5)cccc34)cc2)cc1 ; N#Cc1ccc(-c2cccc(-c3cccc4c3oc3c(-c5cc(-c6cccnc6)cc(-c6cc(-c7cccnc7)cc(-c7cccnc7)c6)c5)cccc34)c2)cc1 ; CC(C)(C)c1cc(-c2cccc3c2oc2c(-c4cc(-c5cccnc5)cc(-c5cccnc5)c4)cccc23)cc(C(C)(C)C)c1]

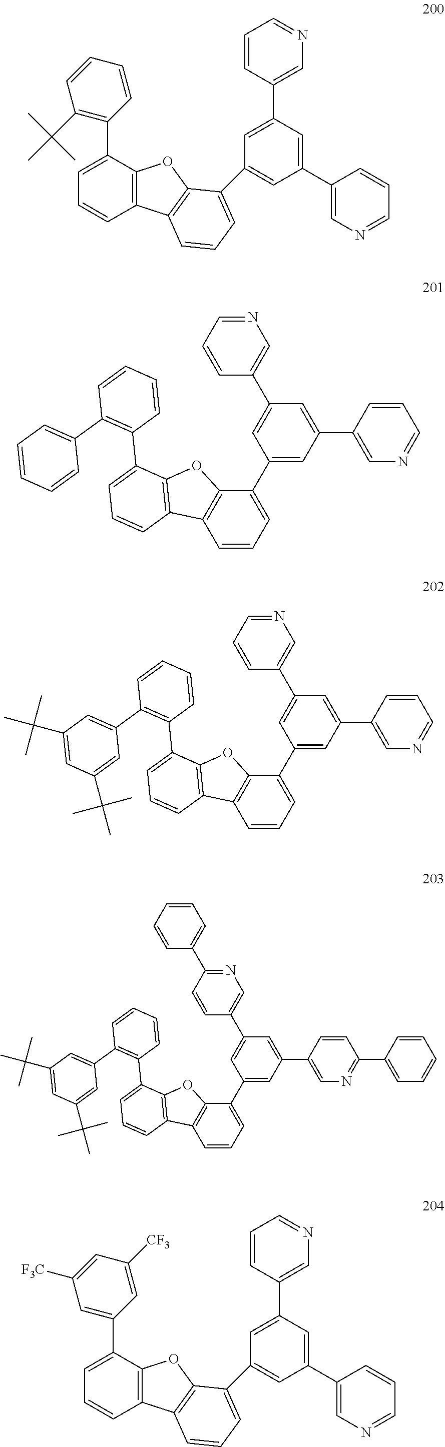 [SMILES: CC(C)(C)c1ccccc1-c1cccc2c1oc1c(-c3cc(-c4cccnc4)cc(-c4cccnc4)c3)cccc12 ; c1ccc(-c2ccccc2-c2cccc3c2oc2c(-c4cc(-c5cccnc5)cc(-c5cccnc5)c4)cccc23)cc1 ; CC(C)(C)c1cc(-c2ccccc2-c2cccc3c2oc2c(-c4cc(-c5cccnc5)cc(-c5cccnc5)c4)cccc23)cc(C(C)(C)C)c1 ; CC(C)(C)c1cc(-c2ccccc2-c2cccc3c2oc2c(-c4cc(-c5ccc(-c6ccccc6)nc5)cc(-c5ccc(-c6ccccc6)nc5)c4)cccc23)cc(C(C)(C)C)c1 ; FC(F)(F)c1cc(-c2cccc3c2oc2c(-c4cc(-c5cccnc5)cc(-c5cccnc5)c4)cccc23)cc(C(F)(F)F)c1]

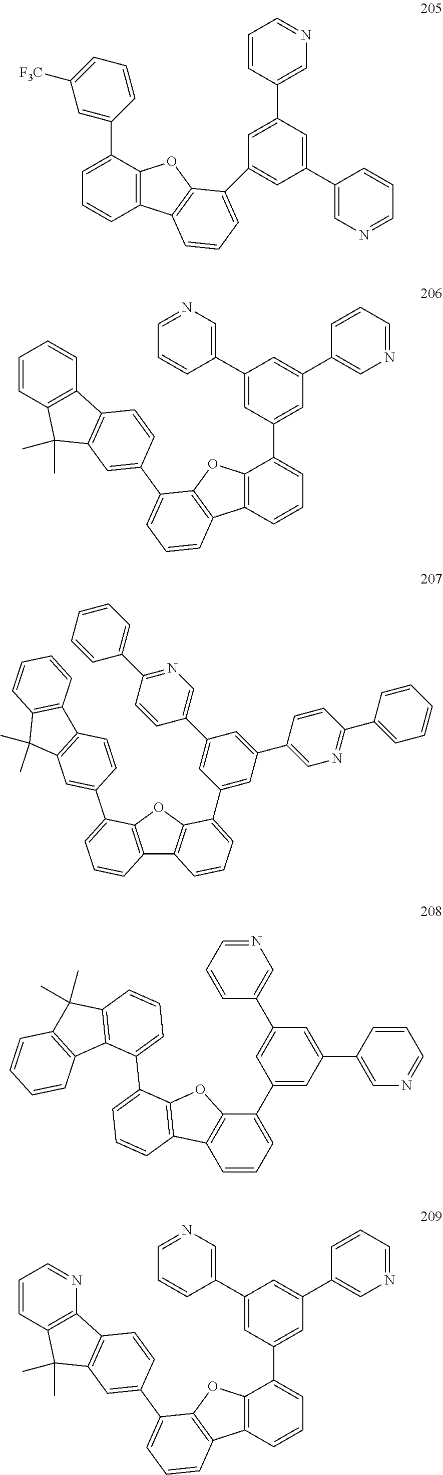 [SMILES: FC(F)(F)c1cccc(-c2cccc3c2oc2c(-c4cc(-c5cccnc5)cc(-c5cccnc5)c4)cccc23)c1 ; CC1(C)c2ccccc2-c2ccc(-c3cccc4c3oc3c(-c5cc(-c6cccnc6)cc(-c6cccnc6)c5)cccc34)cc21 ; CC1(C)c2ccccc2-c2ccc(-c3cccc4c3oc3c(-c5cc(-c6ccc(-c7ccccc7)nc6)cc(-c6ccc(-c7ccccc7)nc6)c5)cccc34)cc21 ; CC1(C)c2ccccc2-c2c(-c3cccc4c3oc3c(-c5cc(-c6cccnc6)cc(-c6cccnc6)c5)cccc34)cccc21 ; CC1(C)c2cc(-c3cccc4c3oc3c(-c5cc(-c6cccnc6)cc(-c6cccnc6)c5)cccc34)ccc2-c2ncccc21]

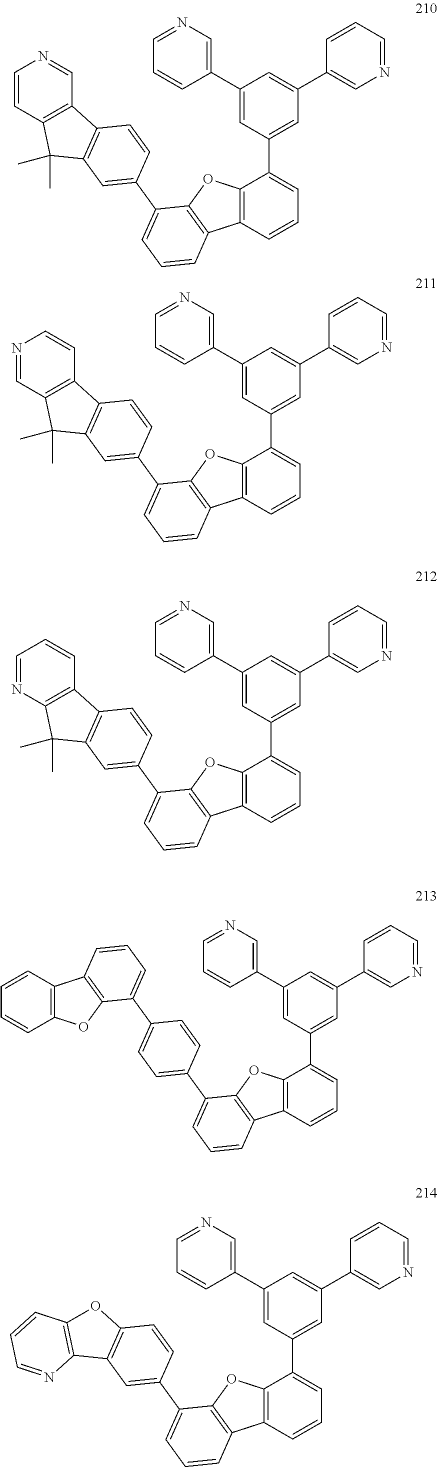

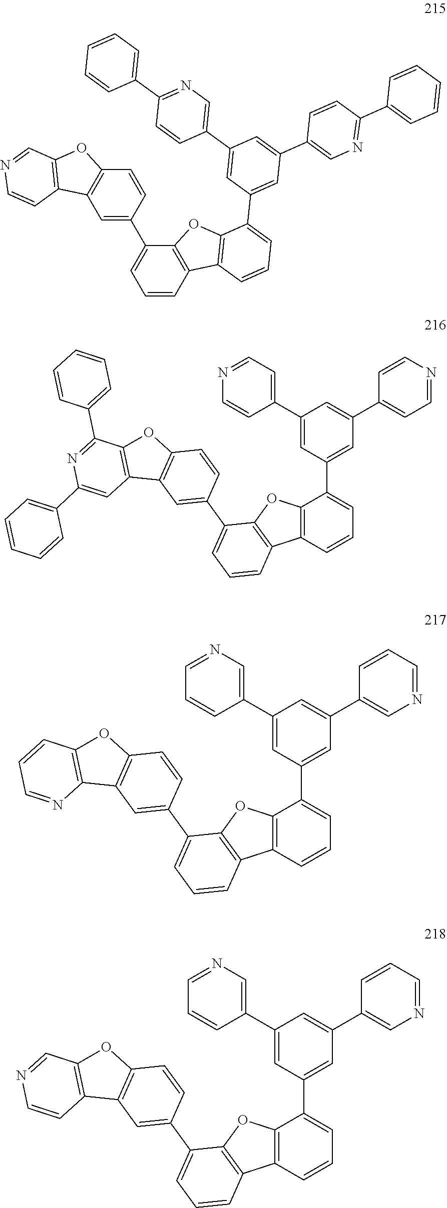 [SMILES: c1ccc(-c2ccc(-c3cc(-c4ccc(-c5ccccc5)nc4)cc(-c4cccc5c4oc4c(-c6ccc7oc8cnccc8c7c6)cccc45)c3)cn2)cc1 ; c1ccc(-c2cc3c(oc4ccc(-c5cccc6c5oc5c(-c7cc(-c8ccncc8)cc(-c8ccncc8)c7)cccc56)cc43)c(-c3ccccc3)n2)cc1 ; c1cncc(-c2cc(-c3cccnc3)cc(-c3cccc4c3oc3c(-c5ccc6oc7cccnc7c6c5)cccc34)c2)c1 ; c1cncc(-c2cc(-c3cccnc3)cc(-c3cccc4c3oc3c(-c5ccc6oc7cnccc7c6c5)cccc34)c2)c1]

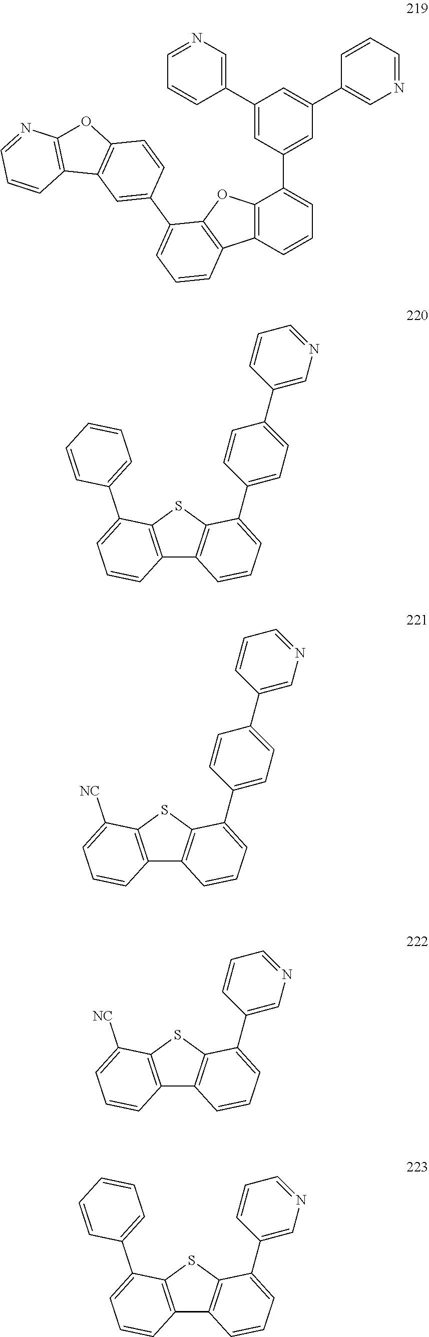 [SMILES: c1cncc(-c2cc(-c3cccnc3)cc(-c3cccc4c3oc3c(-c5ccc6oc7ncccc7c6c5)cccc34)c2)c1 ; c1ccc(-c2cccc3c2sc2c(-c4ccc(-c5cccnc5)cc4)cccc23)cc1 ; N#Cc1cccc2c1sc1c(-c3ccc(-c4cccnc4)cc3)cccc12 ; N#Cc1cccc2c1sc1c(-c3cccnc3)cccc12 ; c1ccc(-c2cccc3c2sc2c(-c4cccnc4)cccc23)cc1]

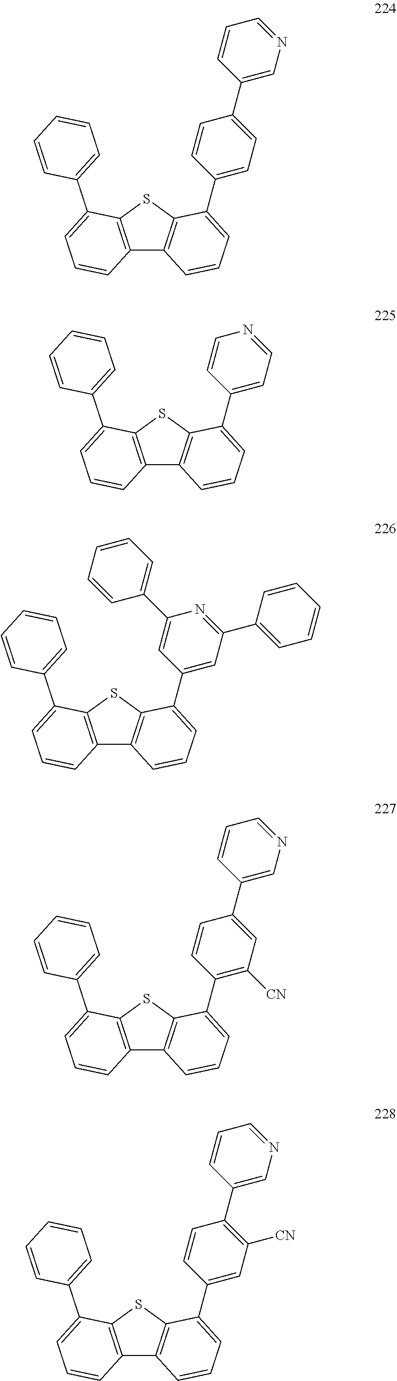 [SMILES: c1ccc(-c2cccc3c2sc2c(-c4ccc(-c5cccnc5)cc4)cccc23)cc1 ; c1ccc(-c2cccc3c2sc2c(-c4ccncc4)cccc23)cc1 ; c1ccc(-c2cc(-c3cccc4c3sc3c(-c5ccccc5)cccc34)cc(-c3ccccc3)n2)cc1 ; N#Cc1cc(-c2cccnc2)ccc1-c1cccc2c1sc1c(-c3ccccc3)cccc12 ; N#Cc1cc(-c2cccc3c2sc2c(-c4ccccc4)cccc23)ccc1-c1cccnc1]

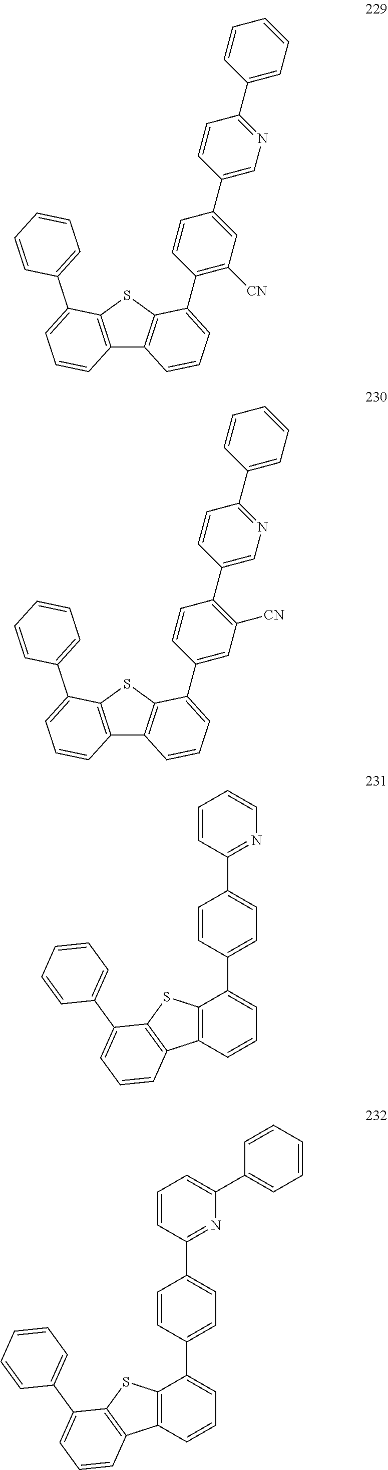 [SMILES: N#Cc1cc(-c2ccc(-c3ccccc3)nc2)ccc1-c1cccc2c1sc1c(-c3ccccc3)cccc12 ; N#Cc1cc(-c2cccc3c2sc2c(-c4ccccc4)cccc23)ccc1-c1ccc(-c2ccccc2)nc1 ; c1ccc(-c2cccc3c2sc2c(-c4ccc(-c5ccccn5)cc4)cccc23)cc1 ; c1ccc(-c2cccc(-c3ccc(-c4cccc5c4sc4c(-c6ccccc6)cccc45)cc3)n2)cc1]

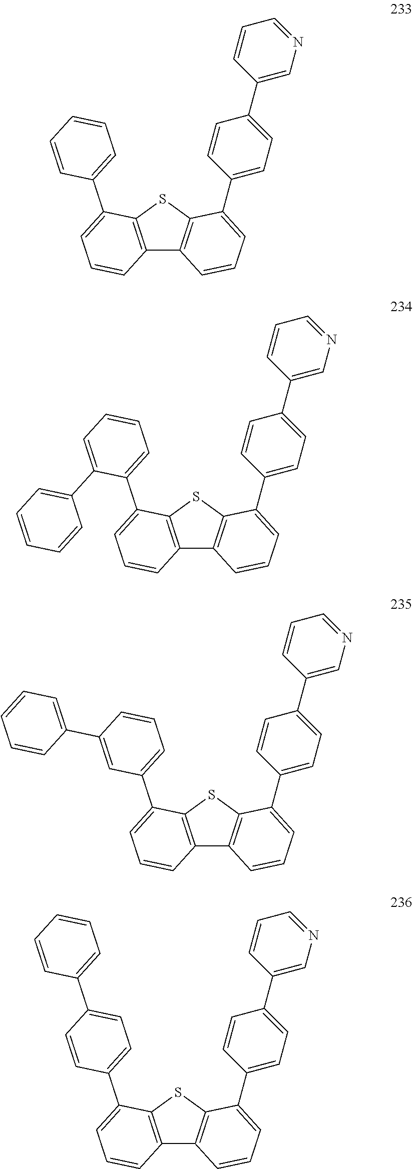 [SMILES: c1ccc(-c2cccc3c2sc2c(-c4ccc(-c5cccnc5)cc4)cccc23)cc1 ; c1ccc(-c2ccccc2-c2cccc3c2sc2c(-c4ccc(-c5cccnc5)cc4)cccc23)cc1 ; c1ccc(-c2cccc(-c3cccc4c3sc3c(-c5ccc(-c6cccnc6)cc5)cccc34)c2)cc1 ; c1ccc(-c2ccc(-c3cccc4c3sc3c(-c5ccc(-c6cccnc6)cc5)cccc34)cc2)cc1]

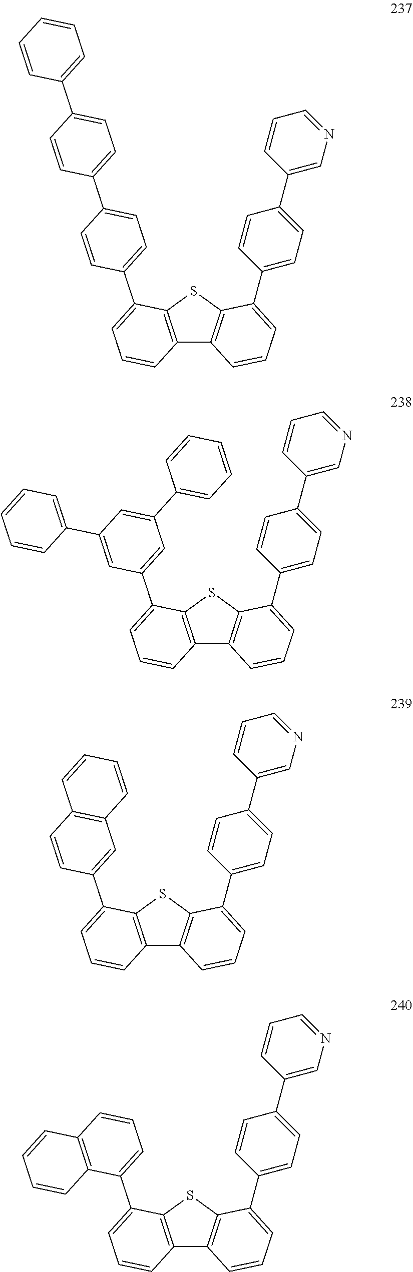 [SMILES: c1ccc(-c2ccc(-c3ccc(-c4cccc5c4sc4c(-c6ccc(-c7cccnc7)cc6)cccc45)cc3)cc2)cc1 ; c1ccc(-c2cc(-c3ccccc3)cc(-c3cccc4c3sc3c(-c5ccc(-c6cccnc6)cc5)cccc34)c2)cc1 ; c1cncc(-c2ccc(-c3cccc4c3sc3c(-c5ccc6ccccc6c5)cccc34)cc2)c1 ; c1cncc(-c2ccc(-c3cccc4c3sc3c(-c5cccc6ccccc56)cccc34)cc2)c1]

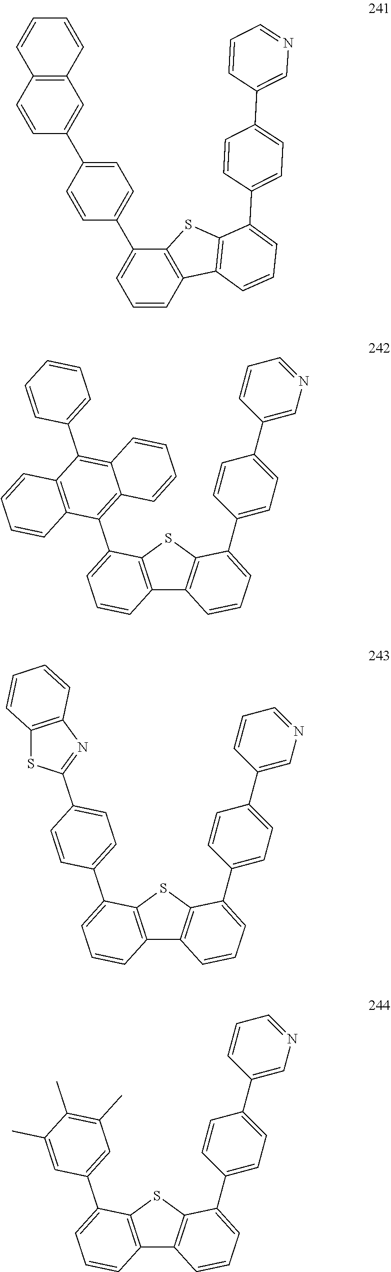 [SMILES: c1cncc(-c2ccc(-c3cccc4c3sc3c(-c5ccc(-c6ccc7ccccc7c6)cc5)cccc34)cc2)c1 ; c1ccc(-c2c3ccccc3c(-c3cccc4c3sc3c(-c5ccc(-c6cccnc6)cc5)cccc34)c3ccccc23)cc1 ; c1cncc(-c2ccc(-c3cccc4c3sc3c(-c5ccc(-c6nc7ccccc7s6)cc5)cccc34)cc2)c1 ; Cc1cc(-c2cccc3c2sc2c(-c4ccc(-c5cccnc5)cc4)cccc23)cc(C)c1C]

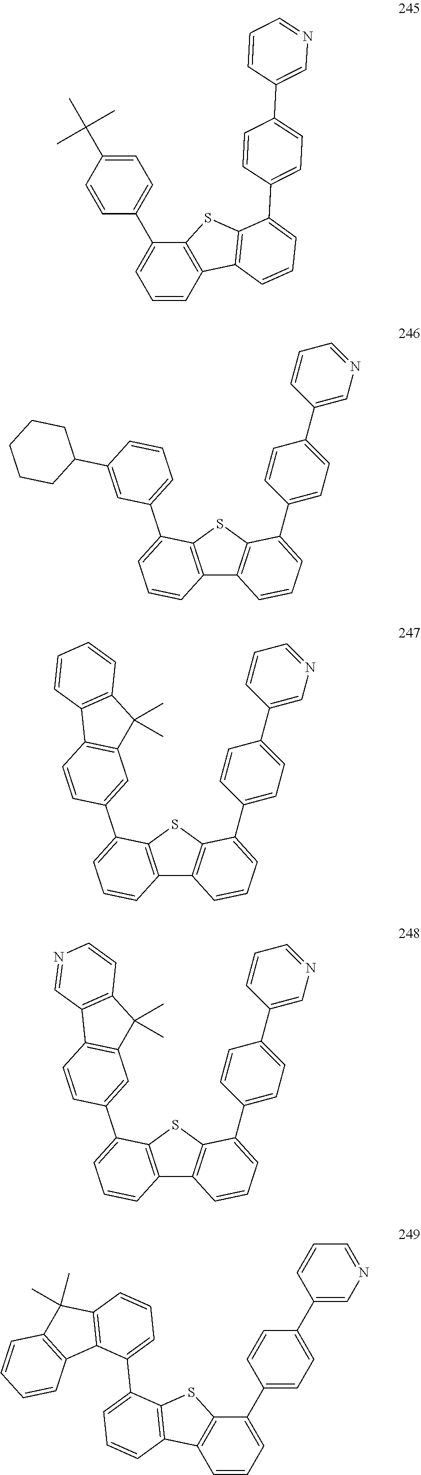 [SMILES: CC(C)(C)c1ccc(-c2cccc3c2sc2c(-c4ccc(-c5cccnc5)cc4)cccc23)cc1 ; c1cncc(-c2ccc(-c3cccc4c3sc3c(-c5cccc(C6CCCCC6)c5)cccc34)cc2)c1 ; CC1(C)c2ccccc2-c2ccc(-c3cccc4c3sc3c(-c5ccc(-c6cccnc6)cc5)cccc34)cc21 ; CC1(C)c2ccncc2-c2ccc(-c3cccc4c3sc3c(-c5ccc(-c6cccnc6)cc5)cccc34)cc21 ; CC1(C)c2ccccc2-c2c(-c3cccc4c3sc3c(-c5ccc(-c6cccnc6)cc5)cccc34)cccc21]

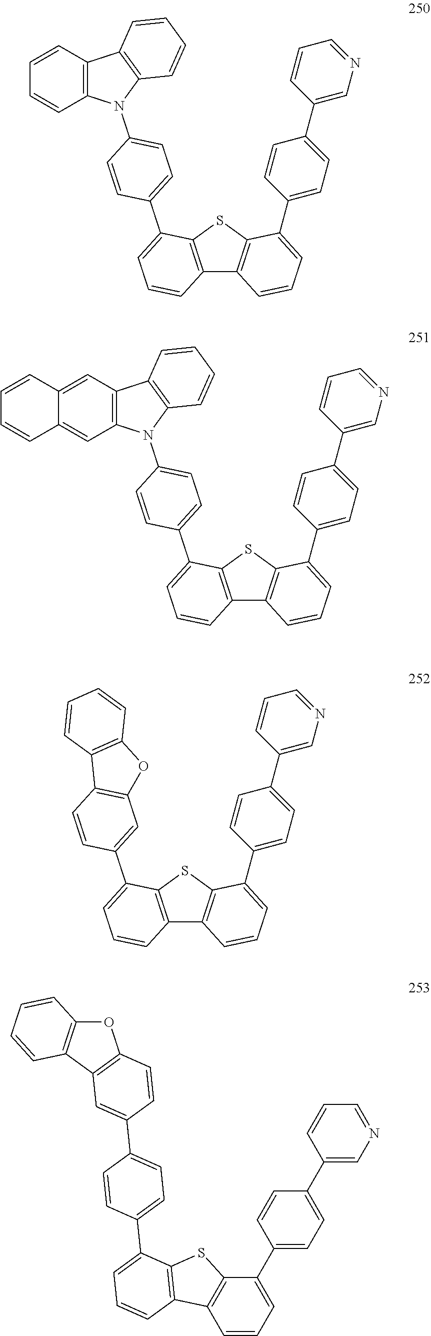 [SMILES: c1cncc(-c2ccc(-c3cccc4c3sc3c(-c5ccc(-n6c7ccccc7c7ccccc76)cc5)cccc34)cc2)c1 ; c1cncc(-c2ccc(-c3cccc4c3sc3c(-c5ccc(-n6c7ccccc7c7cc8ccccc8cc76)cc5)cccc34)cc2)c1 ; c1cncc(-c2ccc(-c3cccc4c3sc3c(-c5ccc6c(c5)oc5ccccc56)cccc34)cc2)c1 ; c1cncc(-c2ccc(-c3cccc4c3sc3c(-c5ccc(-c6ccc7oc8ccccc8c7c6)cc5)cccc34)cc2)c1]

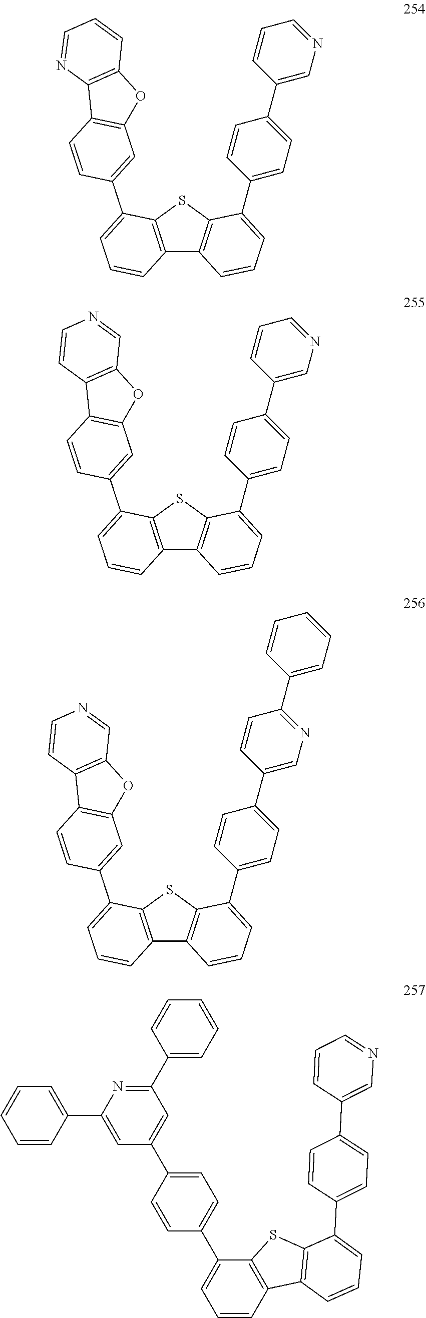

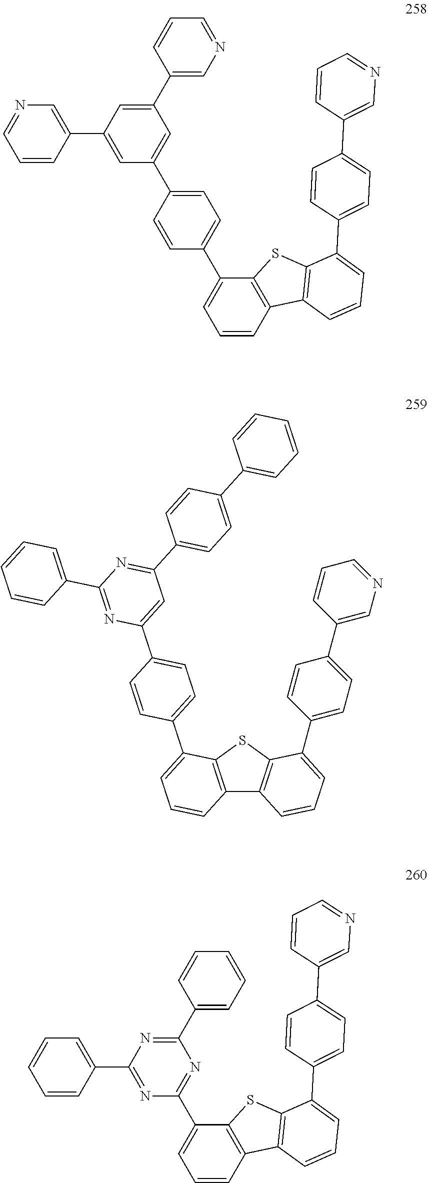 [SMILES: c1cncc(-c2ccc(-c3cccc4c3sc3c(-c5ccc(-c6cc(-c7cccnc7)cc(-c7cccnc7)c6)cc5)cccc34)cc2)c1 ; c1ccc(-c2ccc(-c3cc(-c4ccc(-c5cccc6c5sc5c(-c7ccc(-c8cccnc8)cc7)cccc56)cc4)nc(-c4ccccc4)n3)cc2)cc1 ; c1ccc(-c2nc(-c3ccccc3)nc(-c3cccc4c3sc3c(-c5ccc(-c6cccnc6)cc5)cccc34)n2)cc1]

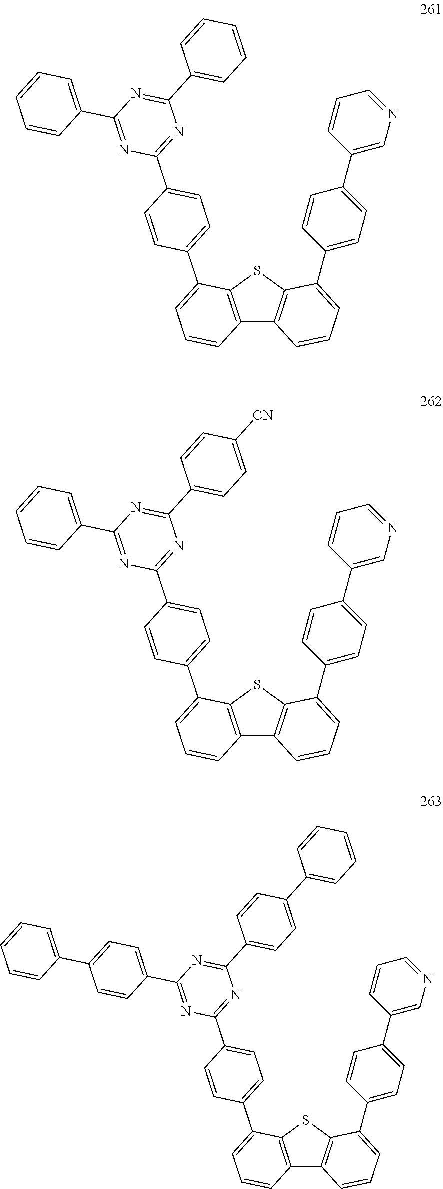 [SMILES: c1ccc(-c2nc(-c3ccccc3)nc(-c3ccc(-c4cccc5c4sc4c(-c6ccc(-c7cccnc7)cc6)cccc45)cc3)n2)cc1 ; N#Cc1ccc(-c2nc(-c3ccccc3)nc(-c3ccc(-c4cccc5c4sc4c(-c6ccc(-c7cccnc7)cc6)cccc45)cc3)n2)cc1 ; c1ccc(-c2ccc(-c3nc(-c4ccc(-c5ccccc5)cc4)nc(-c4ccc(-c5cccc6c5sc5c(-c7ccc(-c8cccnc8)cc7)cccc56)cc4)n3)cc2)cc1]

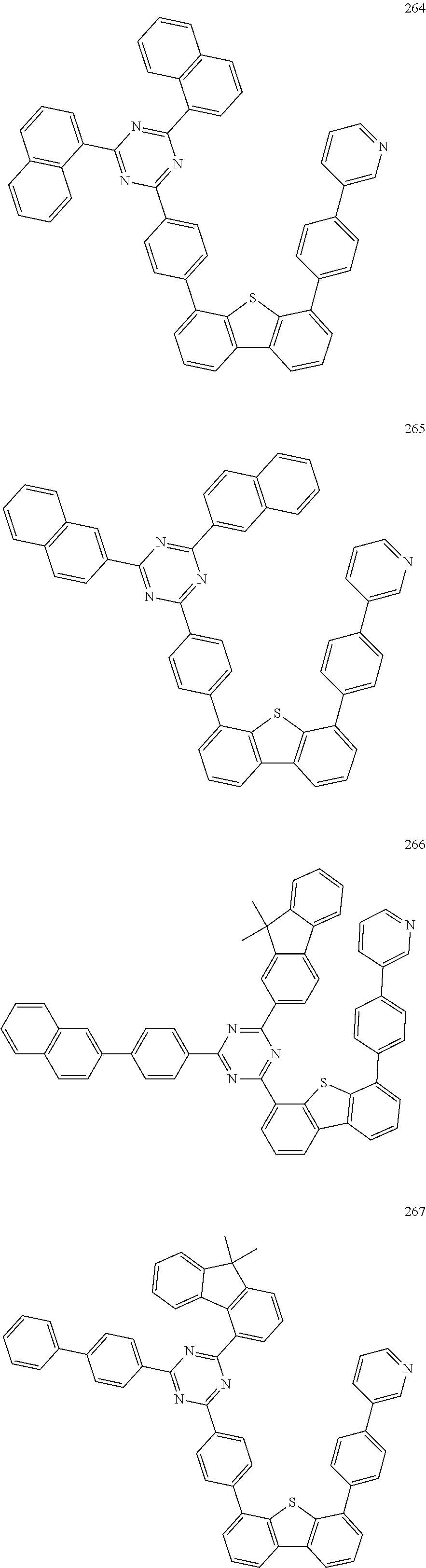 [SMILES: c1cncc(-c2ccc(-c3cccc4c3sc3c(-c5ccc(-c6nc(-c7cccc8ccccc78)nc(-c7cccc8ccccc78)n6)cc5)cccc34)cc2)c1 ; c1cncc(-c2ccc(-c3cccc4c3sc3c(-c5ccc(-c6nc(-c7ccc8ccccc8c7)nc(-c7ccc8ccccc8c7)n6)cc5)cccc34)cc2)c1 ; CC1(C)c2ccccc2-c2ccc(-c3nc(-c4ccc(-c5ccc6ccccc6c5)cc4)nc(-c4cccc5c4sc4c(-c6ccc(-c7cccnc7)cc6)cccc45)n3)cc21 ; CC1(C)c2ccccc2-c2c(-c3nc(-c4ccc(-c5ccccc5)cc4)nc(-c4ccc(-c5cccc6c5sc5c(-c7ccc(-c8cccnc8)cc7)cccc56)cc4)n3)cccc21]

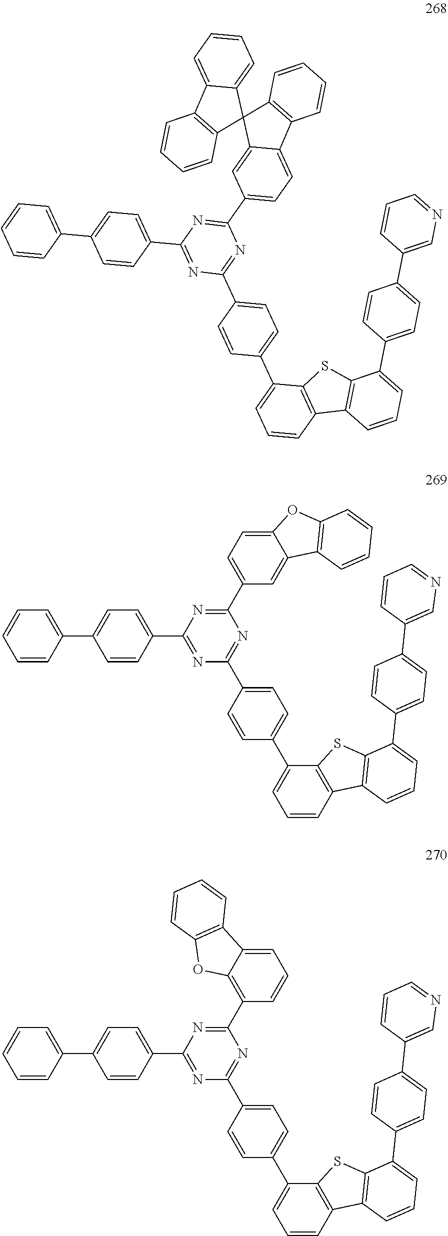 [SMILES: c1ccc(-c2ccc(-c3nc(-c4ccc(-c5cccc6c5sc5c(-c7ccc(-c8cccnc8)cc7)cccc56)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5cccc6c5sc5c(-c7ccc(-c8cccnc8)cc7)cccc56)cc4)nc(-c4ccc5oc6ccccc6c5c4)n3)cc2)cc1 ; c1ccc(-c2ccc(-c3nc(-c4ccc(-c5cccc6c5sc5c(-c7ccc(-c8cccnc8)cc7)cccc56)cc4)nc(-c4cccc5c4oc4ccccc45)n3)cc2)cc1]

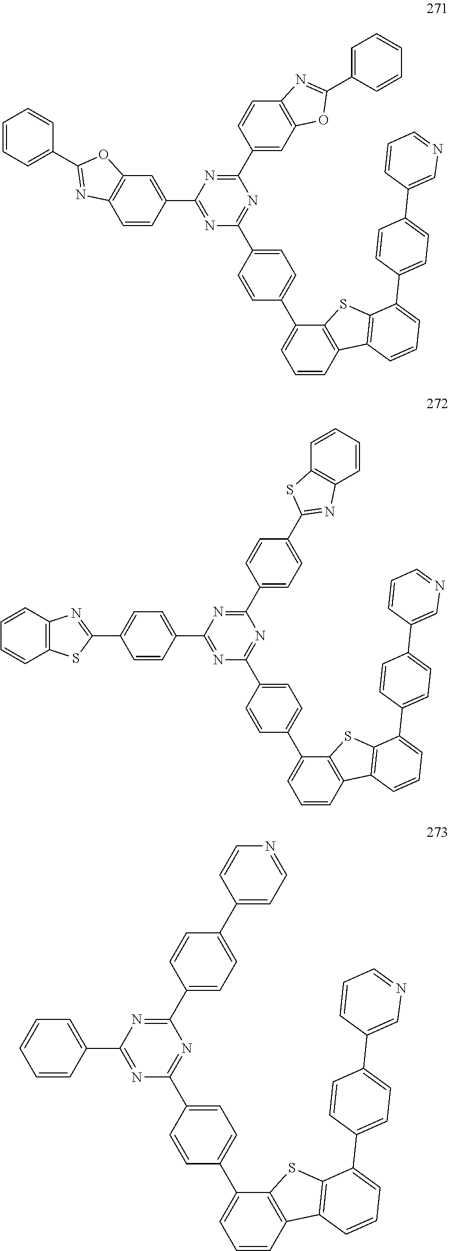 [SMILES: c1ccc(-c2nc3ccc(-c4nc(-c5ccc(-c6cccc7c6sc6c(-c8ccc(-c9cccnc9)cc8)cccc67)cc5)nc(-c5ccc6nc(-c7ccccc7)oc6c5)n4)cc3o2)cc1 ; c1cncc(-c2ccc(-c3cccc4c3sc3c(-c5ccc(-c6nc(-c7ccc(-c8nc9ccccc9s8)cc7)nc(-c7ccc(-c8nc9ccccc9s8)cc7)n6)cc5)cccc34)cc2)c1 ; c1ccc(-c2nc(-c3ccc(-c4ccncc4)cc3)nc(-c3ccc(-c4cccc5c4sc4c(-c6ccc(-c7cccnc7)cc6)cccc45)cc3)n2)cc1]

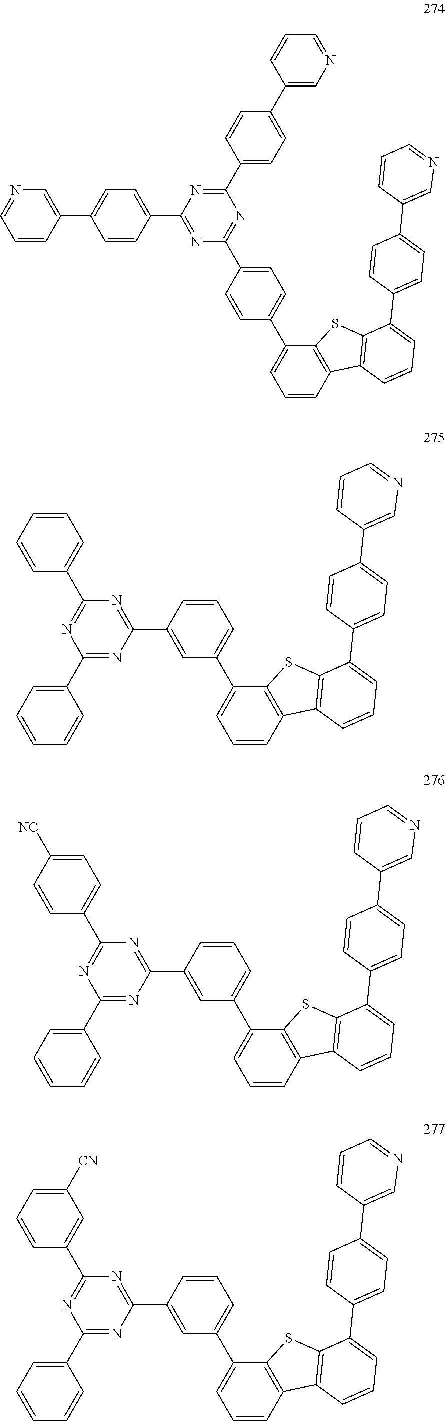 [SMILES: c1cncc(-c2ccc(-c3nc(-c4ccc(-c5cccnc5)cc4)nc(-c4ccc(-c5cccc6c5sc5c(-c7ccc(-c8cccnc8)cc7)cccc56)cc4)n3)cc2)c1 ; c1ccc(-c2nc(-c3ccccc3)nc(-c3cccc(-c4cccc5c4sc4c(-c6ccc(-c7cccnc7)cc6)cccc45)c3)n2)cc1 ; N#Cc1ccc(-c2nc(-c3ccccc3)nc(-c3cccc(-c4cccc5c4sc4c(-c6ccc(-c7cccnc7)cc6)cccc45)c3)n2)cc1 ; N#Cc1cccc(-c2nc(-c3ccccc3)nc(-c3cccc(-c4cccc5c4sc4c(-c6ccc(-c7cccnc7)cc6)cccc45)c3)n2)c1]

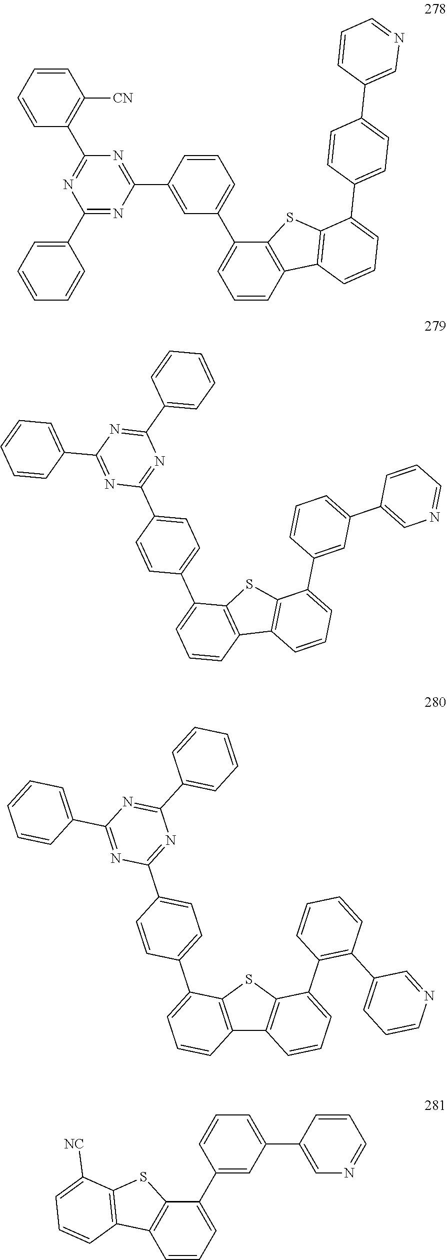 [SMILES: N#Cc1ccccc1-c1nc(-c2ccccc2)nc(-c2cccc(-c3cccc4c3sc3c(-c5ccc(-c6cccnc6)cc5)cccc34)c2)n1 ; c1ccc(-c2nc(-c3ccccc3)nc(-c3ccc(-c4cccc5c4sc4c(-c6cccc(-c7cccnc7)c6)cccc45)cc3)n2)cc1 ; c1ccc(-c2nc(-c3ccccc3)nc(-c3ccc(-c4cccc5c4sc4c(-c6ccccc6-c6cccnc6)cccc45)cc3)n2)cc1 ; N#Cc1cccc2c1sc1c(-c3cccc(-c4cccnc4)c3)cccc12]

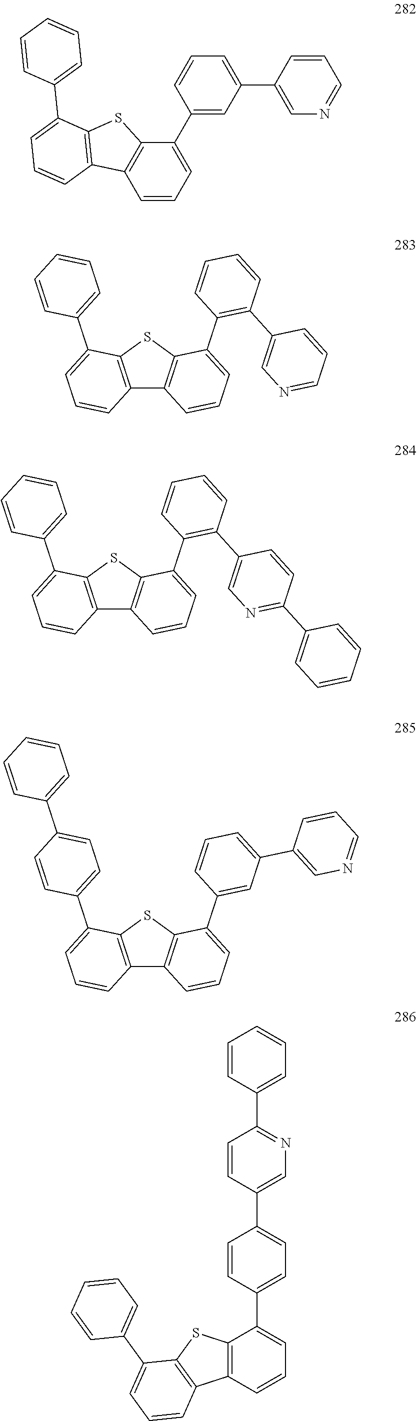 [SMILES: c1ccc(-c2cccc3c2sc2c(-c4cccc(-c5cccnc5)c4)cccc23)cc1 ; c1ccc(-c2cccc3c2sc2c(-c4ccccc4-c4cccnc4)cccc23)cc1 ; c1ccc(-c2ccc(-c3ccccc3-c3cccc4c3sc3c(-c5ccccc5)cccc34)cn2)cc1 ; c1ccc(-c2ccc(-c3cccc4c3sc3c(-c5cccc(-c6cccnc6)c5)cccc34)cc2)cc1 ; c1ccc(-c2ccc(-c3ccc(-c4cccc5c4sc4c(-c6ccccc6)cccc45)cc3)cn2)cc1]

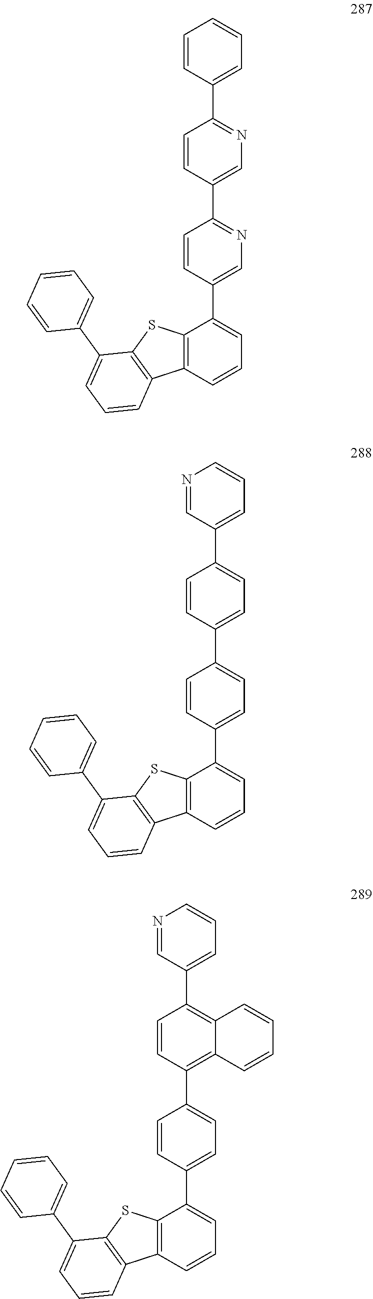 [SMILES: c1ccc(-c2ccc(-c3ccc(-c4cccc5c4sc4c(-c6ccccc6)cccc45)cn3)cn2)cc1 ; c1ccc(-c2cccc3c2sc2c(-c4ccc(-c5ccc(-c6cccnc6)cc5)cc4)cccc23)cc1 ; c1ccc(-c2cccc3c2sc2c(-c4ccc(-c5ccc(-c6cccnc6)c6ccccc56)cc4)cccc23)cc1]

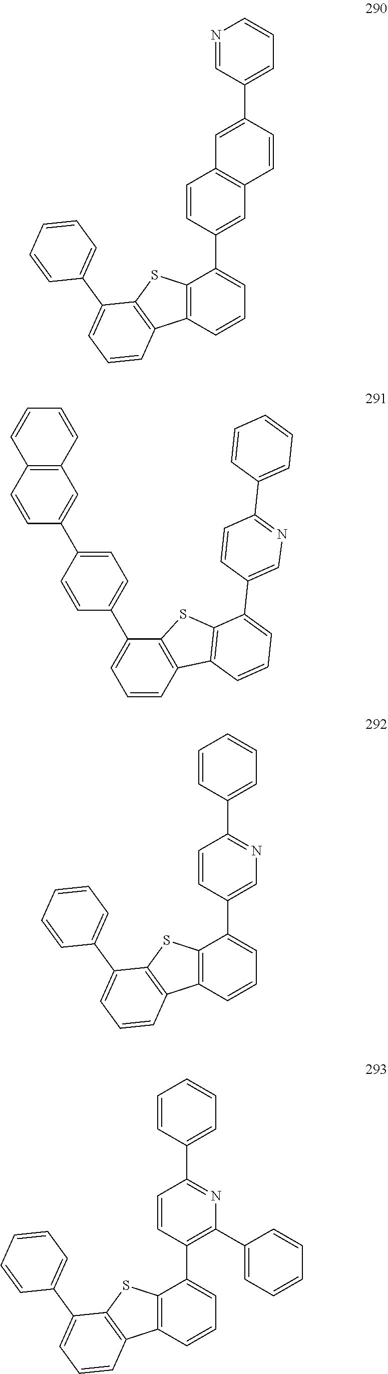 [SMILES: c1ccc(-c2cccc3c2sc2c(-c4ccc5cc(-c6cccnc6)ccc5c4)cccc23)cc1 ; c1ccc(-c2ccc(-c3cccc4c3sc3c(-c5ccc(-c6ccc7ccccc7c6)cc5)cccc34)cn2)cc1 ; c1ccc(-c2ccc(-c3cccc4c3sc3c(-c5ccccc5)cccc34)cn2)cc1 ; c1ccc(-c2ccc(-c3cccc4c3sc3c(-c5ccccc5)cccc34)c(-c3ccccc3)n2)cc1]

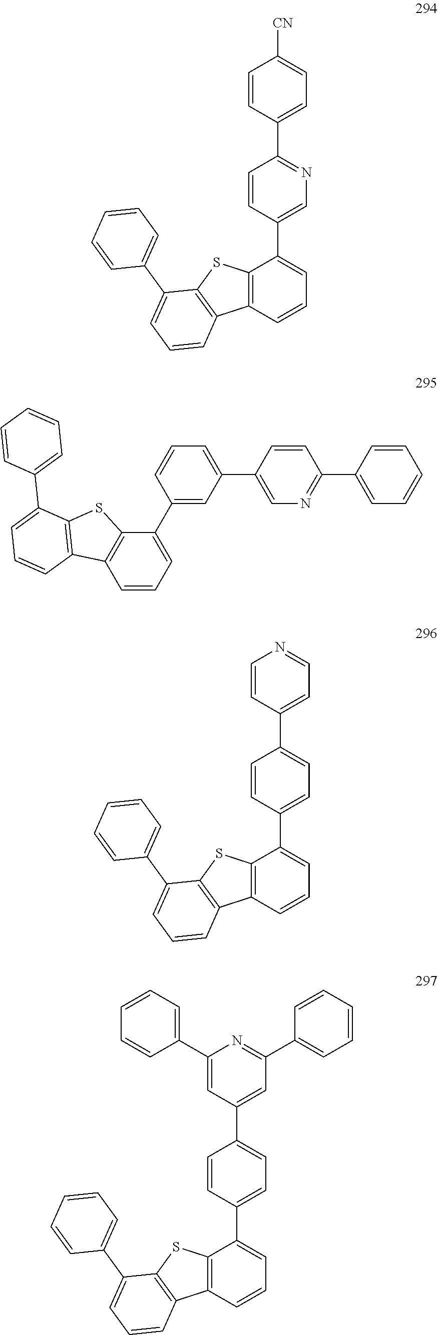 [SMILES: N#Cc1ccc(-c2ccc(-c3cccc4c3sc3c(-c5ccccc5)cccc34)cn2)cc1 ; c1ccc(-c2ccc(-c3cccc(-c4cccc5c4sc4c(-c6ccccc6)cccc45)c3)cn2)cc1 ; c1ccc(-c2cccc3c2sc2c(-c4ccc(-c5ccncc5)cc4)cccc23)cc1 ; c1ccc(-c2cc(-c3ccc(-c4cccc5c4sc4c(-c6ccccc6)cccc45)cc3)cc(-c3ccccc3)n2)cc1]

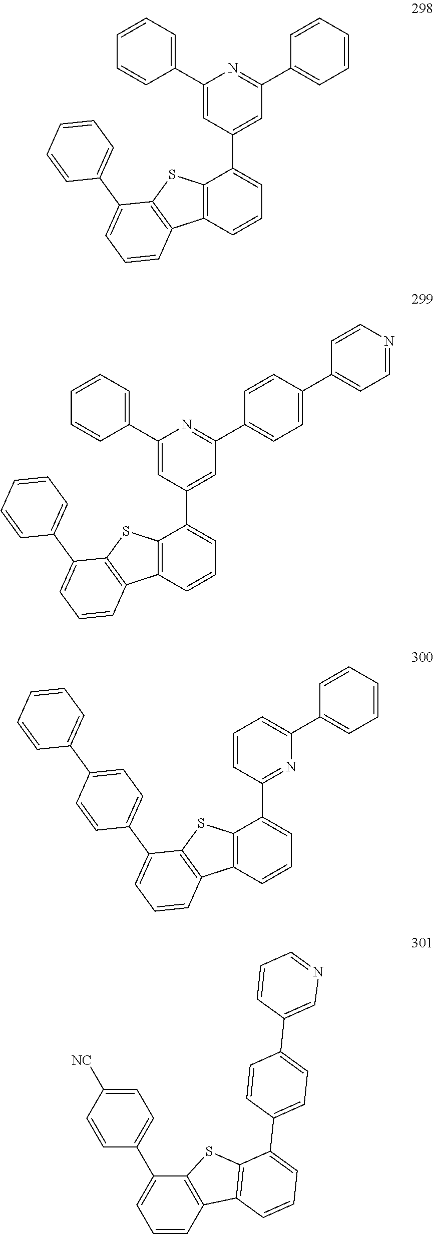 [SMILES: c1ccc(-c2cc(-c3cccc4c3sc3c(-c5ccccc5)cccc34)cc(-c3ccccc3)n2)cc1 ; c1ccc(-c2cc(-c3cccc4c3sc3c(-c5ccccc5)cccc34)cc(-c3ccc(-c4ccncc4)cc3)n2)cc1 ; c1ccc(-c2ccc(-c3cccc4c3sc3c(-c5cccc(-c6ccccc6)n5)cccc34)cc2)cc1 ; N#Cc1ccc(-c2cccc3c2sc2c(-c4ccc(-c5cccnc5)cc4)cccc23)cc1]

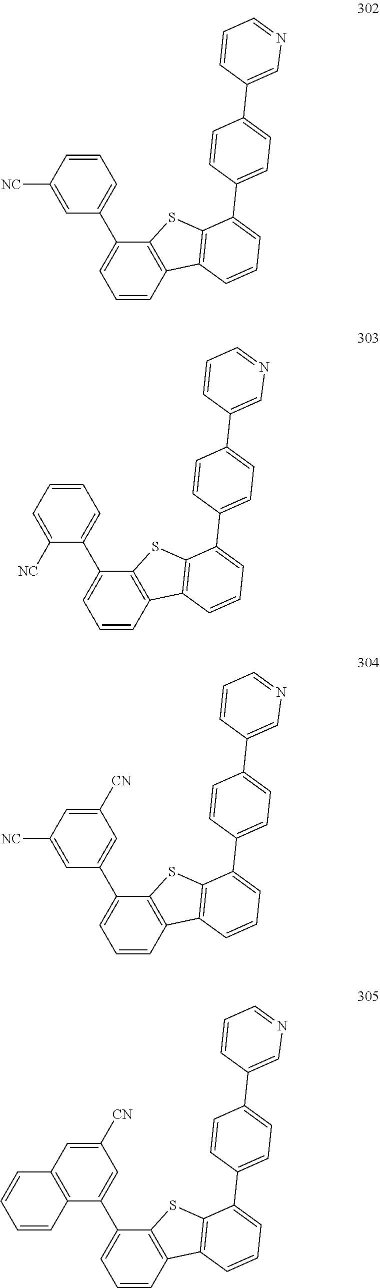 [SMILES: N#Cc1cccc(-c2cccc3c2sc2c(-c4ccc(-c5cccnc5)cc4)cccc23)c1 ; N#Cc1ccccc1-c1cccc2c1sc1c(-c3ccc(-c4cccnc4)cc3)cccc12 ; N#Cc1cc(C#N)cc(-c2cccc3c2sc2c(-c4ccc(-c5cccnc5)cc4)cccc23)c1 ; N#Cc1cc(-c2cccc3c2sc2c(-c4ccc(-c5cccnc5)cc4)cccc23)c2ccccc2c1]

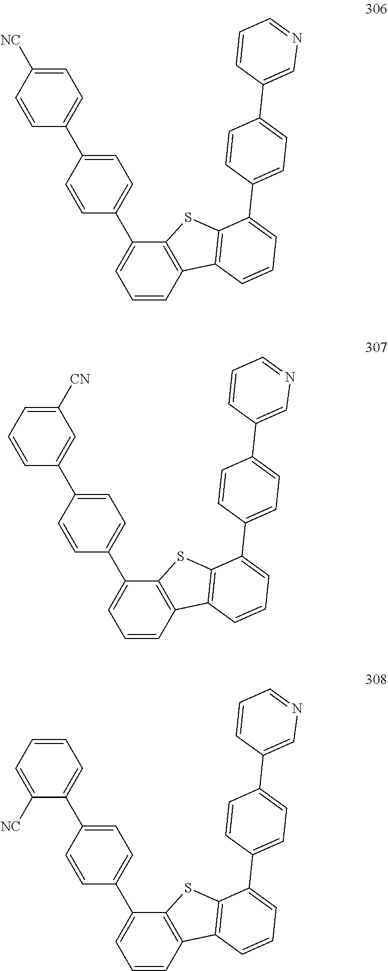 [SMILES: N#Cc1ccc(-c2ccc(-c3cccc4c3sc3c(-c5ccc(-c6cccnc6)cc5)cccc34)cc2)cc1 ; N#Cc1cccc(-c2ccc(-c3cccc4c3sc3c(-c5ccc(-c6cccnc6)cc5)cccc34)cc2)c1 ; N#Cc1ccccc1-c1ccc(-c2cccc3c2sc2c(-c4ccc(-c5cccnc5)cc4)cccc23)cc1]

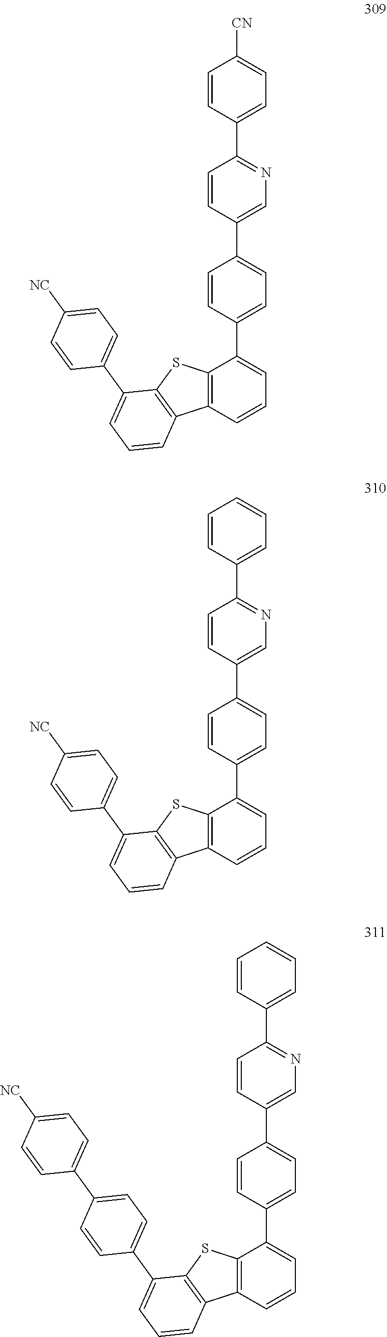 [SMILES: N#Cc1ccc(-c2ccc(-c3ccc(-c4cccc5c4sc4c(-c6ccc(C#N)cc6)cccc45)cc3)cn2)cc1 ; N#Cc1ccc(-c2cccc3c2sc2c(-c4ccc(-c5ccc(-c6ccccc6)nc5)cc4)cccc23)cc1 ; N#Cc1ccc(-c2ccc(-c3cccc4c3sc3c(-c5ccc(-c6ccc(-c7ccccc7)nc6)cc5)cccc34)cc2)cc1]

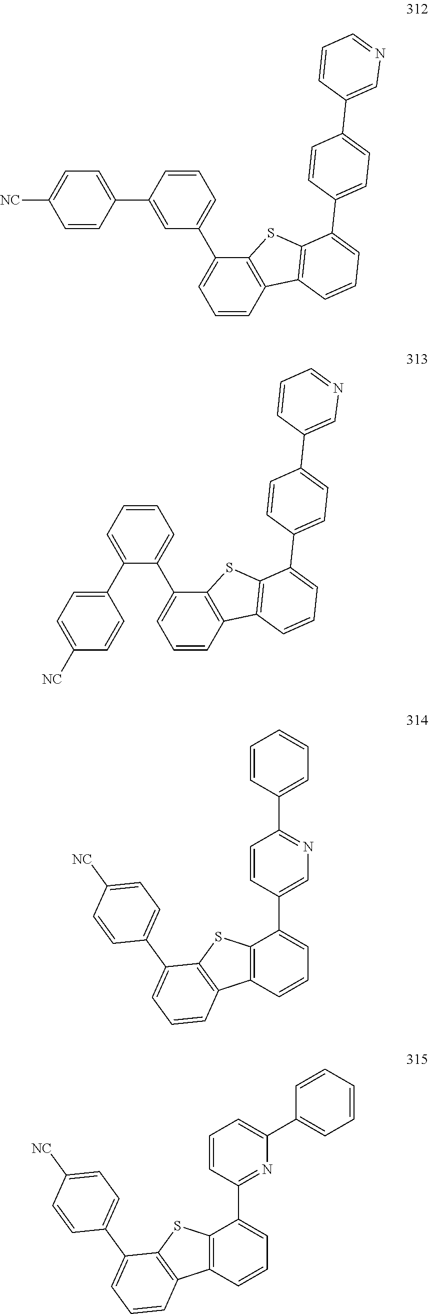 [SMILES: N#Cc1ccc(-c2cccc(-c3cccc4c3sc3c(-c5ccc(-c6cccnc6)cc5)cccc34)c2)cc1 ; N#Cc1ccc(-c2ccccc2-c2cccc3c2sc2c(-c4ccc(-c5cccnc5)cc4)cccc23)cc1 ; N#Cc1ccc(-c2cccc3c2sc2c(-c4ccc(-c5ccccc5)nc4)cccc23)cc1 ; N#Cc1ccc(-c2cccc3c2sc2c(-c4cccc(-c5ccccc5)n4)cccc23)cc1]

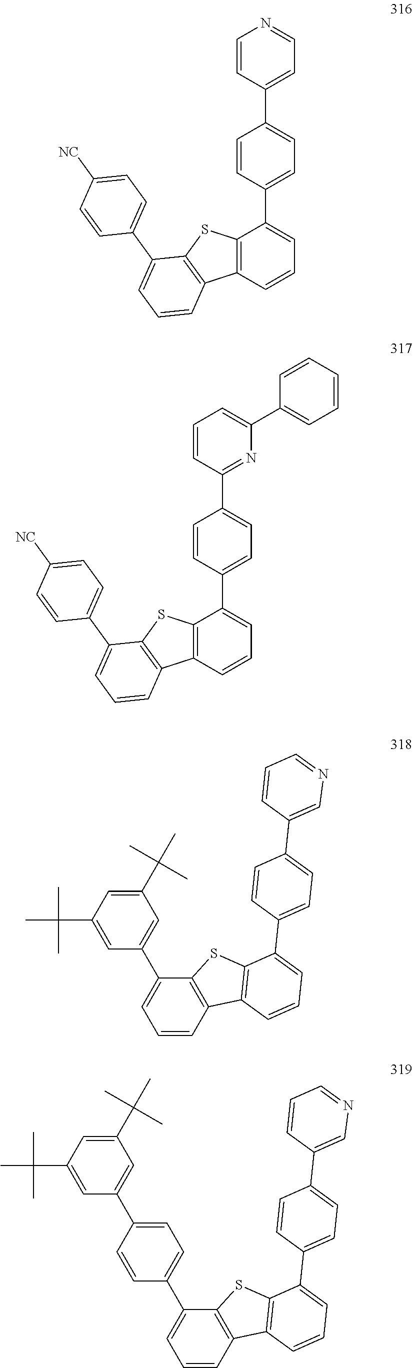

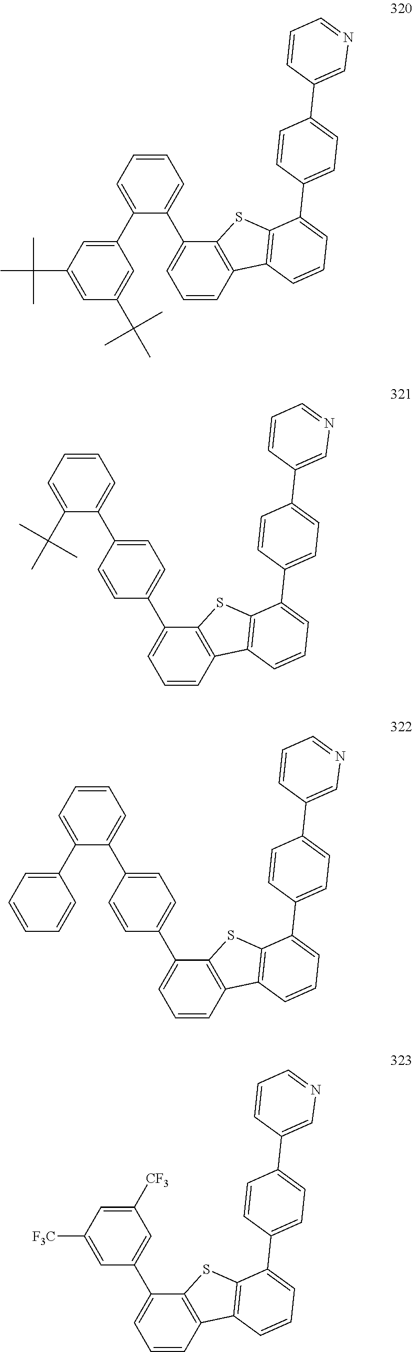 [SMILES: CC(C)(C)c1cc(-c2ccccc2-c2cccc3c2sc2c(-c4ccc(-c5cccnc5)cc4)cccc23)cc(C(C)(C)C)c1 ; CC(C)(C)c1ccccc1-c1ccc(-c2cccc3c2sc2c(-c4ccc(-c5cccnc5)cc4)cccc23)cc1 ; c1ccc(-c2ccccc2-c2ccc(-c3cccc4c3sc3c(-c5ccc(-c6cccnc6)cc5)cccc34)cc2)cc1 ; FC(F)(F)c1cc(-c2cccc3c2sc2c(-c4ccc(-c5cccnc5)cc4)cccc23)cc(C(F)(F)F)c1]

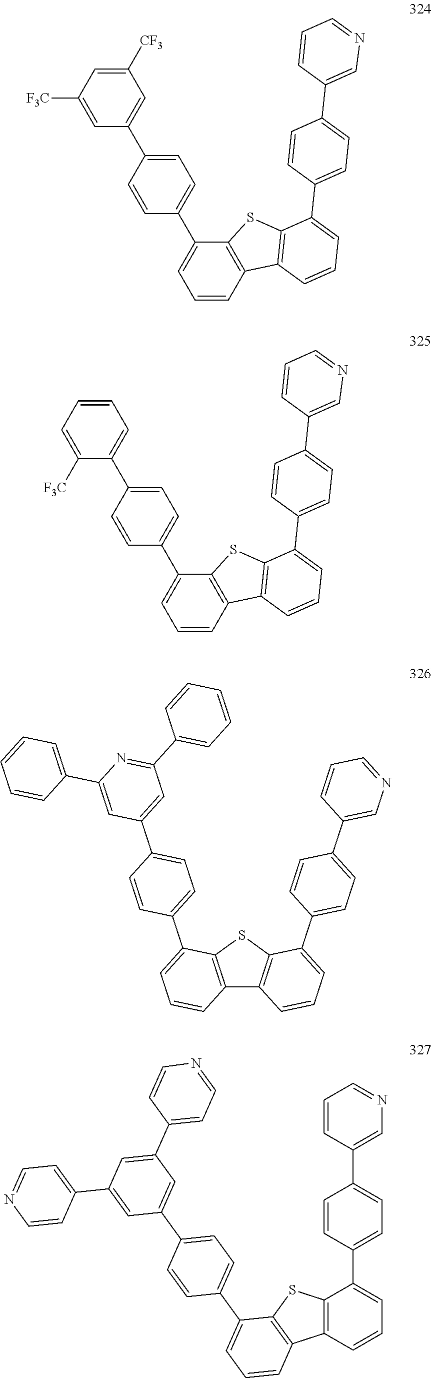 [SMILES: FC(F)(F)c1cc(-c2ccc(-c3cccc4c3sc3c(-c5ccc(-c6cccnc6)cc5)cccc34)cc2)cc(C(F)(F)F)c1 ; FC(F)(F)c1ccccc1-c1ccc(-c2cccc3c2sc2c(-c4ccc(-c5cccnc5)cc4)cccc23)cc1 ; c1ccc(-c2cc(-c3ccc(-c4cccc5c4sc4c(-c6ccc(-c7cccnc7)cc6)cccc45)cc3)cc(-c3ccccc3)n2)cc1 ; c1cncc(-c2ccc(-c3cccc4c3sc3c(-c5ccc(-c6cc(-c7ccncc7)cc(-c7ccncc7)c6)cc5)cccc34)cc2)c1]

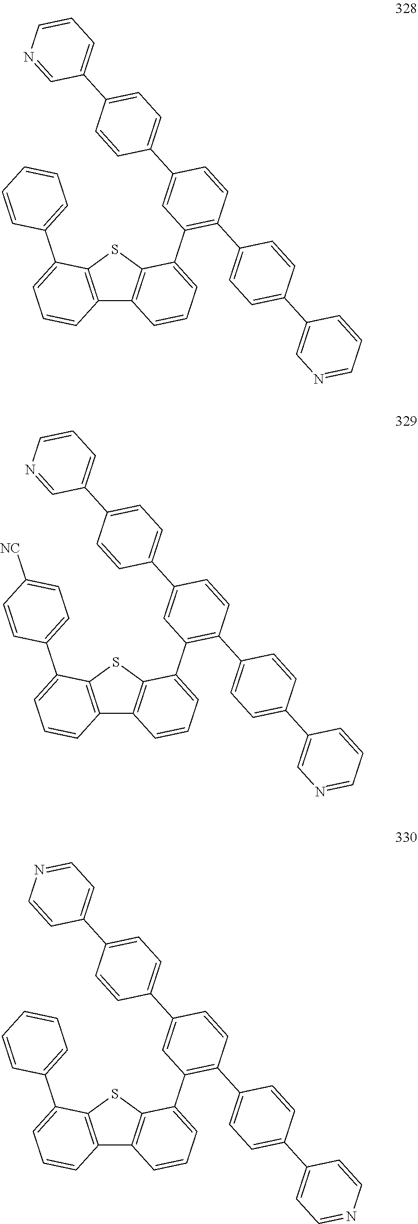 [SMILES: c1ccc(-c2cccc3c2sc2c(-c4cc(-c5ccc(-c6cccnc6)cc5)ccc4-c4ccc(-c5cccnc5)cc4)cccc23)cc1 ; N#Cc1ccc(-c2cccc3c2sc2c(-c4cc(-c5ccc(-c6cccnc6)cc5)ccc4-c4ccc(-c5cccnc5)cc4)cccc23)cc1 ; c1ccc(-c2cccc3c2sc2c(-c4cc(-c5ccc(-c6ccncc6)cc5)ccc4-c4ccc(-c5ccncc5)cc4)cccc23)cc1]

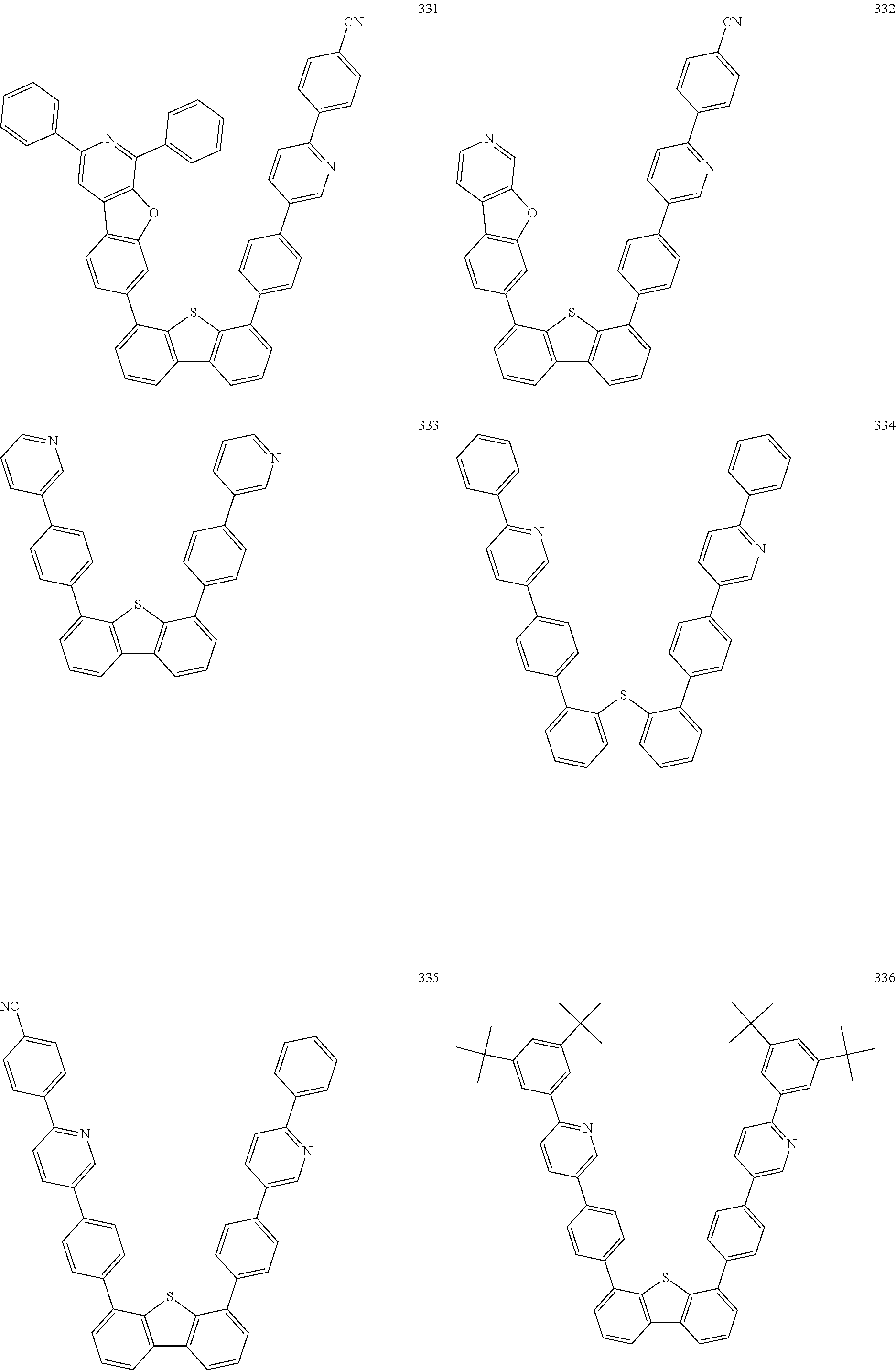

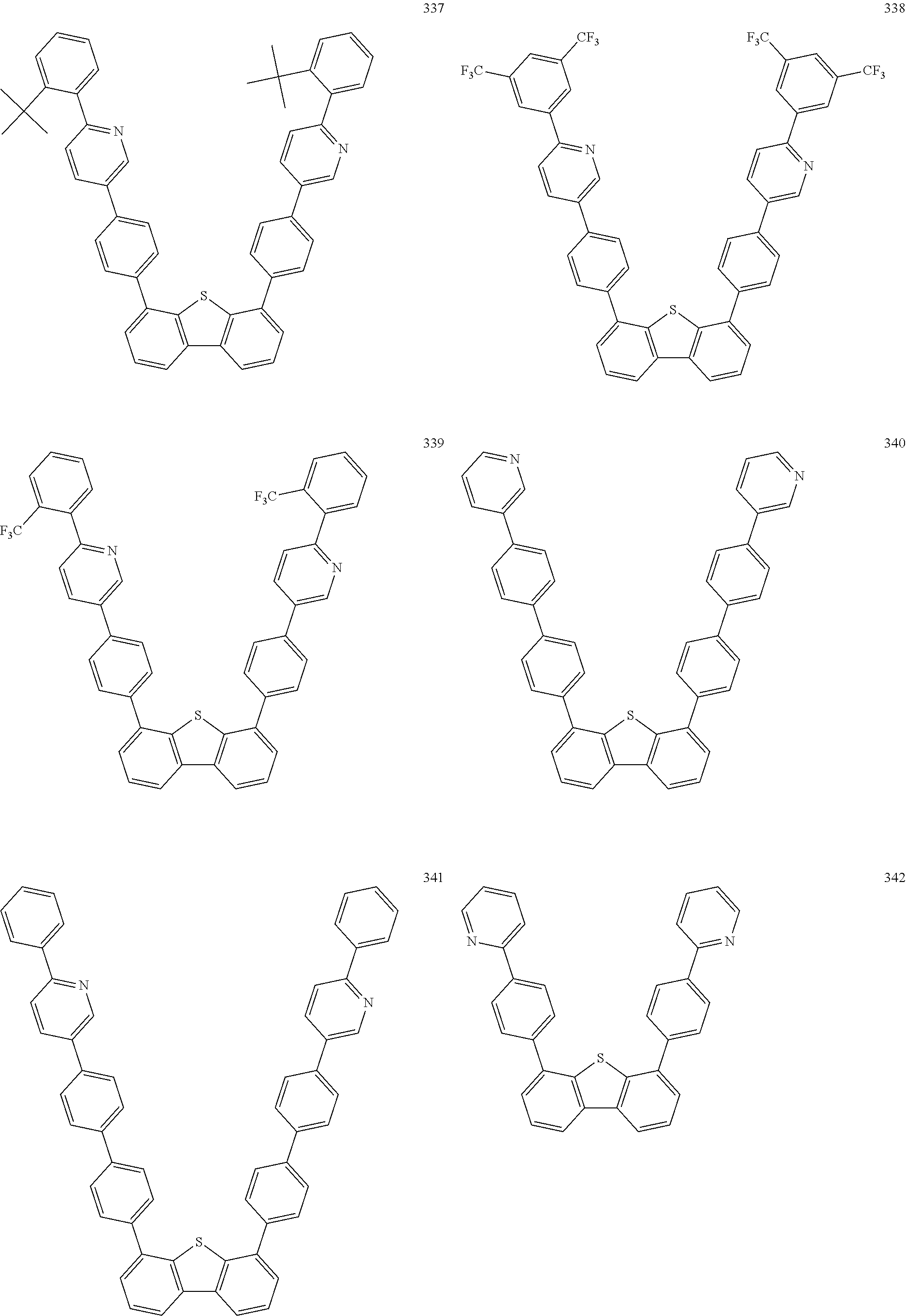

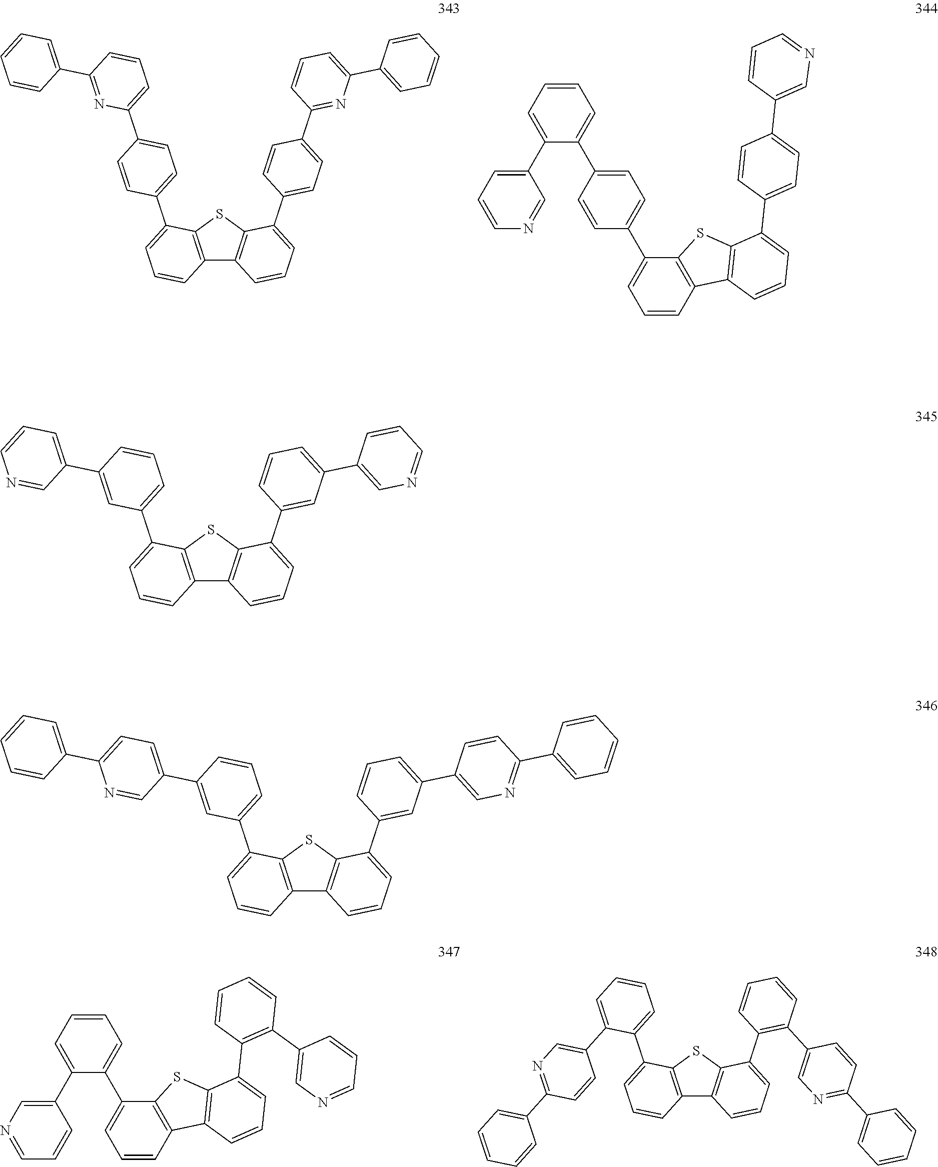 [SMILES: c1ccc(-c2cccc(-c3ccc(-c4cccc5c4sc4c(-c6ccc(-c7cccc(-c8ccccc8)n7)cc6)cccc45)cc3)n2)cc1 ; c1cncc(-c2ccc(-c3cccc4c3sc3c(-c5ccc(-c6ccccc6-c6cccnc6)cc5)cccc34)cc2)c1 ; c1cncc(-c2cccc(-c3cccc4c3sc3c(-c5cccc(-c6cccnc6)c5)cccc34)c2)c1 ; c1ccc(-c2ccc(-c3cccc(-c4cccc5c4sc4c(-c6cccc(-c7ccc(-c8ccccc8)nc7)c6)cccc45)c3)cn2)cc1 ; c1cncc(-c2ccccc2-c2cccc3c2sc2c(-c4ccccc4-c4cccnc4)cccc23)c1 ; c1ccc(-c2ccc(-c3ccccc3-c3cccc4c3sc3c(-c5ccccc5-c5ccc(-c6ccccc6)nc5)cccc34)cn2)cc1]

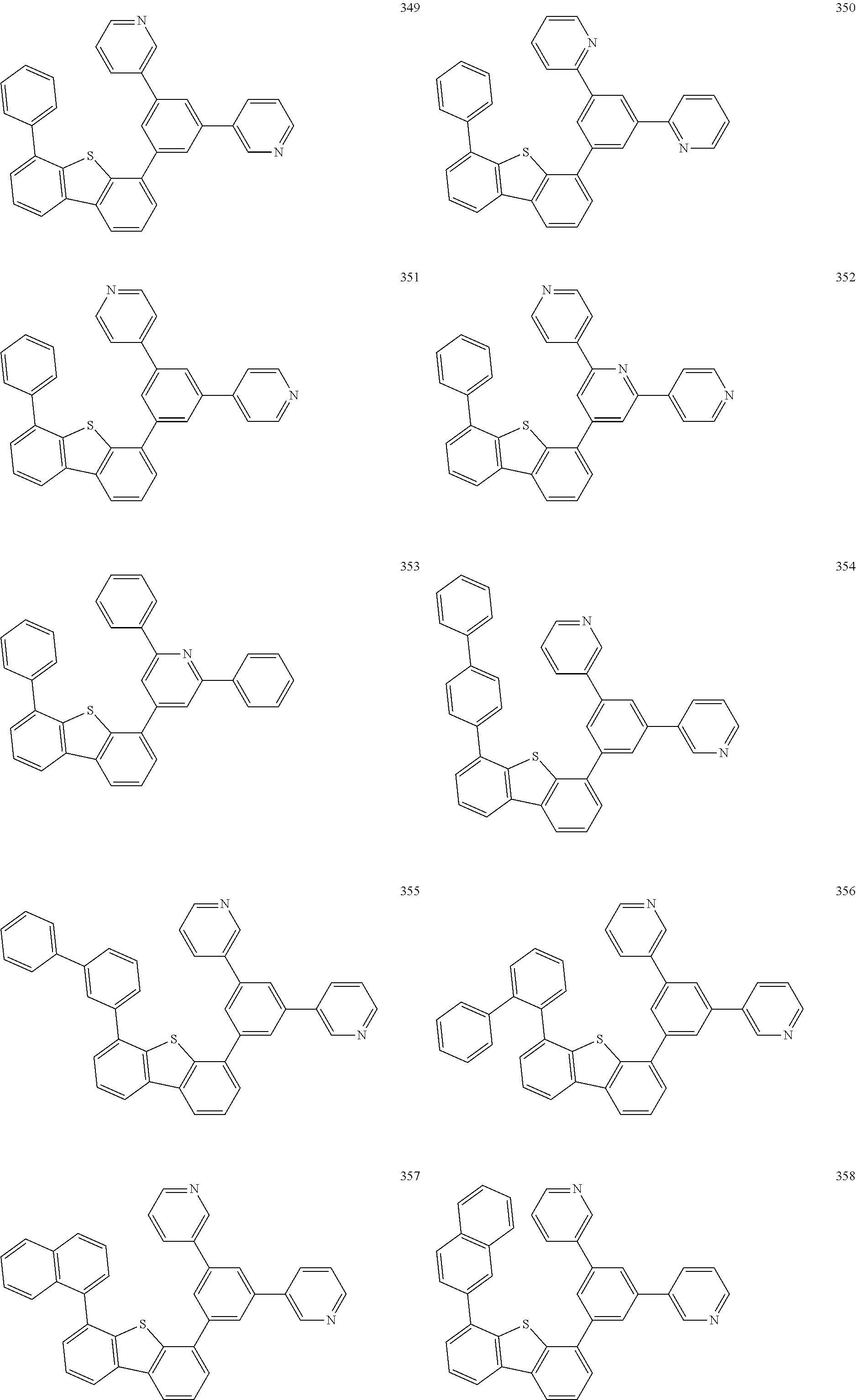

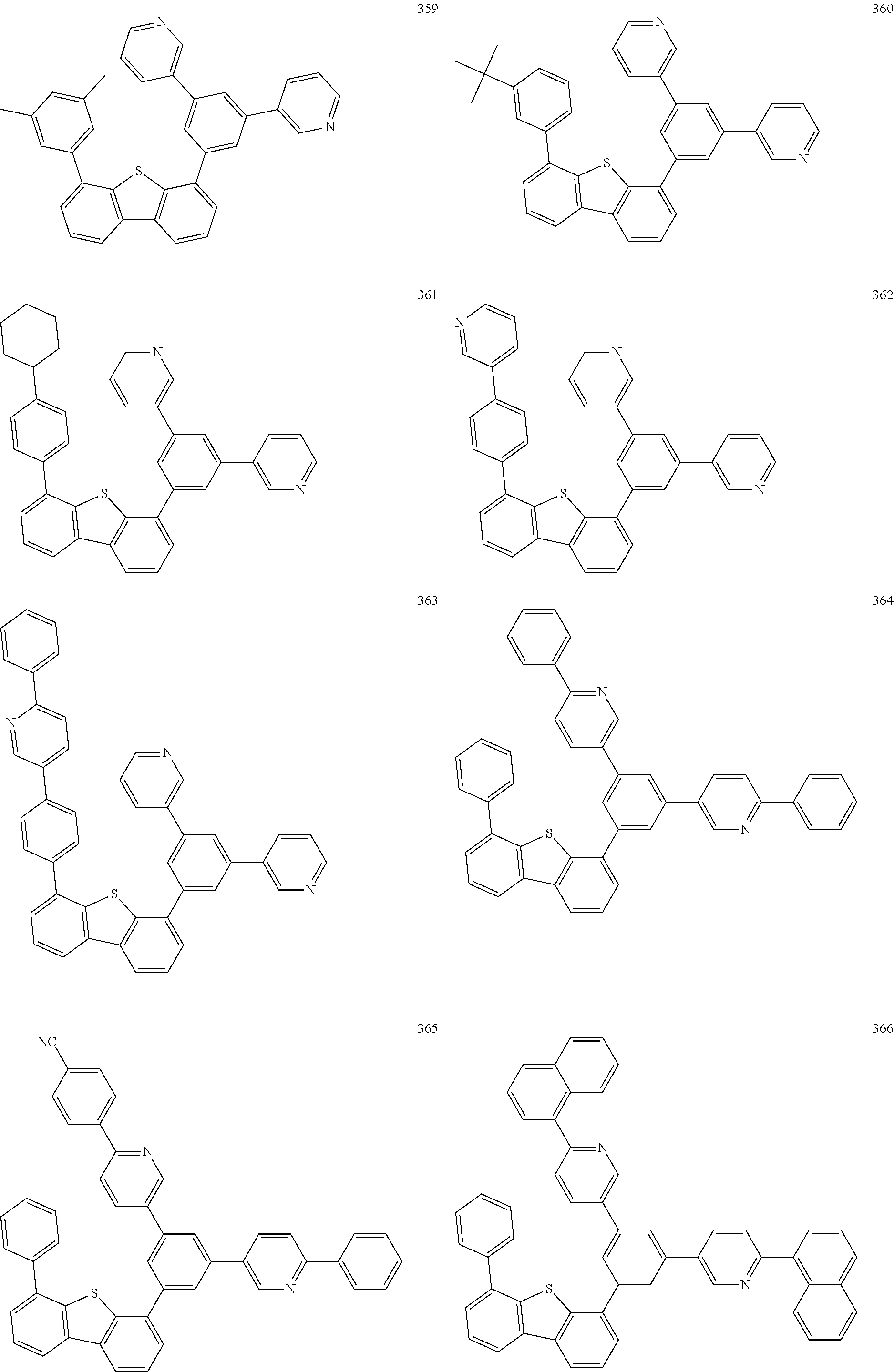

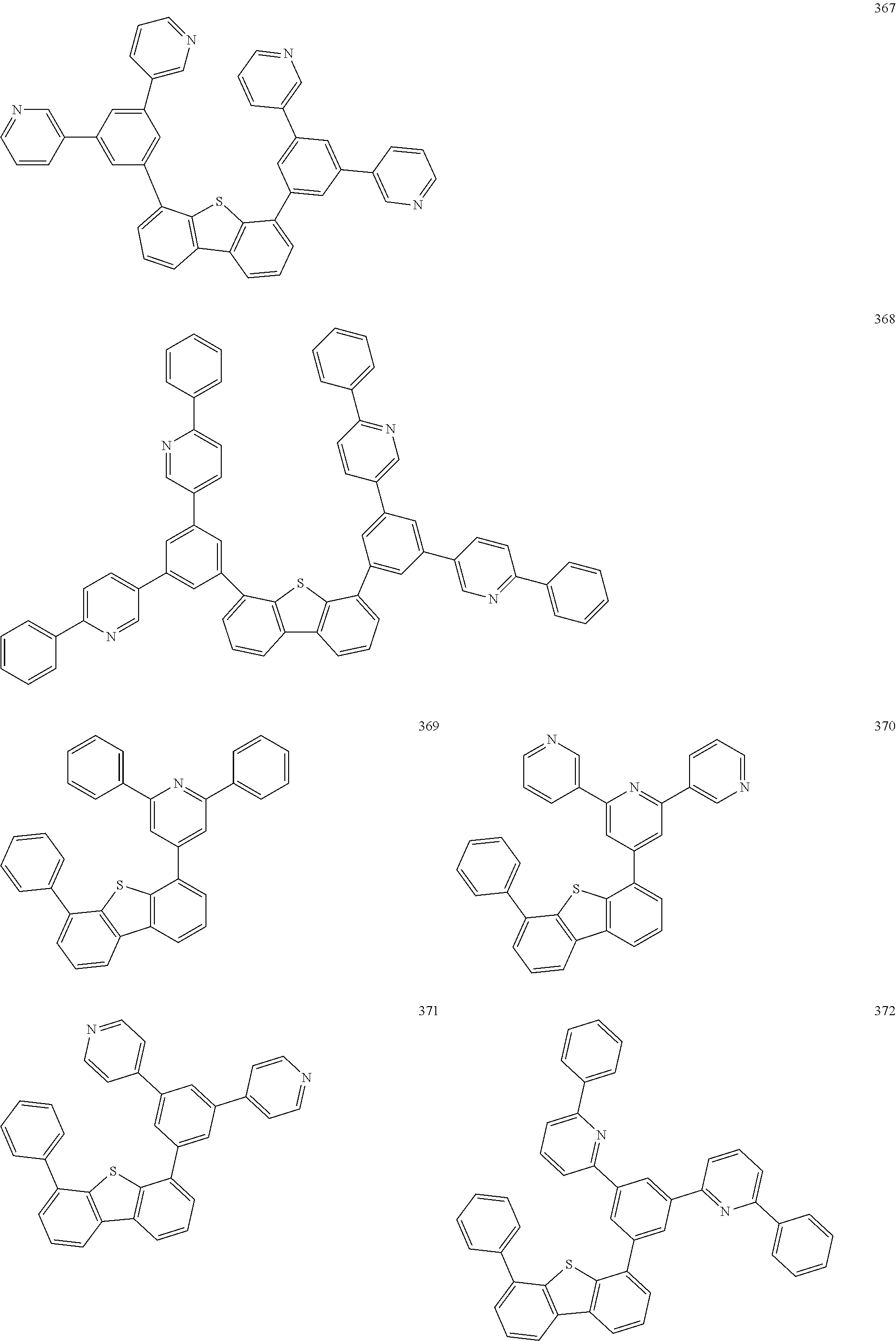

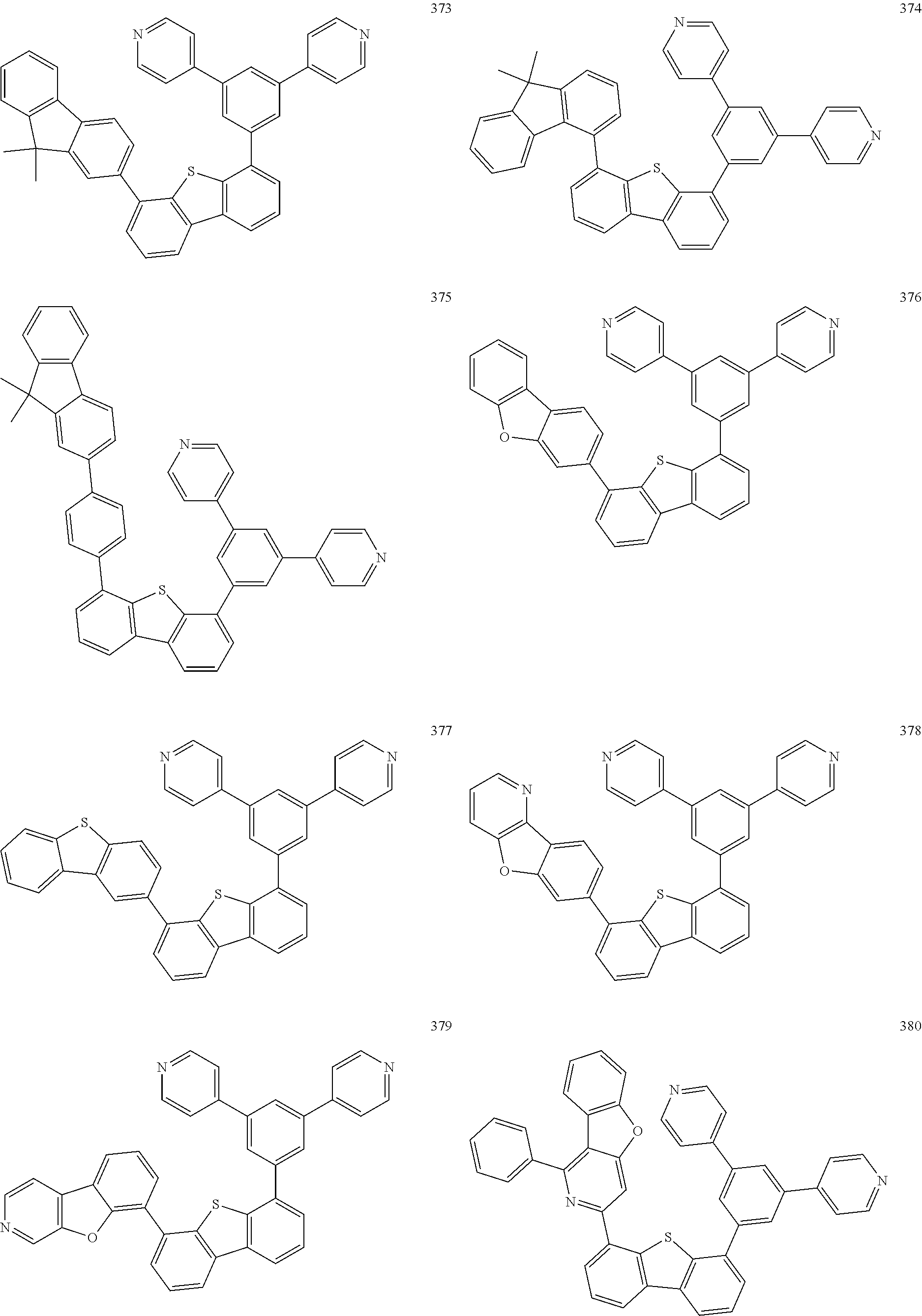

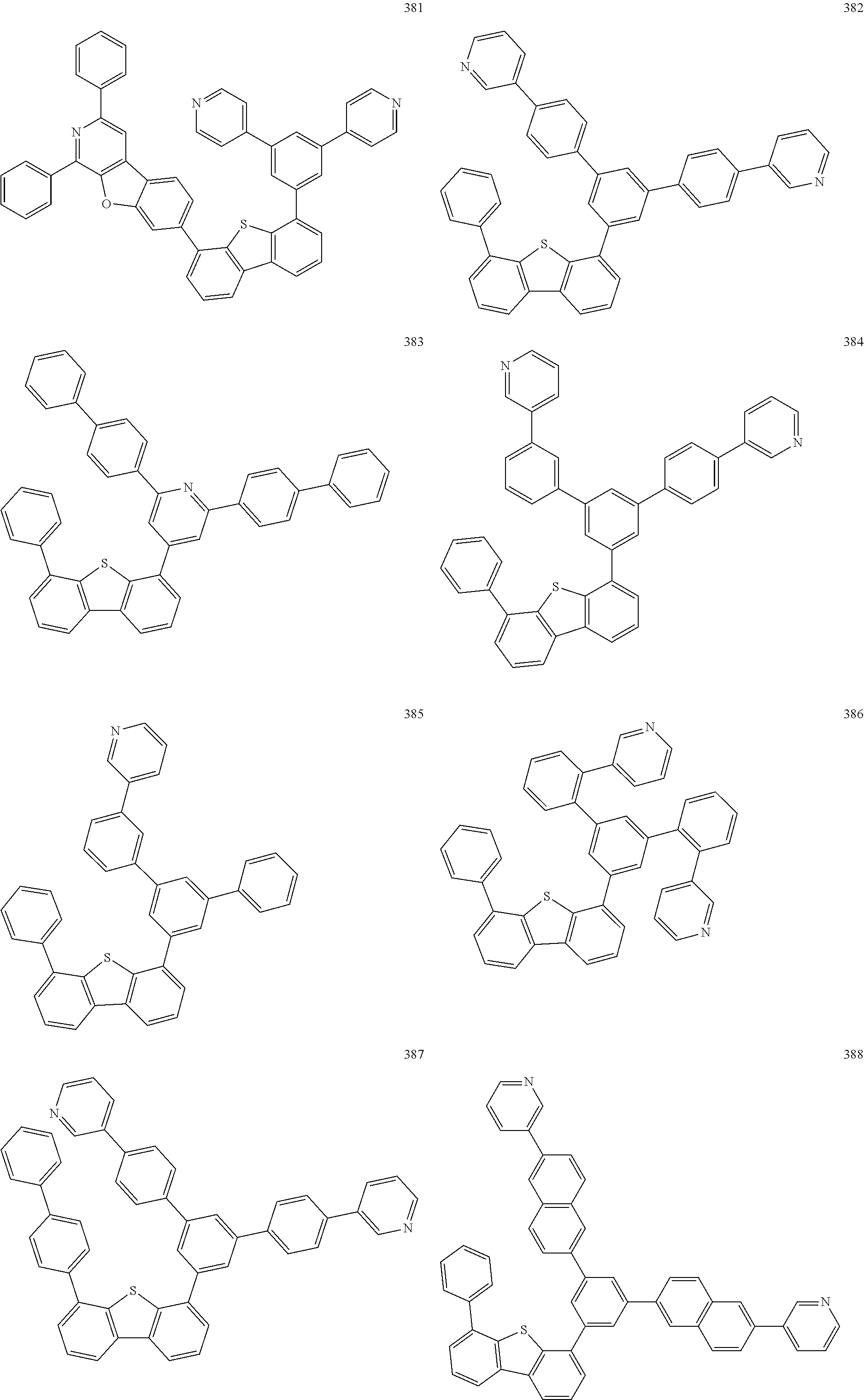

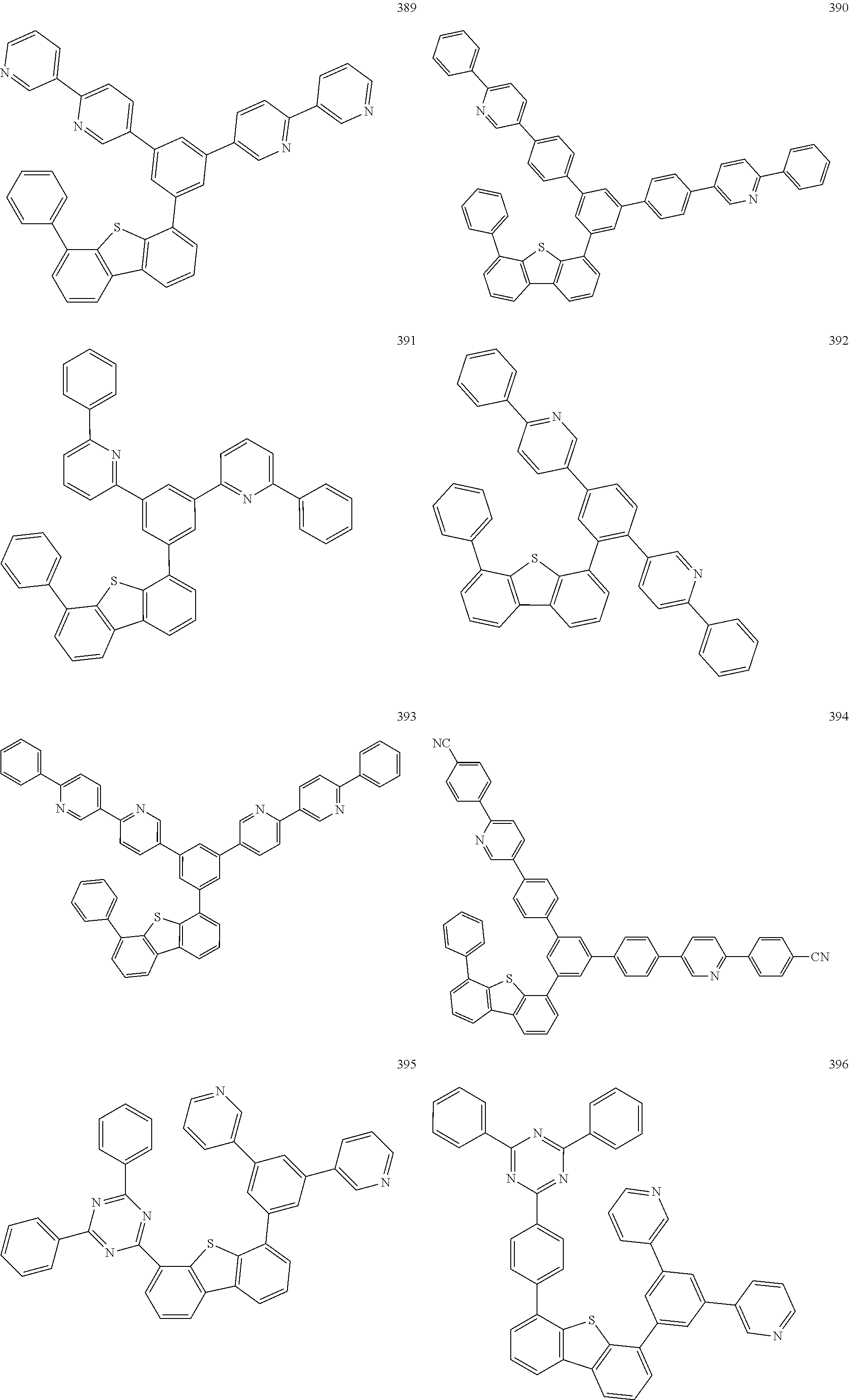

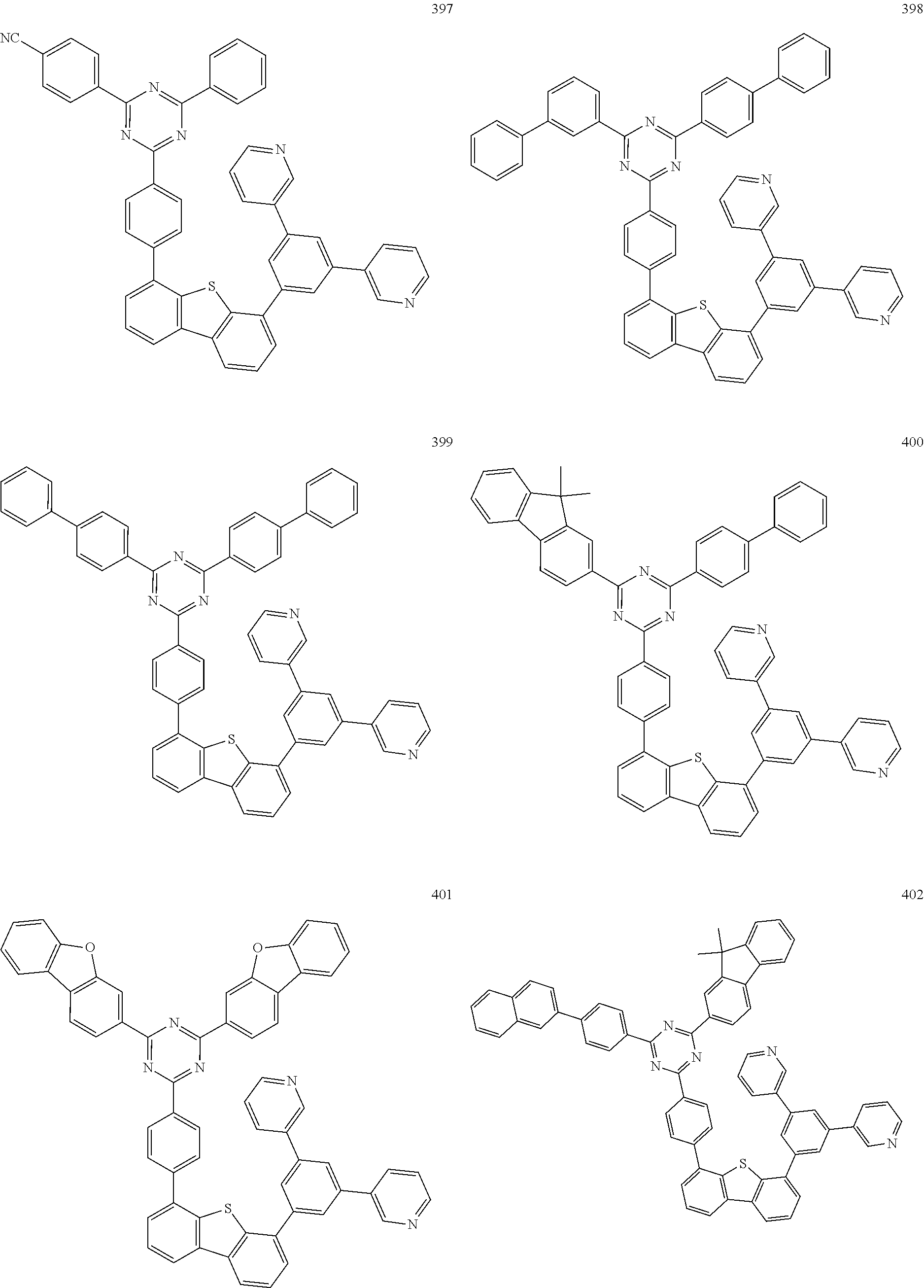

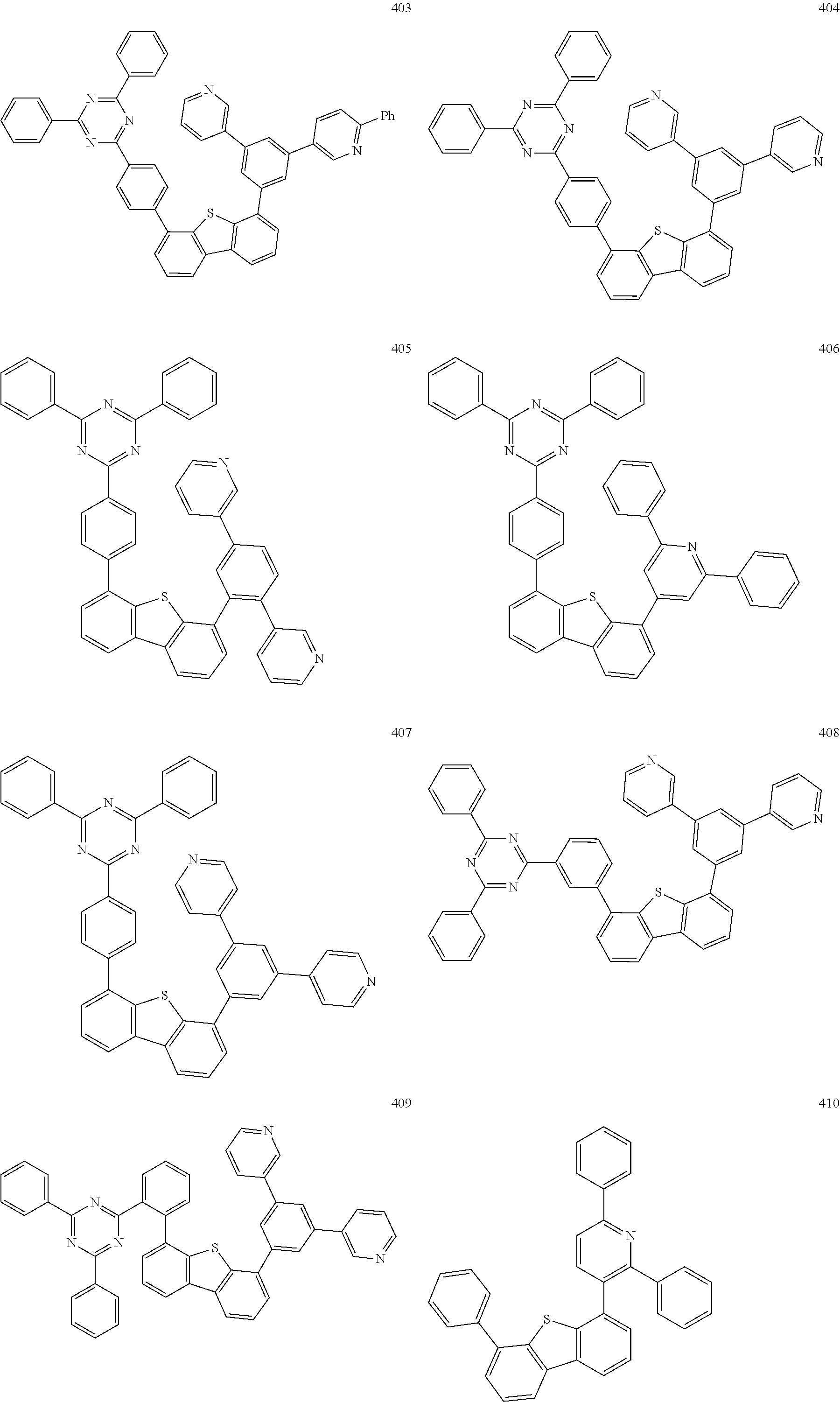

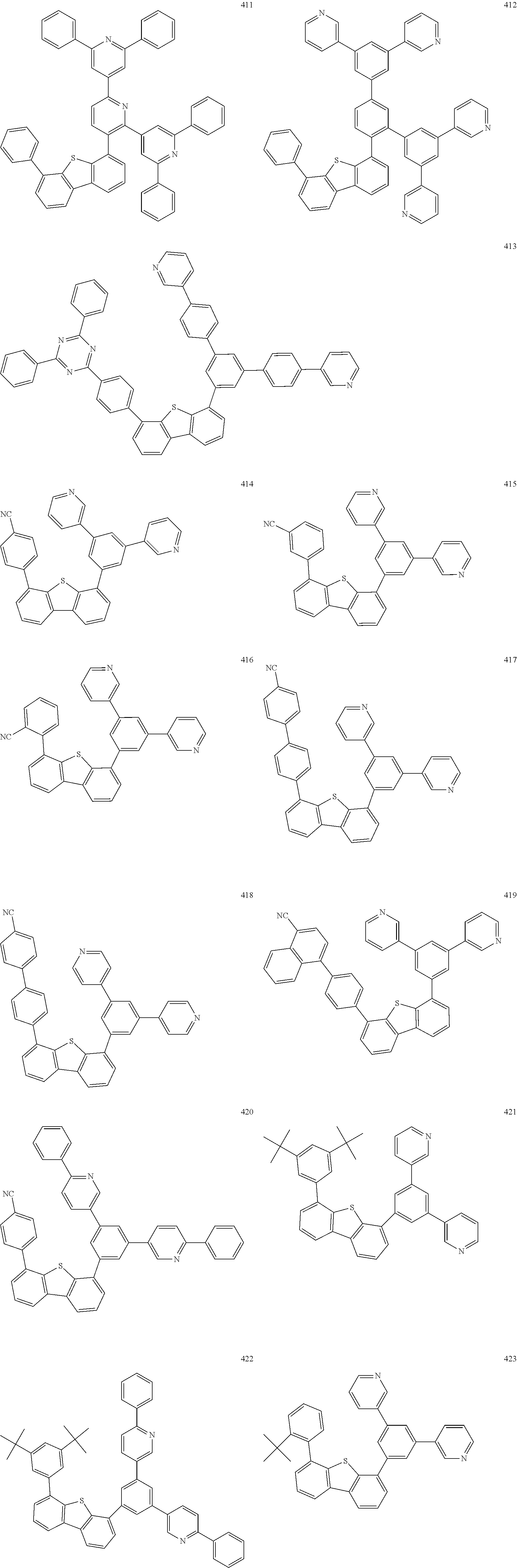

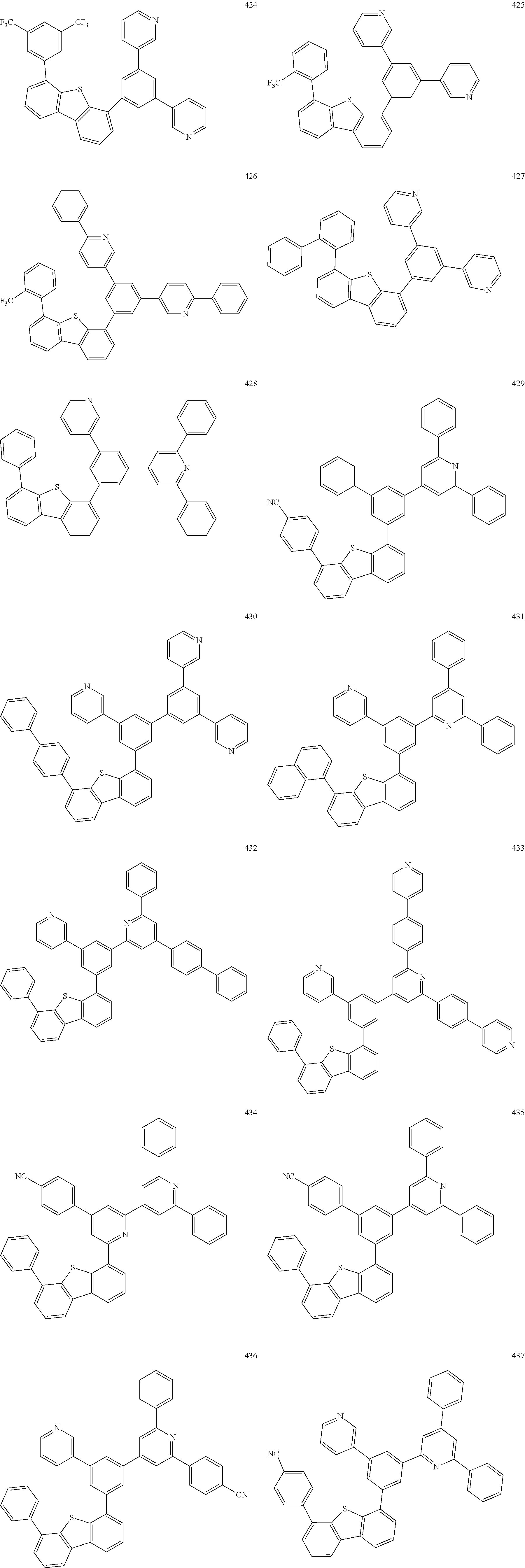

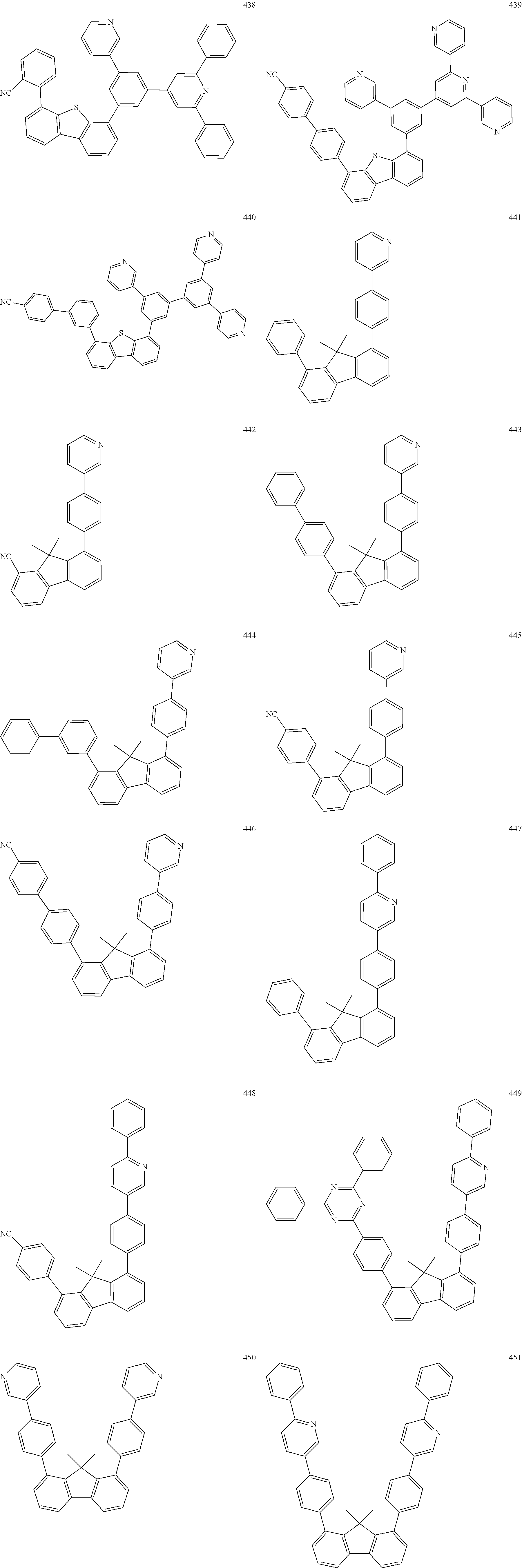

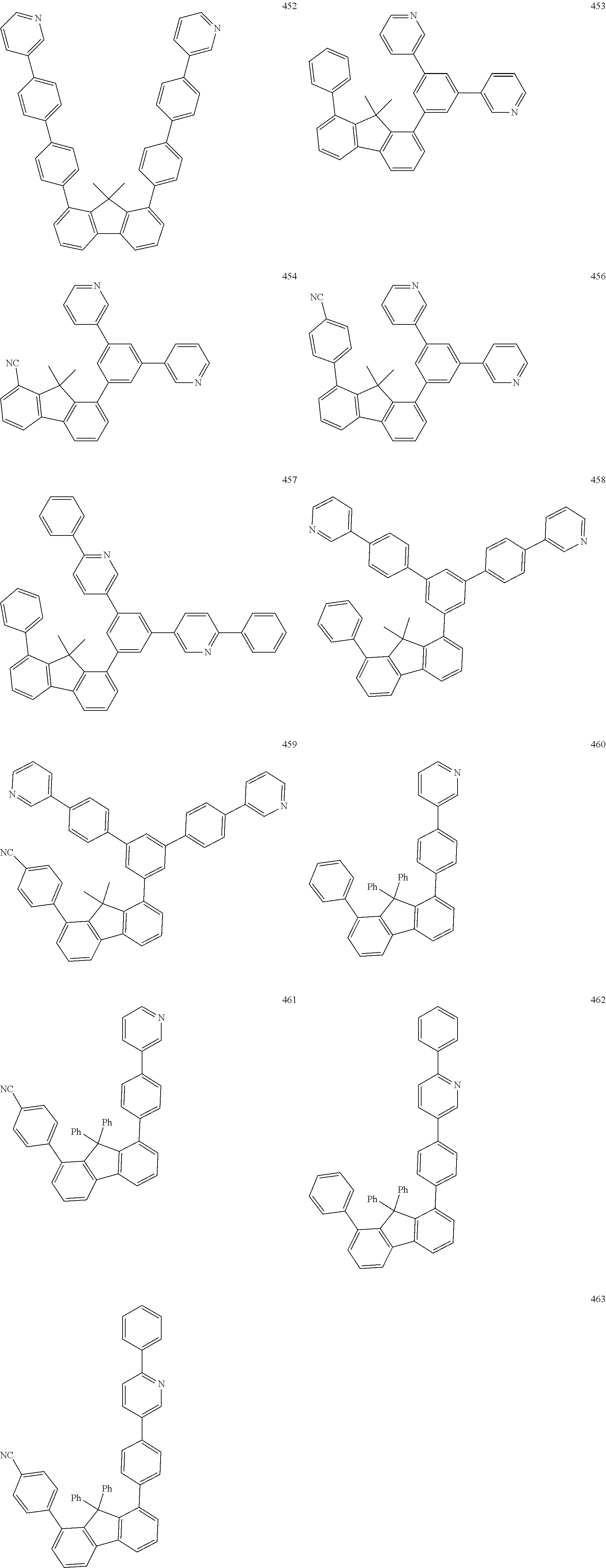

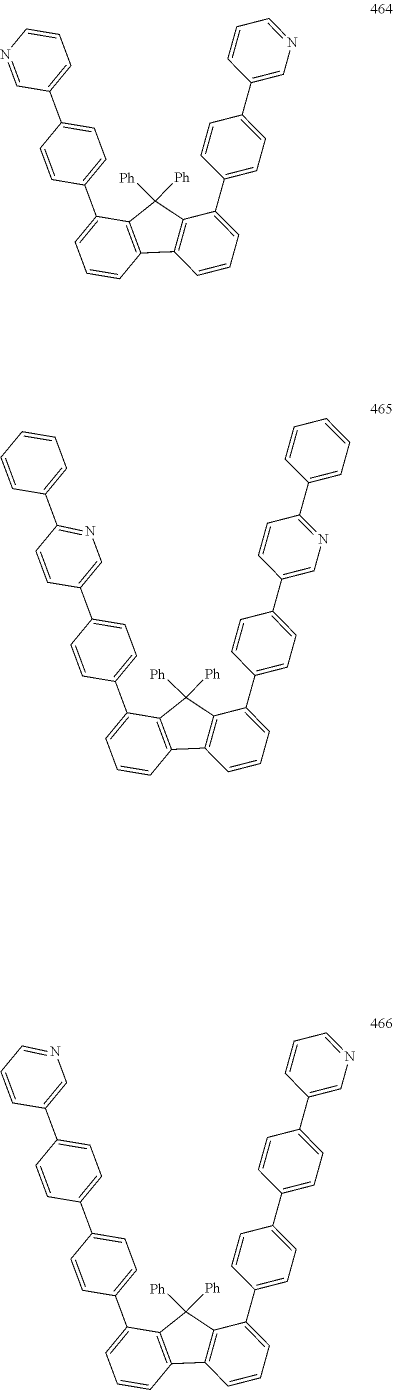 [SMILES: c1ccc(C2(c3ccccc3)c3c(-c4ccc(-c5cccnc5)cc4)cccc3-c3cccc(-c4ccc(-c5cccnc5)cc4)c32)cc1 ; c1ccc(-c2ccc(-c3ccc(-c4cccc5c4C(c4ccccc4)(c4ccccc4)c4c(-c6ccc(-c7ccc(-c8ccccc8)nc7)cc6)cccc4-5)cc3)cn2)cc1 ; c1ccc(C2(c3ccccc3)c3c(-c4ccc(-c5ccc(-c6cccnc6)cc5)cc4)cccc3-c3cccc(-c4ccc(-c5ccc(-c6cccnc6)cc5)cc4)c32)cc1]

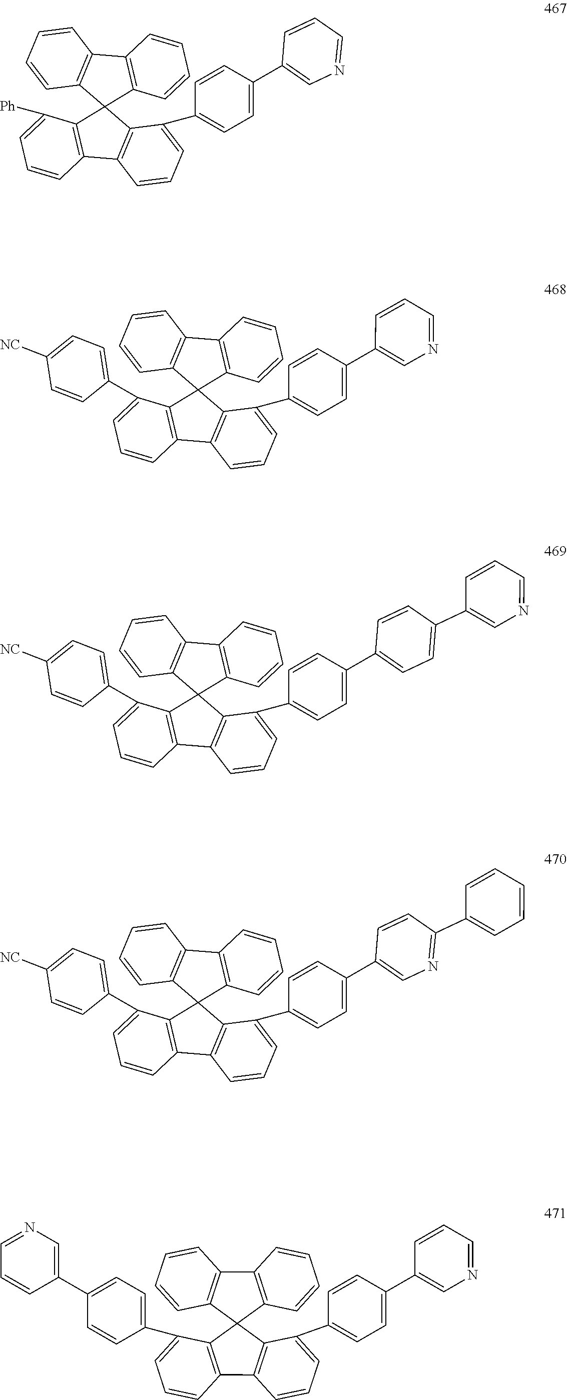 [SMILES: c1ccc(-c2cccc3c2C2(c4ccccc4-c4ccccc42)c2c(-c4ccc(-c5cccnc5)cc4)cccc2-3)cc1 ; N#Cc1ccc(-c2cccc3c2C2(c4ccccc4-c4ccccc42)c2c(-c4ccc(-c5cccnc5)cc4)cccc2-3)cc1 ; N#Cc1ccc(-c2cccc3c2C2(c4ccccc4-c4ccccc42)c2c(-c4ccc(-c5ccc(-c6cccnc6)cc5)cc4)cccc2-3)cc1 ; N#Cc1ccc(-c2cccc3c2C2(c4ccccc4-c4ccccc42)c2c(-c4ccc(-c5ccc(-c6ccccc6)nc5)cc4)cccc2-3)cc1 ; c1cncc(-c2ccc(-c3cccc4c3C3(c5ccccc5-c5ccccc53)c3c(-c5ccc(-c6cccnc6)cc5)cccc3-4)cc2)c1]

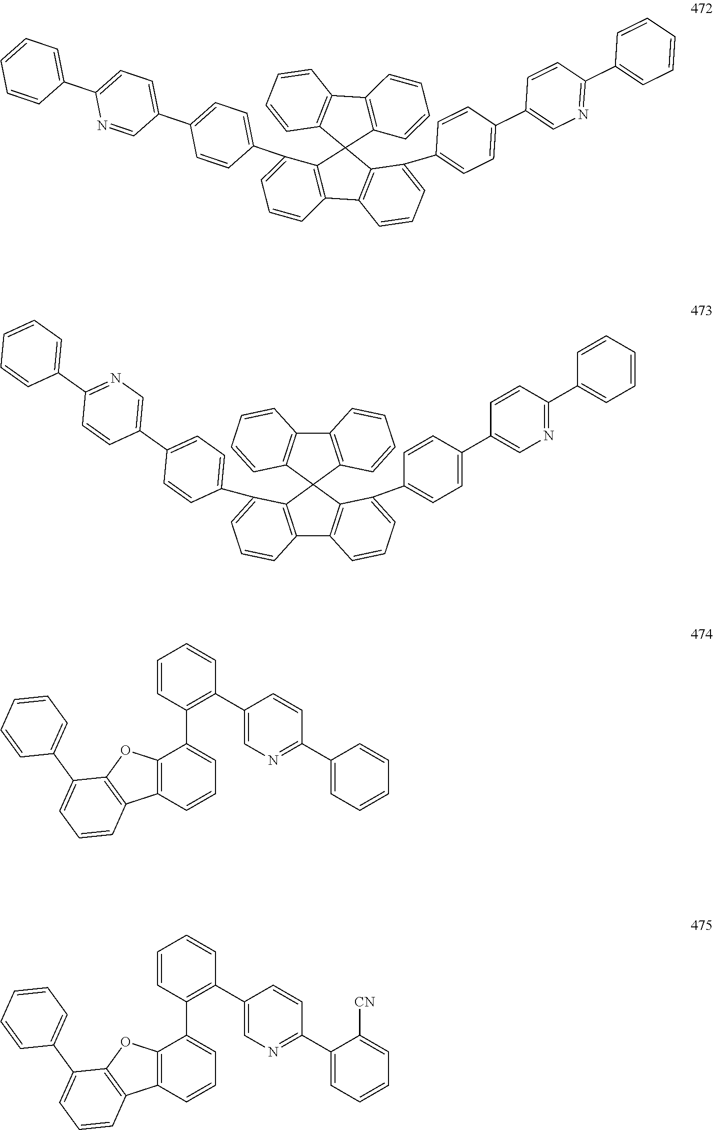 [SMILES: c1ccc(-c2ccc(-c3ccc(-c4cccc5c4C4(c6ccccc6-c6ccccc64)c4c(-c6ccc(-c7ccc(-c8ccccc8)nc7)cc6)cccc4-5)cc3)cn2)cc1 ; c1ccc(-c2ccc(-c3ccc(-c4cccc5c4C4(c6ccccc6-c6ccccc64)c4c(-c6ccc(-c7ccc(-c8ccccc8)nc7)cc6)cccc4-5)cc3)cn2)cc1 ; c1ccc(-c2ccc(-c3ccccc3-c3cccc4c3oc3c(-c5ccccc5)cccc34)cn2)cc1 ; N#Cc1ccccc1-c1ccc(-c2ccccc2-c2cccc3c2oc2c(-c4ccccc4)cccc23)cn1]

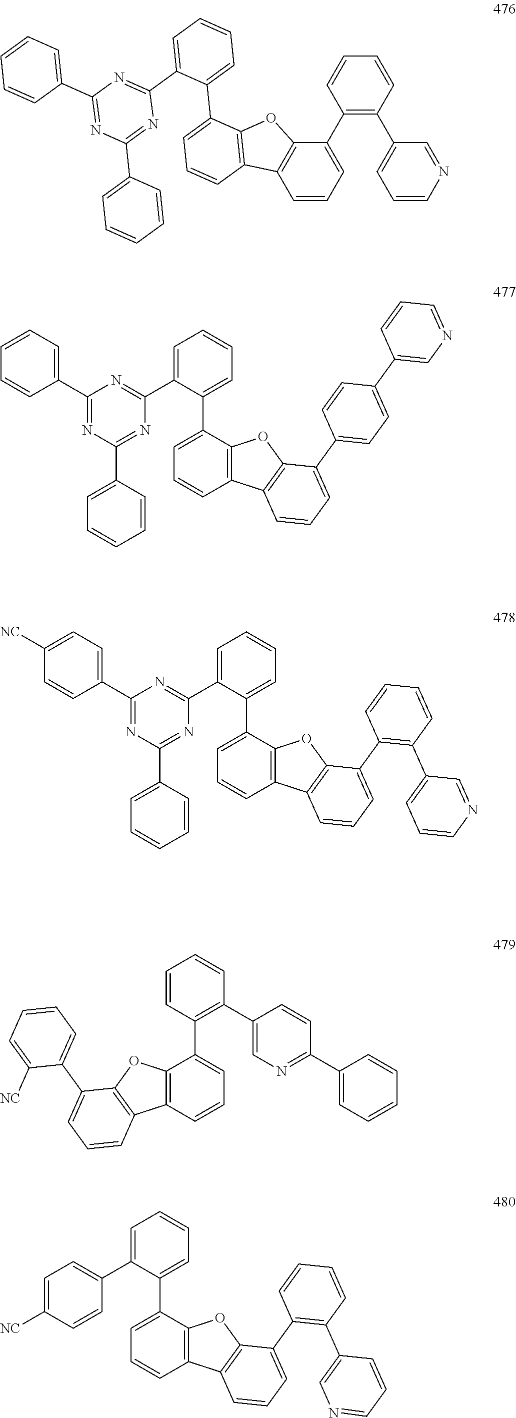 [SMILES: c1ccc(-c2nc(-c3ccccc3)nc(-c3ccccc3-c3cccc4c3oc3c(-c5ccccc5-c5cccnc5)cccc34)n2)cc1 ; c1ccc(-c2nc(-c3ccccc3)nc(-c3ccccc3-c3cccc4c3oc3c(-c5ccc(-c6cccnc6)cc5)cccc34)n2)cc1 ; N#Cc1ccc(-c2nc(-c3ccccc3)nc(-c3ccccc3-c3cccc4c3oc3c(-c5ccccc5-c5cccnc5)cccc34)n2)cc1 ; N#Cc1ccccc1-c1cccc2c1oc1c(-c3ccccc3-c3ccc(-c4ccccc4)nc3)cccc12 ; N#Cc1ccc(-c2ccccc2-c2cccc3c2oc2c(-c4ccccc4-c4cccnc4)cccc23)cc1]

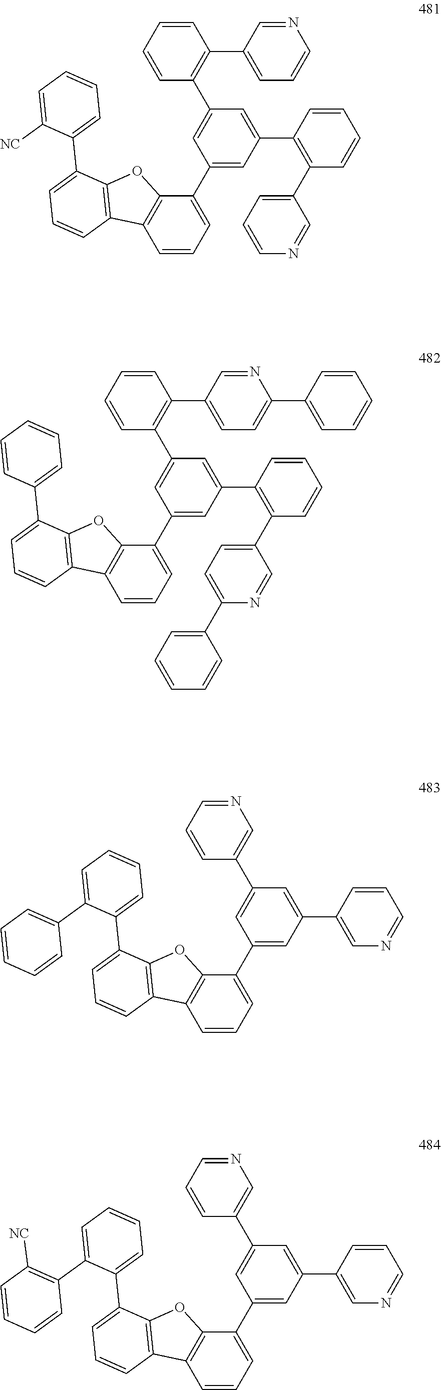 [SMILES: N#Cc1ccccc1-c1cccc2c1oc1c(-c3cc(-c4ccccc4-c4cccnc4)cc(-c4ccccc4-c4cccnc4)c3)cccc12 ; c1ccc(-c2ccc(-c3ccccc3-c3cc(-c4ccccc4-c4ccc(-c5ccccc5)nc4)cc(-c4cccc5c4oc4c(-c6ccccc6)cccc45)c3)cn2)cc1 ; c1ccc(-c2ccccc2-c2cccc3c2oc2c(-c4cc(-c5cccnc5)cc(-c5cccnc5)c4)cccc23)cc1 ; N#Cc1ccccc1-c1ccccc1-c1cccc2c1oc1c(-c3cc(-c4cccnc4)cc(-c4cccnc4)c3)cccc12]

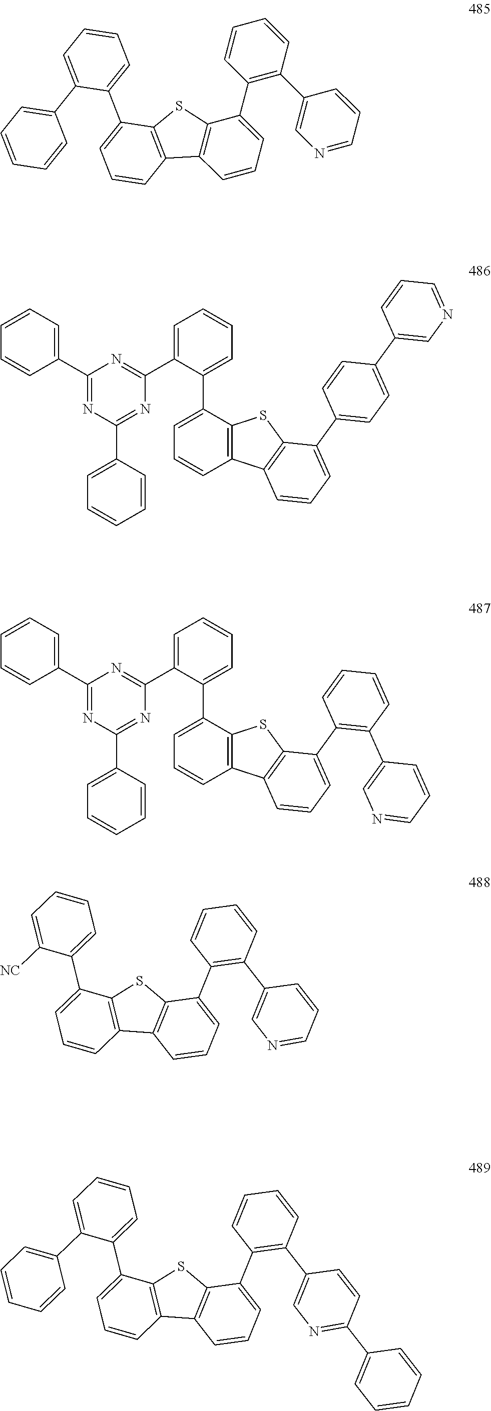 [SMILES: c1ccc(-c2ccccc2-c2cccc3c2sc2c(-c4ccccc4-c4cccnc4)cccc23)cc1 ; c1ccc(-c2nc(-c3ccccc3)nc(-c3ccccc3-c3cccc4c3sc3c(-c5ccc(-c6cccnc6)cc5)cccc34)n2)cc1 ; c1ccc(-c2nc(-c3ccccc3)nc(-c3ccccc3-c3cccc4c3sc3c(-c5ccccc5-c5cccnc5)cccc34)n2)cc1 ; N#Cc1ccccc1-c1cccc2c1sc1c(-c3ccccc3-c3cccnc3)cccc12 ; c1ccc(-c2ccc(-c3ccccc3-c3cccc4c3sc3c(-c5ccccc5-c5ccccc5)cccc34)cn2)cc1]

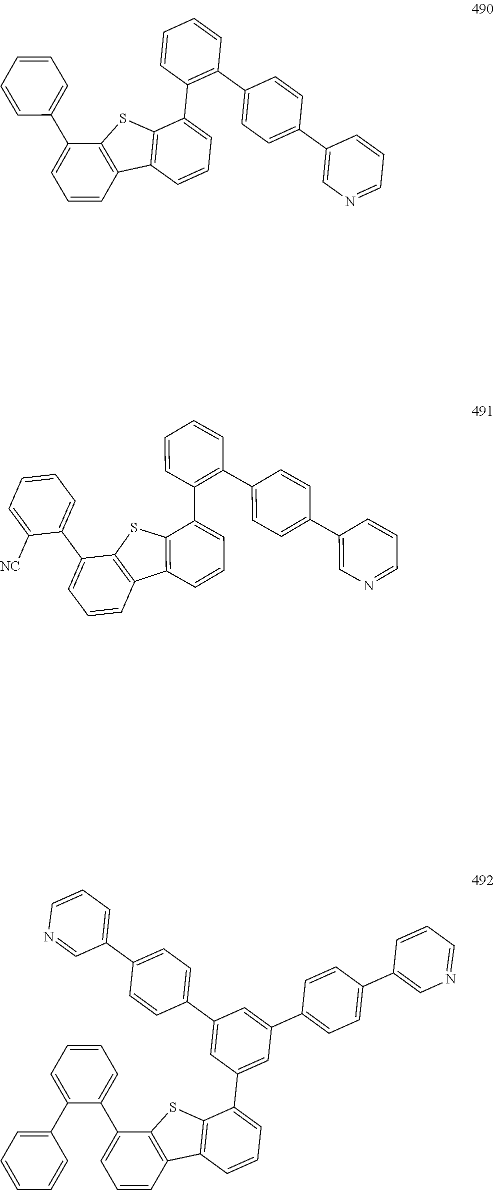 [SMILES: c1ccc(-c2cccc3c2sc2c(-c4ccccc4-c4ccc(-c5cccnc5)cc4)cccc23)cc1 ; N#Cc1ccccc1-c1cccc2c1sc1c(-c3ccccc3-c3ccc(-c4cccnc4)cc3)cccc12 ; c1ccc(-c2ccccc2-c2cccc3c2sc2c(-c4cc(-c5ccc(-c6cccnc6)cc5)cc(-c5ccc(-c6cccnc6)cc5)c4)cccc23)cc1]

As such, the organic compound according to an embodiment of the present invention may synthesize an organic compound having various characteristics using a characteristic skeleton exhibiting inherent characteristics and moieties having inherent characteristics introduced into the skeleton. As a result, the organic compound according to an embodiment of the present invention may be employed as materials for various organic layers, such as a light emitting layer, a hole transport layer, an electron transport layer, an electron blocking layer, and a hole blocking layer, achieving further improved luminescent properties such as a luminous efficiency of the device as an electron transport material.

In addition, the compound of an embodiment of the present invention may be applied to a device according to a general method for manufacturing an organic light emitting device.

An organic light emitting device according to an embodiment of the present invention may include a first electrode, a second electrode, and an organic layer arranged therebetween. The organic light emitting device may be manufactured using a general device manufacturing method and material, except that the organic compound of an embodiment of the present invention is used to form the organic layer of the device.

The organic layer of the organic light emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

Preferred structures of the organic layers of the organic light emitting according to an embodiment of the present invention will be explained in more detail in the examples to be described later.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light emitting device may be fabricated by depositing a cathode material, organic layer materials, and an anode material in this order on a substrate. The organic layers may have a multilayer structure including a hole injecting layer, a hole transport layer, a light emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light emitting device.

### MODE FOR CARRYING OUT THE DISCLOSURE

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 1

### (1) Preparative Example 1: Synthesis of Compound 1

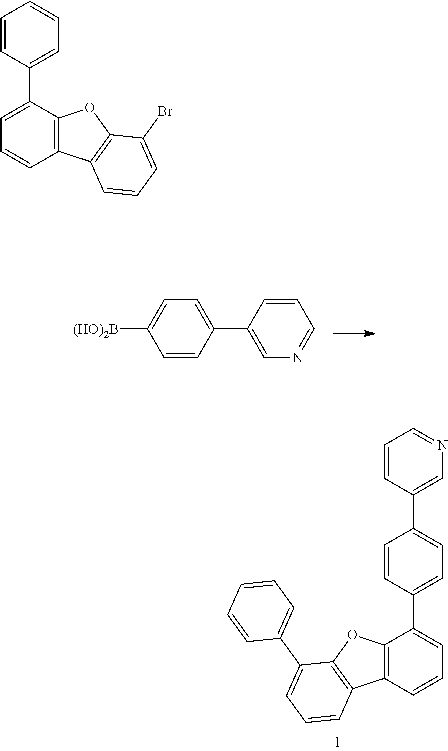 [SMILES: Brc1cccc2c1oc1c(-c3ccccc3)cccc12 ; OB(O)c1ccc(-c2cccnc2)cc1 ; c1ccc(-c2cccc3c2oc2c(-c4ccc(-c5cccnc5)cc4)cccc23)cc1]

Toluene 140 mL, EtOH 35 mL, and H₂O 35 mL were added to 4-Bromo-6-phenyldibenzofuran (10.0 g, 0.031 mol), 4-(Pyridin-3-yl)phenylboronic acid (7.4 g, 0.037 mol), K₂CO₃ (12.8 g, 0.093 mol), Pd(PPh₃)₄ (0.7 g, 0.0006 mol), and then were reacted with stirring at 100° ° C. for 6 hours. After completion of the reaction, the reaction mixture was extracted, concentrated, and then subjected to column and recrystallization to obtain 9.1 g (yield 74.0%) of Compound 1.

LC/MS: m/z=397[(M+1)⁺]

### Synthesis Example 2: Synthesis of Compound 10

### (1) Preparative Example 1: Synthesis of Intermediate 10-1

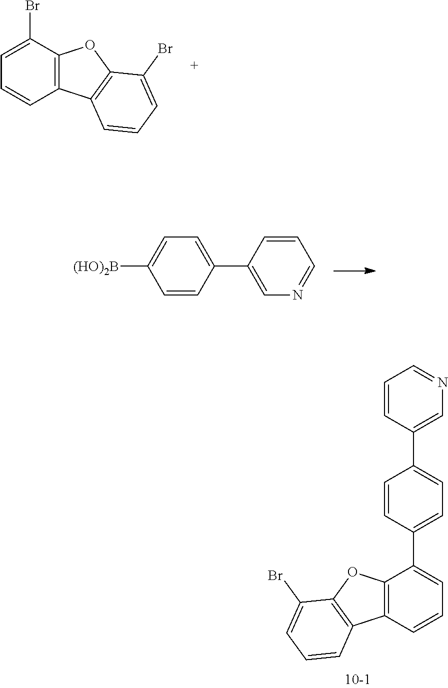 [SMILES: Brc1cccc2c1oc1c(Br)cccc12 ; OB(O)c1ccc(-c2cccnc2)cc1 ; Brc1cccc2c1oc1c(-c3ccc(-c4cccnc4)cc3)cccc12]

Toluene 140 mL, EtOH 35 mL, and H₂O 35 mL were added to 4,6-Dibromodibenzofuran (10.0 g, 0.031 mol), 4-(Pyridin-3-yl)phenylboronic acid (7.3 g, 0.037 mol), K₂CO₃ (12.8 g, 0.093 mol), Pd(PPh₃)₄ (0.7 g, 0.0006 mol), and then were reacted with stirring at 100° ° C. for 6 hours. After completion of the reaction, the reaction mixture was extracted, concentrated, and then subjected to column and recrystallization to obtain 6.3 g (yield 51.3%) of Intermediate 10-1.

### (2) Preparative Example 2: Synthesis of Compound 10

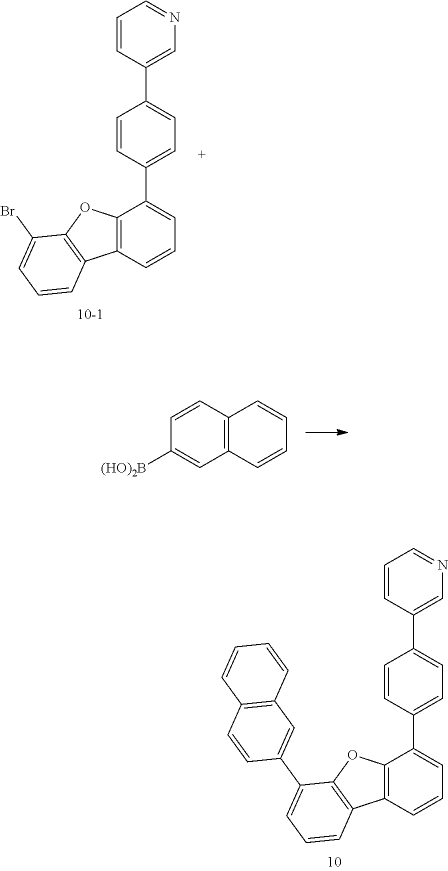 [SMILES: Brc1cccc2c1oc1c(-c3ccc(-c4cccnc4)cc3)cccc12 ; OB(O)c1ccc2ccccc2c1 ; c1cncc(-c2ccc(-c3cccc4c3oc3c(-c5ccc6ccccc6c5)cccc34)cc2)c1]

Toluene 200 mL, EtOH 50 mL, and H₂O 50 mL were added to Intermediate 10-1 (10.0 g, 0.025 mol), (2-Naphthalenyl)boronic acid (5.2 g, 0.030 mol), K₂CO₃ (10.4 g, 0.075 mol), Pd(PPh₃)₄ (0.6 g, 0.0005 mol), and then were reacted with stirring at 100° C. for 6 hours. After completion of the reaction, the reaction mixture was extracted, concentrated, and then subjected to column and recrystallization to obtain 8.1 g (yield 72.5%) of Compound 10.

LC/MS: m/z=447[(M+1)⁺]

### Synthesis Example 3: Synthesis of Compound 25

### (1) Preparative Example 1: Synthesis of Compound 25

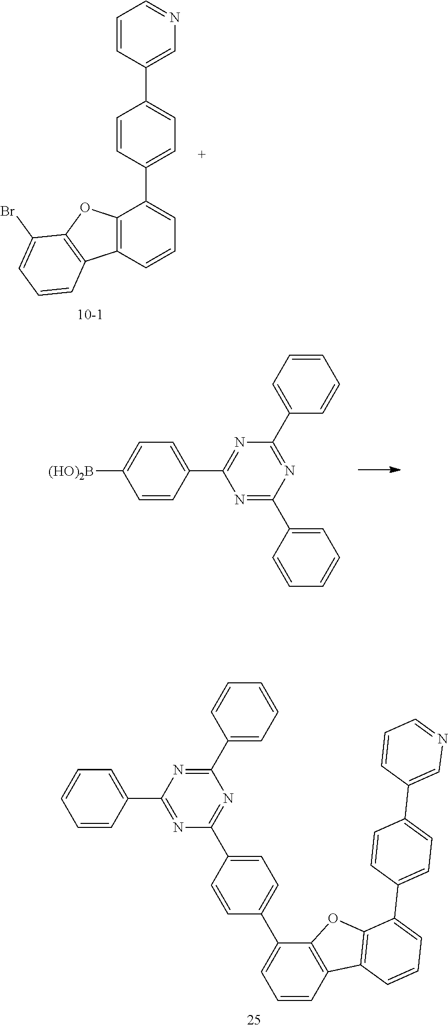 [SMILES: Brc1cccc2c1oc1c(-c3ccc(-c4cccnc4)cc3)cccc12 ; OB(O)c1ccc(-c2nc(-c3ccccc3)nc(-c3ccccc3)n2)cc1 ; c1ccc(-c2nc(-c3ccccc3)nc(-c3ccc(-c4cccc5c4oc4c(-c6ccc(-c7cccnc7)cc6)cccc45)cc3)n2)cc1]

Toluene 200 mL, EtOH 50 mL, and H₂O 50 mL were added to Intermediate 10-1 (10.0 g, 0.025 mol), 4-(4,6-Diphenyl-1,3,5-triazin-2-yl)phenylboronic acid (10.6 g, 0.030 mol), K₂CO₃ (10.4 g, 0.075 mol), Pd(PPh₃)₄ (0.6 g, 0.0005 mol), and then were reacted with stirring at 100° C. for 6 hours. After completion of the reaction, the reaction mixture was extracted, concentrated, and then subjected to column and recrystallization to obtain 10.5 g (yield 66.9%) of Compound 25.

LC/MS: m/z=628[(M+1)⁺]

### Synthesis Example 4: Synthesis of Compound 44

### (1) Preparative Example 1: Synthesis of Compound 44

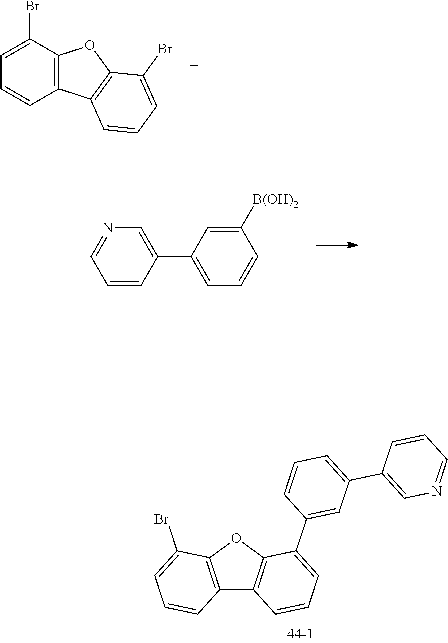 [SMILES: Brc1cccc2c1oc1c(Br)cccc12 ; OB(O)c1cccc(-c2cccnc2)c1 ; Brc1cccc2c1oc1c(-c3cccc(-c4cccnc4)c3)cccc12]

Toluene 200 mL, EtOH 50 mL, and H₂O 50 mL were added to 4,6-Dibromodibenzofuran (10.0 g, 0.031 mol), (3-(Pyridin-3-yl)phenyl)boronic acid (7.3 g, 0.037 mol), K₂CO₃ (12.7 g, 0.092 mol), Pd(PPh₃)₄ (0.7 g, 0.0006 mol), and then were reacted with stirring at 100° C. for 6 hours. After completion of the reaction, the reaction mixture was extracted, concentrated, and then subjected to column and recrystallization to obtain 6.2 g (yield 50.5%) of Intermediate 44-1.

### (2) Preparative Example 2: Synthesis of Compound 44

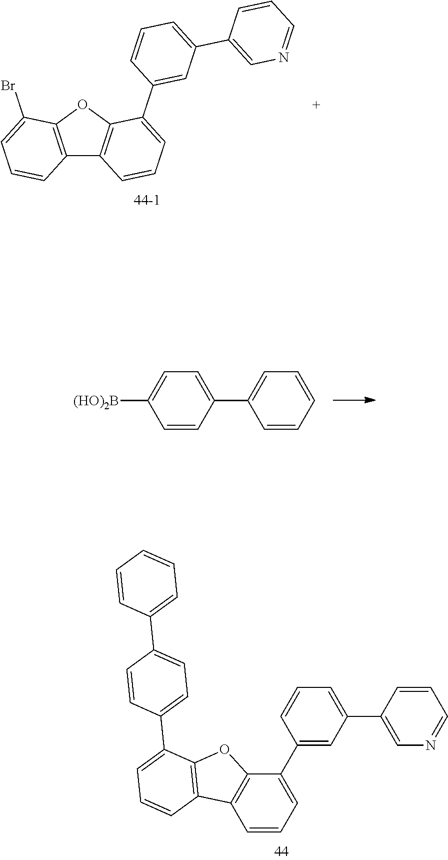 [SMILES: Brc1cccc2c1oc1c(-c3cccc(-c4cccnc4)c3)cccc12 ; OB(O)c1ccc(-c2ccccc2)cc1 ; c1ccc(-c2ccc(-c3cccc4c3oc3c(-c5cccc(-c6cccnc6)c5)cccc34)cc2)cc1]

Toluene 200 mL, EtOH 50 mL, and H₂O 50 mL were added to Intermediate 44-1 (10.0 g, 0.025 mol), (1,1′-Biphenyl)-4-boronic acid (5.9 g, 0.030 mol), K₂CO₃ (10.4 g, 0.075 mol), Pd(PPh₃)₄ (0.6 g, 0.0005 mol), and then were reacted with stirring at 100° C. for 6 hours. After completion of the reaction, the reaction mixture was extracted, concentrated, and then subjected to column and recrystallization to obtain 8.9 g (yield 75.2%) of Compound 44.

LC/MS: m/z=473[(M+1)⁺]

### Synthesis Example 5: Synthesis of Compound 58

### (1) Preparative Example 1: Synthesis of Compound 58

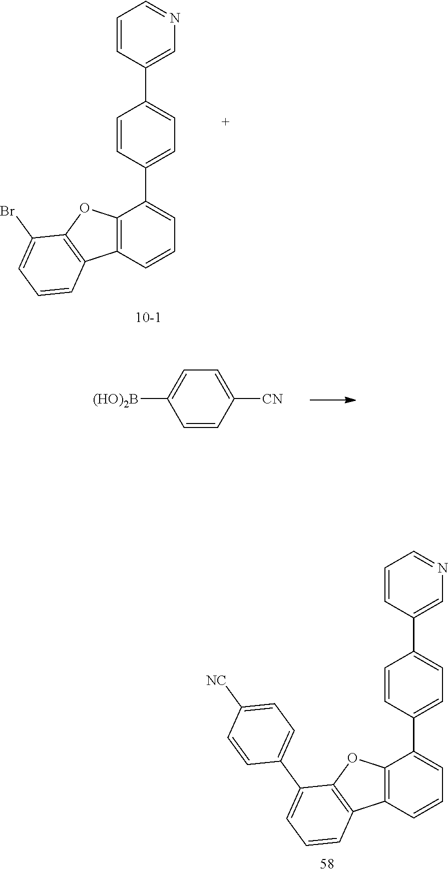 [SMILES: Brc1cccc2c1oc1c(-c3ccc(-c4cccnc4)cc3)cccc12 ; N#Cc1ccc(B(O)O)cc1 ; N#Cc1ccc(-c2cccc3c2oc2c(-c4ccc(-c5cccnc5)cc4)cccc23)cc1]

Toluene 200 mL, EtOH 50 mL, and H₂O 50 mL were added to Intermediate 10-1 (10.0 g, 0.025 mol), 4-Cyanobenzeneboronic acid (4.4 g, 0.030 mol), K₂CO₃ (10.4 g, 0.075 mol), Pd(PPh₃)₄ (0.6 g, 0.0005 mol), and then were reacted with stirring at 100° C. for 6 hours. After completion of the reaction, the reaction mixture was extracted, concentrated, and then subjected to column and recrystallization to obtain 6.3 g (yield 59.7%) of Compound 58.

LC/MS: m/z=422[(M+1)⁺]

### Synthesis Example 6: Synthesis of Compound 76

### (1) Preparative Example 1: Synthesis of Intermediate 76-1

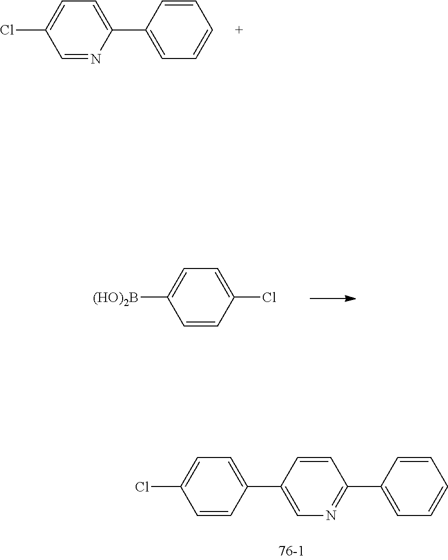 [SMILES: Clc1ccc(-c2ccccc2)nc1 ; OB(O)c1ccc(Cl)cc1 ; Clc1ccc(-c2ccc(-c3ccccc3)nc2)cc1]

Toluene 200 mL, EtOH 50 mL, and H₂O 50 mL were added to 5-Bromo-2-phenylpyridine (10.0 g, 0.043 mol), (4-Chlorophenyl)boronic acid (8.0 g, 0.051 mol), K₂CO₃ (17.7 g, 0.128 mol), Pd(PPh₃)₄ (1.0 g, 0.001 mol), and then were reacted with stirring at 100° C. for 6 hours. After completion of the reaction, the reaction mixture was extracted, concentrated, and then subjected to column and recrystallization to obtain 7.5 g (yield 66.1%) of Intermediate 76-1.

### (2) Preparative Example 2: Synthesis of Compound 76

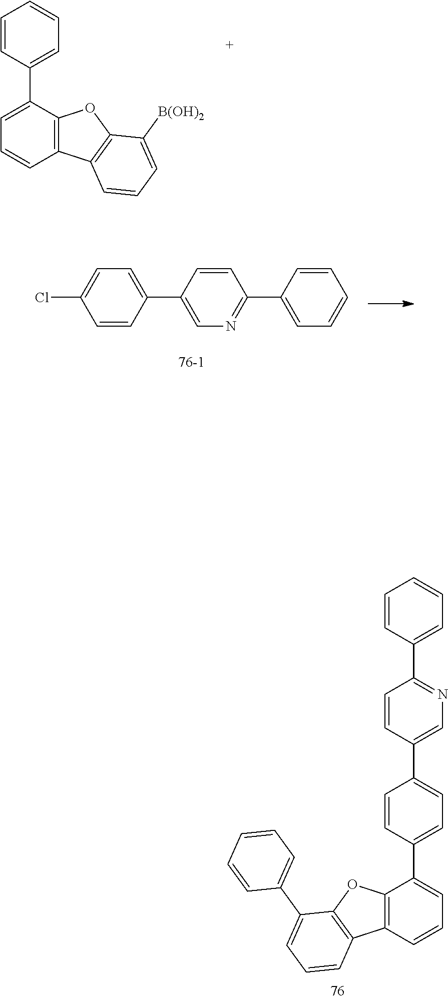 [SMILES: OB(O)c1cccc2c1oc1c(-c3ccccc3)cccc12 ; Clc1ccc(-c2ccc(-c3ccccc3)nc2)cc1 ; c1ccc(-c2ccc(-c3ccc(-c4cccc5c4oc4c(-c6ccccc6)cccc45)cc3)cn2)cc1]

6-Phenyldibenzofuran-4-boronic acid (10.0 g, 0.035 mol), Intermediate 76-1 (11.1 g, 0.042 mol), K₂CO₃ (14.4 g, 0.104 mol), catalyst Pd((OAc)₂ (2.0 g, 0.002 mol), ligand X-Phos (1.7 g, 0.004 mol), THF 200 ml, and H₂O 50 mL were mixed and reacted with stirring at 90ºC for 6 hours. After completion of the reaction, the reaction mixture was extracted, concentrated, and then subjected to column and recrystallization to obtain 11.2 g (yield 68.1%) of Compound 76.

LC/MS: m/z=473[(M+1)⁺]

### Synthesis Example 7: Synthesis of Compound 92

### (1) Preparative Example 1: Synthesis of Compound 92

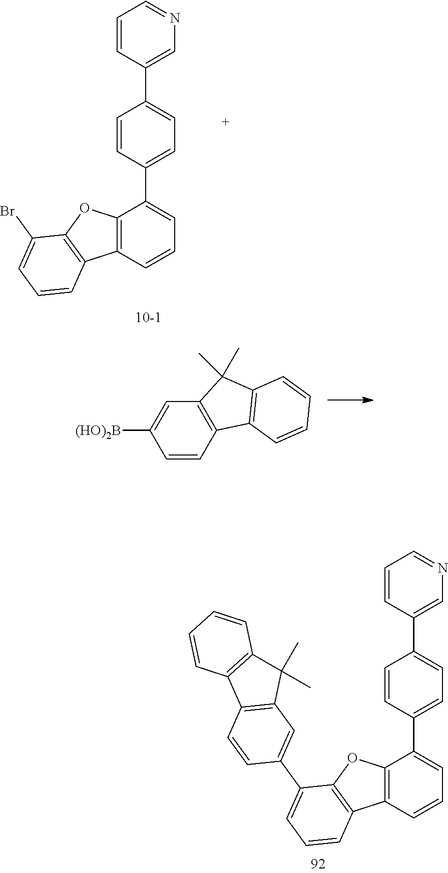 [SMILES: Brc1cccc2c1oc1c(-c3ccc(-c4cccnc4)cc3)cccc12 ; CC1(C)c2ccccc2-c2ccc(B(O)O)cc21 ; CC1(C)c2ccccc2-c2ccc(-c3cccc4c3oc3c(-c5ccc(-c6cccnc6)cc5)cccc34)cc21]

Toluene 200 mL, EtOH 50 mL, and H₂O 50 mL were added to Intermediate 10-1 (10.0 g, 0.025 mol), 9,9-Dimethyl-2-fluoreneboronic acid (7.1 g, 0.030 mol), K₂CO₃ (10.4 g, 0.075 mol), Pd(PPh₃)₄ (0.6 g, 0.0005 mol), and then were reacted with stirring at 100° C. for 6 hours. After completion of the reaction, the reaction mixture was extracted, concentrated, and then subjected to column and recrystallization to obtain 9.6 g (yield 74.8%) of Compound 92.

LC/MS: m/z=513[(M+1)⁺]

### Synthesis Example 8: Synthesis of Compound 104

### (1) Preparative Example 1: Synthesis of Compound 104

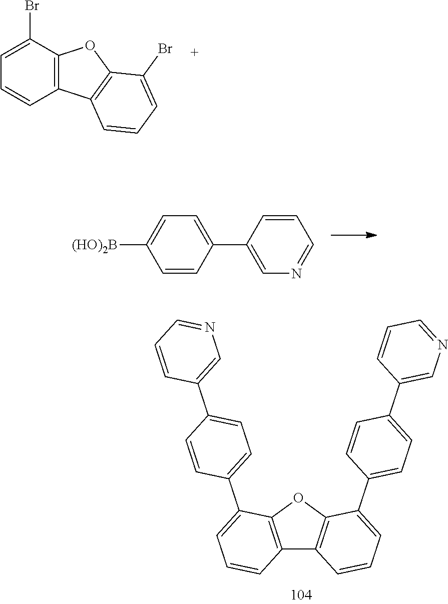 [SMILES: Brc1cccc2c1oc1c(Br)cccc12 ; OB(O)c1ccc(-c2cccnc2)cc1 ; c1cncc(-c2ccc(-c3cccc4c3oc3c(-c5ccc(-c6cccnc6)cc5)cccc34)cc2)c1]

Toluene 140 mL, EtOH 35 mL, and H₂O 35 mL were added to 4,6-Dibromodibenzofuran (10.0 g, 0.031 mol), 4-(Pyridin-3-yl)phenylboronic acid (14.7 g, 0.074 mol), K₂CO₃ (25.4 g, 0.184 mol), Pd(PPh₃)₄ (0.7 g, 0.0006 mol), and then were reacted with stirring at 100ᵢÆEC for 6 hours. After completion of the reaction, the reaction mixture was extracted, concentrated, and then subjected to column and recrystallization to obtain 10.8 g (yield 74.2%) of Compound 104.

LC/MS: m/z=474[(M+1)*]

### Synthesis Example 9: Synthesis of Compound 105

### (1) Preparative Example 1: Synthesis of Intermediate 105-1

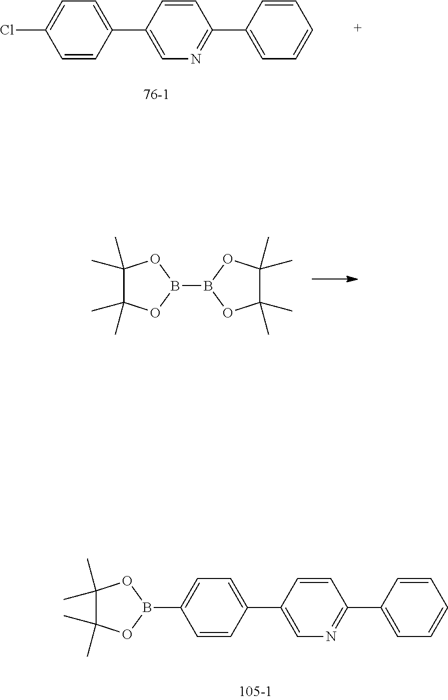

Dioxane 200 ml was added to Intermediate 76-1 (10.0 g, 0.038 mol), Bis(pinacolato)diboron (11.5 g, 0.045 mol), KOAc (11.1 g, 0.113 mol), Pd(dppf)Cl₂ (1.4 g, 0.002 mol), XPhos (2.7 g, 0.006 mol), and then was reacted with stirring at 100° C. for 12 hours. After completion of the reaction, the reaction mixture was extracted, concentrated, and then subjected to column and recrystallization to obtain 9.3 g (yield 69.2%) of Intermediate 105-1.

### (2) Preparative Example 2: Synthesis of Compound 105

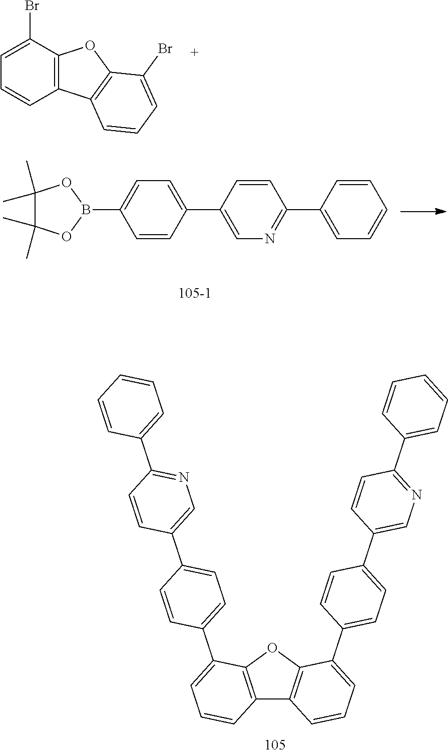 [SMILES: Brc1cccc2c1oc1c(Br)cccc12 ; CC1(C)OB(c2ccc(-c3ccc(-c4ccccc4)nc3)cc2)OC1(C)C ; c1ccc(-c2ccc(-c3ccc(-c4cccc5c4oc4c(-c6ccc(-c7ccc(-c8ccccc8)nc7)cc6)cccc45)cc3)cn2)cc1]

Toluene 140 mL, EtOH 35 mL, and H₂O 35 mL were added to 4,6-Dibromodibenzofuran (10.0 g, 0.031 mol), Intermediate 105-1 (26.3 g, 0.074 mol), K₂CO₃ (25.4 g, 0.184 mol), Pd(PPh₃)₄ (0.7 g, 0.0006 mol), and then were reacted with stirring at 100° ° C. for 6 hours. After completion of the reaction, the reaction mixture was extracted, concentrated, and then subjected to column and recrystallization to obtain 13.3 g (yield 69.2%) of Compound 105.

LC/MS: m/z=626[(M+1)⁺]

### Synthesis Example 10: Synthesis of Compound 127

### (1) Preparative Example 1: Synthesis of Compound 127

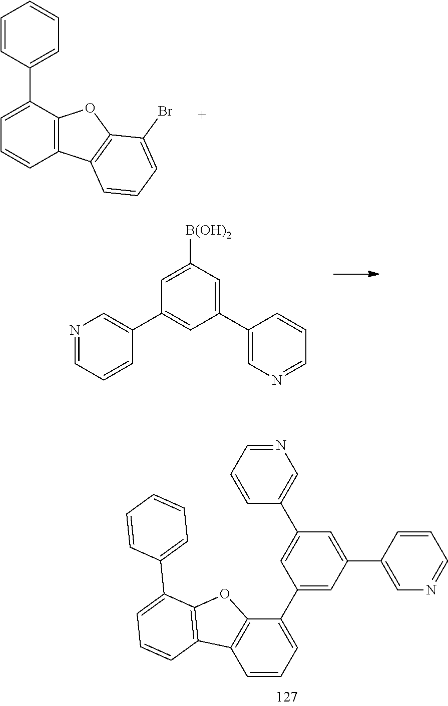 [SMILES: Brc1cccc2c1oc1c(-c3ccccc3)cccc12 ; OB(O)c1cc(-c2cccnc2)cc(-c2cccnc2)c1 ; c1ccc(-c2cccc3c2oc2c(-c4cc(-c5cccnc5)cc(-c5cccnc5)c4)cccc23)cc1]

Toluene 140 mL, EtOH 35 mL, and H₂O 35 mL were added to 4-Bromo-6-phenyldibenzofuran (10.0 g, 0.031 mol), B-(3,5-Di-3-pyridinylphenyl)boronic acid (10.3 g, 0.037 mol), K₂CO₃ (12.8 g, 0.093 mol), Pd(PPh₃)₄ (0.7 g, 0.0006 mol), and then were reacted with stirring at 100° C. for 6 hours. After completion of the reaction, the reaction mixture was extracted, concentrated, and then subjected to column and recrystallization to obtain 10.1 g (yield 68.8%) of Compound 127.

LC/MS: m/z=474[(M+1)⁺]

### Synthesis Example 11: Synthesis of Compound 147

### (1) Preparative Example 1: Synthesis of Intermediate 147-1

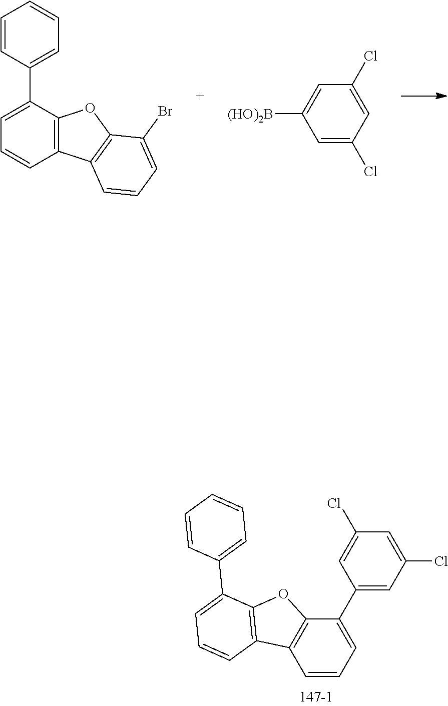 [SMILES: Brc1cccc2c1oc1c(-c3ccccc3)cccc12 ; OB(O)c1cc(Cl)cc(Cl)c1 ; Clc1cc(Cl)cc(-c2cccc3c2oc2c(-c4ccccc4)cccc23)c1]

Toluene 140 mL, EtOH 35 mL, and H₂O 35 mL were added to 4-Bromo-6-phenyldibenzofuran (10.0 g, 0.031 mol), 3,5-Dichlorophenylboronic acid (7.1 g, 0.037 mol), K₂CO₃ (12.8 g, 0.093 mol), Pd(PPh₃)₄ (0.7 g, 0.0006 mol), and then were reacted with stirring at 100° C. for 6 hours. After completion of the reaction, the reaction mixture was extracted, concentrated, and then subjected to column and recrystallization to obtain 9.2 g (yield 76.4%) of Intermediate 147-1.

### (2) Preparative Example 2: Synthesis of Compound 147

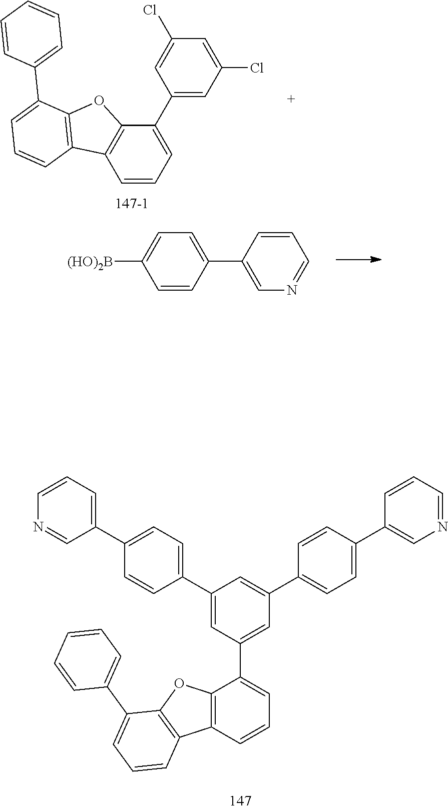 [SMILES: Clc1cc(Cl)cc(-c2cccc3c2oc2c(-c4ccccc4)cccc23)c1 ; OB(O)c1ccc(-c2cccnc2)cc1 ; c1ccc(-c2cccc3c2oc2c(-c4cc(-c5ccc(-c6cccnc6)cc5)cc(-c5ccc(-c6cccnc6)cc5)c4)cccc23)cc1]

Intermediate 147-1 (10.0 g, 0.026 mol), 4-(Pyridin-3-yl)phenylboronic acid (42.3 g, 0.062 mol), K₂CO₃ (17.8 g, 0.128 mol), catalyst Pd((OAc)₂ (1.5 g, 0.001 mol), ligand X-Phos (1.2 g, 0.003 mol), THF 200 ml, and H₂O 50 mL were mixed and reacted with stirring at 90ºC for 6 hours. After completion of the reaction, the reaction mixture was extracted, concentrated, and then subjected to column and recrystallization to obtain 20.7 g (yield 66.2%) of Compound 147.

LC/MS: m/z=626[(M+1)⁺]

### Synthesis Example 12: Synthesis of Compound 175

### (1) Preparative Example 1: Synthesis of Compound 175

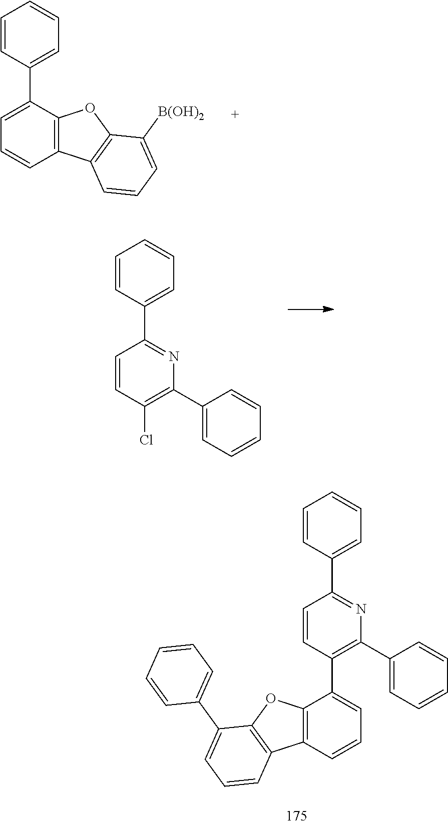

Toluene 140 mL, EtOH 35 mL, and H₂O 35 mL were added to 6-Phenyldibenzofuran-4-boronic acid (10.0 g, 0.035 mol), 3-Chloro-2,6-diphenylpyridine (11.1 g, 0.042 mol), K₂CO₃ (14.4 g, 0.104 mol), Pd(PPh₃)₄ (0.8 g, 0.0007 mol), and then were reacted with stirring at 100° C. for 6 hours. After completion of the reaction, the reaction mixture was extracted, concentrated, and then subjected to column and recrystallization to obtain 11.8 g (yield 71.8%) of Compound 175.

LC/MS: m/z=473[(M+1)⁺]

### Synthesis Example 13: Synthesis of Compound 220

### (1) Preparative Example 1: Synthesis of Compound 220

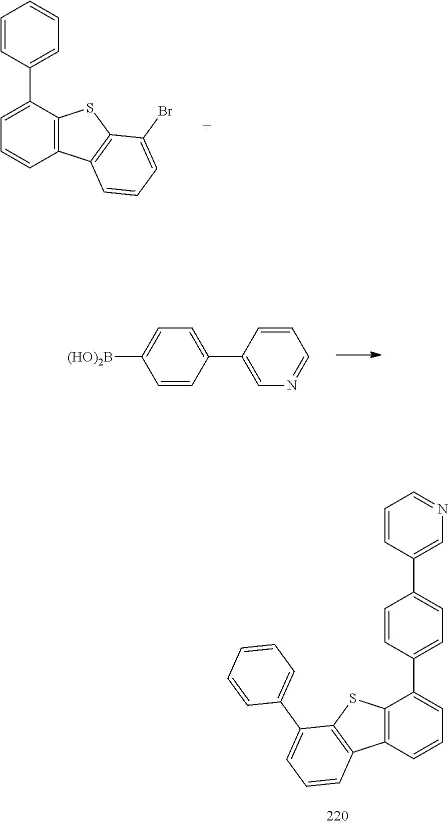 [SMILES: Brc1cccc2c1sc1c(-c3ccccc3)cccc12 ; OB(O)c1ccc(-c2cccnc2)cc1 ; c1ccc(-c2cccc3c2sc2c(-c4ccc(-c5cccnc5)cc4)cccc23)cc1]

Toluene 140 mL, EtOH 35 mL, and H₂O 35 mL were added to 4-Bromo-6-phenyldibenzothiophene (10.0 g, 0.030 mol), 4-(Pyridin-3-yl)phenylboronic acid (7.0 g, 0.035 mol), K₂CO₃ (12.2 g, 0.088 mol), Pd(PPh₃)₄ (0.7 g, 0.0006 mol), and then were reacted with stirring at 100° C. for 6 hours. After completion of the reaction, the reaction mixture was extracted, concentrated, and then subjected to column and recrystallization to obtain 9.6 g (yield 78.8%) of Compound 220.

LC/MS: m/z=413[(M+1)⁺]

### Synthesis Example 14: Synthesis of Compound 414

### (1) Preparative Example 1: Synthesis of Intermediate 414-1

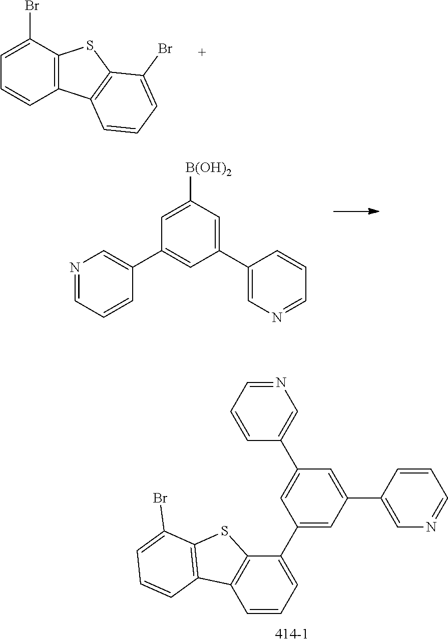 [SMILES: Brc1cccc2c1sc1c(Br)cccc12 ; OB(O)c1cc(-c2cccnc2)cc(-c2cccnc2)c1 ; Brc1cccc2c1sc1c(-c3cc(-c4cccnc4)cc(-c4cccnc4)c3)cccc12]

Toluene 140 mL, EtOH 35 mL, and H₂O 35 mL were added to 4,6-Dibromodibenzothiophene (10.0 g, 0.031 mol), B-(3,5-Di-3-pyridinylphenyl)boronic acid (10.2 g, 0.037 mol), K₂CO₃ (12.8 g, 0.093 mol), Pd(PPh₃)₄ (0.7 g, 0.0006 mol), and then were reacted with stirring at 100° ° C. for 6 hours. After completion of the reaction, the reaction mixture was extracted, concentrated, and then subjected to column and recrystallization to obtain 7.3 g (yield 47.9%) of Intermediate 414-1.

### (2) Preparative Example 2: Synthesis of Compound 414

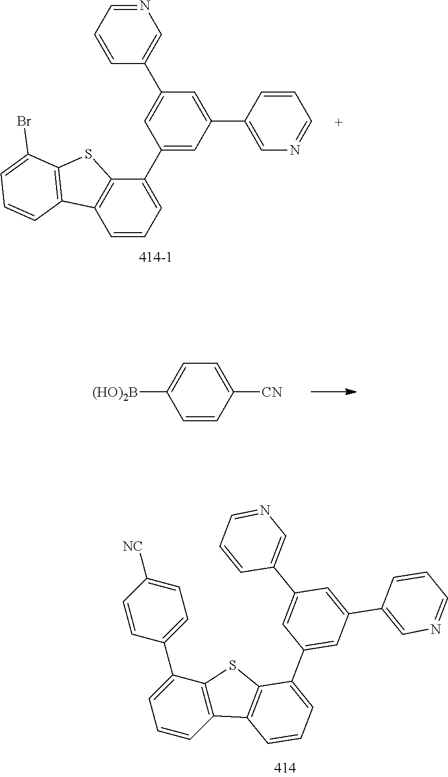 [SMILES: Brc1cccc2c1sc1c(-c3cc(-c4cccnc4)cc(-c4cccnc4)c3)cccc12 ; N#Cc1ccc(B(O)O)cc1 ; N#Cc1ccc(-c2cccc3c2sc2c(-c4cc(-c5cccnc5)cc(-c5cccnc5)c4)cccc23)cc1]

Toluene 140 mL, EtOH 35 mL, and H₂O 35 mL were added to Intermediate 414-1 (10.0 g, 0.020 mol), 4-Cyanobenzeneboronic acid (3.6 g, 0.024 mol), K₂CO₃ (12.1 g, 0.088 mol), Pd(PPh₃)₄ (0.7 g, 0.0006 mol), and then were reacted with stirring at 100° ° C. for 6 hours. After completion of the reaction, the reaction mixture was extracted, concentrated, and then subjected to column and recrystallization to obtain 11.4 g (yield 68.2%) of Compound 414.

LC/MS: m/z=515[(M+1)⁺]

### Synthesis Example 15: Synthesis of Compound 421

### (1) Preparative Example 1: Synthesis of Compound 421

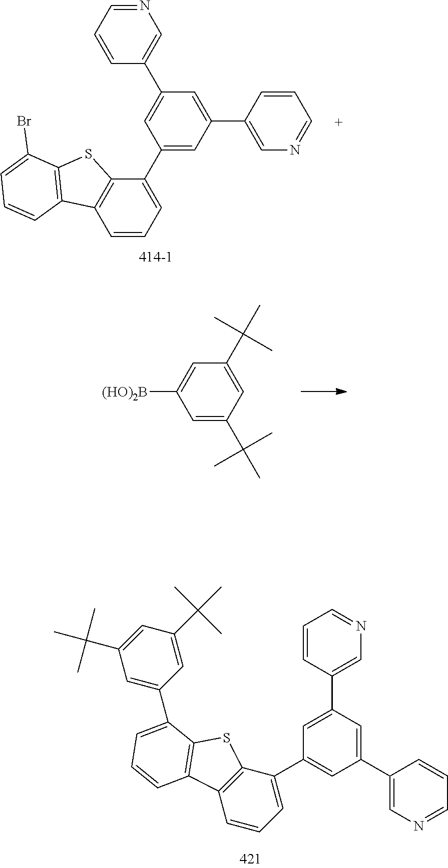 [SMILES: Brc1cccc2c1sc1c(-c3cc(-c4cccnc4)cc(-c4cccnc4)c3)cccc12 ; CC(C)(C)c1cc(B(O)O)cc(C(C)(C)C)c1 ; CC(C)(C)c1cc(-c2cccc3c2sc2c(-c4cc(-c5cccnc5)cc(-c5cccnc5)c4)cccc23)cc(C(C)(C)C)c1]

Toluene 140 mL, EtOH 35 mL, and H₂O 35 mL were added to Intermediate 414-1 (10.0 g, 0.020 mol), 3,5-Di-tert-butylbenzeneboronic acid (5.7 g, 0.024 mol), K₂CO₃ (8.4 g, 0.061 mol), Pd(PPh₃)₄ (0.5 g, 0.0004 mol), and then were reacted with stirring at 100° C. for 6 hours. After completion of the reaction, the reaction mixture was extracted, concentrated, and then subjected to column and recrystallization to obtain 7.7 g (yield 63.0%) of Compound 421.

LC/MS: m/z=602[(M+1)⁺]

### Device Examples (ETL)

In an example of the present invention, a ITO transparent electrode was patterned on a glass substrate having dimensions of 25 mm×25 mm×0.7 mm to have a light emitting area of 2 mm×2 mm using an ITO glass substrate to which the ITO transparent electrode was attached, followed by cleaning. After the substrate was mounted in a vacuum chamber, organic materials and metals were deposited in the following structure on the ITO at a base pressure of 1×10⁻⁶ torr or higher.

### Device Examples 1 to 83

After fabricating an organic light emitting device with the following device structure employing the compounds implemented according to an embodiment of the present invention as a material for an electron transport layer, the luminescent and driving properties of the compound implemented according to an embodiment of the present invention were measured.

ITO/Hole injecting layer (HAT-CN, 5 nm)/Hole transport layer (α-NPB, 100 nm)/Electron blocking layer (EBL1 10 nm)/Light emitting layer (20 nm)/Electron transport layer (201:Liq, 30 nm)/LiF (1 nm)/Al (100 nm)

A 5 nm thick hole injecting layer was deposited on the ITO transparent electrode using HAT-CN and then a 100 nm thick hole transport layer was formed into a film using α-NPB. A 10 nm thick electron blocking layer was deposited using EBL1. Further, a 20 nm thick light emitting layer was co-deposited using BH1 as a host compound and BD1 as a dopant compound, a 30 nm thick electron transport layer (doped with Liq) was deposited using the compound according to an embodiment of the present invention listed in Table 1 below, and then 1 nm thick LiF was formed into a film to form the electron injecting layer. Thereafter, 100 nm thick Al was formed into a film to fabricate an organic light emitting device.

### Device Comparative Example 1

An organic light emitting device for Device Comparative Example 1 was fabricated in the same manner, except that ET1 was used instead of the compound according to an embodiment of the present invention as a material for an electron transport layer in the device structure of the above Examples.

### Device Comparative Example 2

An organic light emitting device for Device Comparative Example 2 was fabricated in the same manner, except that ET2 was used instead of the compound according to an embodiment of the present invention as a material for an electron transport layer in the device structures of the above Examples.

### Experimental Example 1: Luminescent Properties of Device Examples 1 to 83

Voltages, currents, and luminous efficiencies of the organic light emitting devices fabricated according to the above Examples and Comparative Examples were measured using Source meter (Model 237, Keithley) and a spectroradiometer (PR-650, Photo Research). Result values at 1000 nit are shown in Table 1 below.

TABLE 1

| | Electron | | | | |
|---|---|---|---|---|---|
| Examples | transport layer | V | cd/A | CIEx | CIEy |
| 1 | Formula 1 | 3.9 | 7.7 | 0.132 | 0.147 |
| 2 | Formula 2 | 3.8 | 8.1 | 0.134 | 0.148 |
| 3 | Formula 3 | 4.1 | 7.4 | 0.132 | 0.148 |
| 4 | Formula 4 | 3.9 | 7.7 | 0.132 | 0.147 |
| 5 | Formula 5 | 3.7 | 7.8 | 0.129 | 0.153 |
| 6 | Formula 6 | 3.7 | 7.7 | 0.131 | 0.152 |
| 7 | Formula 7 | 4.1 | 7.8 | 0.133 | 0.145 |
| 8 | Formula 8 | 3.6 | 8.0 | 0.131 | 0.149 |
| 9 | Formula 9 | 3.6 | 7.6 | 0.132 | 0.142 |
| 10 | Formula 10 | 4.1 | 7.9 | 0.133 | 0.145 |
| 11 | Formula 11 | 3.8 | 7.5 | 0.132 | 0.148 |
| 12 | Formula 12 | 3.9 | 8.1 | 0.131 | 0.147 |
| 13 | Formula 19 | 3.9 | 7.4 | 0.132 | 0.148 |
| 14 | Formula 20 | 4.0 | 7.5 | 0.134 | 0.145 |
| 15 | Formula 21 | 3.7 | 7.9 | 0.133 | 0.146 |
| 16 | Formula 24 | 4.1 | 7.7 | 0.132 | 0.147 |
| 17 | Formula 25 | 3.8 | 7.5 | 0.131 | 0.153 |
| 18 | Formula 26 | 4.1 | 7.4 | 0.132 | 0.148 |
| 19 | Formula 32 | 4.1 | 8.2 | 0.133 | 0.145 |
| 20 | Formula 34 | 3.9 | 7.5 | 0.133 | 0.152 |
| 21 | Formula 36 | 4.0 | 7.2 | 0.133 | 0.146 |
| 22 | Formula 37 | 4.3 | 7.5 | 0.133 | 0.146 |
| 23 | Formula 38 | 3.8 | 8.2 | 0.131 | 0.149 |
| 24 | Formula 42 | 3.9 | 7.3 | 0.133 | 0.147 |
| 25 | Formula 44 | 4.0 | 7.3 | 0.136 | 0.144 |
| 26 | Formula 47 | 4.1 | 7.5 | 0.132 | 0.148 |
| 27 | Formula 50 | 4.0 | 7.7 | 0.137 | 0.147 |
| 28 | Formula 55 | 3.8 | 8.1 | 0.131 | 0.142 |
| 29 | Formula 56 | 3.9 | 7.7 | 0.134 | 0.147 |
| 30 | Formula 58 | 3.8 | 7.6 | 0.132 | 0.143 |
| 31 | Formula 63 | 4.0 | 7.5 | 0.136 | 0.145 |
| 32 | Formula 70 | 3.9 | 7.5 | 0.138 | 0.146 |
| 33 | Formula 71 | 3.8 | 7.4 | 0.133 | 0.151 |
| 34 | Formula 72 | 4.1 | 7.8 | 0.130 | 0.135 |
| 35 | Formula 76 | 3.9 | 8.3 | 0.131 | 0.152 |
| 36 | Formula 78 | 3.7 | 7.3 | 0.133 | 0.148 |
| 37 | Formula 82 | 3.9 | 7.8 | 0.132 | 0.148 |
| 38 | Formula 83 | 3.9 | 7.5 | 0.131 | 0.145 |
| 39 | Formula 86 | 4.0 | 7.9 | 0.132 | 0.146 |
| 40 | Formula 89 | 3.8 | 7.2 | 0.131 | 0.142 |
| 41 | Formula 90 | 3.7 | 7.5 | 0.13 | 0.145 |
| 42 | Formula 101 | 3.7 | 7.8 | 0.131 | 0.146 |
| 43 | Formula 102 | 4.2 | 7.5 | 0.131 | 0.147 |
| 44 | Formula 103 | 3.9 | 7.5 | 0.132 | 0.148 |
| 45 | Formula 104 | 4.0 | 7.4 | 0.136 | 0.143 |
| 46 | Formula 105 | 3.9 | 7.2 | 0.137 | 0.152 |
| 47 | Formula 107 | 3.7 | 7.5 | 0.138 | 0.147 |
| 48 | Formula 108 | 4.0 | 7.5 | 0.131 | 0.142 |
| 49 | Formula 111 | 4.1 | 7.7 | 0.135 | 0.146 |
| 50 | Formula 114 | 3.6 | 7.9 | 0.132 | 0.142 |
| 51 | Formula 120 | 3.7 | 7.5 | 0.133 | 0.145 |
| 52 | Formula 124 | 3.7 | 7.8 | 0.131 | 0.146 |
| 53 | Formula 125 | 4.2 | 7.5 | 0.134 | 0.149 |
| 54 | Formula 127 | 3.9 | 8.2 | 0.133 | 0.146 |
| 55 | Formula 129 | 4.0 | 7.4 | 0.138 | 0.151 |
| 56 | Formula 147 | 3.9 | 7.2 | 0.137 | 0.153 |
| 57 | Formula 175 | 3.7 | 7.5 | 0.138 | 0.147 |
| 58 | Formula 220 | 4.0 | 7.5 | 0.131 | 0.142 |
| 59 | Formula 223 | 4.1 | 7.7 | 0.135 | 0.146 |
| 60 | Formula 224 | 3.6 | 8.3 | 0.132 | 0.142 |
| 61 | Formula 226 | 4.0 | 7.4 | 0.131 | 0.143 |
| 62 | Formula 231 | 4.0 | 7.5 | 0.130 | 0.144 |
| 63 | Formula 250 | 3.9 | 7.9 | 0.139 | 0.147 |
| 64 | Formula 261 | 3.8 | 7.5 | 0.133 | 0.145 |
| 65 | Formula 276 | 4.0 | 8.0 | 0.131 | 0.142 |
| 66 | Formula 282 | 3.9 | 7.6 | 0.139 | 0.146 |
| 67 | Formula 283 | 4.3 | 7.7 | 0.132 | 0.142 |
| 68 | Formula 333 | 4.0 | 7.5 | 0.131 | 0.143 |
| 69 | Formula 334 | 3.7 | 7.5 | 0.130 | 0.144 |
| 70 | Formula 340 | 4.2 | 7.6 | 0.131 | 0.142 |
| 71 | Formula 342 | 4.0 | 7.9 | 0.131 | 0.147 |
| 72 | Formula 382 | 3.8 | 8.1 | 0.131 | 0.143 |
| 73 | Formula 411 | 3.9 | 7.3 | 0.131 | 0.145 |
| 74 | Formula 414 | 3.8 | 7.5 | 0.133 | 0.146 |
| 75 | Formula 417 | 3.7 | 7.3 | 0.130 | 0.145 |
| 76 | Formula 441 | 4.0 | 7.8 | 0.134 | 0.146 |
| 77 | Formula 450 | 3.8 | 7.5 | 0.131 | 0.145 |
| 78 | Formula 460 | 3.7 | 7.9 | 0.129 | 0.144 |
| 79 | Formula 464 | 3.7 | 7.2 | 0.139 | 0.144 |
| 80 | Formula 465 | 3.6 | 7.5 | 0.131 | 0.147 |
| 81 | Formula 469 | 3.6 | 7.5 | 0.132 | 0.148 |
| 82 | Formula 471 | 4.1 | 7.5 | 0.131 | 0.147 |
| 83 | Formula 472 | 3.8 | 7.7 | 0.134 | 0.149 |
| Comparative | ET1 | 4.7 | 6.6 | 0.135 | 0.151 |
| Example 1 | | | | | |
| Comparative | ET2 | 4.8 | 6.9 | 0.137 | 0.155 |
| Example 2 | | | | | |

As seen from the results in Table 1, it can be confirmed that when the compound according to an embodiment of the present invention was applied to the device as a material for the electron transport layer, the luminescent properties such as low-voltage driving characteristic, the luminous efficiency, and the quantum efficiency are much more excellent than those of the devices (Comparative Examples 1 and 2) which employ the compound used for the electron transport material of the related art and a compound compared with the characteristic structure of the compound according to an embodiment of the present invention, respectively.

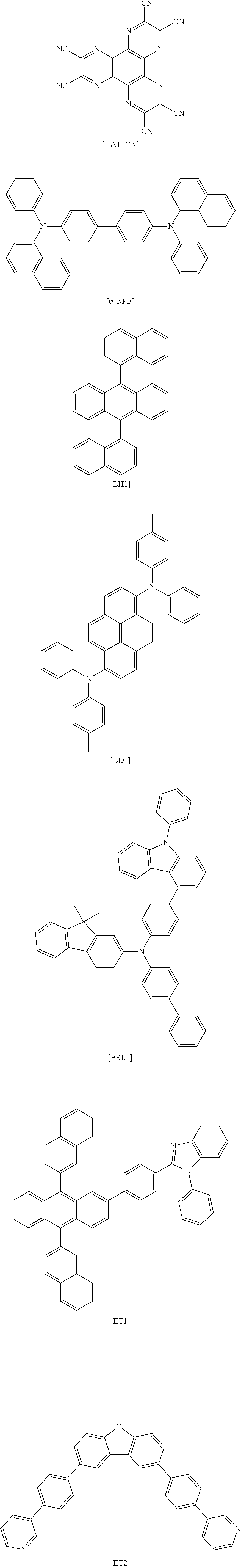

### INDUSTRIAL APPLICABILITY

When an organic compound according to an embodiment of the present invention is employed as a material for an organic layer such as an electron transport layer in the organic light emitting device, it is possible to achieve luminescent properties such as low-voltage driving of the device and excellent luminous efficiency, thus the present invention can be industrially utilized in various lighting, display devices and the like.

1. An organic compound represented by Formula I below: 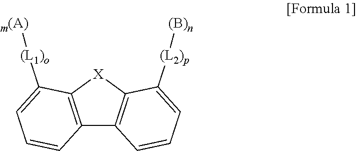
wherein in Formula I above, X is O, S, or CR₁R₂,
R₁ and R₂ are each independently selected from hydrogen, deuterium, a substituted or unsubstituted C1 to C20 alkyl group, a substituted or unsubstituted C3 to C20 cycloalkyl group, a substituted or unsubstituted C6 to C30 aryl group, and a substituted or unsubstituted C2 to C30 heteroaryl group,
the R₁ and R₂ are bonded to each other to form a substituted or aromatic ring,
L₁ and L₂ are a single bond or selected from a substituted or unsubstituted C1 to C30 arylene group, and a substituted or unsubstituted C3 to C30 heteroarylene group, o and p are each independently an integer of 0 to 4, when o and p are 2 or larger, the plurality of L₁ and L₂ are the same or different from each other,
A and B are the same or different from each other and a halogen group, a cyano group, a substituted or unsubstituted C1 to C20 alkyl group, a substituted or unsubstituted C3 to C20 cycloalkyl group, a substituted or unsubstituted C1 to C20 heterocycloalkyl group, a substituted or unsubstituted C6 to C30 aryl group, and a substituted or unsubstituted C2 to C30 heteroaryl group,
n and m are integers of 1 and 2 and when n and m are 2 or larger, the plurality of A and B are the same or different from each other,
at least one or more of A and B are represented by Structural Formula 1 below: 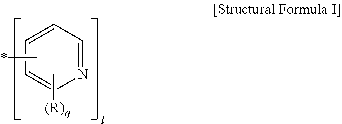
in Structural Formula 1 above,
R is selected from hydrogen, deuterium, halogen, a cyano group, a substituted or unsubstituted C1 to C20 alkyl group, a substituted or unsubstituted C3 to C20 cycloalkyl group, a substituted or unsubstituted C1 to C20 heterocycloalkyl group, a substituted or unsubstituted C6 to C30 aryl group, a substituted or unsubstituted C2 to C30 heteroaryl group, a substituted or unsubstituted C1 to C20 alkyl amine group, a substituted or unsubstituted C6 to C30 aryl amine group, a substituted or unsubstituted C1 to C20 alkyl silyl group, a substituted or unsubstituted C6 to C30 aryl silyl group, and a substituted or unsubstituted C1 to C20 alkoxy group,
q is an integer of 0 to 4 and when q is 2 or larger, the plurality of R is the same or different from each other, and
l is an integer of 1 to 4 and when 1 is 2 or larger, the plurality of structures in the bracket is the same or different from each other.

2. The organic compound of claim 1, wherein the term “substituted” or “unsubstituted” in the definitions of R, R₁, R₂, L₁, L₂, A, and B indicates substitution with one or more substituents selected from the group consisting of deuterium, a halogen group, a cyano group, a nitro group, a hydroxyl group, an alkyl group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkyl silyl group, and an aryl silyl group, substitution with a substituent to which two or more of the substituents are linked, or having no substituent.

3. The organic compound of claim 1, wherein the Formula I is any one selected from Compound 1 to Compound 492 below.

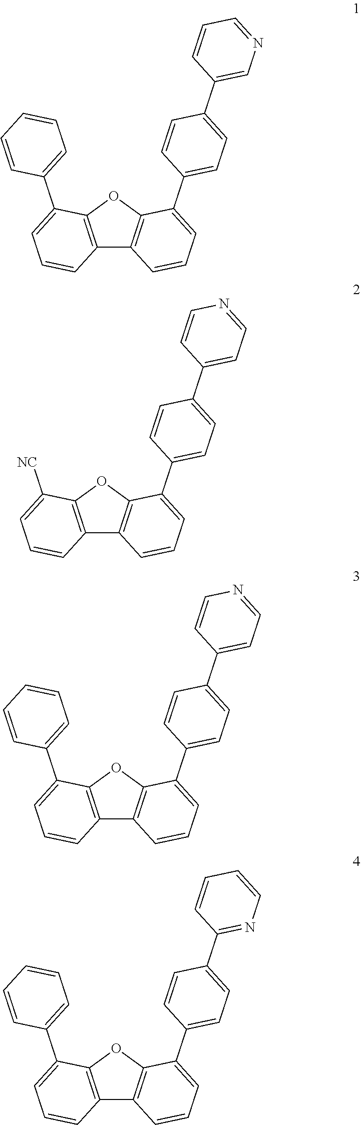

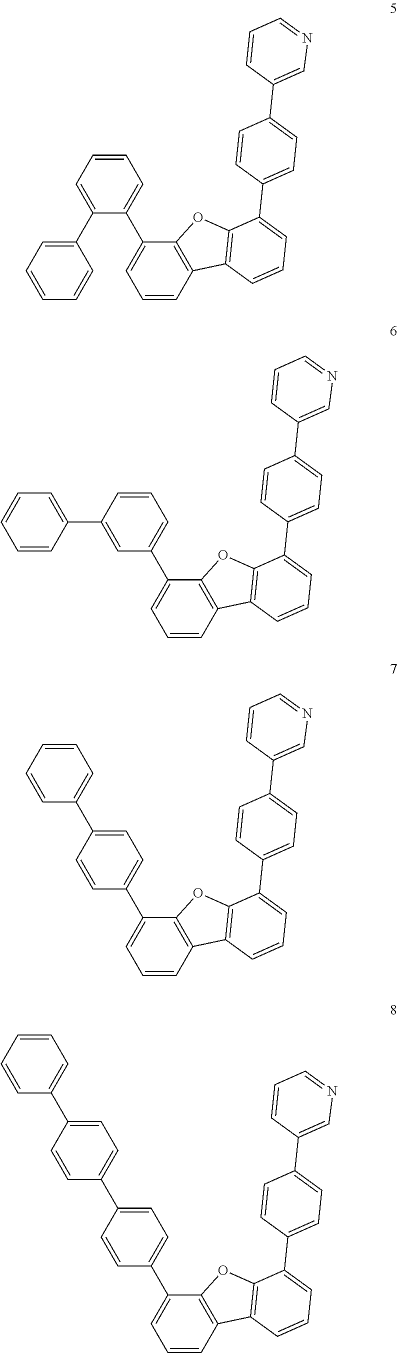 [SMILES: c1ccc(-c2ccccc2-c2cccc3c2oc2c(-c4ccc(-c5cccnc5)cc4)cccc23)cc1 ; c1ccc(-c2cccc(-c3cccc4c3oc3c(-c5ccc(-c6cccnc6)cc5)cccc34)c2)cc1 ; c1ccc(-c2ccc(-c3cccc4c3oc3c(-c5ccc(-c6cccnc6)cc5)cccc34)cc2)cc1 ; c1ccc(-c2ccc(-c3ccc(-c4cccc5c4oc4c(-c6ccc(-c7cccnc7)cc6)cccc45)cc3)cc2)cc1]

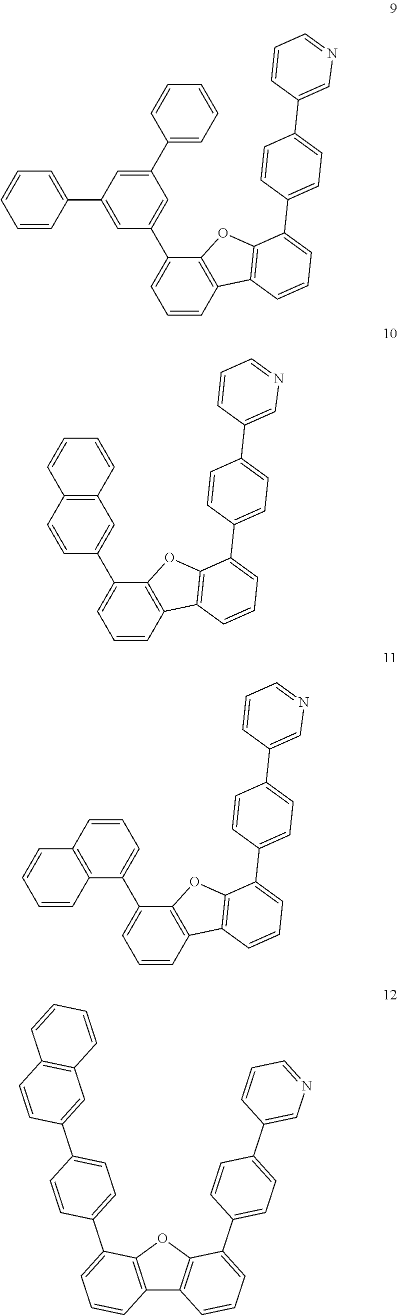 [SMILES: c1ccc(-c2cc(-c3ccccc3)cc(-c3cccc4c3oc3c(-c5ccc(-c6cccnc6)cc5)cccc34)c2)cc1 ; c1cncc(-c2ccc(-c3cccc4c3oc3c(-c5ccc6ccccc6c5)cccc34)cc2)c1 ; c1cncc(-c2ccc(-c3cccc4c3oc3c(-c5cccc6ccccc56)cccc34)cc2)c1 ; c1cncc(-c2ccc(-c3cccc4c3oc3c(-c5ccc(-c6ccc7ccccc7c6)cc5)cccc34)cc2)c1]

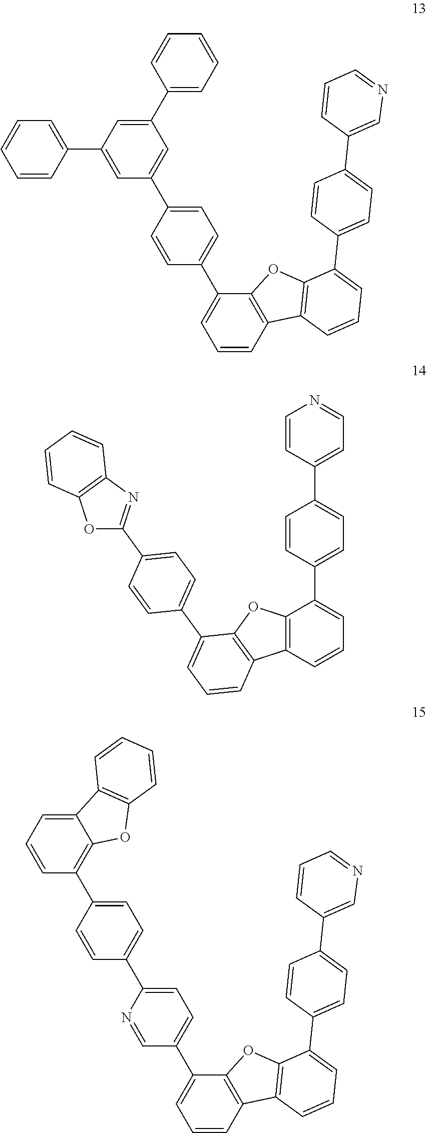 [SMILES: c1ccc(-c2cc(-c3ccccc3)cc(-c3ccc(-c4cccc5c4oc4c(-c6ccc(-c7cccnc7)cc6)cccc45)cc3)c2)cc1 ; c1ccc2oc(-c3ccc(-c4cccc5c4oc4c(-c6ccc(-c7ccncc7)cc6)cccc45)cc3)nc2c1 ; c1cncc(-c2ccc(-c3cccc4c3oc3c(-c5ccc(-c6ccc(-c7cccc8c7oc7ccccc78)cc6)nc5)cccc34)cc2)c1]

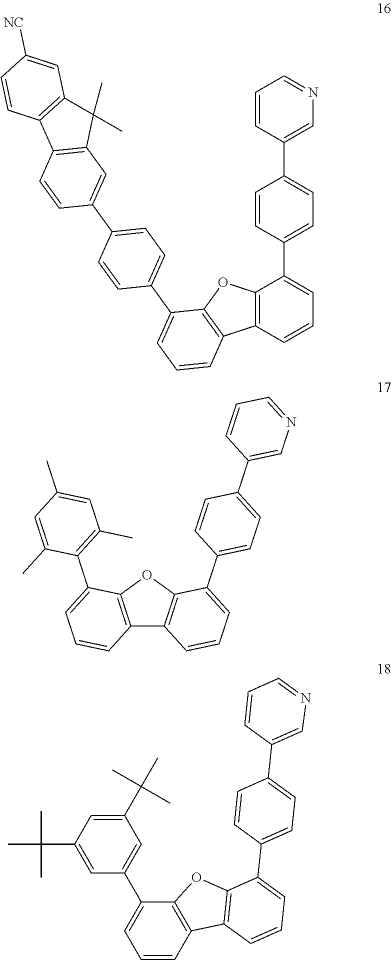

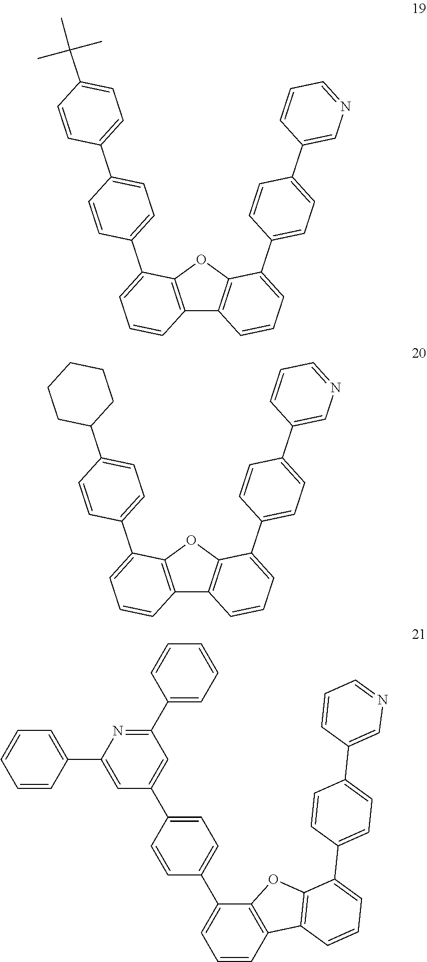

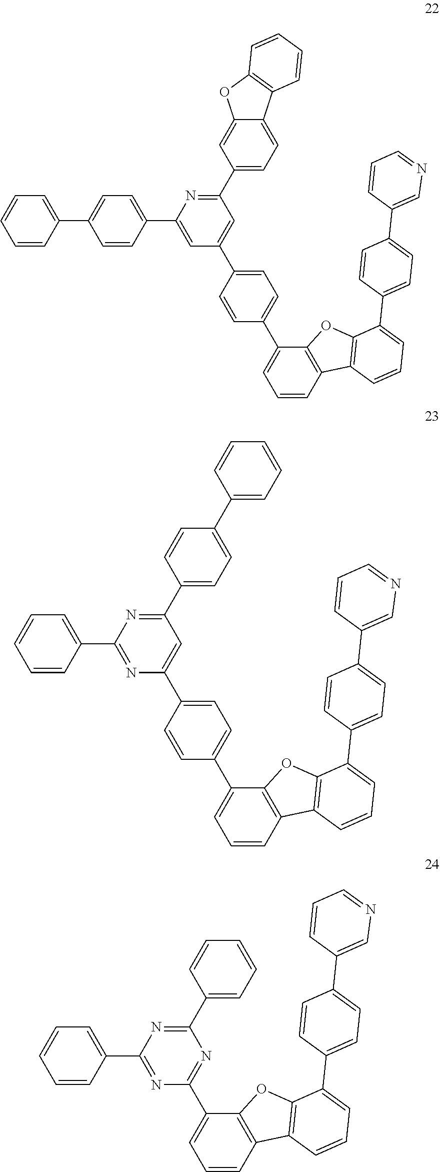

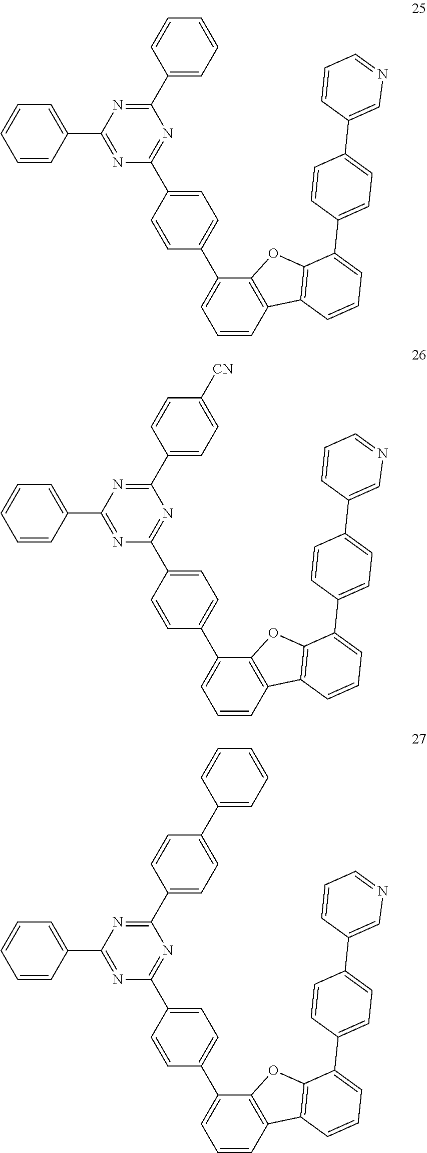

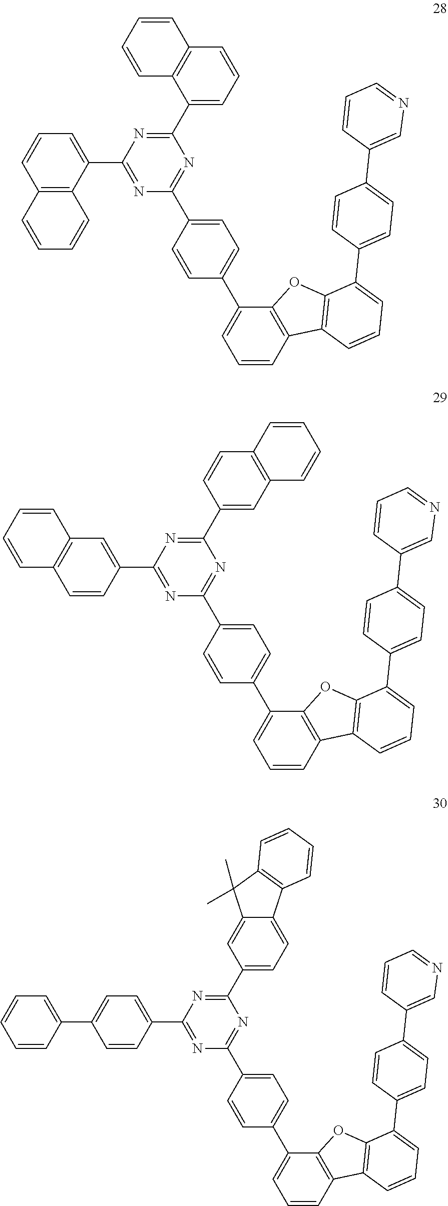 [SMILES: c1cncc(-c2ccc(-c3cccc4c3oc3c(-c5ccc(-c6nc(-c7cccc8ccccc78)nc(-c7cccc8ccccc78)n6)cc5)cccc34)cc2)c1 ; c1cncc(-c2ccc(-c3cccc4c3oc3c(-c5ccc(-c6nc(-c7ccc8ccccc8c7)nc(-c7ccc8ccccc8c7)n6)cc5)cccc34)cc2)c1 ; CC1(C)c2ccccc2-c2ccc(-c3nc(-c4ccc(-c5ccccc5)cc4)nc(-c4ccc(-c5cccc6c5oc5c(-c7ccc(-c8cccnc8)cc7)cccc56)cc4)n3)cc21]

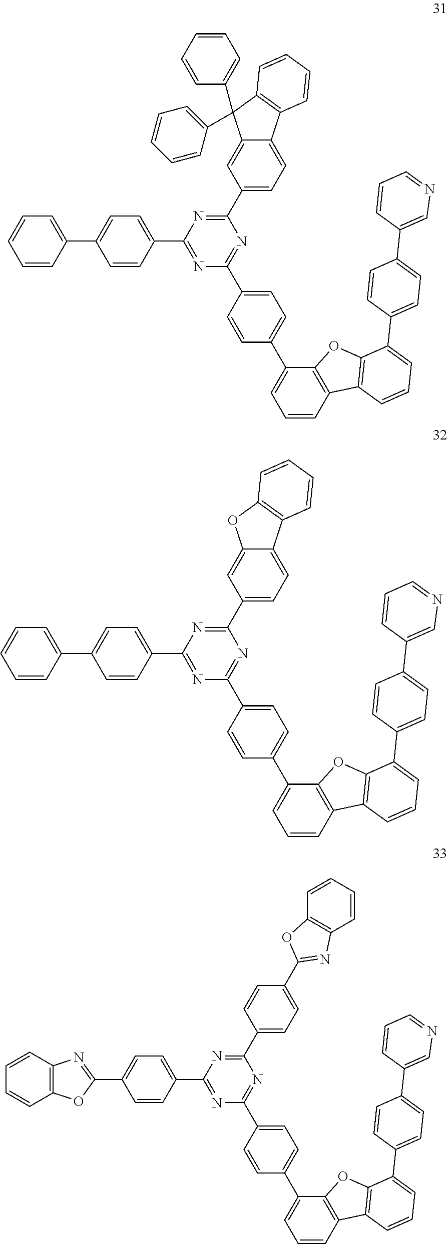 [SMILES: c1ccc(-c2ccc(-c3nc(-c4ccc(-c5cccc6c5oc5c(-c7ccc(-c8cccnc8)cc7)cccc56)cc4)nc(-c4ccc5c(c4)C(c4ccccc4)(c4ccccc4)c4ccccc4-5)n3)cc2)cc1 ; c1ccc(-c2ccc(-c3nc(-c4ccc(-c5cccc6c5oc5c(-c7ccc(-c8cccnc8)cc7)cccc56)cc4)nc(-c4ccc5c(c4)oc4ccccc45)n3)cc2)cc1 ; c1cncc(-c2ccc(-c3cccc4c3oc3c(-c5ccc(-c6nc(-c7ccc(-c8nc9ccccc9o8)cc7)nc(-c7ccc(-c8nc9ccccc9o8)cc7)n6)cc5)cccc34)cc2)c1]

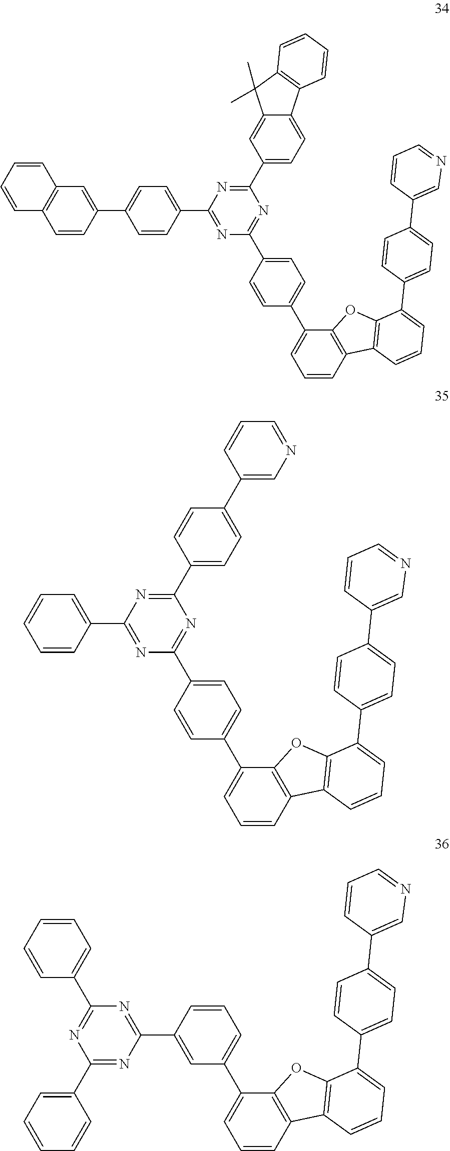 [SMILES: CC1(C)c2ccccc2-c2ccc(-c3nc(-c4ccc(-c5ccc6ccccc6c5)cc4)nc(-c4ccc(-c5cccc6c5oc5c(-c7ccc(-c8cccnc8)cc7)cccc56)cc4)n3)cc21 ; c1ccc(-c2nc(-c3ccc(-c4cccnc4)cc3)nc(-c3ccc(-c4cccc5c4oc4c(-c6ccc(-c7cccnc7)cc6)cccc45)cc3)n2)cc1 ; c1ccc(-c2nc(-c3ccccc3)nc(-c3cccc(-c4cccc5c4oc4c(-c6ccc(-c7cccnc7)cc6)cccc45)c3)n2)cc1]

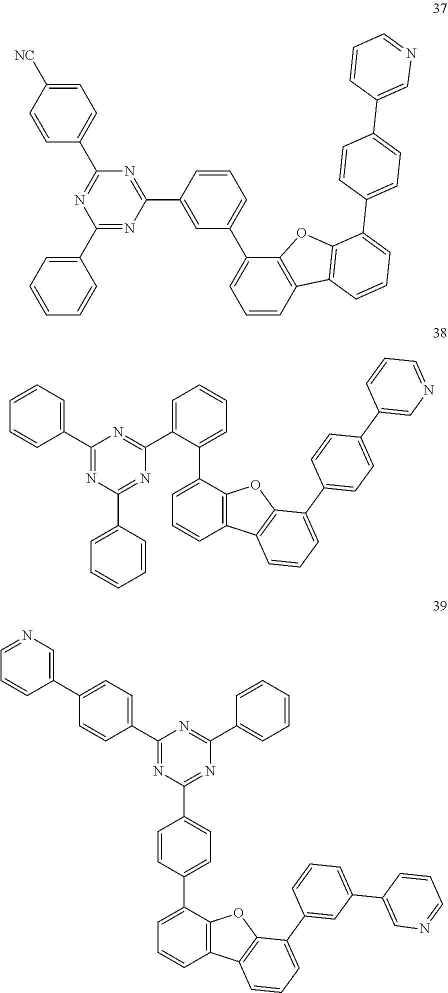 [SMILES: N#Cc1ccc(-c2nc(-c3ccccc3)nc(-c3cccc(-c4cccc5c4oc4c(-c6ccc(-c7cccnc7)cc6)cccc45)c3)n2)cc1 ; c1ccc(-c2nc(-c3ccccc3)nc(-c3ccccc3-c3cccc4c3oc3c(-c5ccc(-c6cccnc6)cc5)cccc34)n2)cc1 ; c1ccc(-c2nc(-c3ccc(-c4cccnc4)cc3)nc(-c3ccc(-c4cccc5c4oc4c(-c6cccc(-c7cccnc7)c6)cccc45)cc3)n2)cc1]

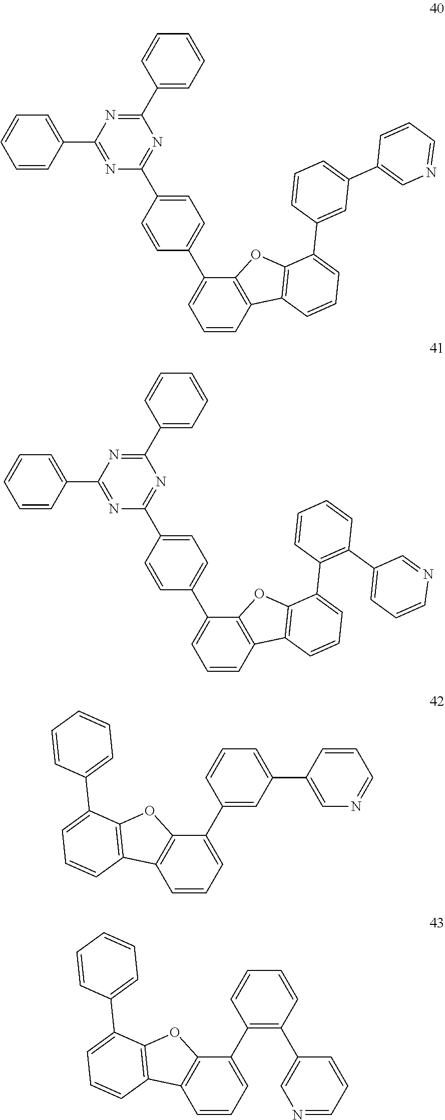

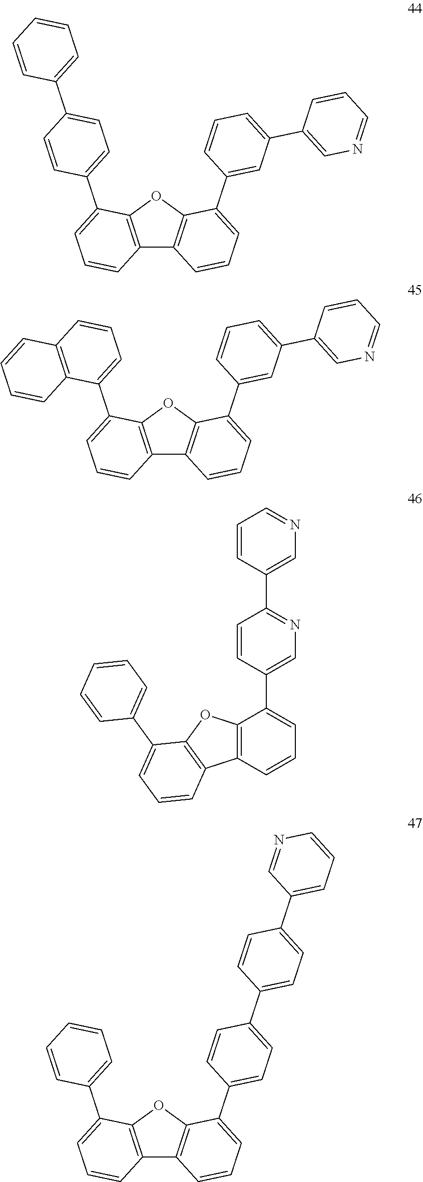

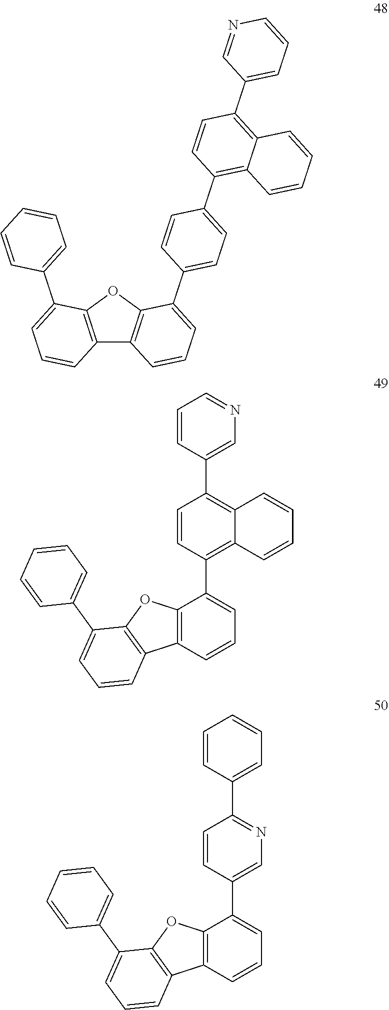

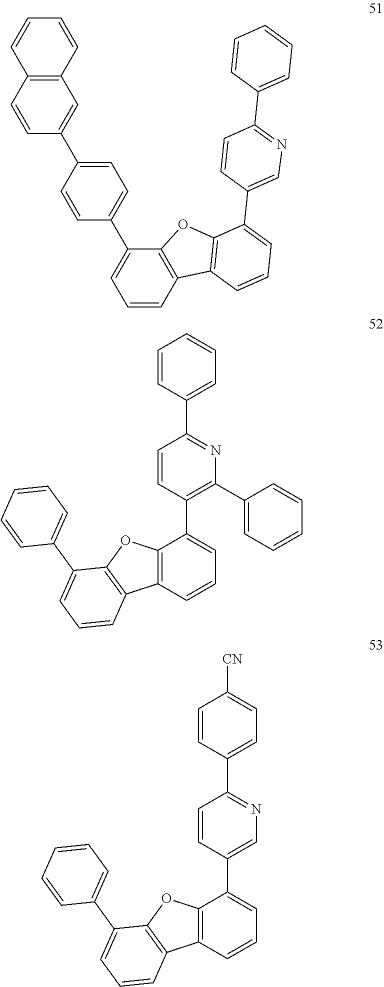

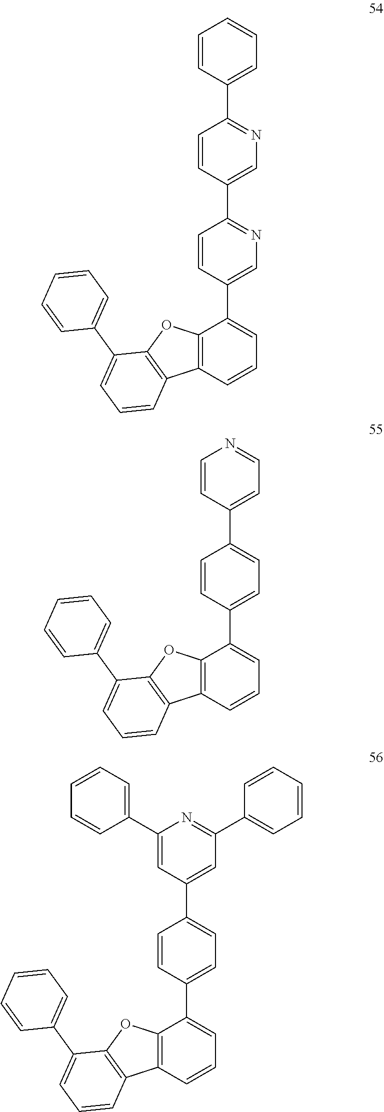

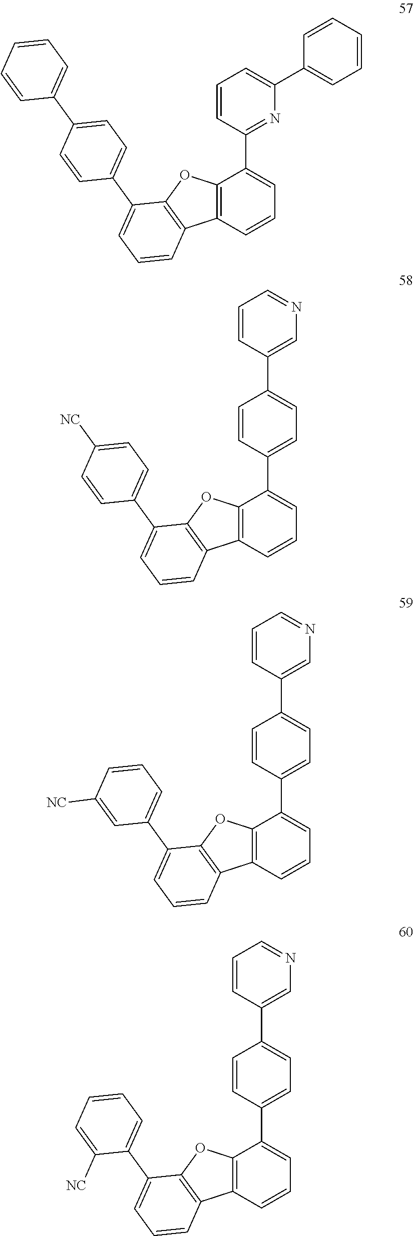 [SMILES: c1ccc(-c2ccc(-c3cccc4c3oc3c(-c5cccc(-c6ccccc6)n5)cccc34)cc2)cc1 ; N#Cc1ccc(-c2cccc3c2oc2c(-c4ccc(-c5cccnc5)cc4)cccc23)cc1 ; N#Cc1cccc(-c2cccc3c2oc2c(-c4ccc(-c5cccnc5)cc4)cccc23)c1 ; N#Cc1ccccc1-c1cccc2c1oc1c(-c3ccc(-c4cccnc4)cc3)cccc12]

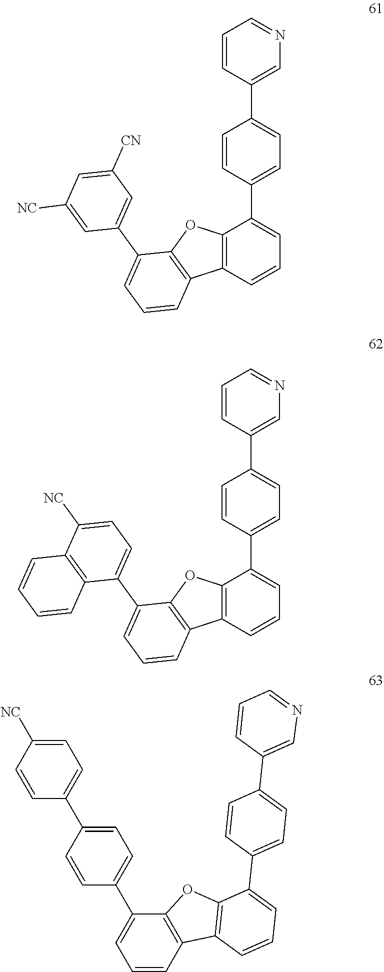 [SMILES: N#Cc1cc(C#N)cc(-c2cccc3c2oc2c(-c4ccc(-c5cccnc5)cc4)cccc23)c1 ; N#Cc1ccc(-c2cccc3c2oc2c(-c4ccc(-c5cccnc5)cc4)cccc23)c2ccccc12 ; N#Cc1ccc(-c2ccc(-c3cccc4c3oc3c(-c5ccc(-c6cccnc6)cc5)cccc34)cc2)cc1]

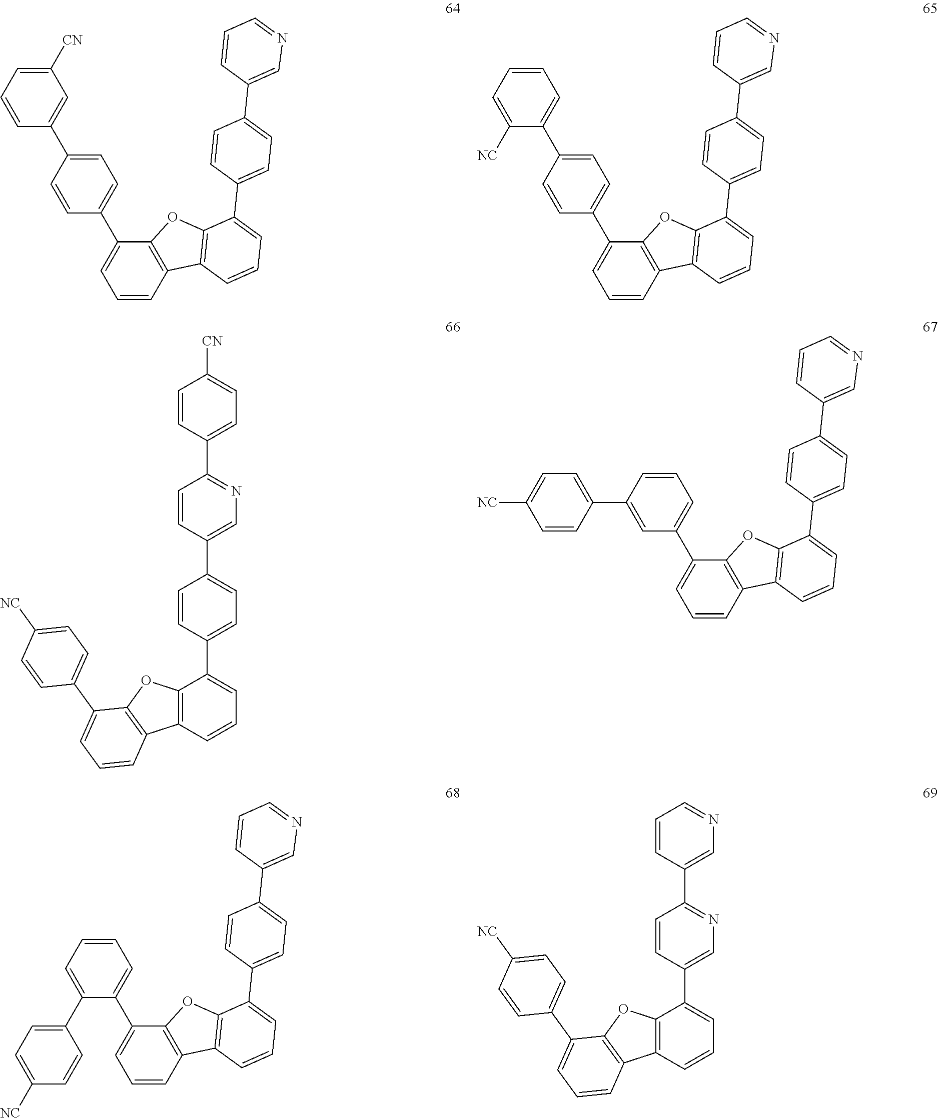 [SMILES: N#Cc1cccc(-c2ccc(-c3cccc4c3oc3c(-c5ccc(-c6cccnc6)cc5)cccc34)cc2)c1 ; N#Cc1ccccc1-c1ccc(-c2cccc3c2oc2c(-c4ccc(-c5cccnc5)cc4)cccc23)cc1 ; N#Cc1ccc(-c2ccc(-c3ccc(-c4cccc5c4oc4c(-c6ccc(C#N)cc6)cccc45)cc3)cn2)cc1 ; N#Cc1ccc(-c2cccc(-c3cccc4c3oc3c(-c5ccc(-c6cccnc6)cc5)cccc34)c2)cc1 ; N#Cc1ccc(-c2ccccc2-c2cccc3c2oc2c(-c4ccc(-c5cccnc5)cc4)cccc23)cc1 ; N#Cc1ccc(-c2cccc3c2oc2c(-c4ccc(-c5cccnc5)nc4)cccc23)cc1]

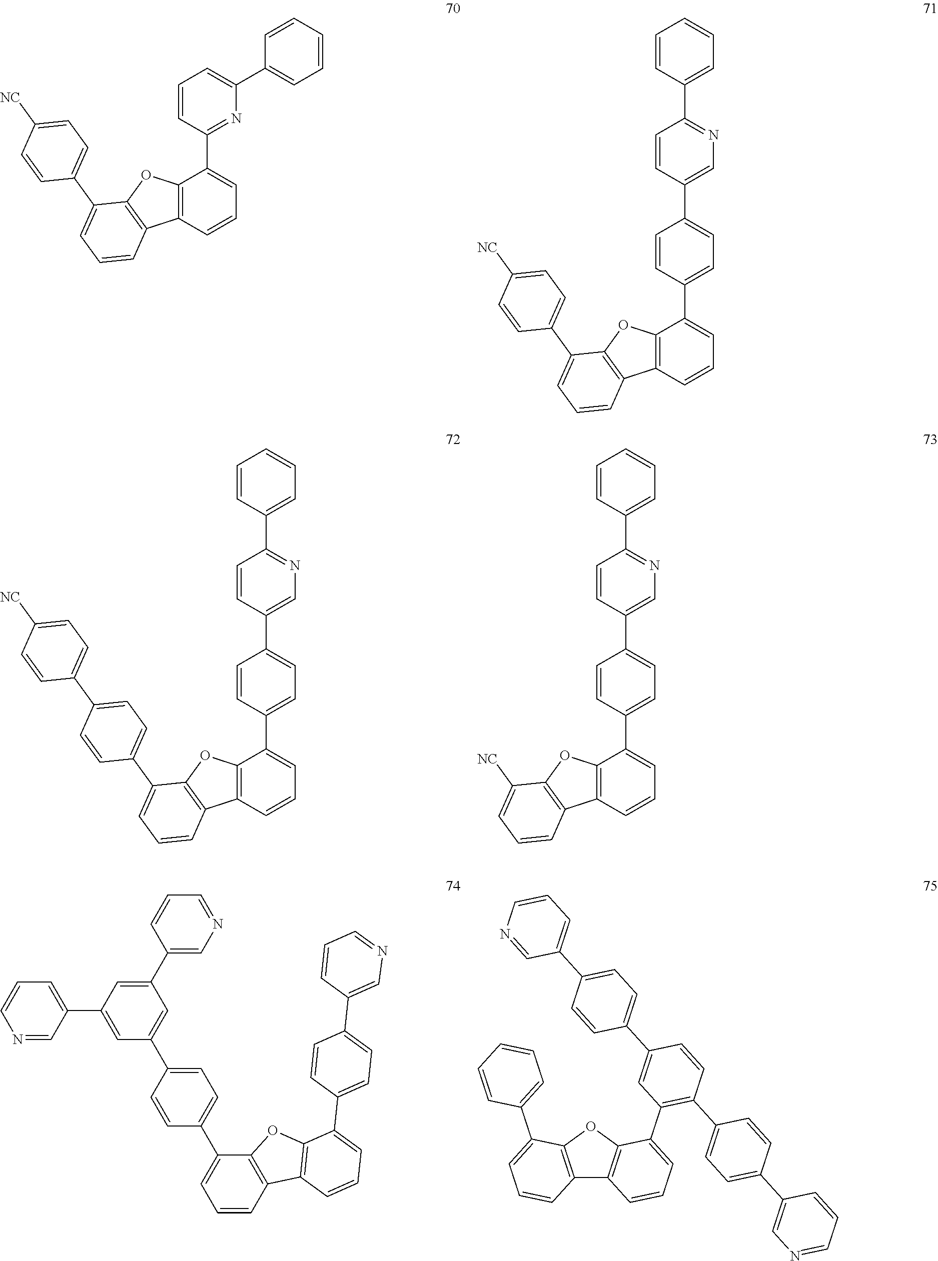

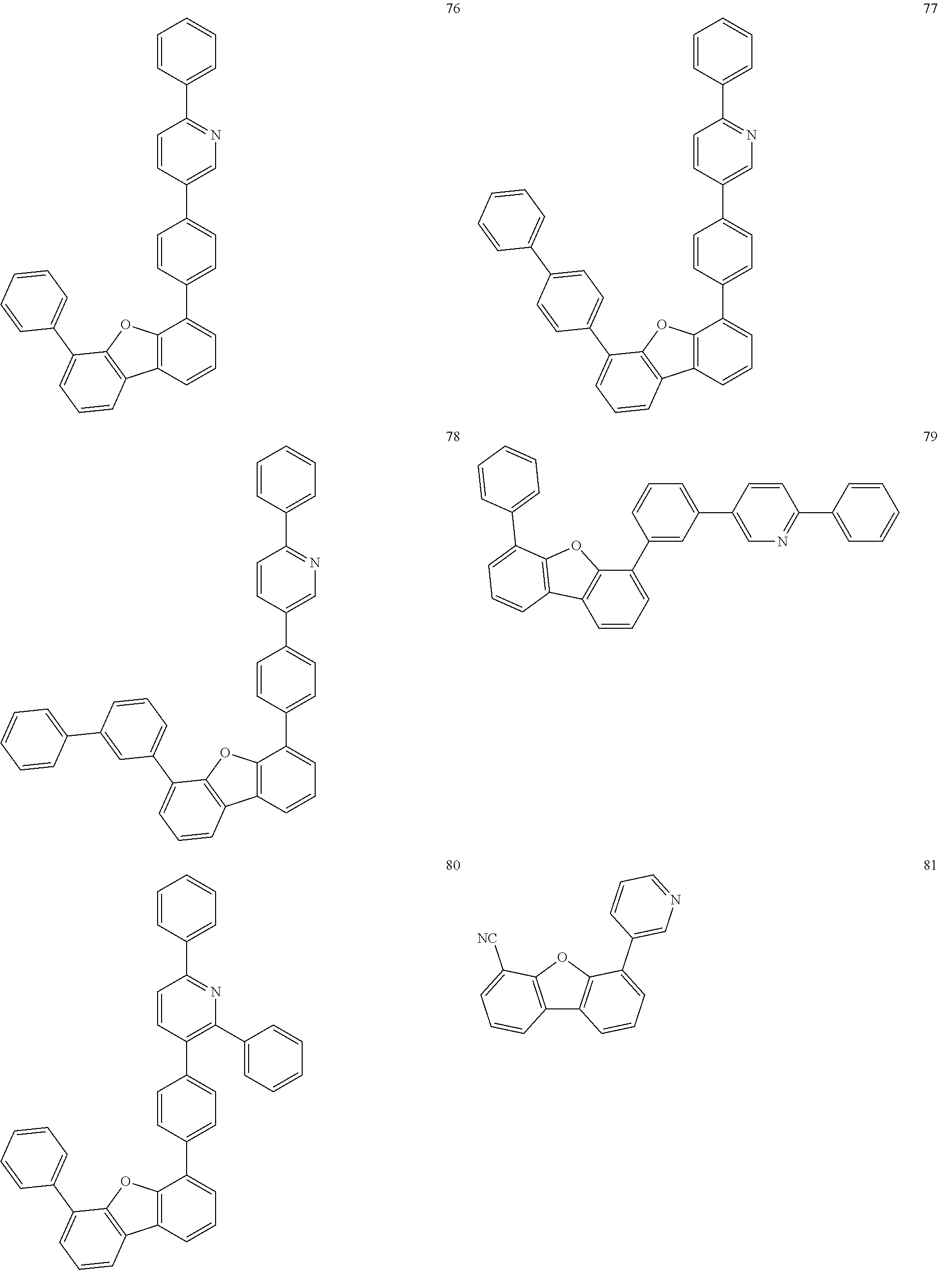

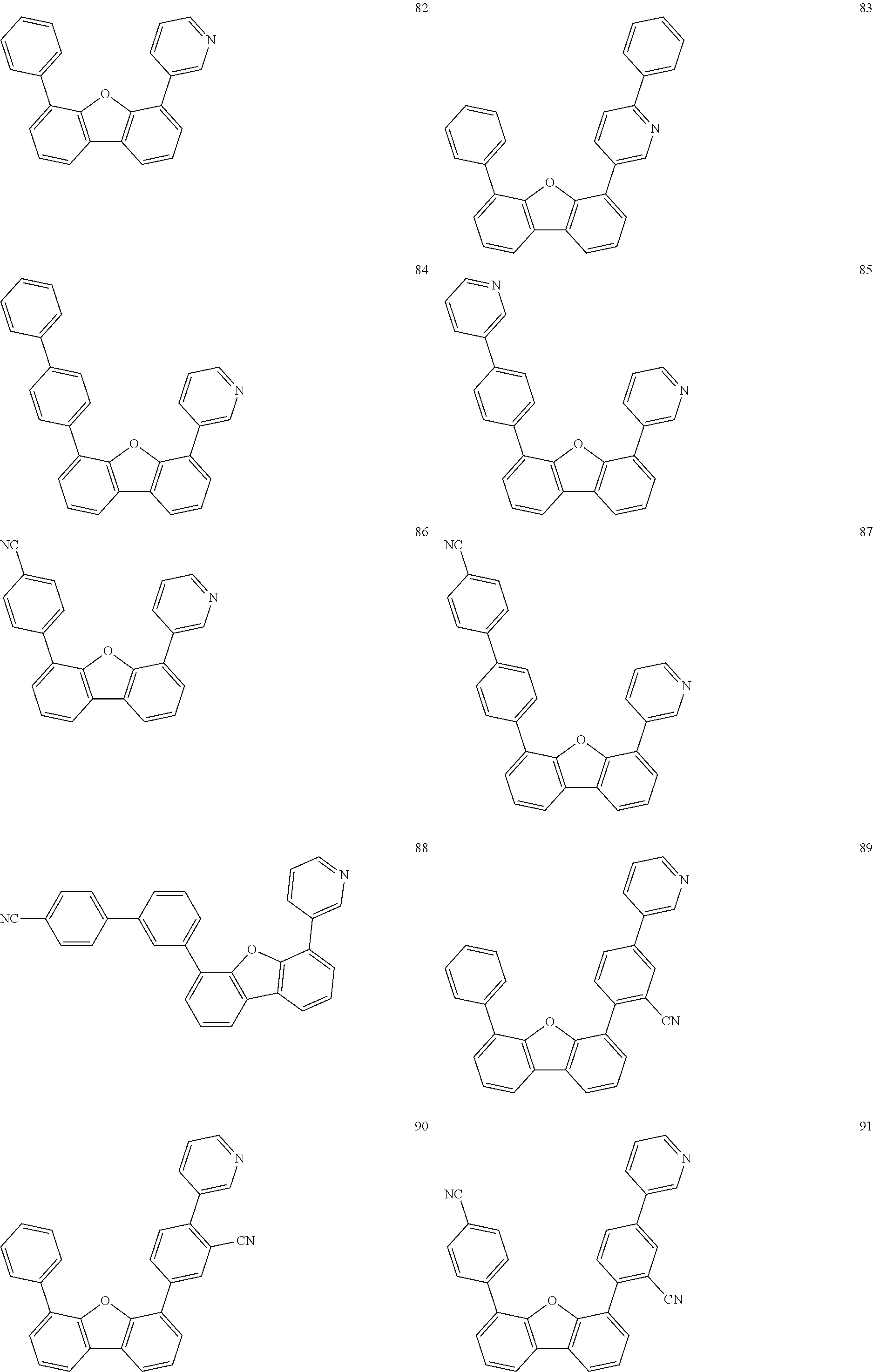

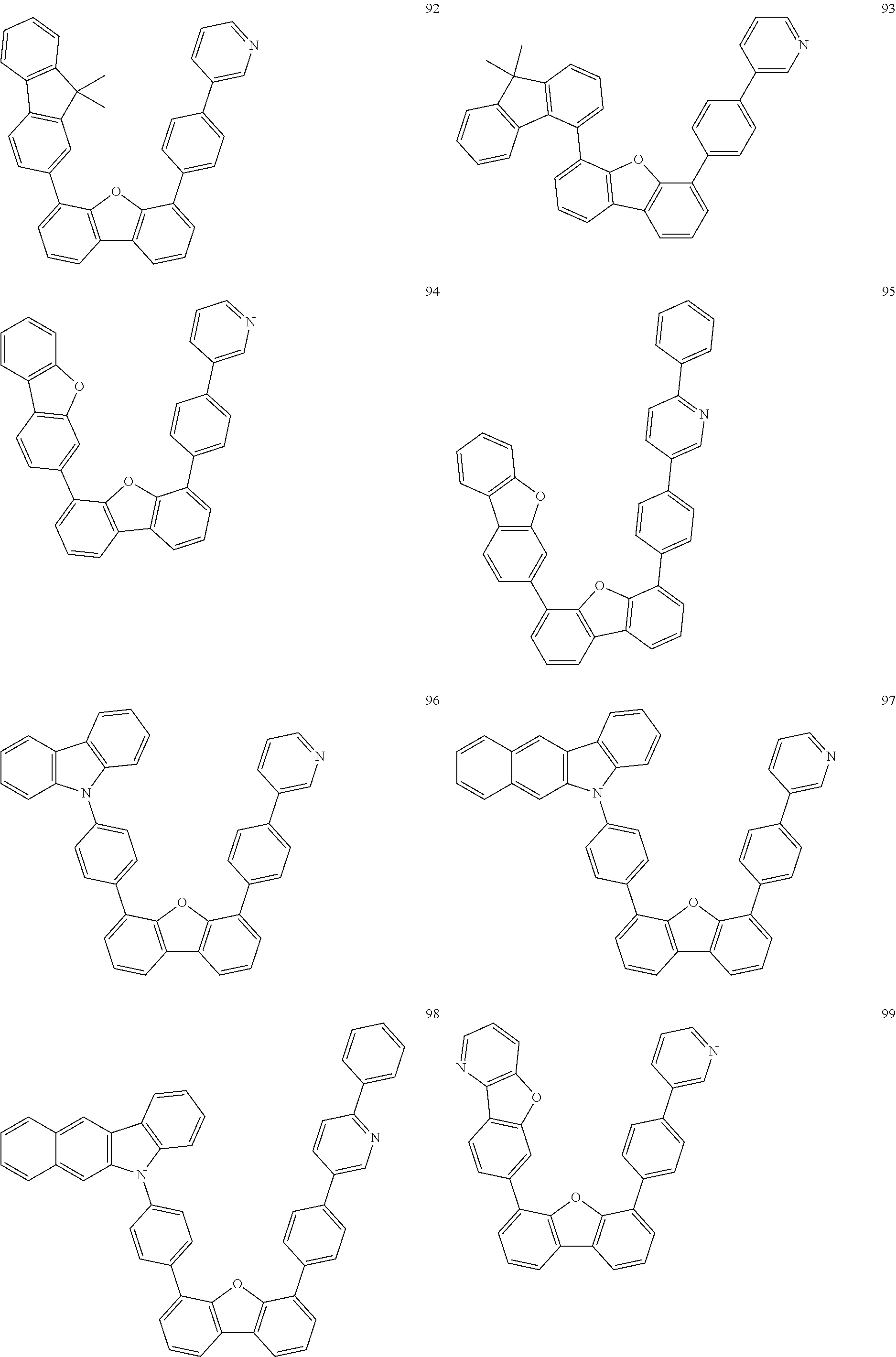

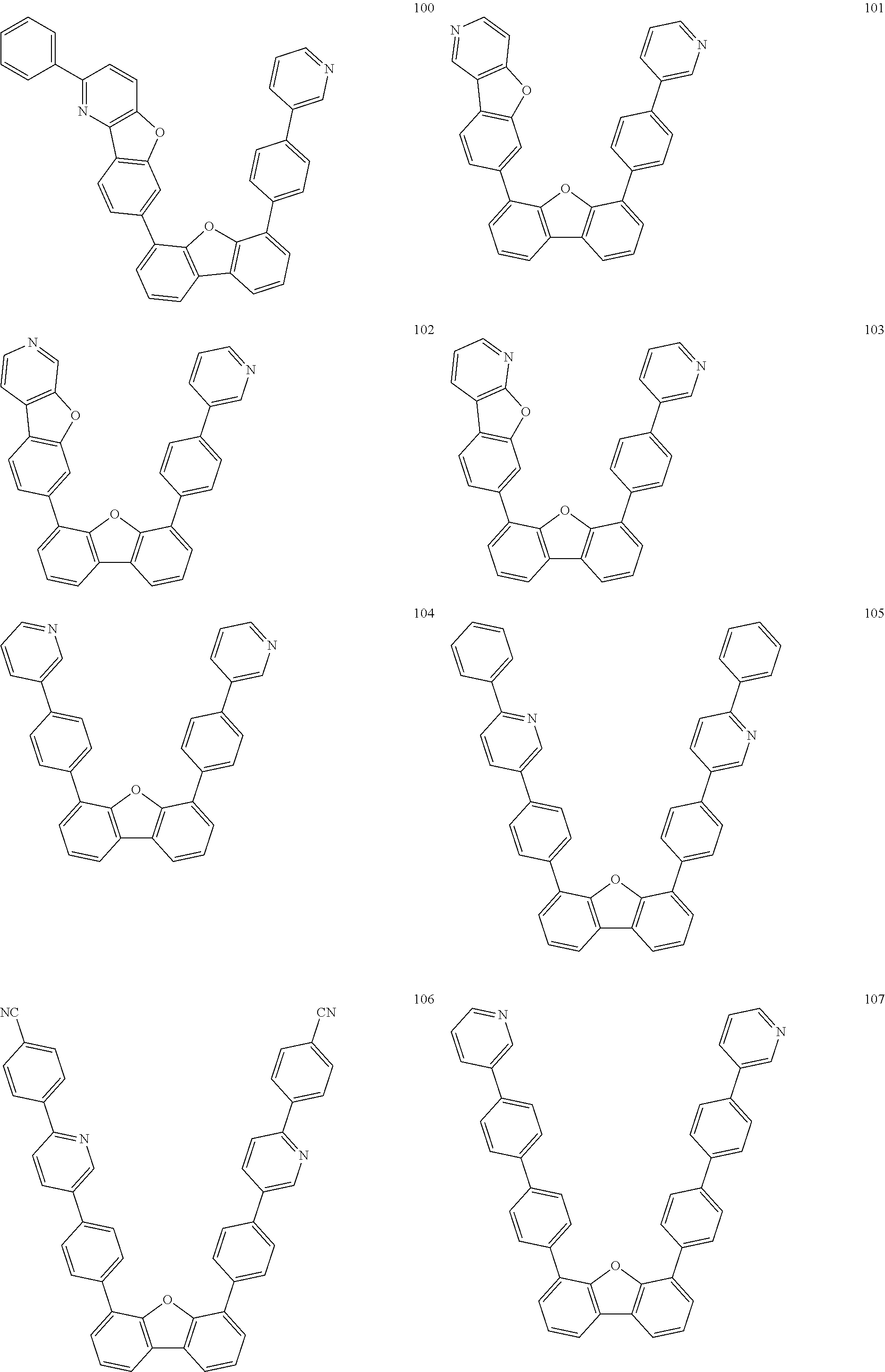

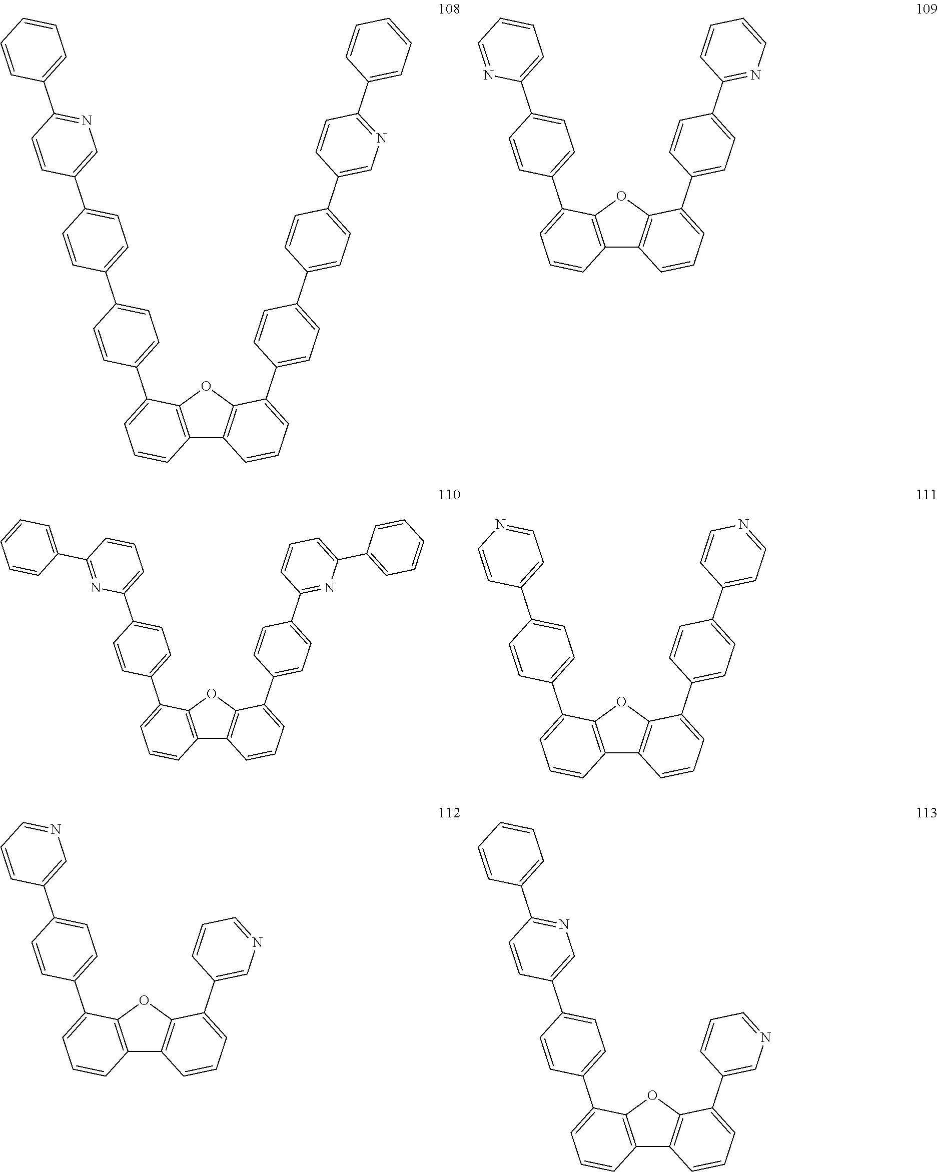 [SMILES: c1ccc(-c2ccc(-c3ccc(-c4ccc(-c5cccc6c5oc5c(-c7ccc(-c8ccc(-c9ccc(-c%10ccccc%10)nc9)cc8)cc7)cccc56)cc4)cc3)cn2)cc1 ; c1ccc(-c2ccc(-c3cccc4c3oc3c(-c5ccc(-c6ccccn6)cc5)cccc34)cc2)nc1 ; c1ccc(-c2cccc(-c3ccc(-c4cccc5c4oc4c(-c6ccc(-c7cccc(-c8ccccc8)n7)cc6)cccc45)cc3)n2)cc1 ; c1cc(-c2ccc(-c3ccncc3)cc2)c2oc3c(-c4ccc(-c5ccncc5)cc4)cccc3c2c1 ; c1cncc(-c2ccc(-c3cccc4c3oc3c(-c5cccnc5)cccc34)cc2)c1 ; c1ccc(-c2ccc(-c3ccc(-c4cccc5c4oc4c(-c6cccnc6)cccc45)cc3)cn2)cc1]

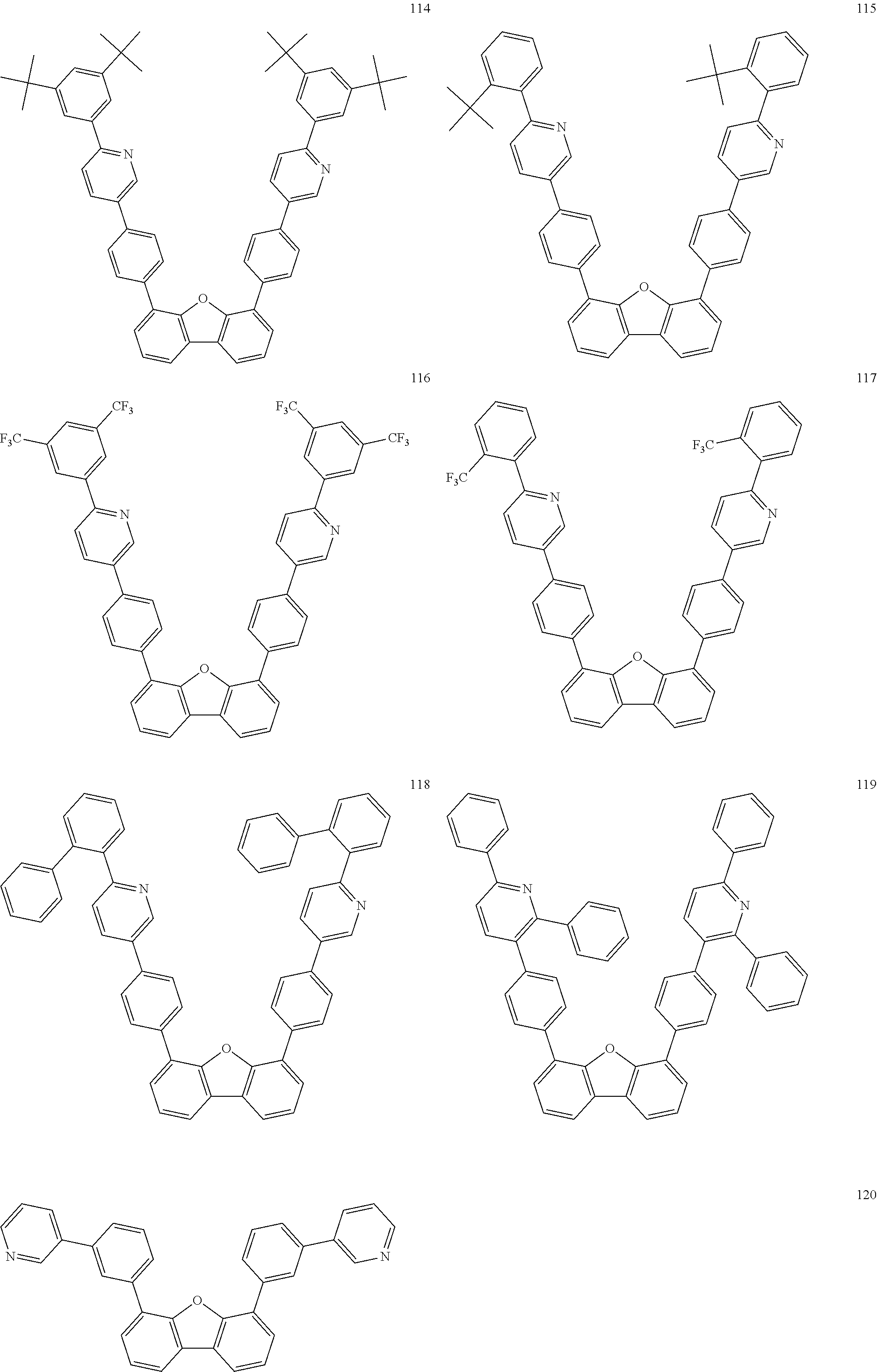

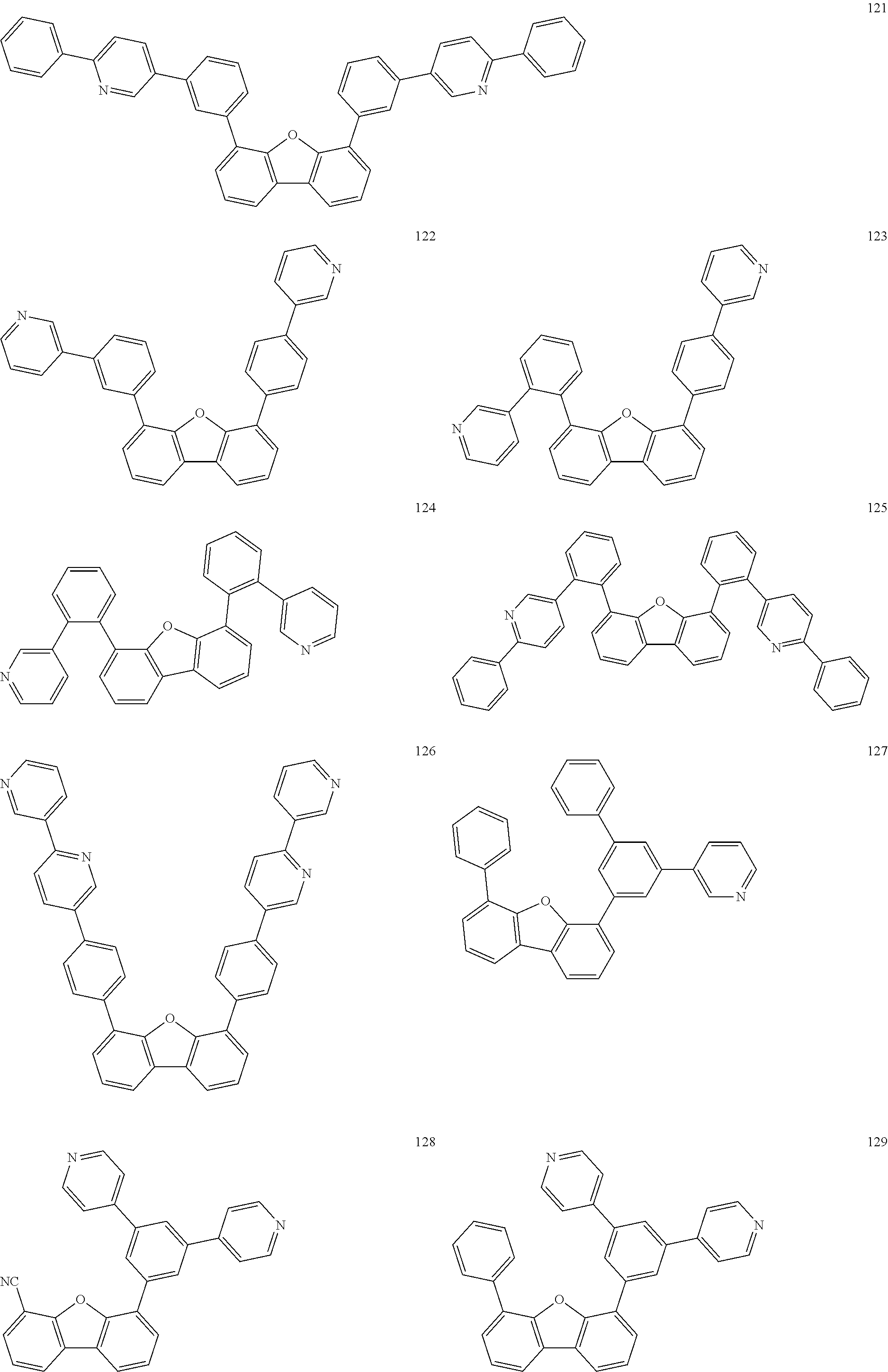

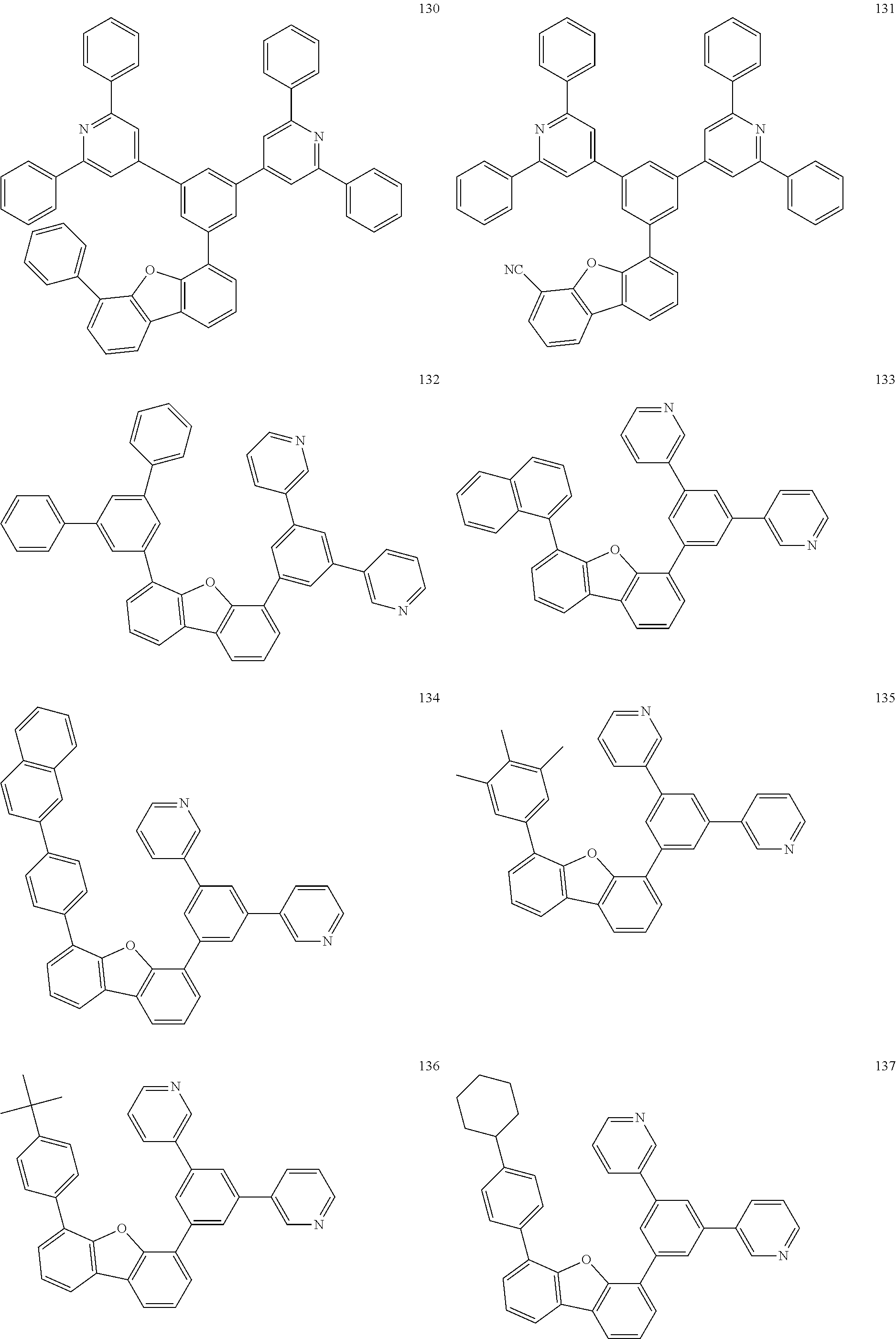

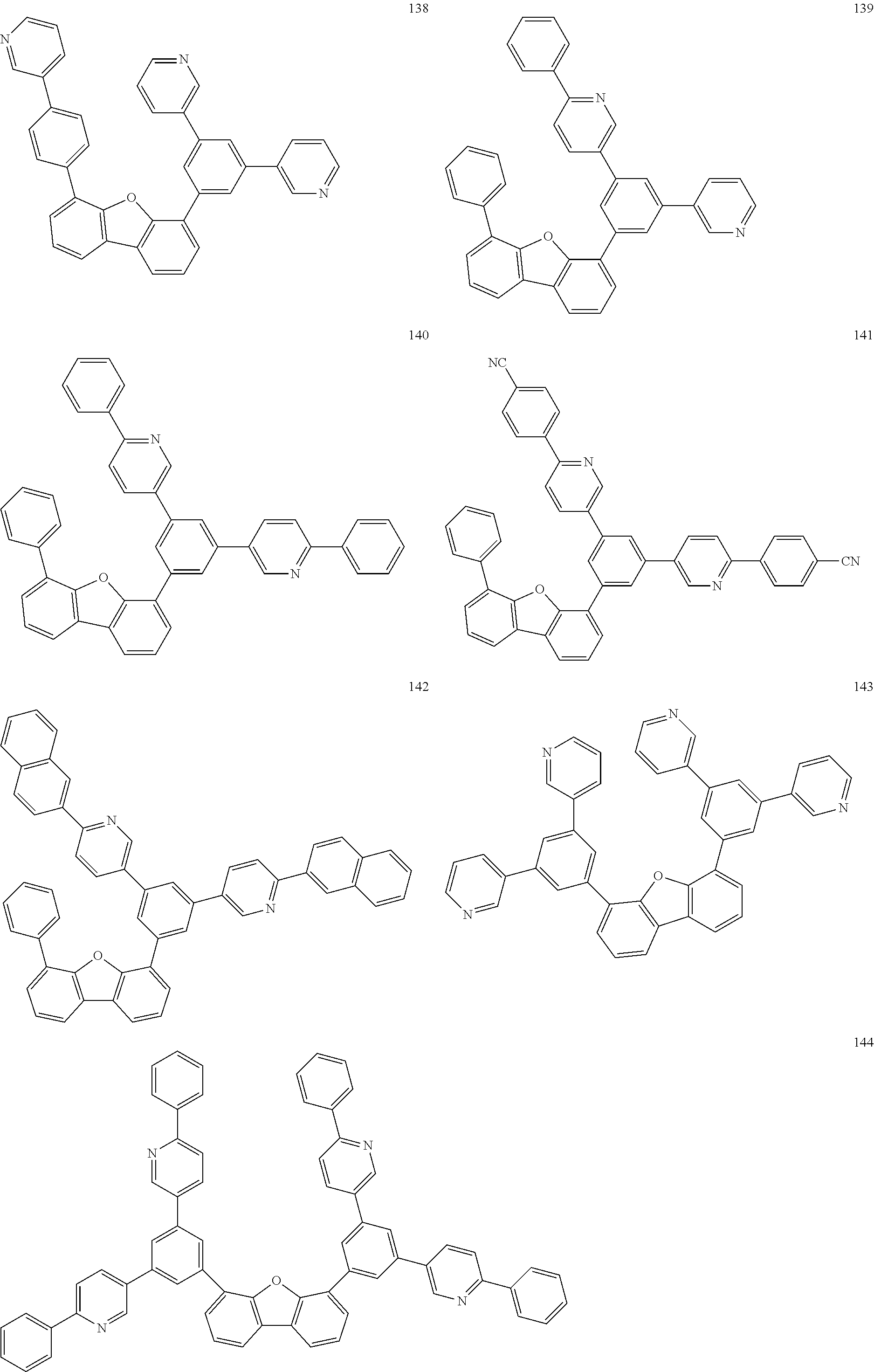

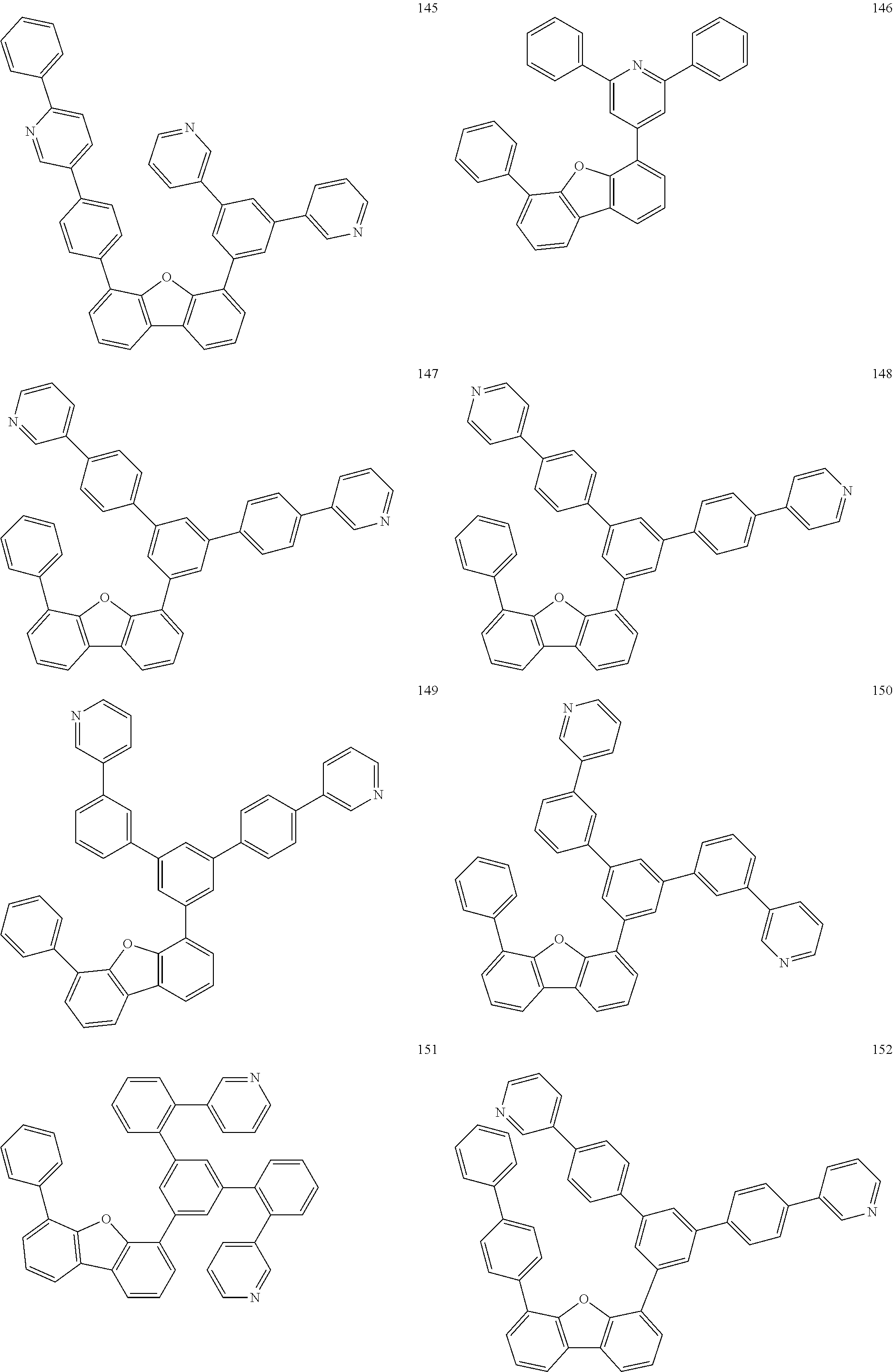

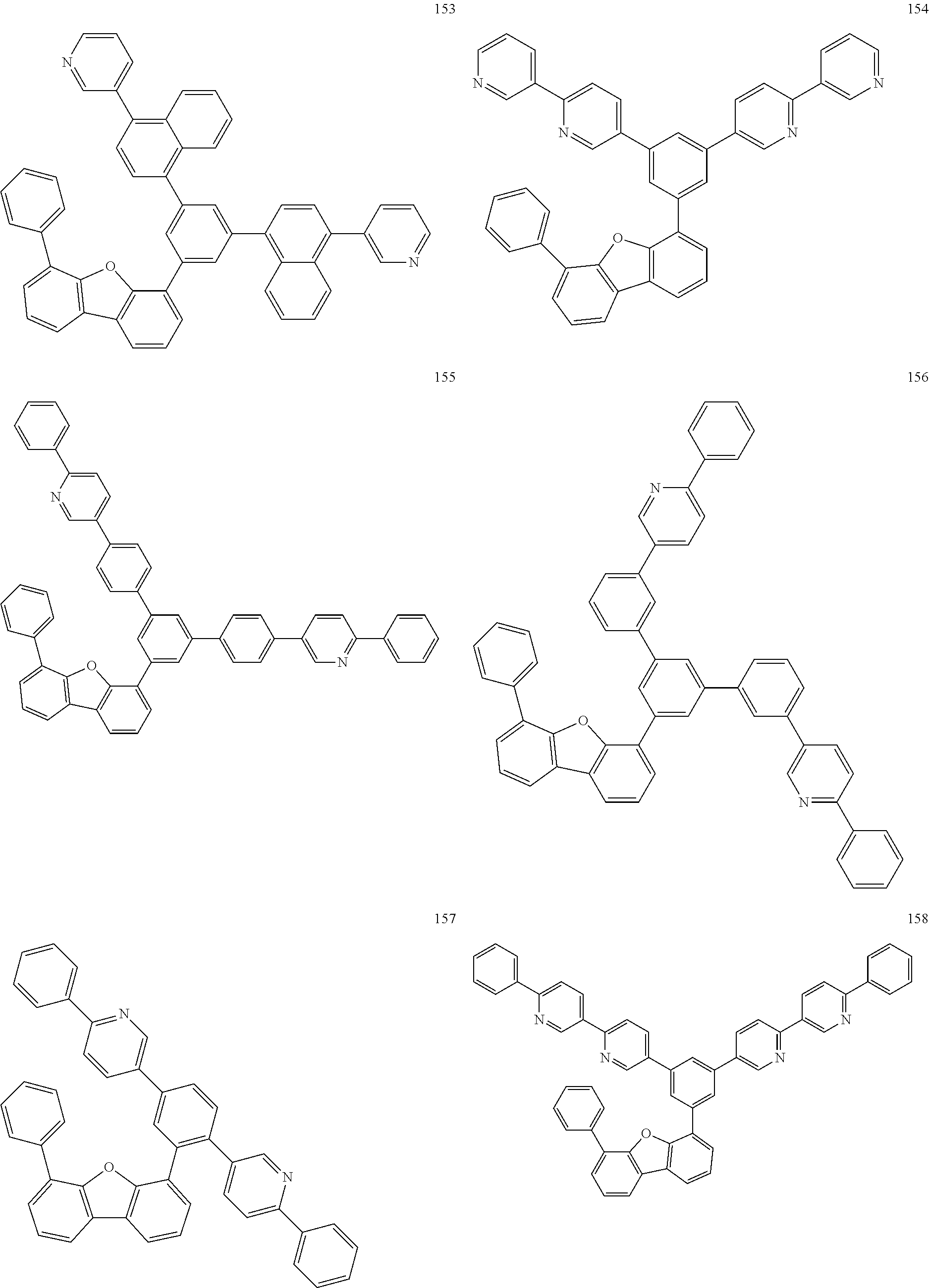

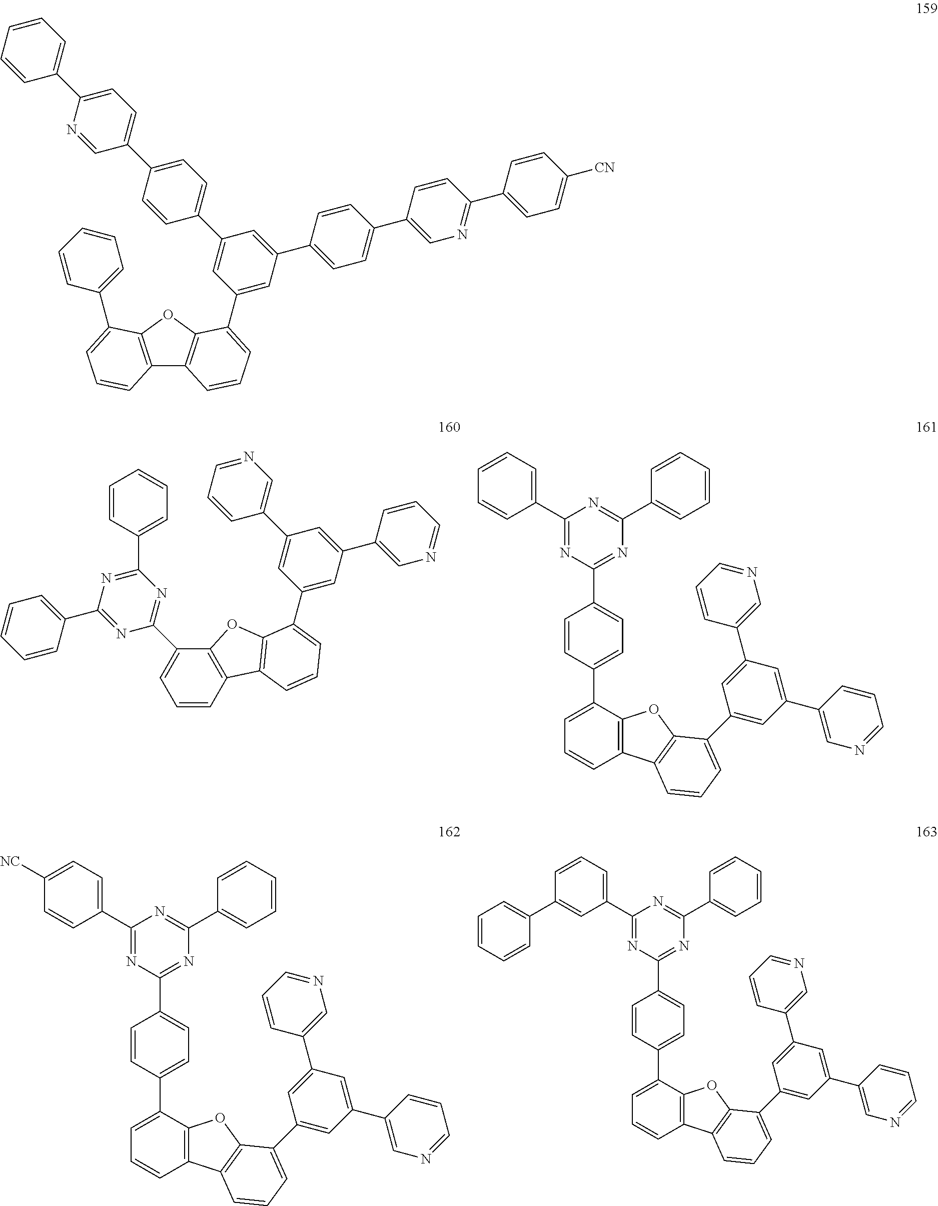

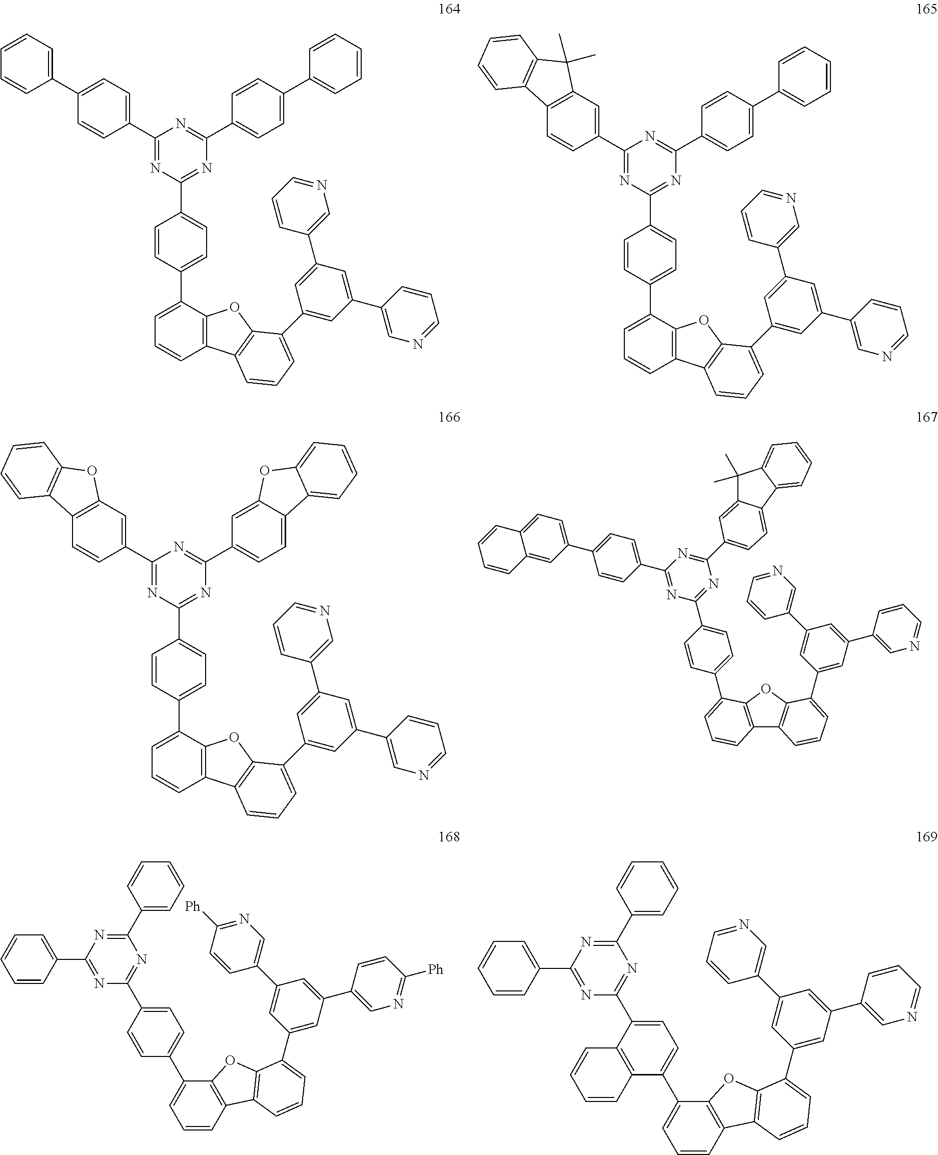

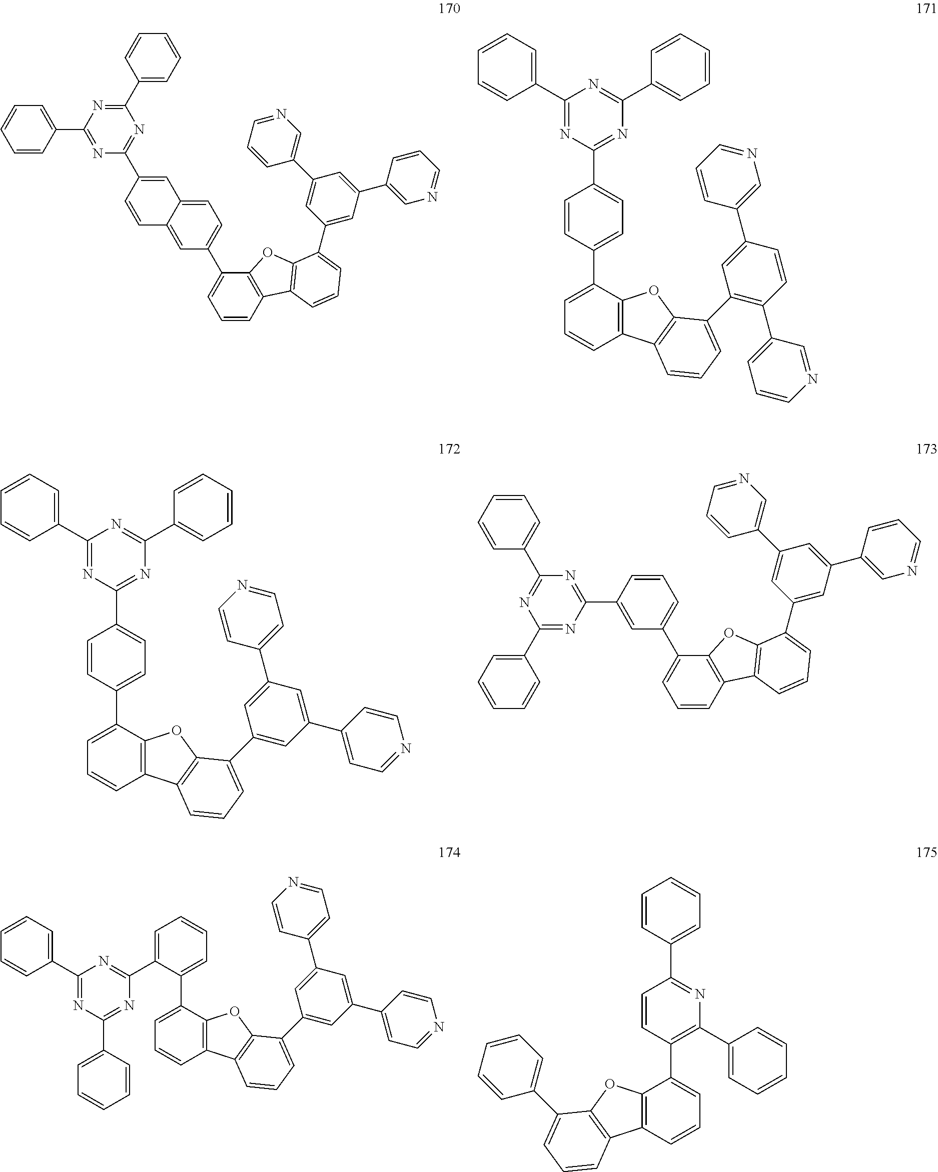

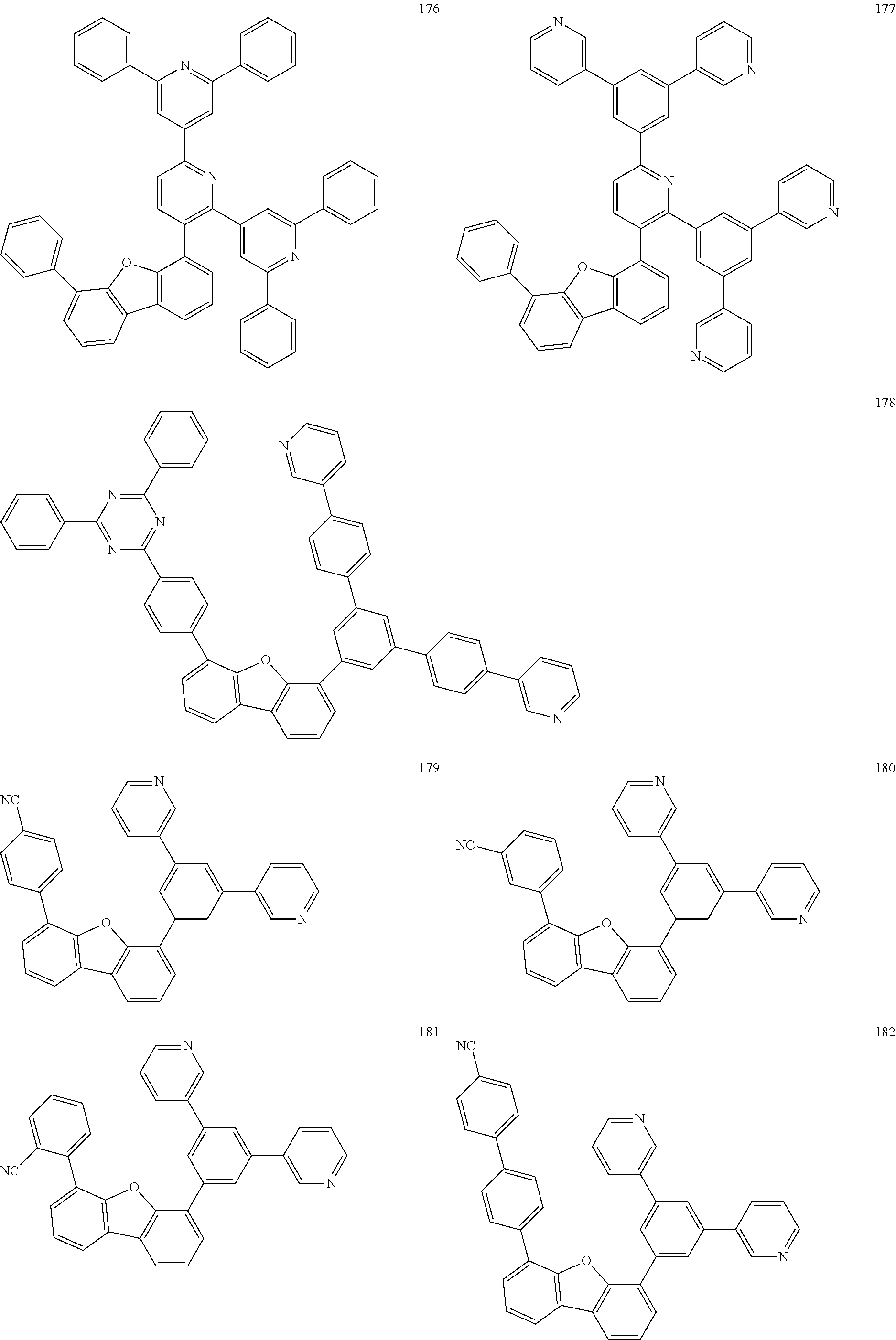

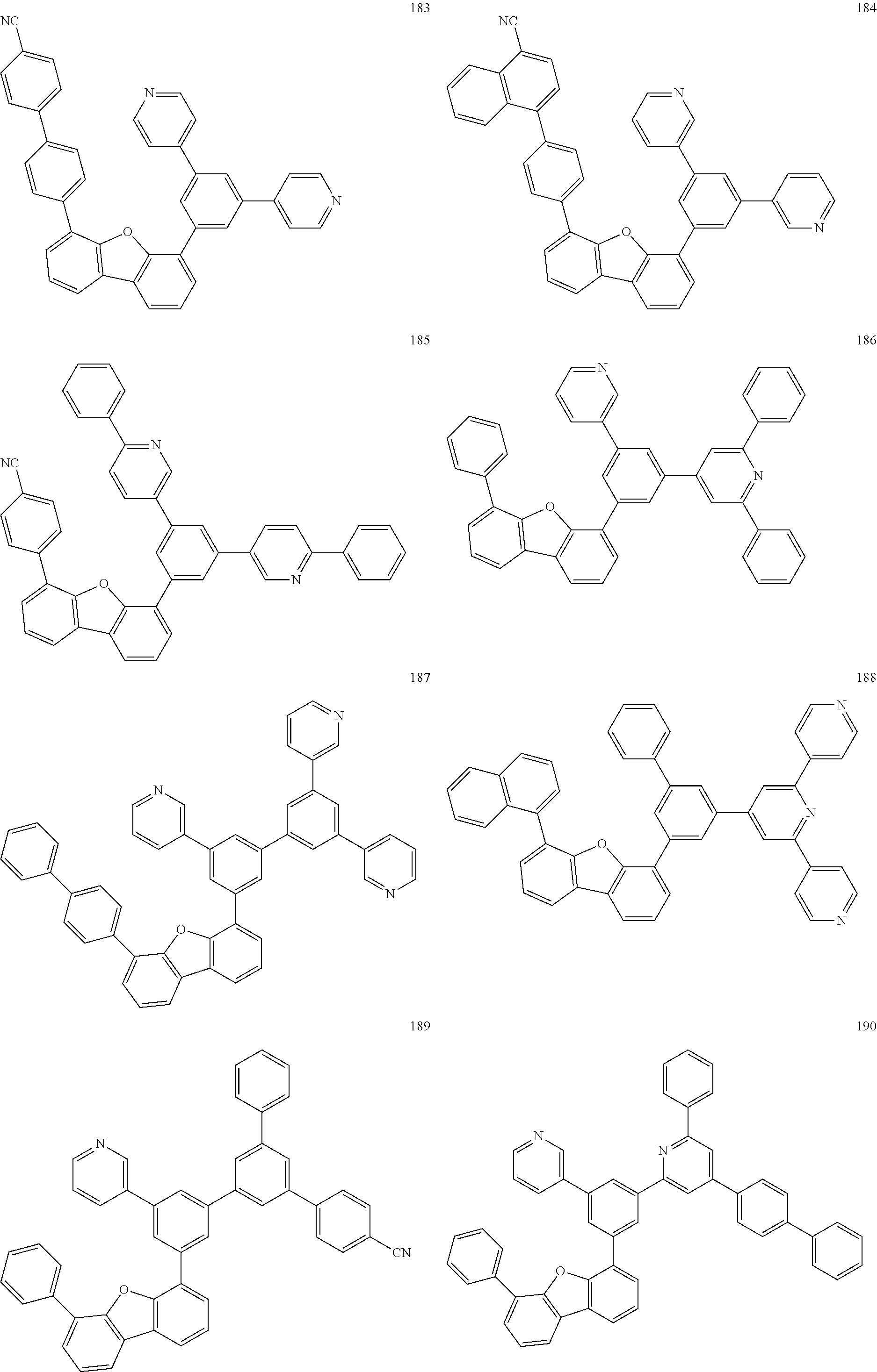

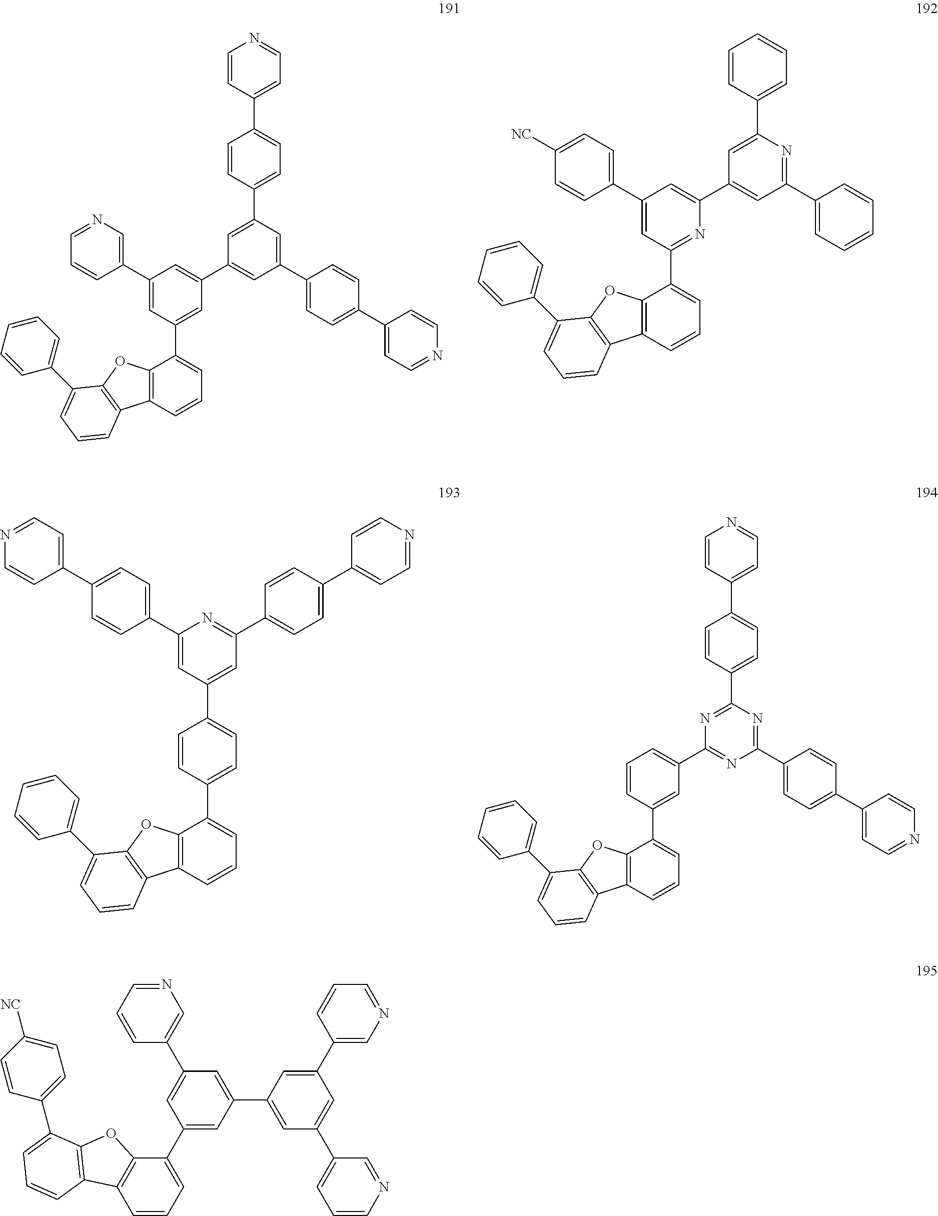 [SMILES: c1ccc(-c2cccc3c2oc2c(-c4cc(-c5cccnc5)cc(-c5cc(-c6ccc(-c7ccncc7)cc6)cc(-c6ccc(-c7ccncc7)cc6)c5)c4)cccc23)cc1 ; N#Cc1ccc(-c2cc(-c3cc(-c4ccccc4)nc(-c4ccccc4)c3)nc(-c3cccc4c3oc3c(-c5ccccc5)cccc34)c2)cc1 ; c1ccc(-c2cccc3c2oc2c(-c4ccc(-c5cc(-c6ccc(-c7ccncc7)cc6)nc(-c6ccc(-c7ccncc7)cc6)c5)cc4)cccc23)cc1 ; c1ccc(-c2cccc3c2oc2c(-c4cccc(-c5nc(-c6ccc(-c7ccncc7)cc6)nc(-c6ccc(-c7ccncc7)cc6)n5)c4)cccc23)cc1 ; N#Cc1ccc(-c2cccc3c2oc2c(-c4cc(-c5cccnc5)cc(-c5cc(-c6cccnc6)cc(-c6cccnc6)c5)c4)cccc23)cc1]

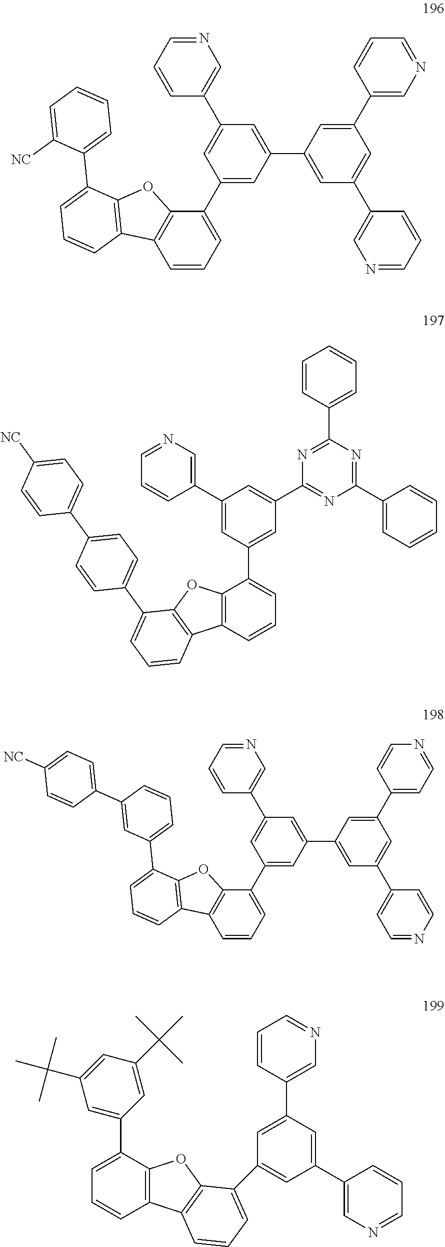 [SMILES: N#Cc1ccccc1-c1cccc2c1oc1c(-c3cc(-c4cccnc4)cc(-c4cc(-c5cccnc5)cc(-c5cccnc5)c4)c3)cccc12 ; N#Cc1ccc(-c2ccc(-c3cccc4c3oc3c(-c5cc(-c6cccnc6)cc(-c6nc(-c7ccccc7)nc(-c7ccccc7)n6)c5)cccc34)cc2)cc1 ; N#Cc1ccc(-c2cccc(-c3cccc4c3oc3c(-c5cc(-c6cccnc6)cc(-c6cc(-c7ccncc7)cc(-c7ccncc7)c6)c5)cccc34)c2)cc1 ; CC(C)(C)c1cc(-c2cccc3c2oc2c(-c4cc(-c5cccnc5)cc(-c5cccnc5)c4)cccc23)cc(C(C)(C)C)c1]

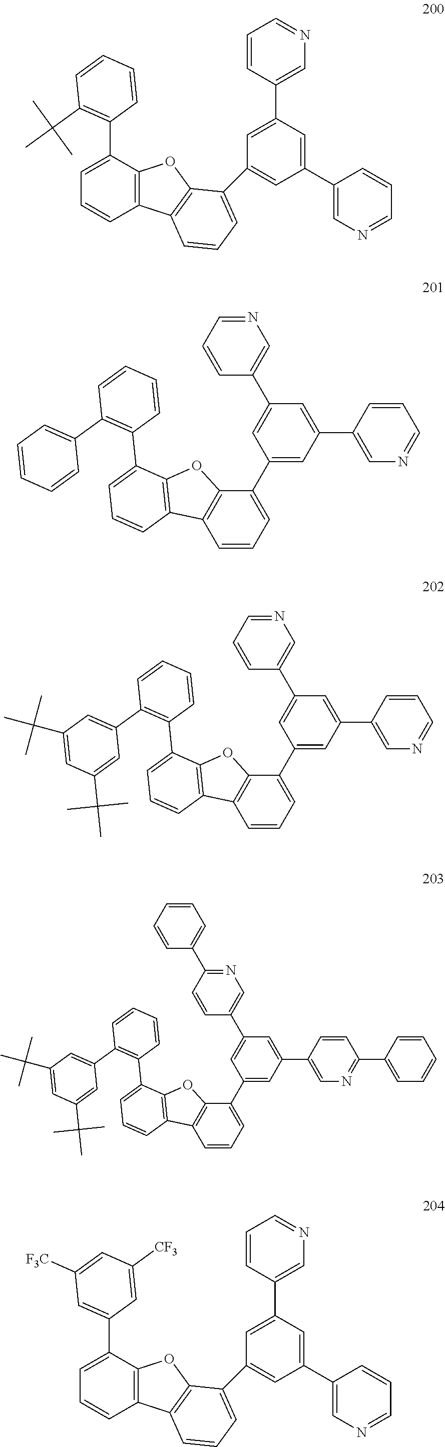 [SMILES: CC(C)(C)c1ccccc1-c1cccc2c1oc1c(-c3cc(-c4cccnc4)cc(-c4cccnc4)c3)cccc12 ; c1ccc(-c2ccccc2-c2cccc3c2oc2c(-c4cc(-c5cccnc5)cc(-c5cccnc5)c4)cccc23)cc1 ; CC(C)(C)c1cc(-c2ccccc2-c2cccc3c2oc2c(-c4cc(-c5cccnc5)cc(-c5cccnc5)c4)cccc23)cc(C(C)(C)C)c1 ; CC(C)(C)c1cc(-c2ccccc2-c2cccc3c2oc2c(-c4cc(-c5ccc(-c6ccccc6)nc5)cc(-c5ccc(-c6ccccc6)nc5)c4)cccc23)cc(C(C)(C)C)c1 ; FC(F)(F)c1cc(-c2cccc3c2oc2c(-c4cc(-c5cccnc5)cc(-c5cccnc5)c4)cccc23)cc(C(F)(F)F)c1]

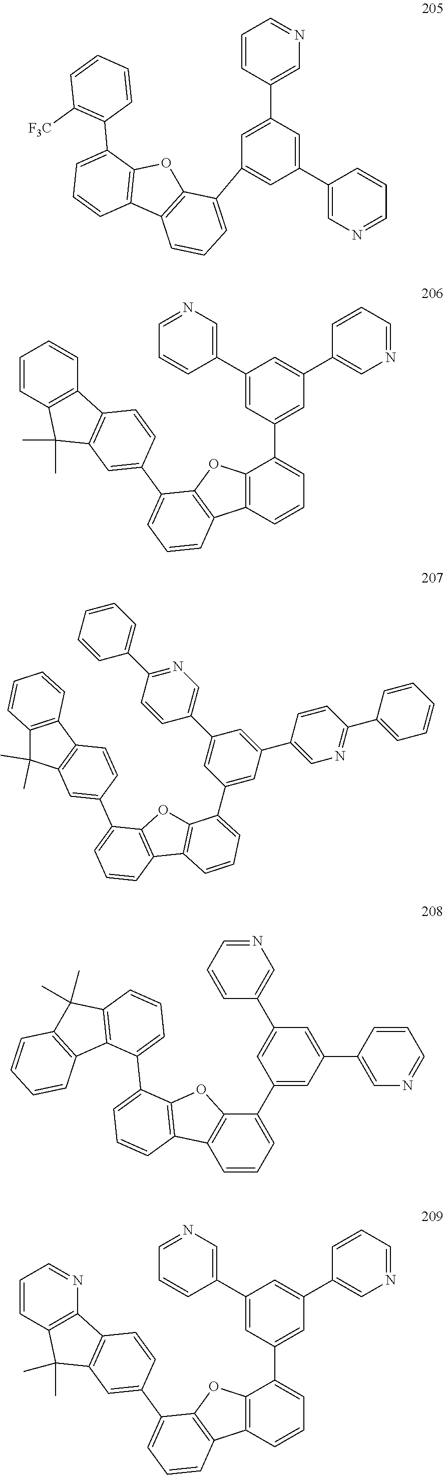 [SMILES: FC(F)(F)c1ccccc1-c1cccc2c1oc1c(-c3cc(-c4cccnc4)cc(-c4cccnc4)c3)cccc12 ; CC1(C)c2ccccc2-c2ccc(-c3cccc4c3oc3c(-c5cc(-c6cccnc6)cc(-c6cccnc6)c5)cccc34)cc21 ; CC1(C)c2ccccc2-c2ccc(-c3cccc4c3oc3c(-c5cc(-c6ccc(-c7ccccc7)nc6)cc(-c6ccc(-c7ccccc7)nc6)c5)cccc34)cc21 ; CC1(C)c2ccccc2-c2c(-c3cccc4c3oc3c(-c5cc(-c6cccnc6)cc(-c6cccnc6)c5)cccc34)cccc21 ; CC1(C)c2cc(-c3cccc4c3oc3c(-c5cc(-c6cccnc6)cc(-c6cccnc6)c5)cccc34)ccc2-c2ncccc21]

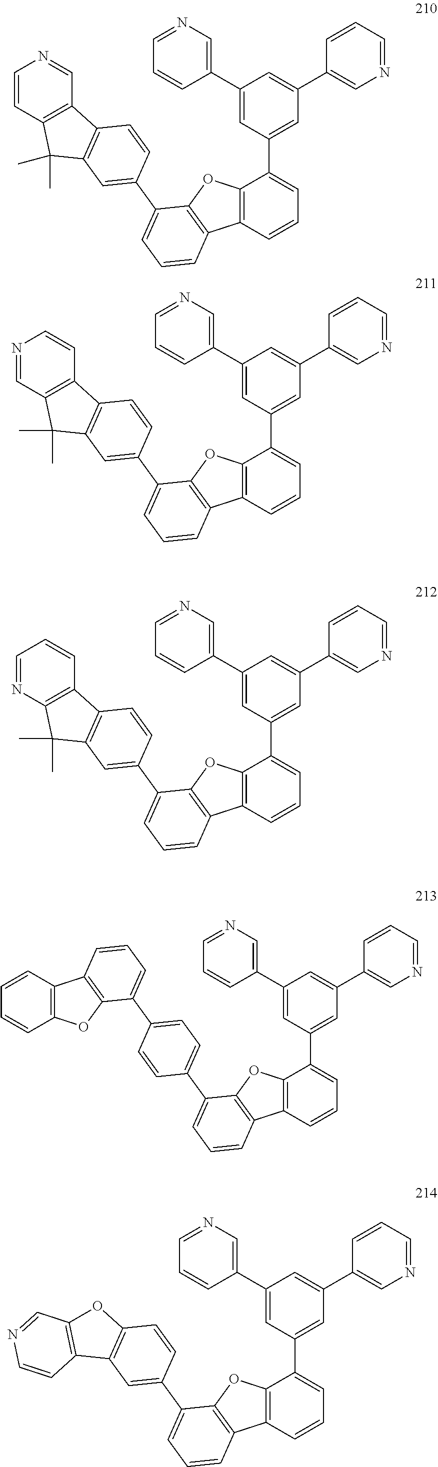

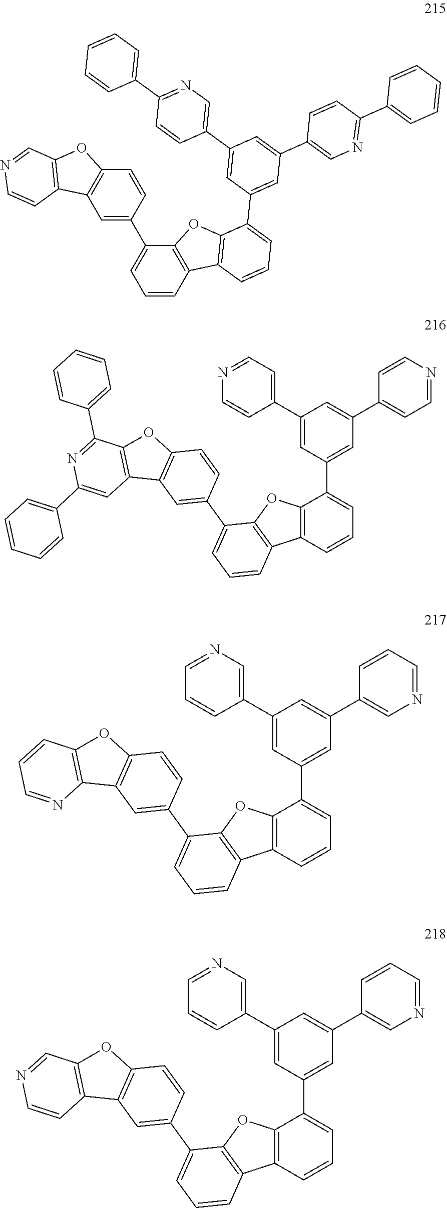 [SMILES: c1ccc(-c2ccc(-c3cc(-c4ccc(-c5ccccc5)nc4)cc(-c4cccc5c4oc4c(-c6ccc7oc8cnccc8c7c6)cccc45)c3)cn2)cc1 ; c1ccc(-c2cc3c(oc4ccc(-c5cccc6c5oc5c(-c7cc(-c8ccncc8)cc(-c8ccncc8)c7)cccc56)cc43)c(-c3ccccc3)n2)cc1 ; c1cncc(-c2cc(-c3cccnc3)cc(-c3cccc4c3oc3c(-c5ccc6oc7cccnc7c6c5)cccc34)c2)c1 ; c1cncc(-c2cc(-c3cccnc3)cc(-c3cccc4c3oc3c(-c5ccc6oc7cnccc7c6c5)cccc34)c2)c1]

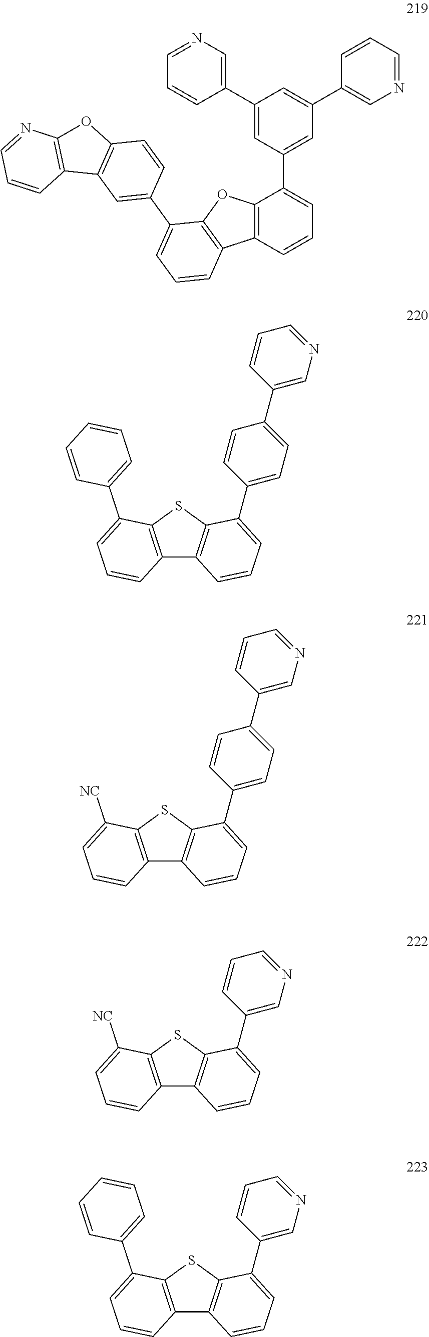 [SMILES: c1cncc(-c2cc(-c3cccnc3)cc(-c3cccc4c3oc3c(-c5ccc6oc7ncccc7c6c5)cccc34)c2)c1 ; c1ccc(-c2cccc3c2sc2c(-c4ccc(-c5cccnc5)cc4)cccc23)cc1 ; N#Cc1cccc2c1sc1c(-c3ccc(-c4cccnc4)cc3)cccc12 ; N#Cc1cccc2c1sc1c(-c3cccnc3)cccc12 ; c1ccc(-c2cccc3c2sc2c(-c4cccnc4)cccc23)cc1]

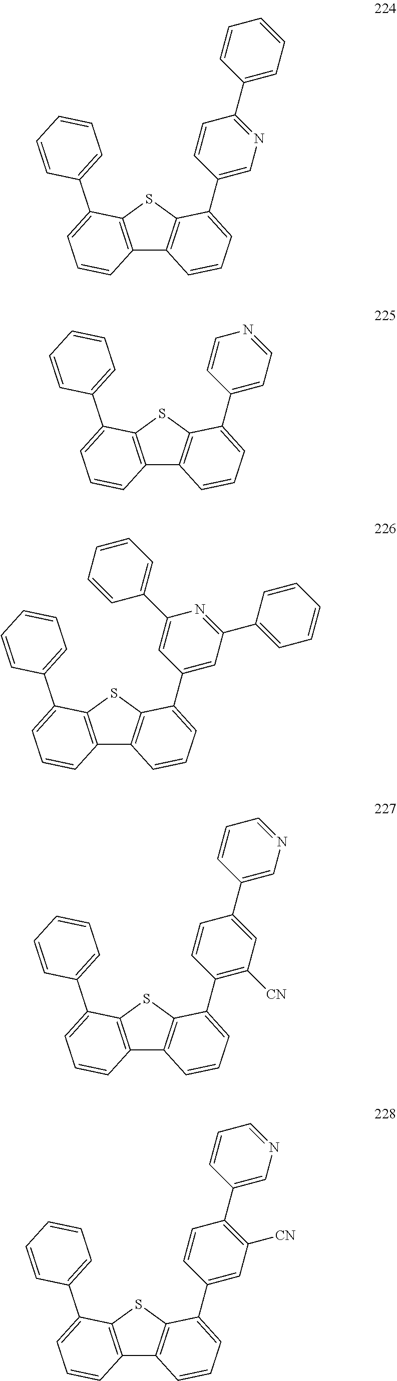 [SMILES: c1ccc(-c2ccc(-c3cccc4c3sc3c(-c5ccccc5)cccc34)cn2)cc1 ; c1ccc(-c2cccc3c2sc2c(-c4ccncc4)cccc23)cc1 ; c1ccc(-c2cc(-c3cccc4c3sc3c(-c5ccccc5)cccc34)cc(-c3ccccc3)n2)cc1 ; N#Cc1cc(-c2cccnc2)ccc1-c1cccc2c1sc1c(-c3ccccc3)cccc12 ; N#Cc1cc(-c2cccc3c2sc2c(-c4ccccc4)cccc23)ccc1-c1cccnc1]

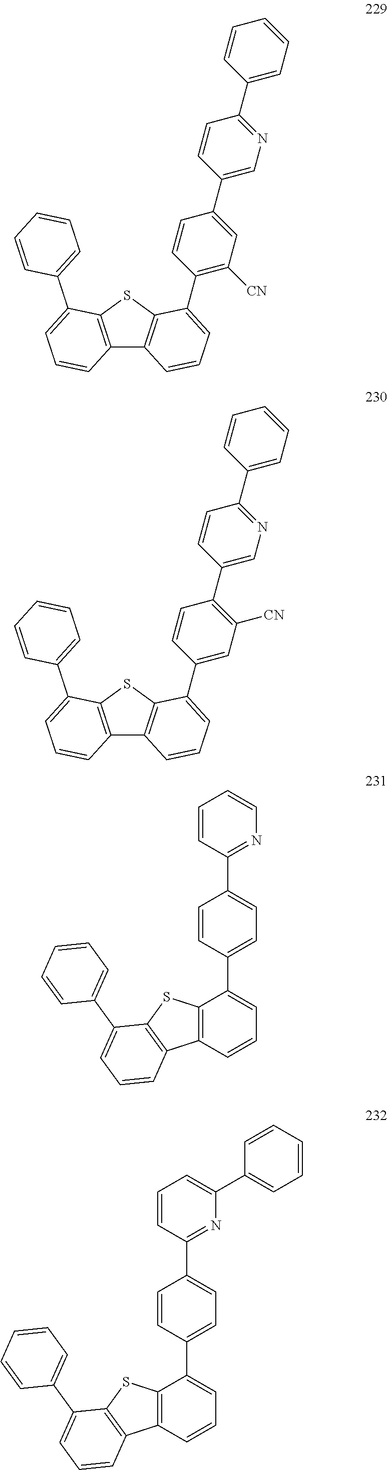 [SMILES: N#Cc1cc(-c2ccc(-c3ccccc3)nc2)ccc1-c1cccc2c1sc1c(-c3ccccc3)cccc12 ; N#Cc1cc(-c2cccc3c2sc2c(-c4ccccc4)cccc23)ccc1-c1ccc(-c2ccccc2)nc1 ; c1ccc(-c2cccc3c2sc2c(-c4ccc(-c5ccccn5)cc4)cccc23)cc1 ; c1ccc(-c2cccc(-c3ccc(-c4cccc5c4sc4c(-c6ccccc6)cccc45)cc3)n2)cc1]

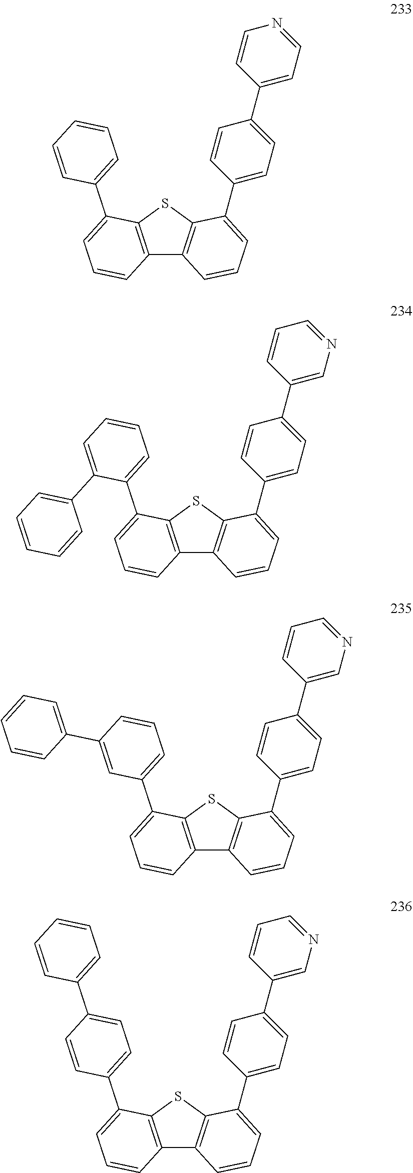 [SMILES: c1ccc(-c2cccc3c2sc2c(-c4ccc(-c5ccncc5)cc4)cccc23)cc1 ; c1ccc(-c2ccccc2-c2cccc3c2sc2c(-c4ccc(-c5cccnc5)cc4)cccc23)cc1 ; c1ccc(-c2cccc(-c3cccc4c3sc3c(-c5ccc(-c6cccnc6)cc5)cccc34)c2)cc1 ; c1ccc(-c2ccc(-c3cccc4c3sc3c(-c5ccc(-c6cccnc6)cc5)cccc34)cc2)cc1]

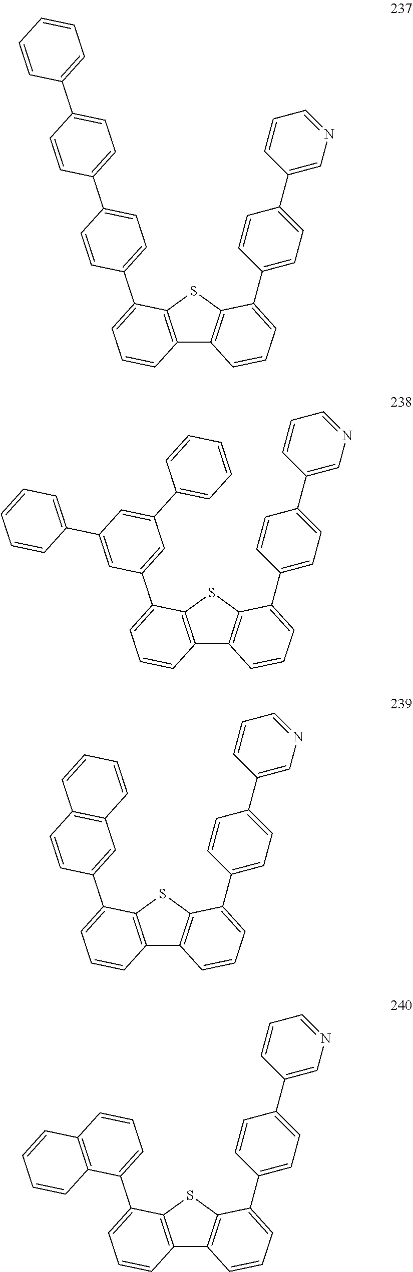 [SMILES: c1ccc(-c2ccc(-c3ccc(-c4cccc5c4sc4c(-c6ccc(-c7cccnc7)cc6)cccc45)cc3)cc2)cc1 ; c1ccc(-c2cc(-c3ccccc3)cc(-c3cccc4c3sc3c(-c5ccc(-c6cccnc6)cc5)cccc34)c2)cc1 ; c1cncc(-c2ccc(-c3cccc4c3sc3c(-c5ccc6ccccc6c5)cccc34)cc2)c1 ; c1cncc(-c2ccc(-c3cccc4c3sc3c(-c5cccc6ccccc56)cccc34)cc2)c1]

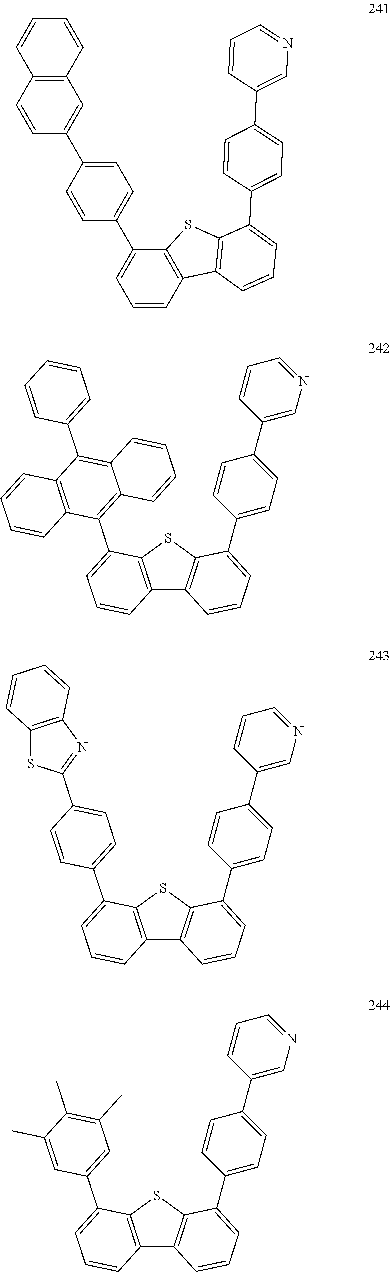 [SMILES: c1cncc(-c2ccc(-c3cccc4c3sc3c(-c5ccc(-c6ccc7ccccc7c6)cc5)cccc34)cc2)c1 ; c1ccc(-c2c3ccccc3c(-c3cccc4c3sc3c(-c5ccc(-c6cccnc6)cc5)cccc34)c3ccccc23)cc1 ; c1cncc(-c2ccc(-c3cccc4c3sc3c(-c5ccc(-c6nc7ccccc7s6)cc5)cccc34)cc2)c1 ; Cc1cc(-c2cccc3c2sc2c(-c4ccc(-c5cccnc5)cc4)cccc23)cc(C)c1C]

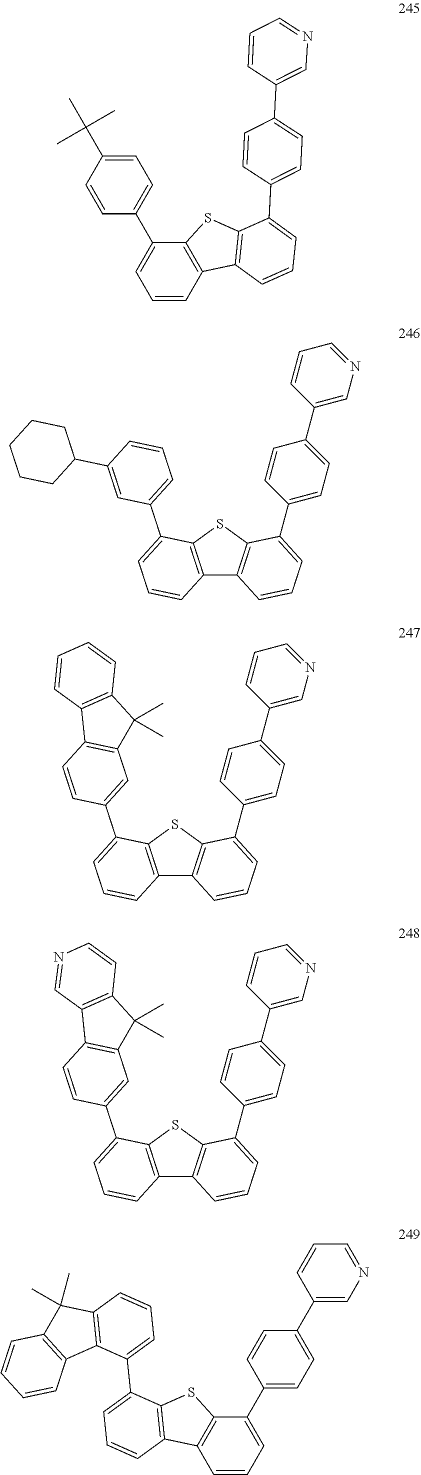 [SMILES: CC(C)(C)c1ccc(-c2cccc3c2sc2c(-c4ccc(-c5cccnc5)cc4)cccc23)cc1 ; c1cncc(-c2ccc(-c3cccc4c3sc3c(-c5cccc(C6CCCCC6)c5)cccc34)cc2)c1 ; CC1(C)c2ccccc2-c2ccc(-c3cccc4c3sc3c(-c5ccc(-c6cccnc6)cc5)cccc34)cc21 ; CC1(C)c2ccncc2-c2ccc(-c3cccc4c3sc3c(-c5ccc(-c6cccnc6)cc5)cccc34)cc21 ; CC1(C)c2ccccc2-c2c(-c3cccc4c3sc3c(-c5ccc(-c6cccnc6)cc5)cccc34)cccc21]

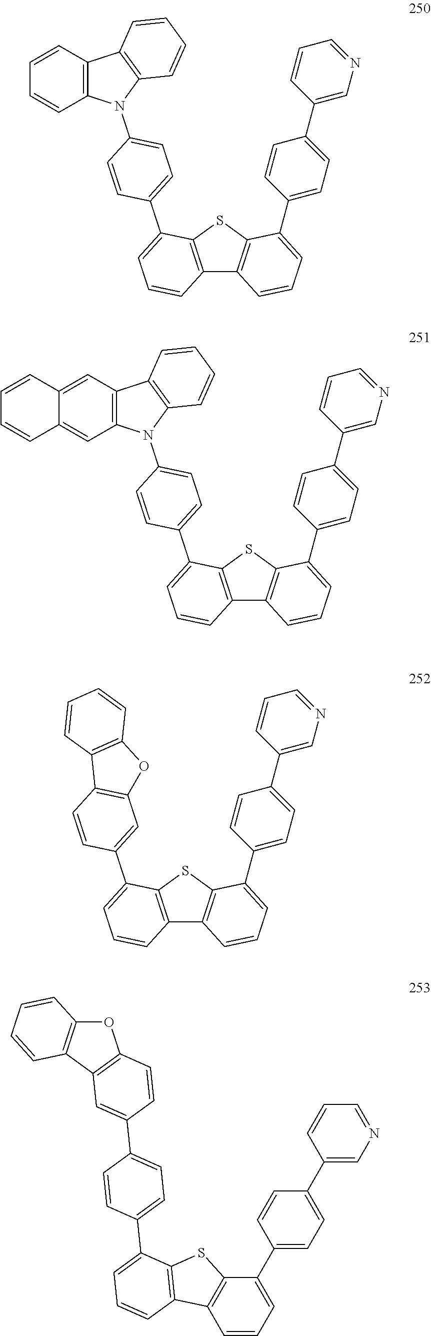 [SMILES: c1cncc(-c2ccc(-c3cccc4c3sc3c(-c5ccc(-n6c7ccccc7c7ccccc76)cc5)cccc34)cc2)c1 ; c1cncc(-c2ccc(-c3cccc4c3sc3c(-c5ccc(-n6c7ccccc7c7cc8ccccc8cc76)cc5)cccc34)cc2)c1 ; c1cncc(-c2ccc(-c3cccc4c3sc3c(-c5ccc6c(c5)oc5ccccc56)cccc34)cc2)c1 ; c1cncc(-c2ccc(-c3cccc4c3sc3c(-c5ccc(-c6ccc7oc8ccccc8c7c6)cc5)cccc34)cc2)c1]

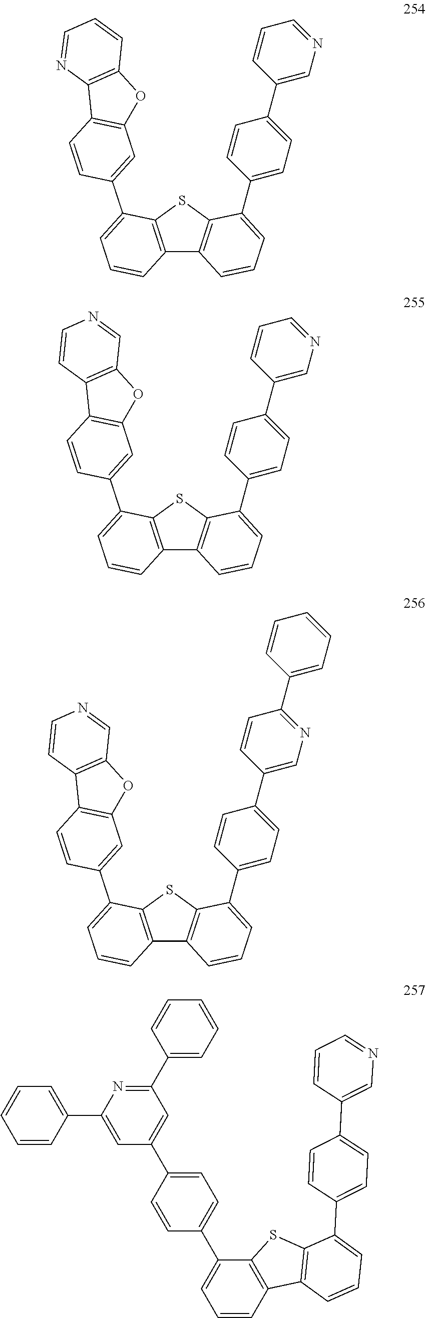

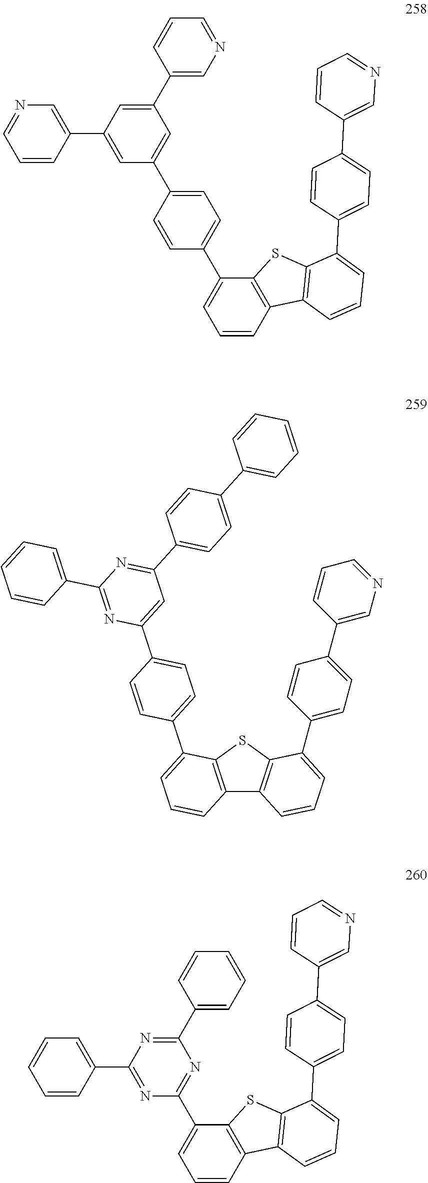 [SMILES: c1cncc(-c2ccc(-c3cccc4c3sc3c(-c5ccc(-c6cc(-c7cccnc7)cc(-c7cccnc7)c6)cc5)cccc34)cc2)c1 ; c1ccc(-c2ccc(-c3cc(-c4ccc(-c5cccc6c5sc5c(-c7ccc(-c8cccnc8)cc7)cccc56)cc4)nc(-c4ccccc4)n3)cc2)cc1 ; c1ccc(-c2nc(-c3ccccc3)nc(-c3cccc4c3sc3c(-c5ccc(-c6cccnc6)cc5)cccc34)n2)cc1]

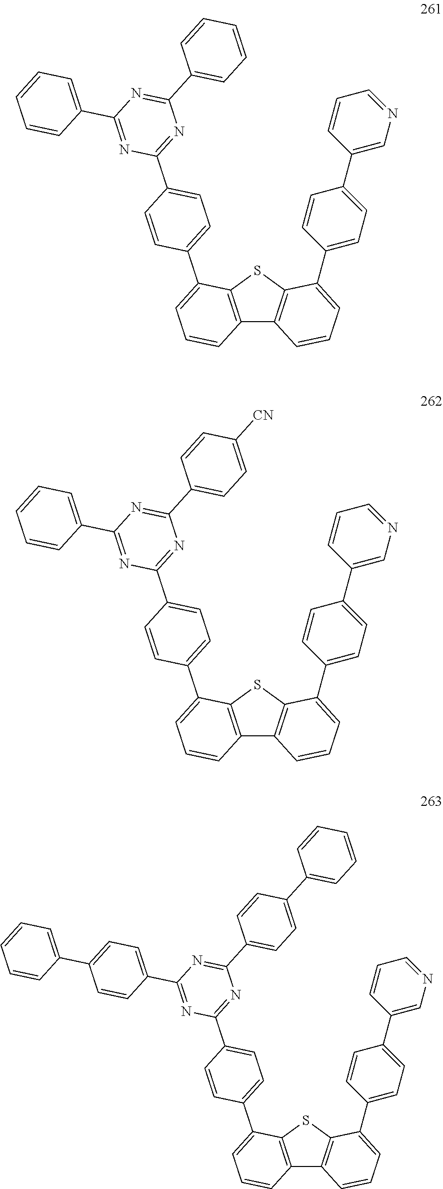 [SMILES: c1ccc(-c2nc(-c3ccccc3)nc(-c3ccc(-c4cccc5c4sc4c(-c6ccc(-c7cccnc7)cc6)cccc45)cc3)n2)cc1 ; N#Cc1ccc(-c2nc(-c3ccccc3)nc(-c3ccc(-c4cccc5c4sc4c(-c6ccc(-c7cccnc7)cc6)cccc45)cc3)n2)cc1 ; c1ccc(-c2ccc(-c3nc(-c4ccc(-c5ccccc5)cc4)nc(-c4ccc(-c5cccc6c5sc5c(-c7ccc(-c8cccnc8)cc7)cccc56)cc4)n3)cc2)cc1]

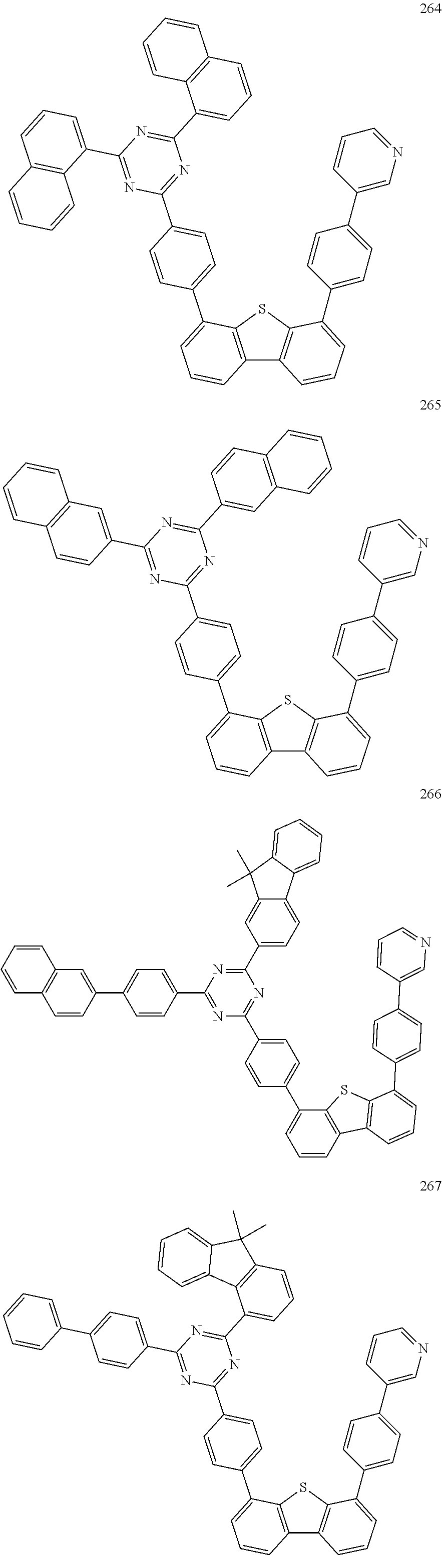 [SMILES: c1cncc(-c2ccc(-c3cccc4c3sc3c(-c5ccc(-c6nc(-c7cccc8ccccc78)nc(-c7cccc8ccccc78)n6)cc5)cccc34)cc2)c1 ; c1cncc(-c2ccc(-c3cccc4c3sc3c(-c5ccc(-c6nc(-c7ccc8ccccc8c7)nc(-c7ccc8ccccc8c7)n6)cc5)cccc34)cc2)c1 ; CC1(C)c2ccccc2-c2ccc(-c3nc(-c4ccc(-c5ccc6ccccc6c5)cc4)nc(-c4ccc(-c5cccc6c5sc5c(-c7ccc(-c8cccnc8)cc7)cccc56)cc4)n3)cc21 ; CC1(C)c2ccccc2-c2c(-c3nc(-c4ccc(-c5ccccc5)cc4)nc(-c4ccc(-c5cccc6c5sc5c(-c7ccc(-c8cccnc8)cc7)cccc56)cc4)n3)cccc21]

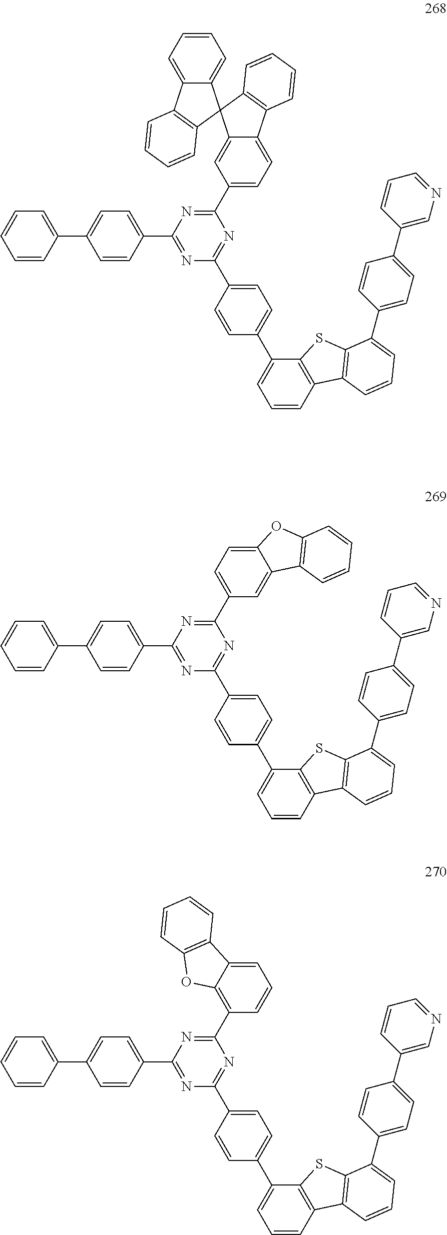 [SMILES: c1ccc(-c2ccc(-c3nc(-c4ccc(-c5cccc6c5sc5c(-c7ccc(-c8cccnc8)cc7)cccc56)cc4)nc(-c4ccc5c(c4)C4(c6ccccc6-c6ccccc64)c4ccccc4-5)n3)cc2)cc1 ; c1ccc(-c2ccc(-c3nc(-c4ccc(-c5cccc6c5sc5c(-c7ccc(-c8cccnc8)cc7)cccc56)cc4)nc(-c4ccc5oc6ccccc6c5c4)n3)cc2)cc1 ; c1ccc(-c2ccc(-c3nc(-c4ccc(-c5cccc6c5sc5c(-c7ccc(-c8cccnc8)cc7)cccc56)cc4)nc(-c4cccc5c4oc4ccccc45)n3)cc2)cc1]

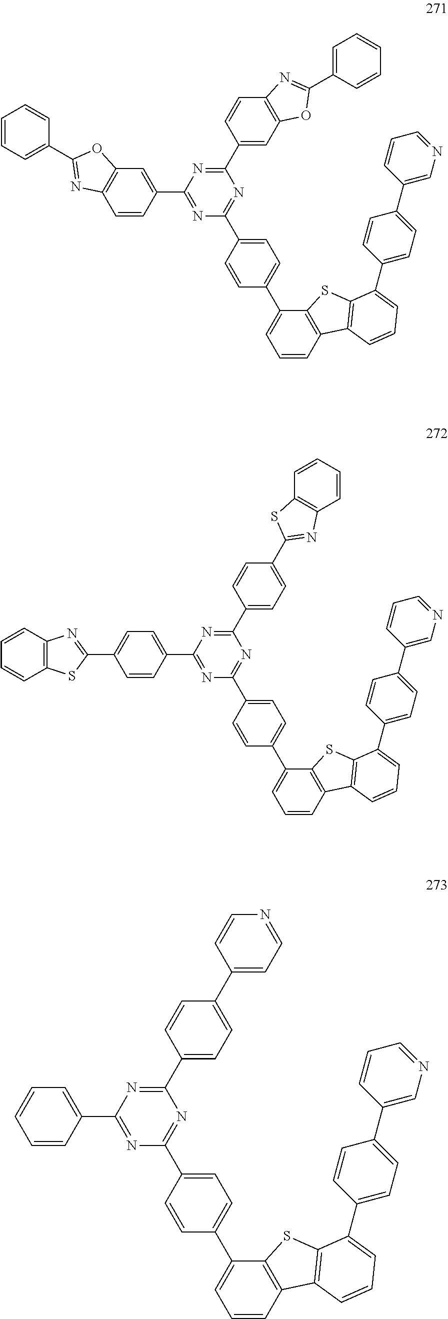 [SMILES: c1ccc(-c2nc3ccc(-c4nc(-c5ccc(-c6cccc7c6sc6c(-c8ccc(-c9cccnc9)cc8)cccc67)cc5)nc(-c5ccc6nc(-c7ccccc7)oc6c5)n4)cc3o2)cc1 ; c1cncc(-c2ccc(-c3cccc4c3sc3c(-c5ccc(-c6nc(-c7ccc(-c8nc9ccccc9s8)cc7)nc(-c7ccc(-c8nc9ccccc9s8)cc7)n6)cc5)cccc34)cc2)c1 ; c1ccc(-c2nc(-c3ccc(-c4ccncc4)cc3)nc(-c3ccc(-c4cccc5c4sc4c(-c6ccc(-c7cccnc7)cc6)cccc45)cc3)n2)cc1]

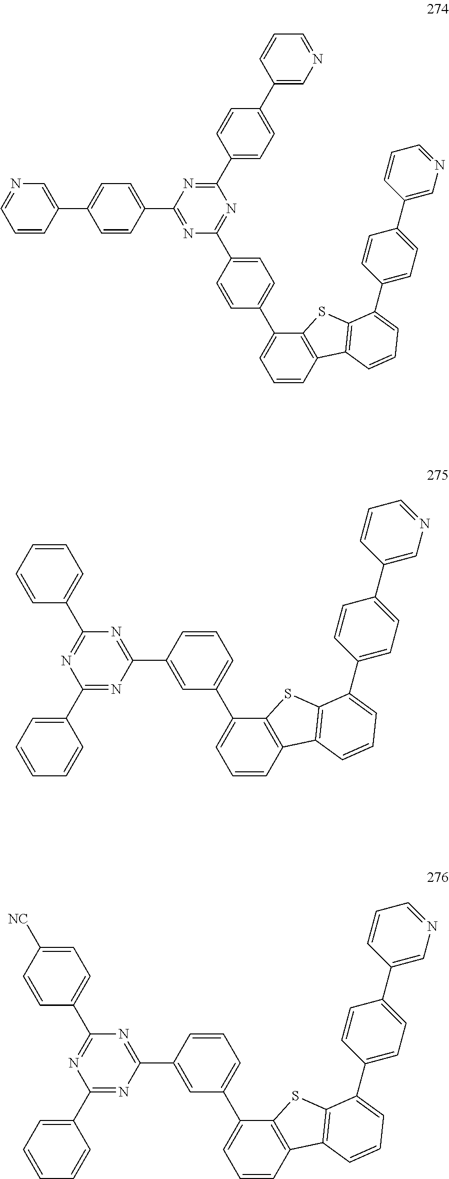 [SMILES: c1cncc(-c2ccc(-c3nc(-c4ccc(-c5cccnc5)cc4)nc(-c4ccc(-c5cccc6c5sc5c(-c7ccc(-c8cccnc8)cc7)cccc56)cc4)n3)cc2)c1 ; c1ccc(-c2nc(-c3ccccc3)nc(-c3cccc(-c4cccc5c4sc4c(-c6ccc(-c7cccnc7)cc6)cccc45)c3)n2)cc1 ; N#Cc1ccc(-c2nc(-c3ccccc3)nc(-c3cccc(-c4cccc5c4sc4c(-c6ccc(-c7cccnc7)cc6)cccc45)c3)n2)cc1]

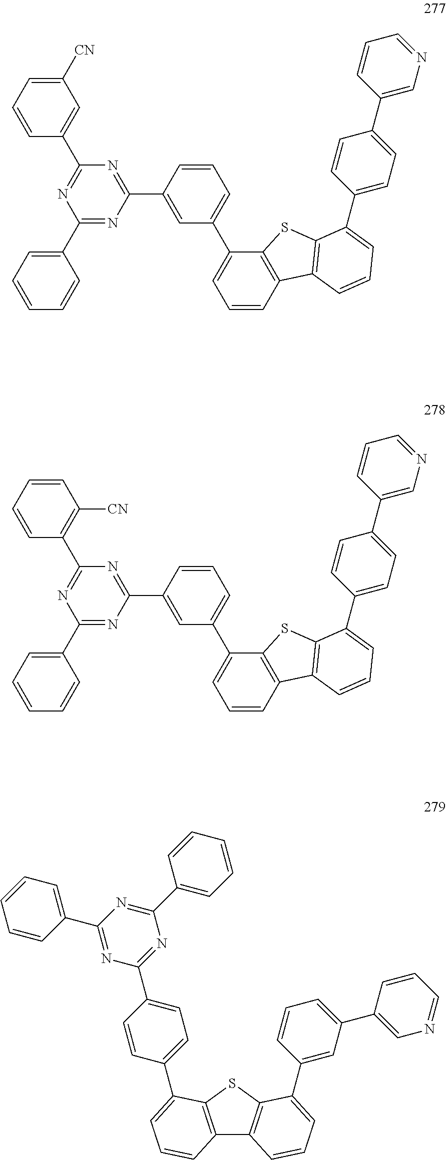 [SMILES: N#Cc1cccc(-c2nc(-c3ccccc3)nc(-c3cccc(-c4cccc5c4sc4c(-c6ccc(-c7cccnc7)cc6)cccc45)c3)n2)c1 ; N#Cc1ccccc1-c1nc(-c2ccccc2)nc(-c2cccc(-c3cccc4c3sc3c(-c5ccc(-c6cccnc6)cc5)cccc34)c2)n1 ; c1ccc(-c2nc(-c3ccccc3)nc(-c3ccc(-c4cccc5c4sc4c(-c6cccc(-c7cccnc7)c6)cccc45)cc3)n2)cc1]

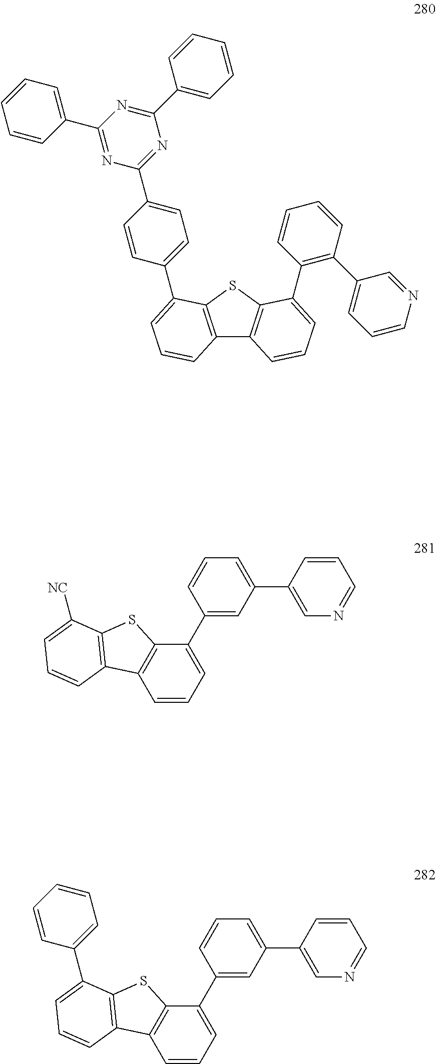 [SMILES: c1ccc(-c2nc(-c3ccccc3)nc(-c3ccc(-c4cccc5c4sc4c(-c6ccccc6-c6cccnc6)cccc45)cc3)n2)cc1 ; N#Cc1cccc2c1sc1c(-c3cccc(-c4cccnc4)c3)cccc12 ; c1ccc(-c2cccc3c2sc2c(-c4cccc(-c5cccnc5)c4)cccc23)cc1]

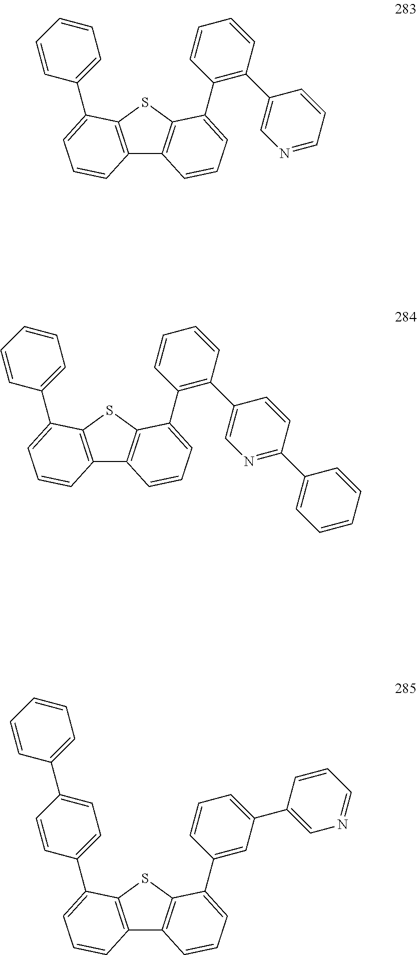 [SMILES: c1ccc(-c2cccc3c2sc2c(-c4ccccc4-c4cccnc4)cccc23)cc1 ; c1ccc(-c2ccc(-c3ccccc3-c3cccc4c3sc3c(-c5ccccc5)cccc34)cn2)cc1 ; c1ccc(-c2ccc(-c3cccc4c3sc3c(-c5cccc(-c6cccnc6)c5)cccc34)cc2)cc1]

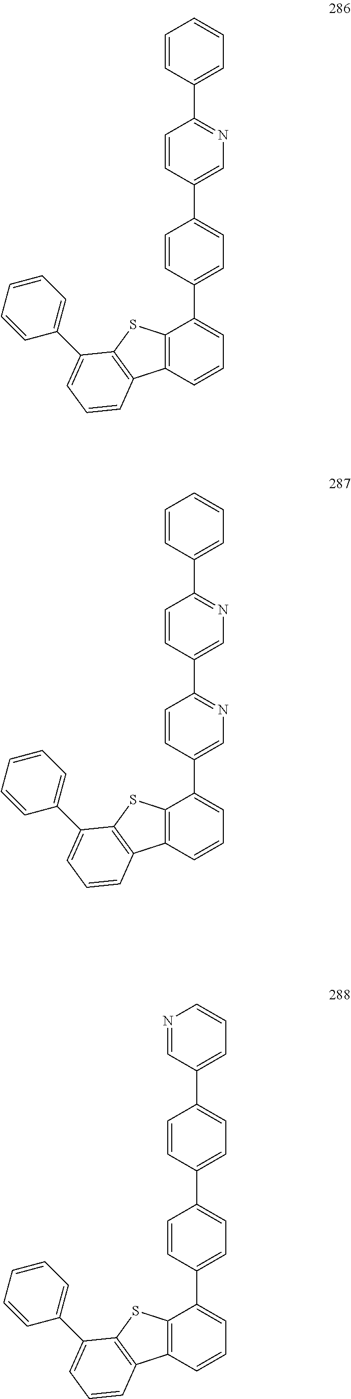 [SMILES: c1ccc(-c2ccc(-c3ccc(-c4cccc5c4sc4c(-c6ccccc6)cccc45)cc3)cn2)cc1 ; c1ccc(-c2ccc(-c3ccc(-c4cccc5c4sc4c(-c6ccccc6)cccc45)cn3)cn2)cc1 ; c1ccc(-c2cccc3c2sc2c(-c4ccc(-c5ccc(-c6cccnc6)cc5)cc4)cccc23)cc1]

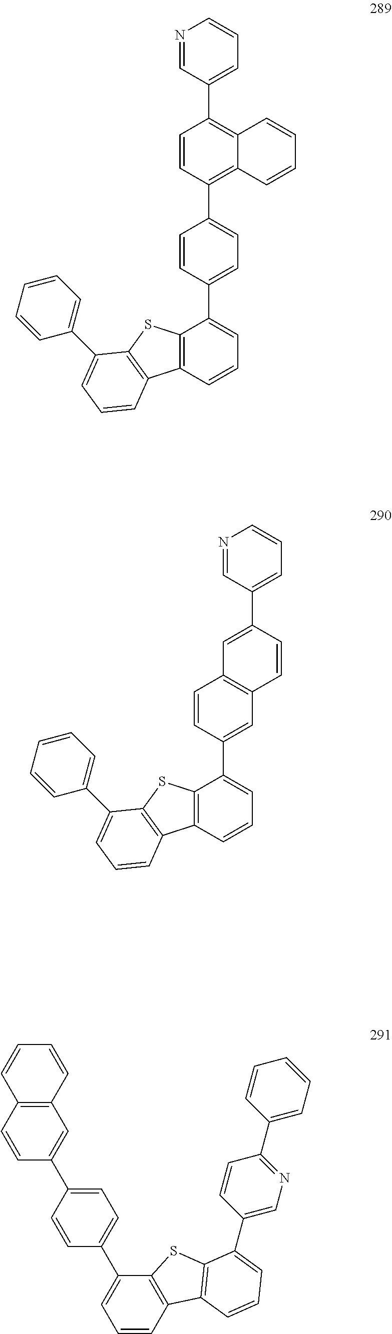 [SMILES: c1ccc(-c2cccc3c2sc2c(-c4ccc(-c5ccc(-c6cccnc6)c6ccccc56)cc4)cccc23)cc1 ; c1ccc(-c2cccc3c2sc2c(-c4ccc5cc(-c6cccnc6)ccc5c4)cccc23)cc1 ; c1ccc(-c2ccc(-c3cccc4c3sc3c(-c5ccc(-c6ccc7ccccc7c6)cc5)cccc34)cn2)cc1]

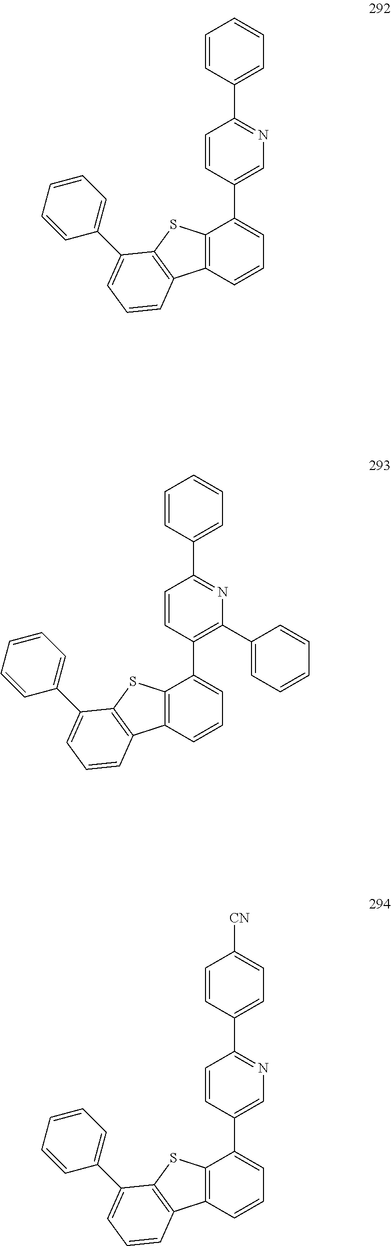 [SMILES: c1ccc(-c2ccc(-c3cccc4c3sc3c(-c5ccccc5)cccc34)cn2)cc1 ; c1ccc(-c2ccc(-c3cccc4c3sc3c(-c5ccccc5)cccc34)c(-c3ccccc3)n2)cc1 ; N#Cc1ccc(-c2ccc(-c3cccc4c3sc3c(-c5ccccc5)cccc34)cn2)cc1]

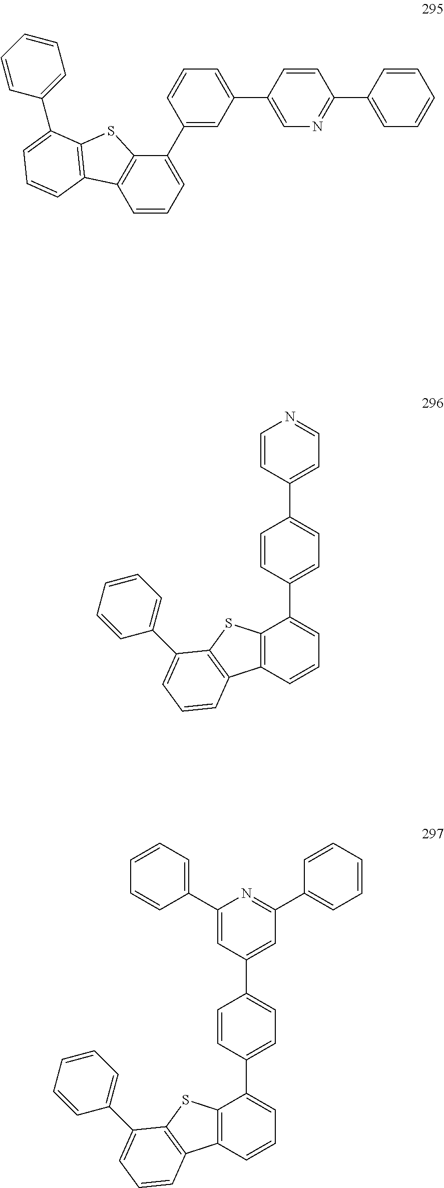 [SMILES: c1ccc(-c2ccc(-c3cccc(-c4cccc5c4sc4c(-c6ccccc6)cccc45)c3)cn2)cc1 ; c1ccc(-c2cccc3c2sc2c(-c4ccc(-c5ccncc5)cc4)cccc23)cc1 ; c1ccc(-c2cc(-c3ccc(-c4cccc5c4sc4c(-c6ccccc6)cccc45)cc3)cc(-c3ccccc3)n2)cc1]

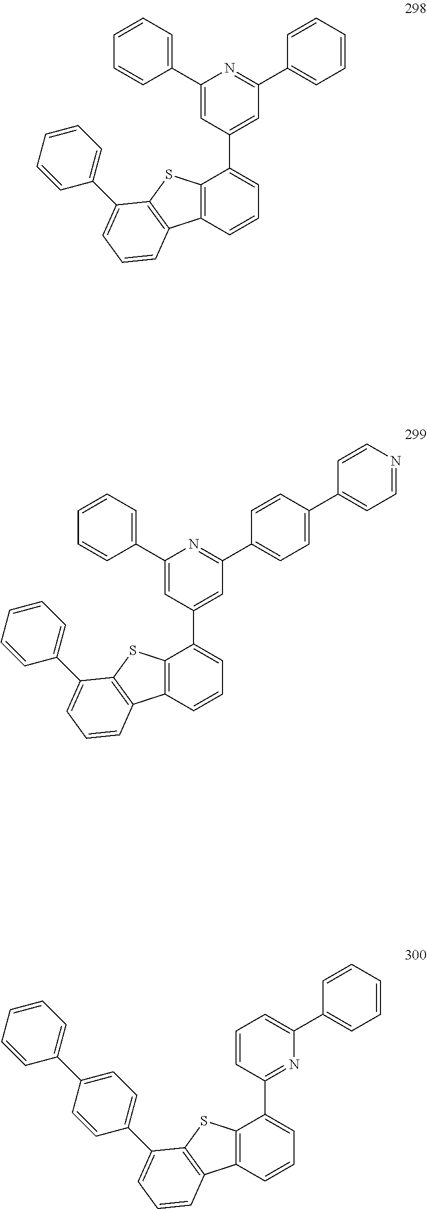 [SMILES: c1ccc(-c2cc(-c3cccc4c3sc3c(-c5ccccc5)cccc34)cc(-c3ccccc3)n2)cc1 ; c1ccc(-c2cc(-c3cccc4c3sc3c(-c5ccccc5)cccc34)cc(-c3ccc(-c4ccncc4)cc3)n2)cc1 ; c1ccc(-c2ccc(-c3cccc4c3sc3c(-c5cccc(-c6ccccc6)n5)cccc34)cc2)cc1]

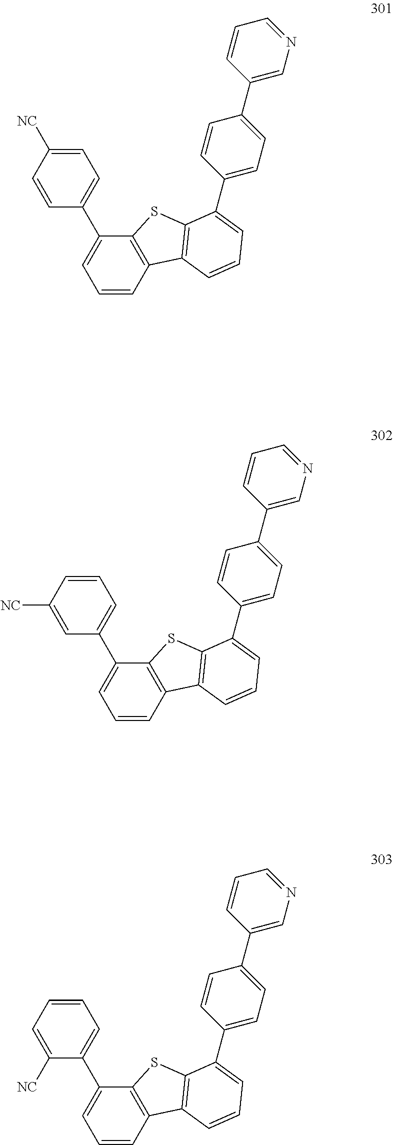 [SMILES: N#Cc1ccc(-c2cccc3c2sc2c(-c4ccc(-c5cccnc5)cc4)cccc23)cc1 ; N#Cc1cccc(-c2cccc3c2sc2c(-c4ccc(-c5cccnc5)cc4)cccc23)c1 ; N#Cc1ccccc1-c1cccc2c1sc1c(-c3ccc(-c4cccnc4)cc3)cccc12]

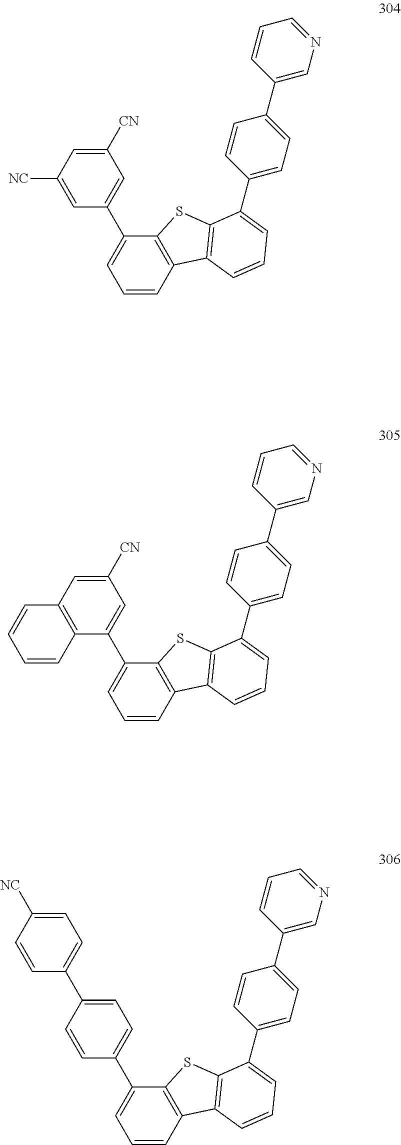 [SMILES: N#Cc1cc(C#N)cc(-c2cccc3c2sc2c(-c4ccc(-c5cccnc5)cc4)cccc23)c1 ; N#Cc1cc(-c2cccc3c2sc2c(-c4ccc(-c5cccnc5)cc4)cccc23)c2ccccc2c1 ; N#Cc1ccc(-c2ccc(-c3cccc4c3sc3c(-c5ccc(-c6cccnc6)cc5)cccc34)cc2)cc1]

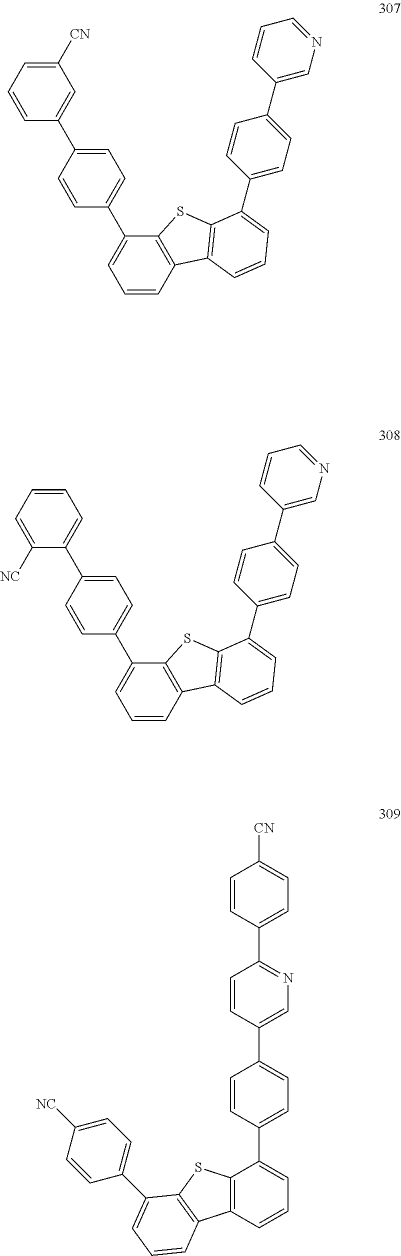 [SMILES: N#Cc1cccc(-c2ccc(-c3cccc4c3sc3c(-c5ccc(-c6cccnc6)cc5)cccc34)cc2)c1 ; N#Cc1ccccc1-c1ccc(-c2cccc3c2sc2c(-c4ccc(-c5cccnc5)cc4)cccc23)cc1 ; N#Cc1ccc(-c2ccc(-c3ccc(-c4cccc5c4sc4c(-c6ccc(C#N)cc6)cccc45)cc3)cn2)cc1]

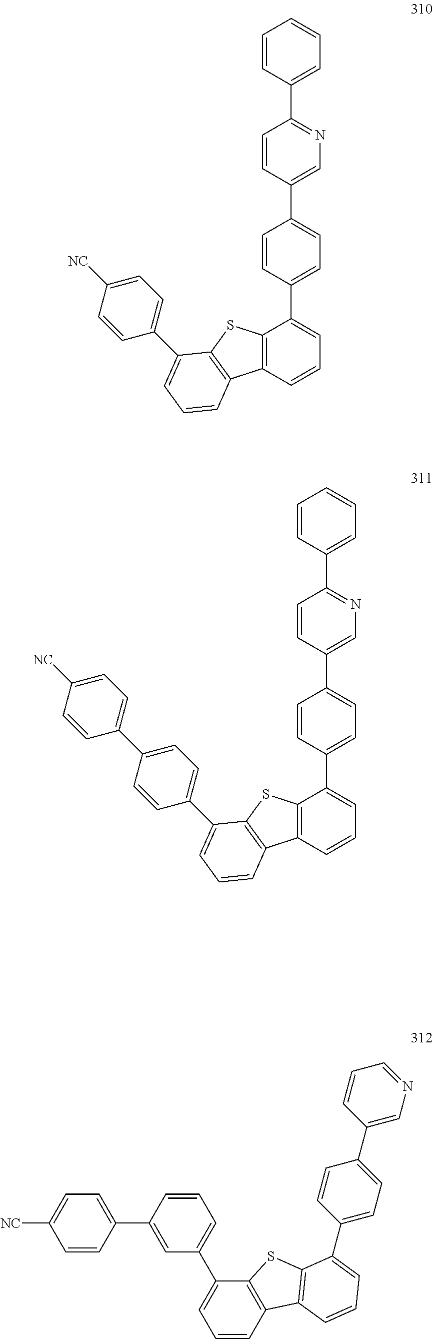 [SMILES: N#Cc1ccc(-c2cccc3c2sc2c(-c4ccc(-c5ccc(-c6ccccc6)nc5)cc4)cccc23)cc1 ; N#Cc1ccc(-c2ccc(-c3cccc4c3sc3c(-c5ccc(-c6ccc(-c7ccccc7)nc6)cc5)cccc34)cc2)cc1 ; N#Cc1ccc(-c2cccc(-c3cccc4c3sc3c(-c5ccc(-c6cccnc6)cc5)cccc34)c2)cc1]

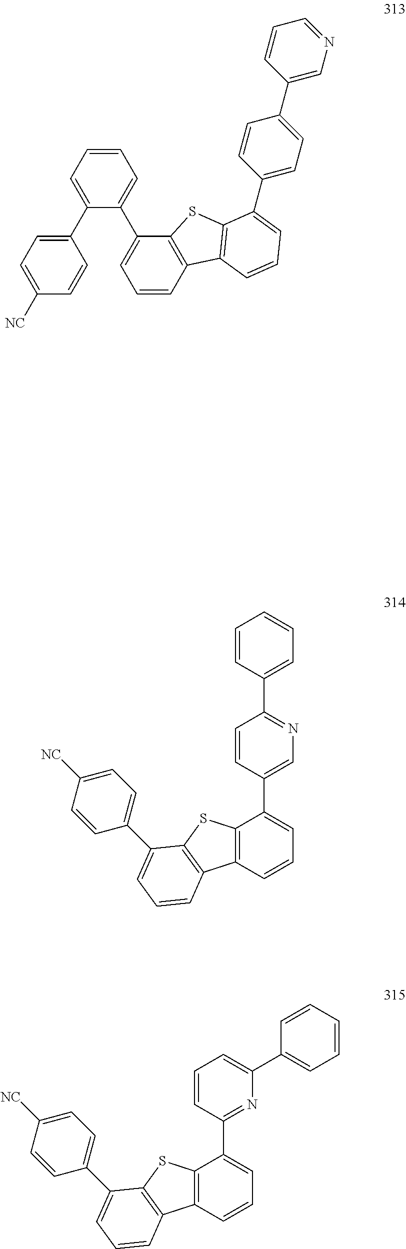 [SMILES: N#Cc1ccc(-c2ccccc2-c2cccc3c2sc2c(-c4ccc(-c5cccnc5)cc4)cccc23)cc1 ; N#Cc1ccc(-c2cccc3c2sc2c(-c4ccc(-c5ccccc5)nc4)cccc23)cc1 ; N#Cc1ccc(-c2cccc3c2sc2c(-c4cccc(-c5ccccc5)n4)cccc23)cc1]

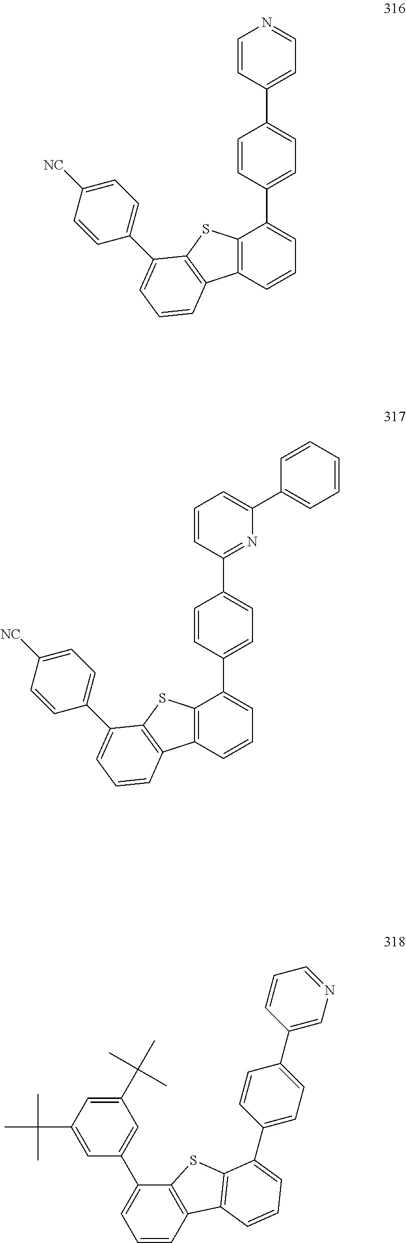 [SMILES: N#Cc1ccc(-c2cccc3c2sc2c(-c4ccc(-c5ccncc5)cc4)cccc23)cc1 ; N#Cc1ccc(-c2cccc3c2sc2c(-c4ccc(-c5cccc(-c6ccccc6)n5)cc4)cccc23)cc1 ; CC(C)(C)c1cc(-c2cccc3c2sc2c(-c4ccc(-c5cccnc5)cc4)cccc23)cc(C(C)(C)C)c1]

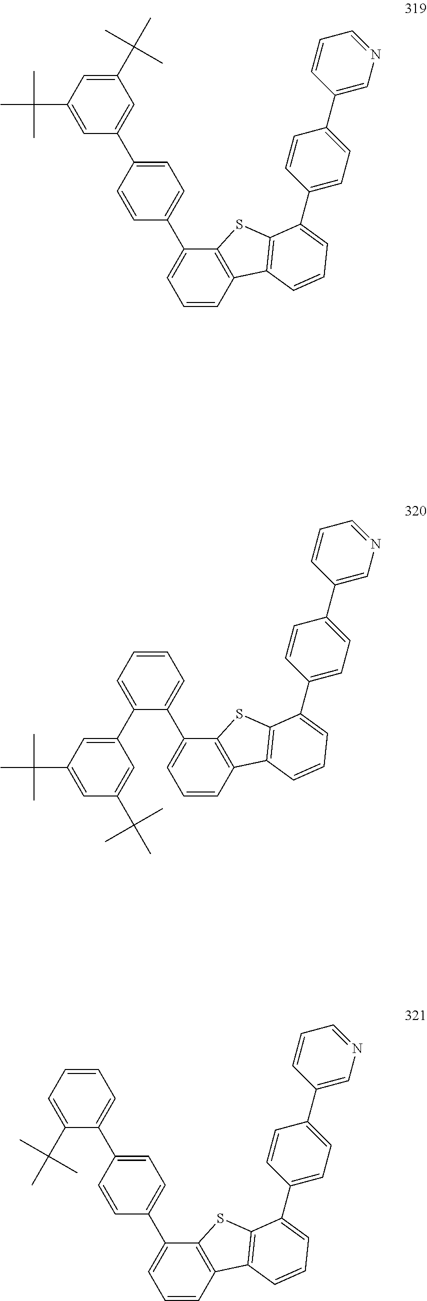 [SMILES: CC(C)(C)c1cc(-c2ccc(-c3cccc4c3sc3c(-c5ccc(-c6cccnc6)cc5)cccc34)cc2)cc(C(C)(C)C)c1 ; CC(C)(C)c1cc(-c2ccccc2-c2cccc3c2sc2c(-c4ccc(-c5cccnc5)cc4)cccc23)cc(C(C)(C)C)c1 ; CC(C)(C)c1ccccc1-c1ccc(-c2cccc3c2sc2c(-c4ccc(-c5cccnc5)cc4)cccc23)cc1]

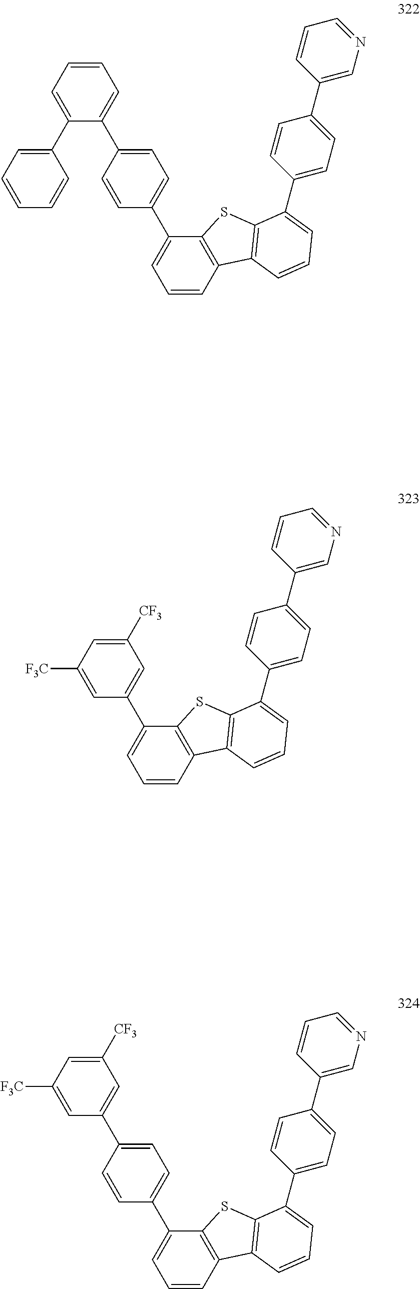 [SMILES: c1ccc(-c2ccccc2-c2ccc(-c3cccc4c3sc3c(-c5ccc(-c6cccnc6)cc5)cccc34)cc2)cc1 ; FC(F)(F)c1cc(-c2cccc3c2sc2c(-c4ccc(-c5cccnc5)cc4)cccc23)cc(C(F)(F)F)c1 ; FC(F)(F)c1cc(-c2ccc(-c3cccc4c3sc3c(-c5ccc(-c6cccnc6)cc5)cccc34)cc2)cc(C(F)(F)F)c1]

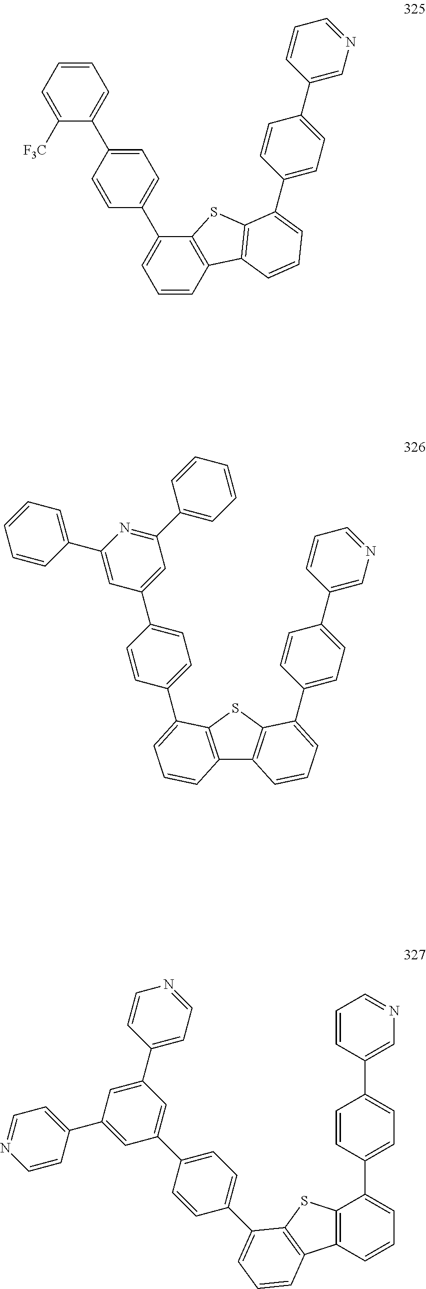 [SMILES: FC(F)(F)c1ccccc1-c1ccc(-c2cccc3c2sc2c(-c4ccc(-c5cccnc5)cc4)cccc23)cc1 ; c1ccc(-c2cc(-c3ccc(-c4cccc5c4sc4c(-c6ccc(-c7cccnc7)cc6)cccc45)cc3)cc(-c3ccccc3)n2)cc1 ; c1cncc(-c2ccc(-c3cccc4c3sc3c(-c5ccc(-c6cc(-c7ccncc7)cc(-c7ccncc7)c6)cc5)cccc34)cc2)c1]

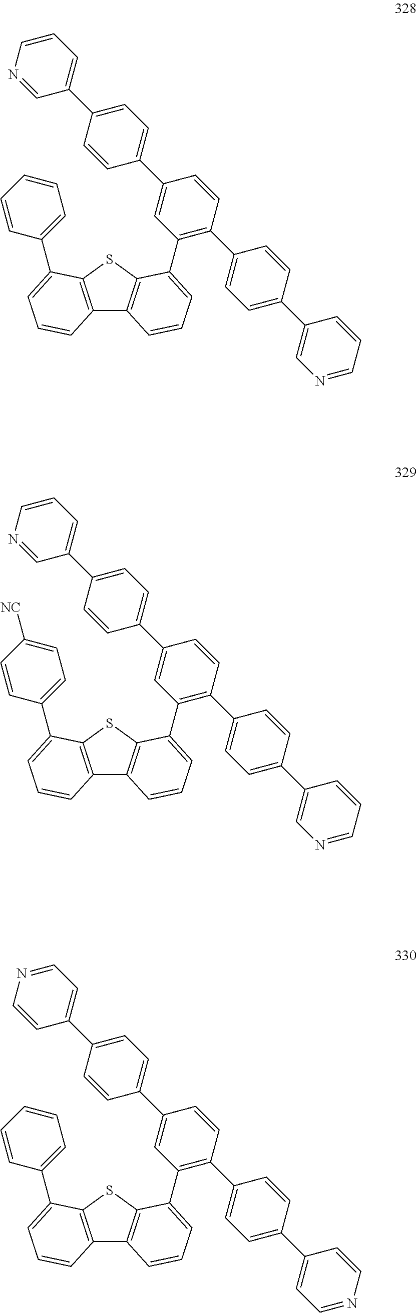 [SMILES: c1ccc(-c2cccc3c2sc2c(-c4cc(-c5ccc(-c6cccnc6)cc5)ccc4-c4ccc(-c5cccnc5)cc4)cccc23)cc1 ; N#Cc1ccc(-c2cccc3c2sc2c(-c4cc(-c5ccc(-c6cccnc6)cc5)ccc4-c4ccc(-c5cccnc5)cc4)cccc23)cc1 ; c1ccc(-c2cccc3c2sc2c(-c4cc(-c5ccc(-c6ccncc6)cc5)ccc4-c4ccc(-c5ccncc5)cc4)cccc23)cc1]

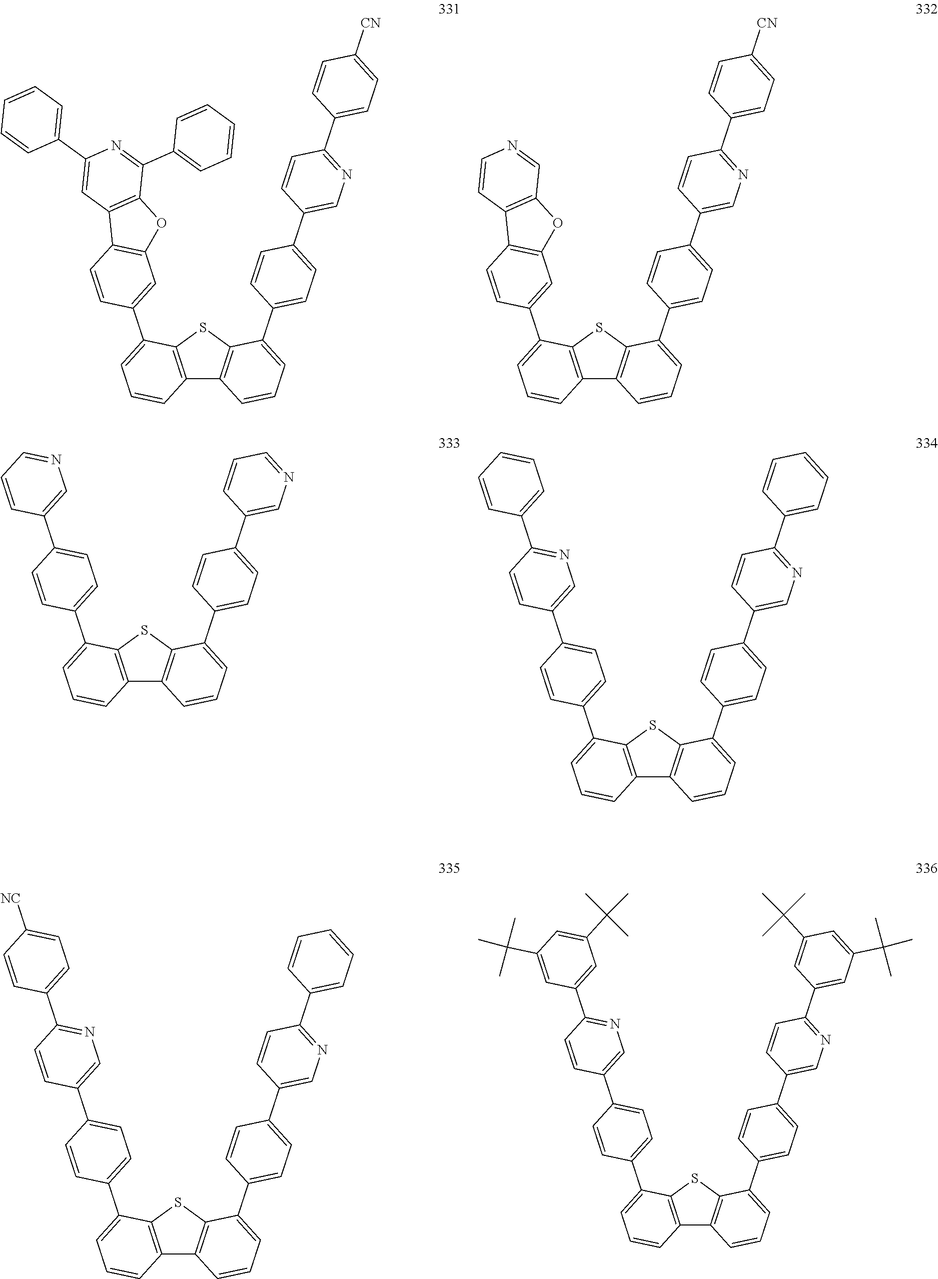

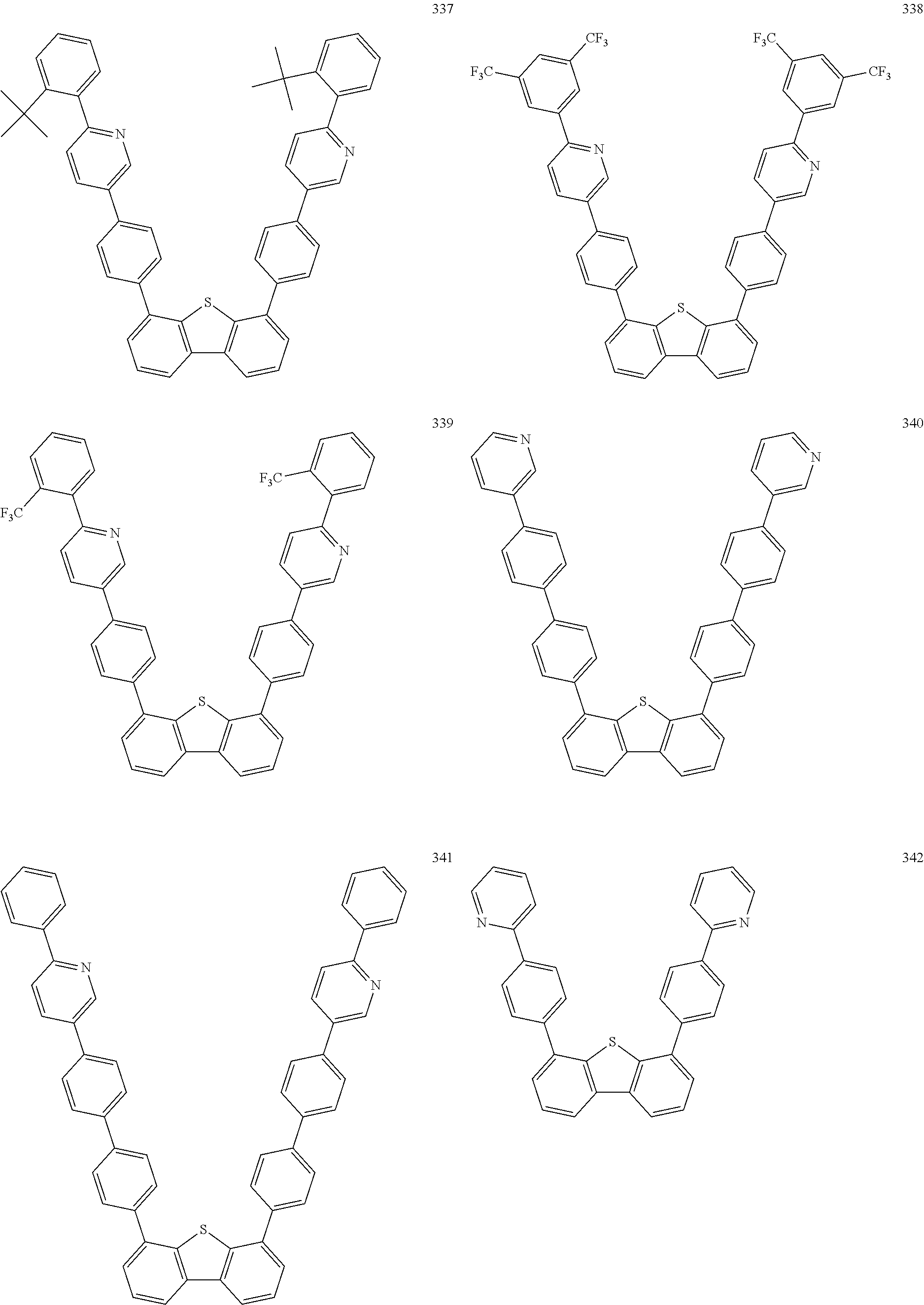

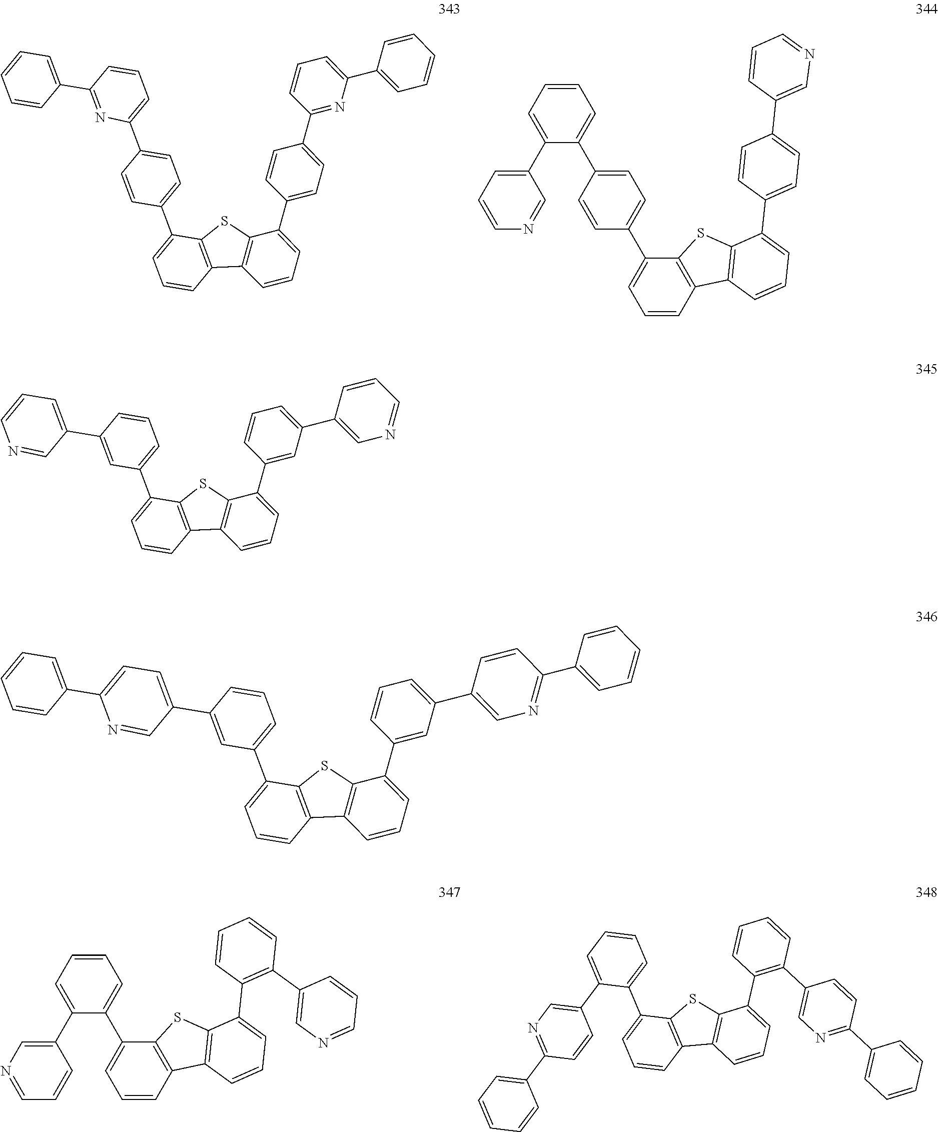 [SMILES: c1ccc(-c2cccc(-c3ccc(-c4cccc5c4sc4c(-c6ccc(-c7cccc(-c8ccccc8)n7)cc6)cccc45)cc3)n2)cc1 ; c1cncc(-c2ccc(-c3cccc4c3sc3c(-c5ccc(-c6ccccc6-c6cccnc6)cc5)cccc34)cc2)c1 ; c1cncc(-c2cccc(-c3cccc4c3sc3c(-c5cccc(-c6cccnc6)c5)cccc34)c2)c1 ; c1ccc(-c2ccc(-c3cccc(-c4cccc5c4sc4c(-c6cccc(-c7ccc(-c8ccccc8)nc7)c6)cccc45)c3)cn2)cc1 ; c1cncc(-c2ccccc2-c2cccc3c2sc2c(-c4ccccc4-c4cccnc4)cccc23)c1 ; c1ccc(-c2ccc(-c3ccccc3-c3cccc4c3sc3c(-c5ccccc5-c5ccc(-c6ccccc6)nc5)cccc34)cn2)cc1]

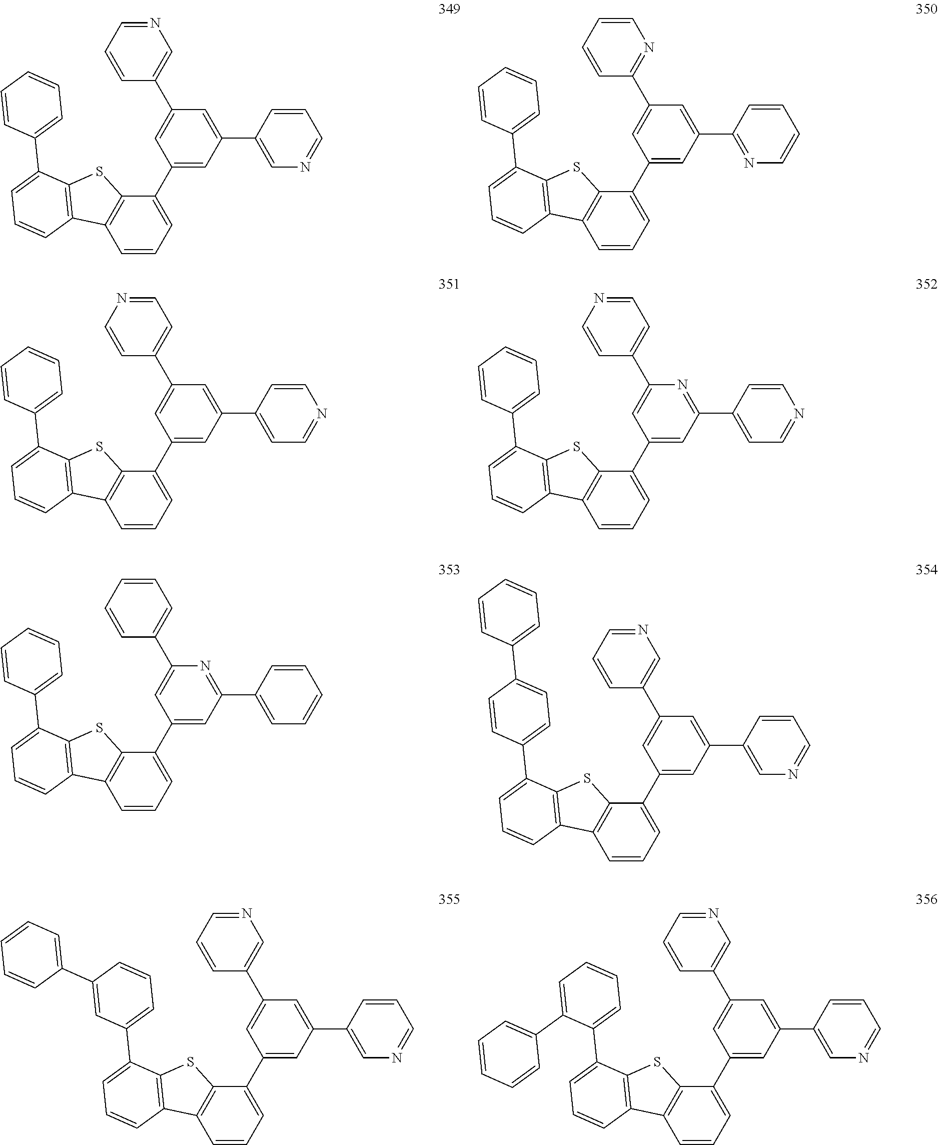

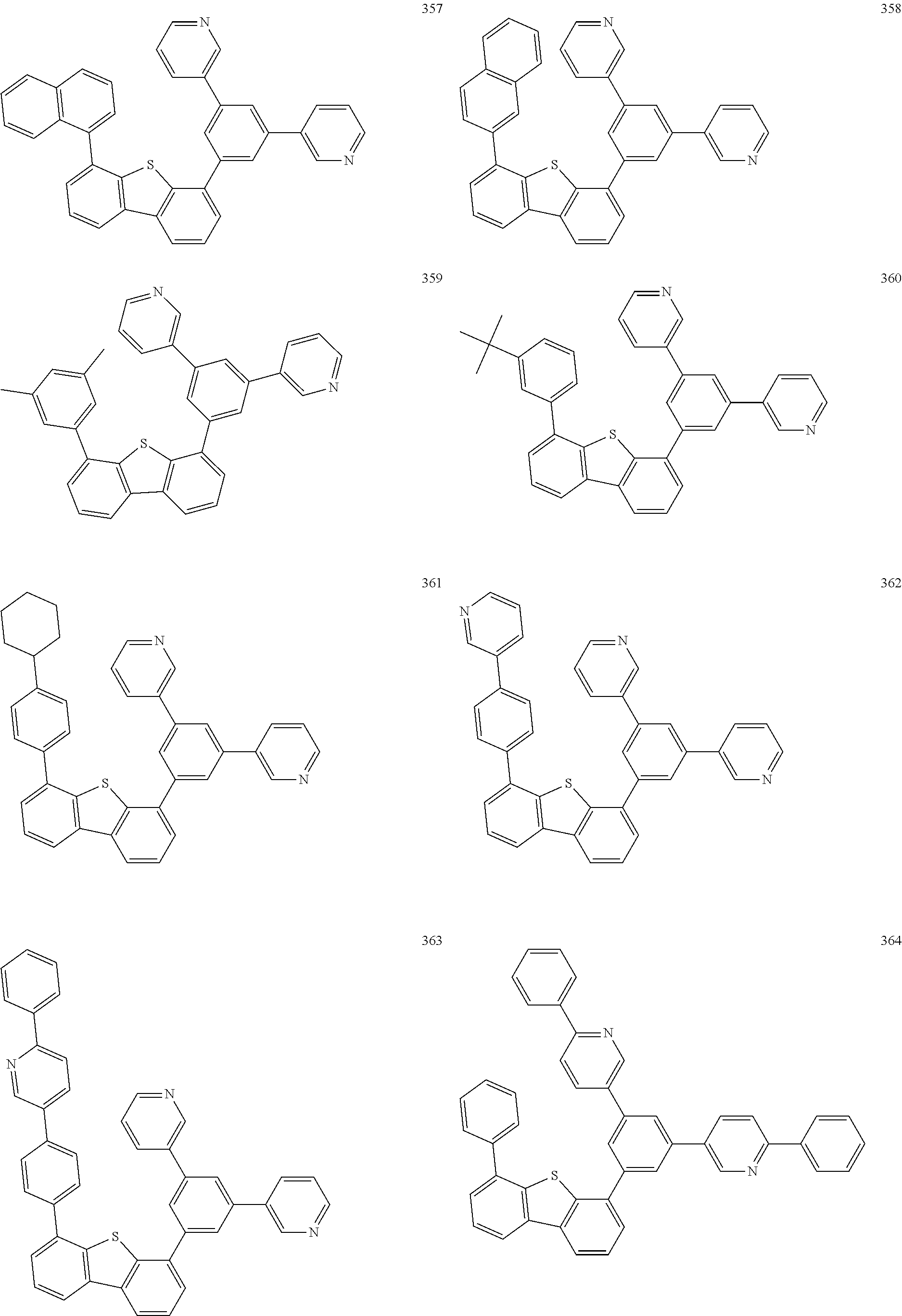

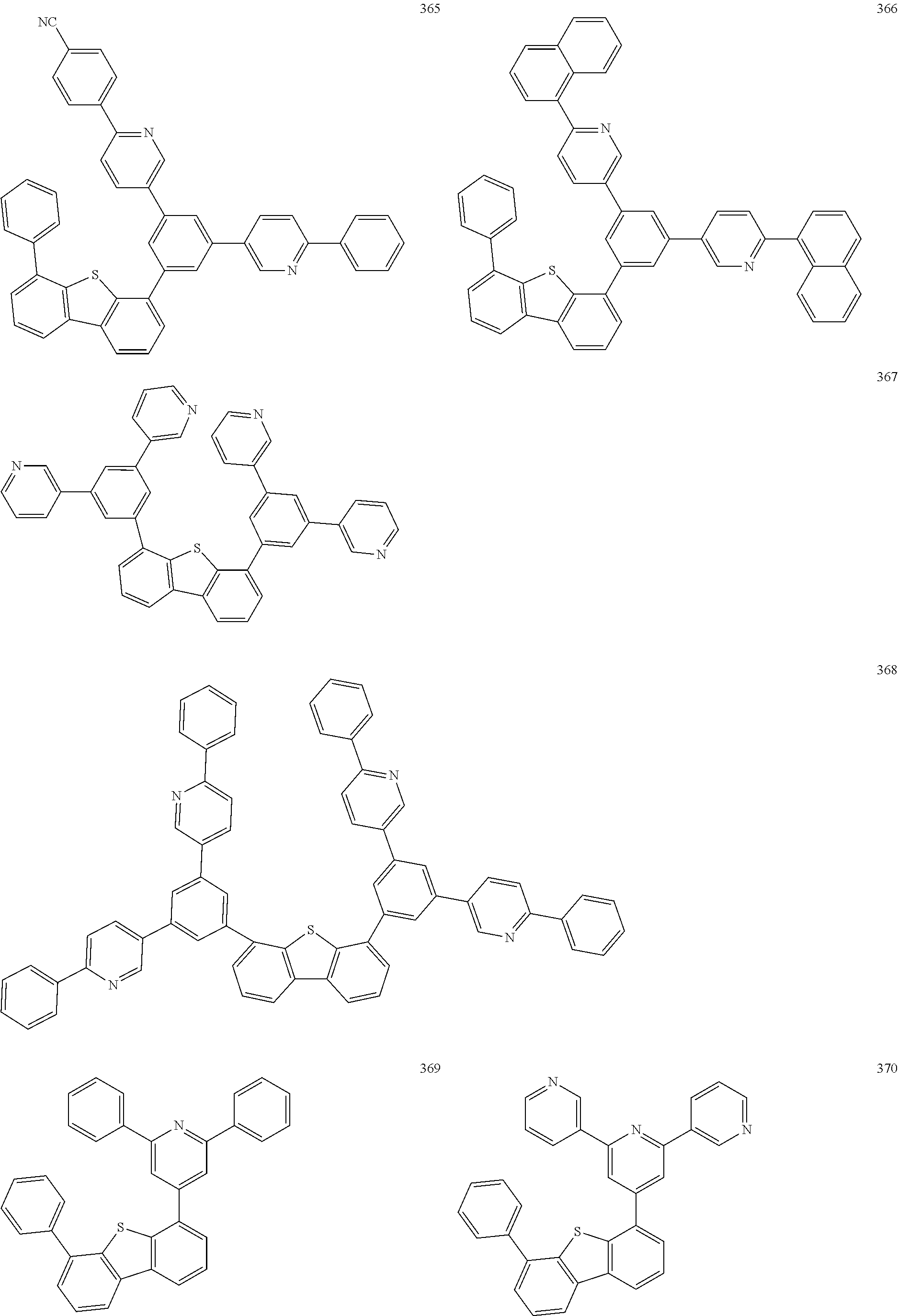

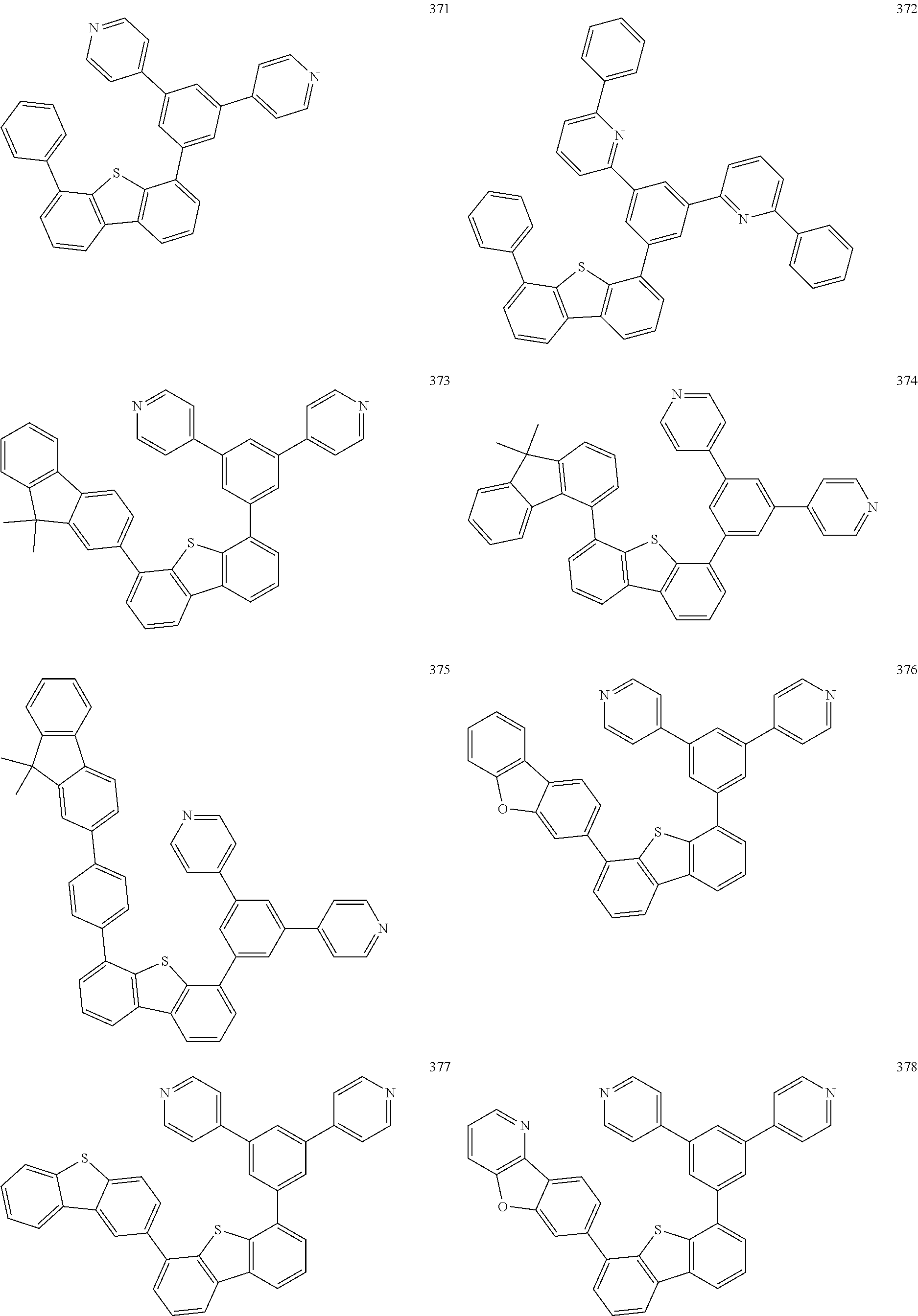

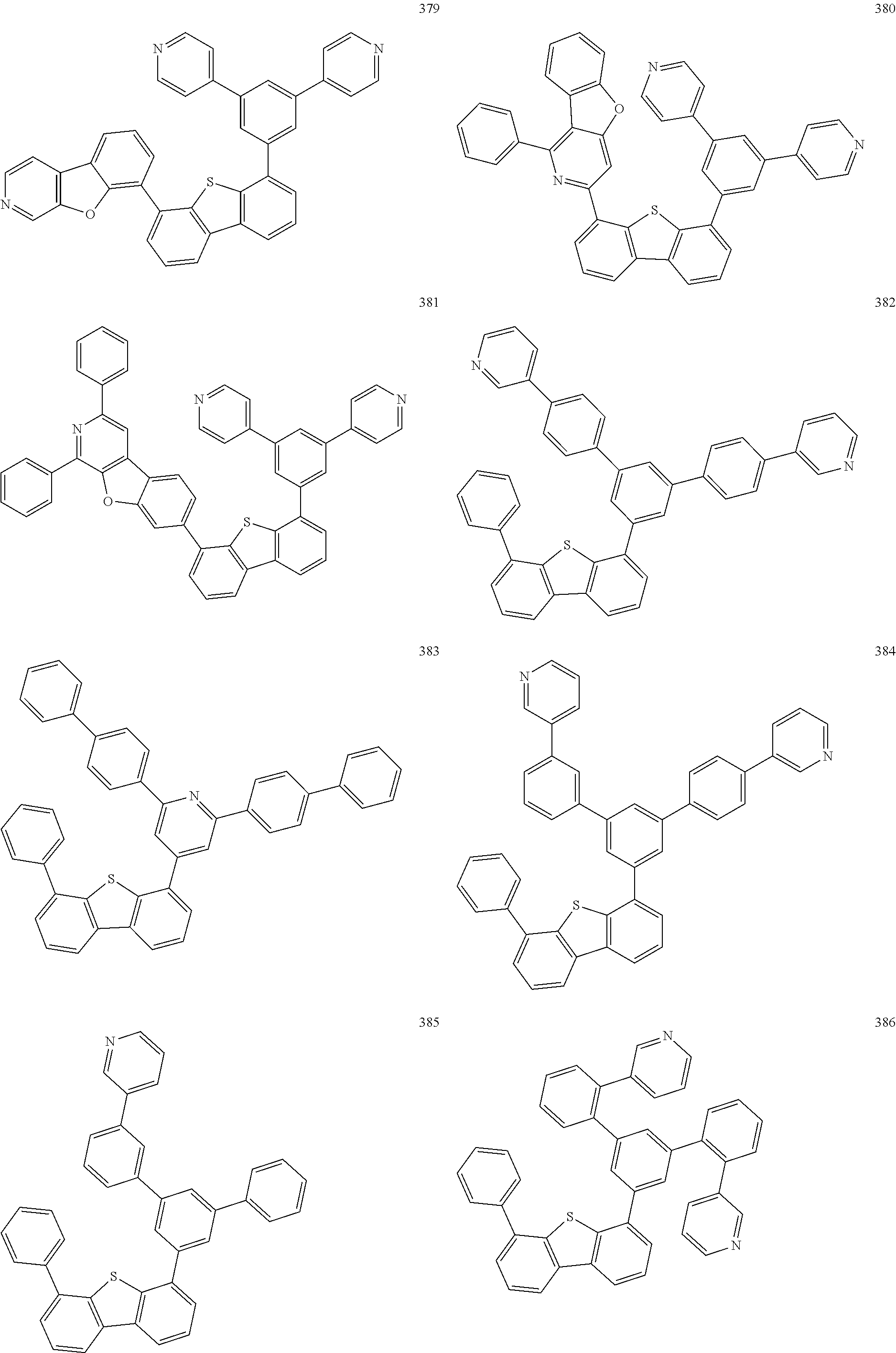

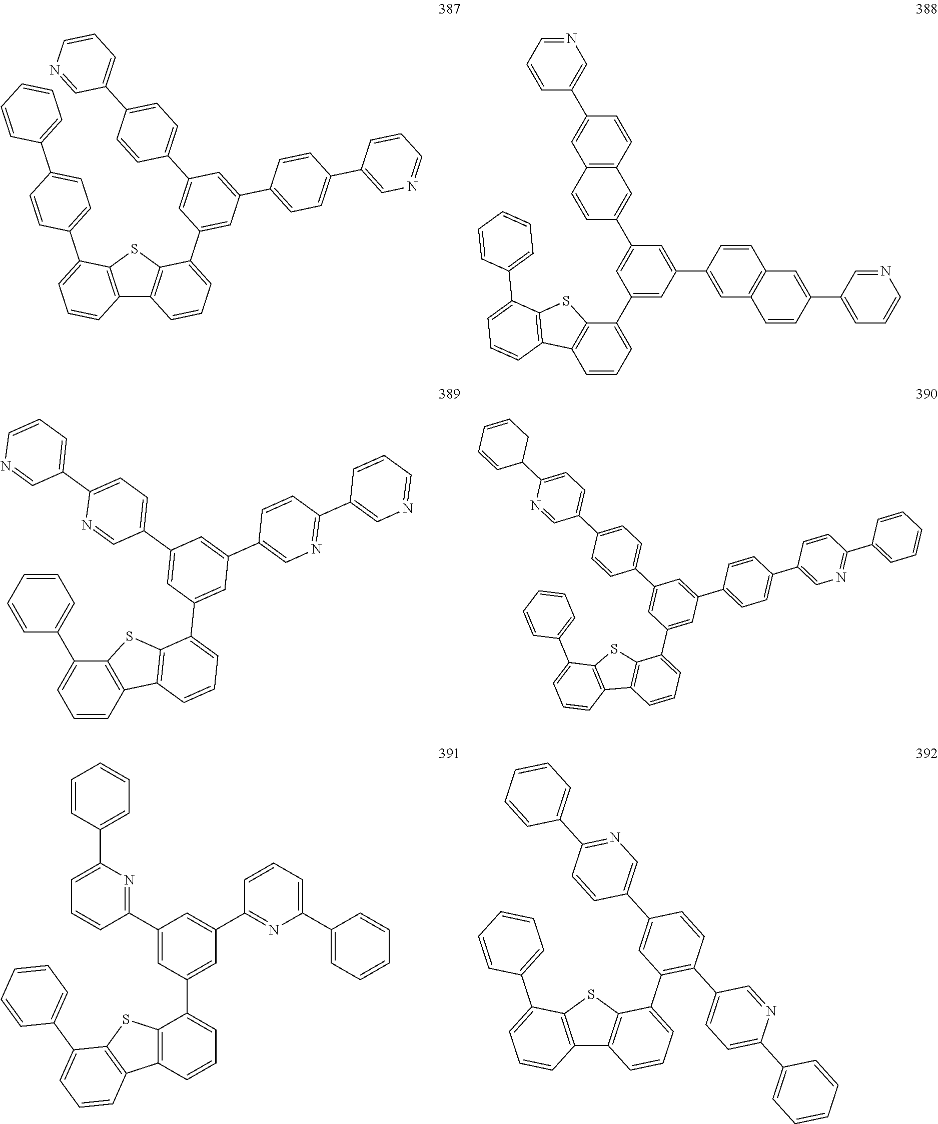

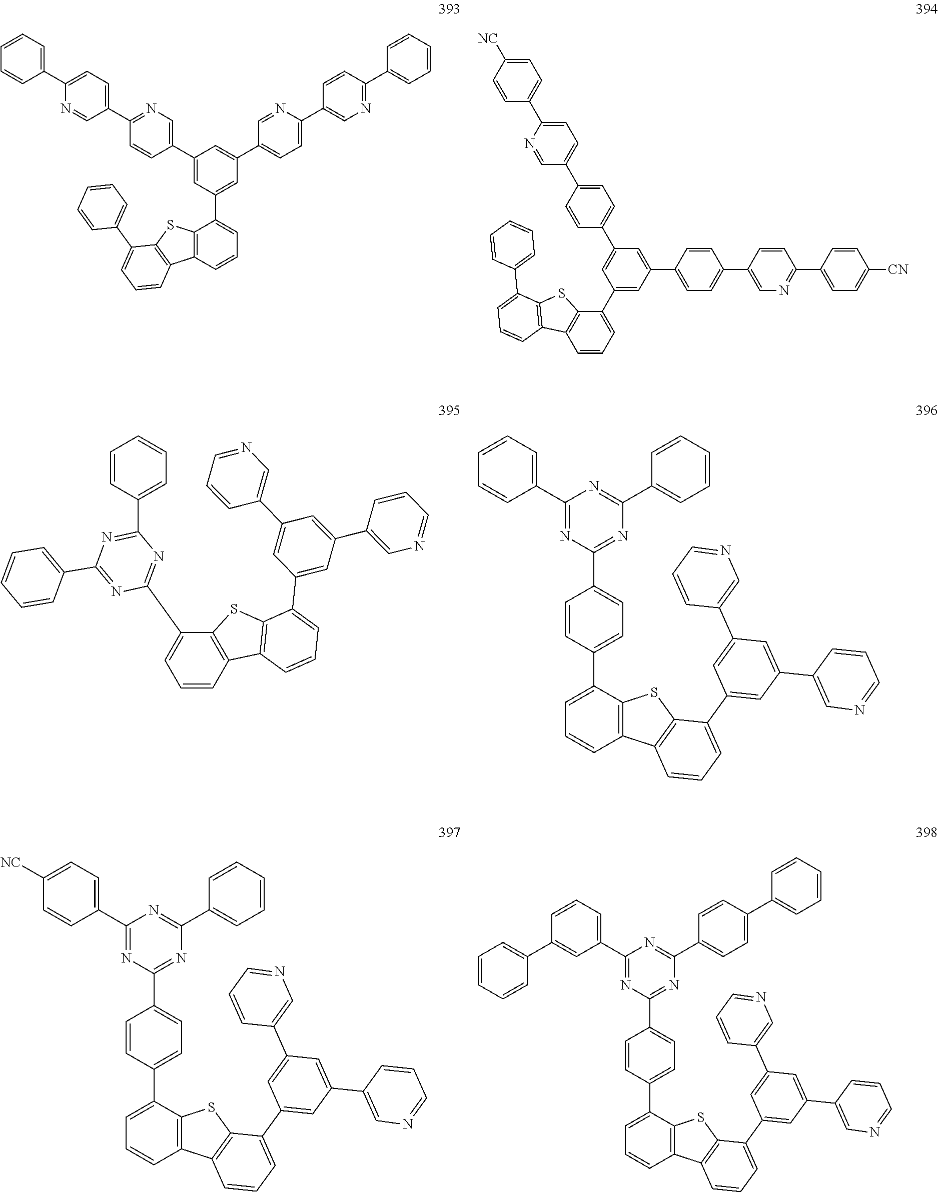

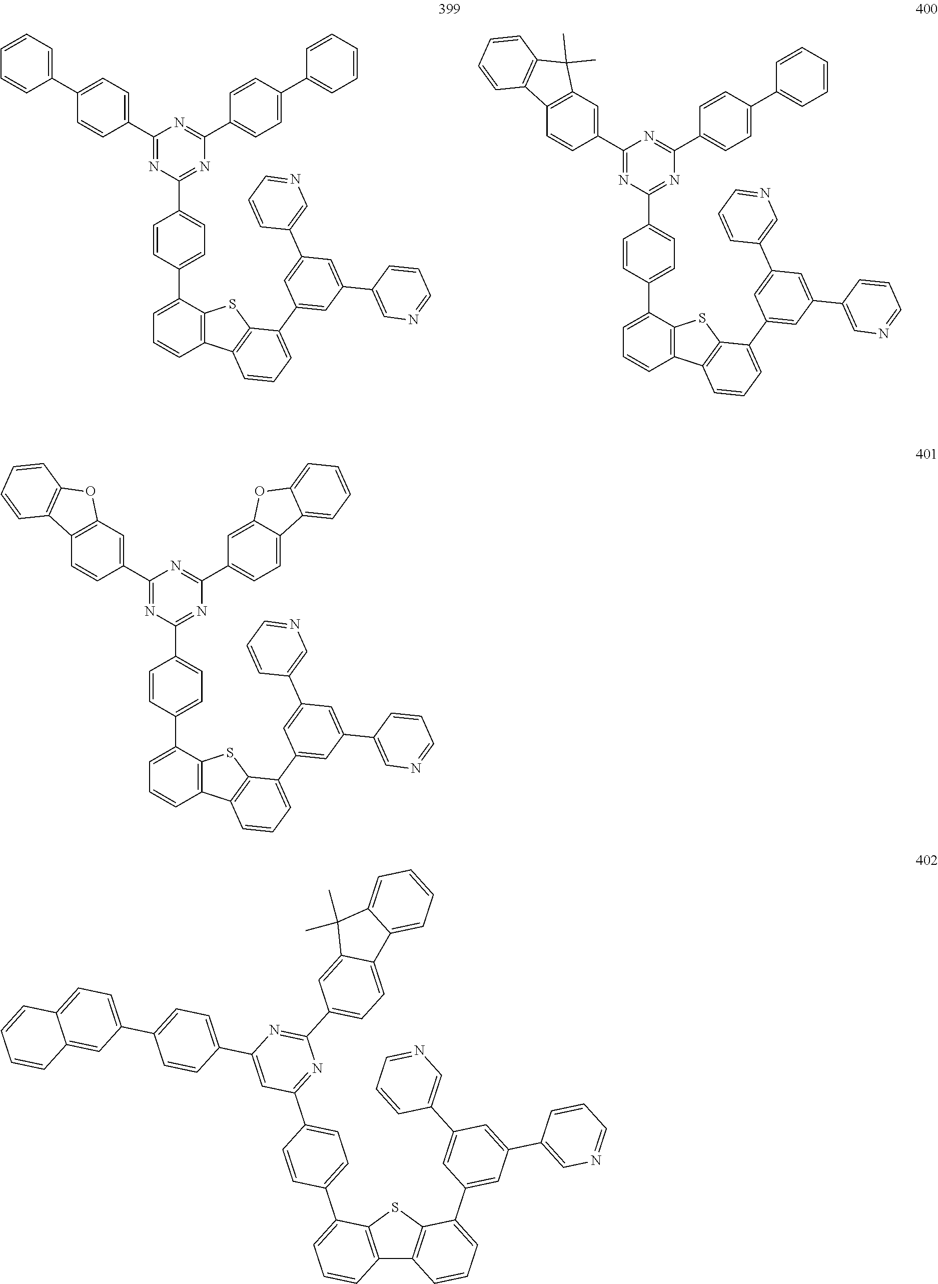

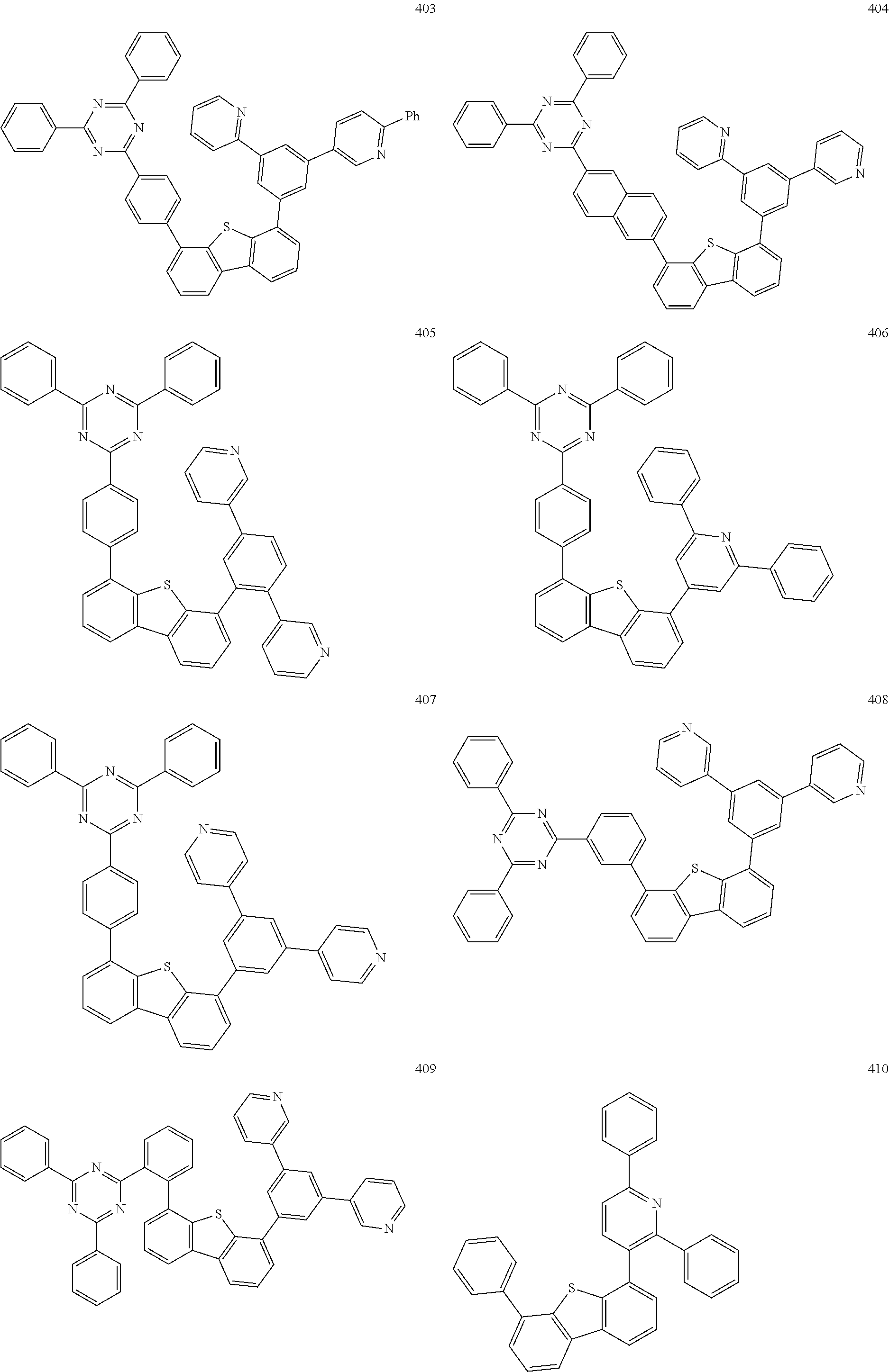

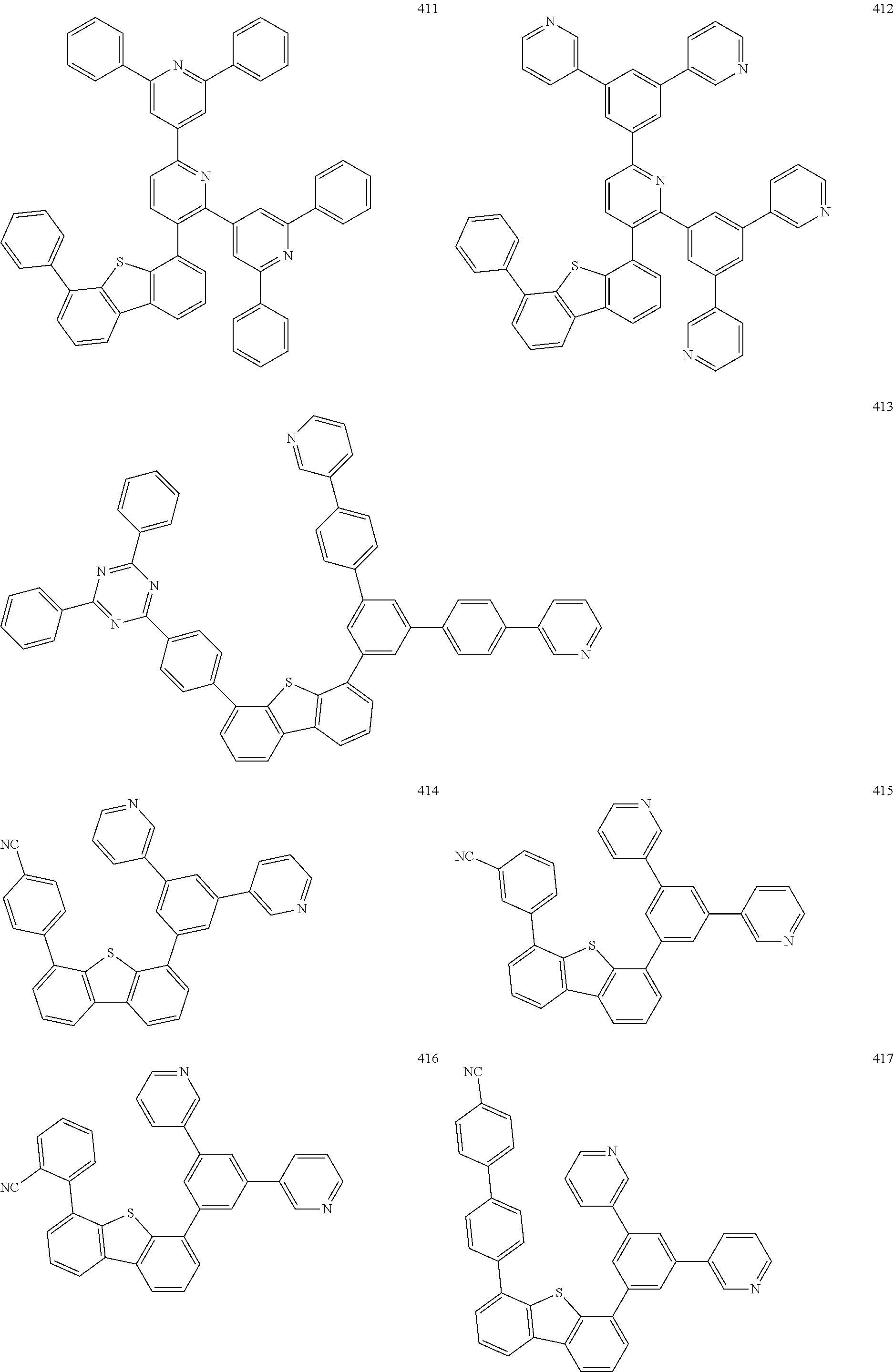

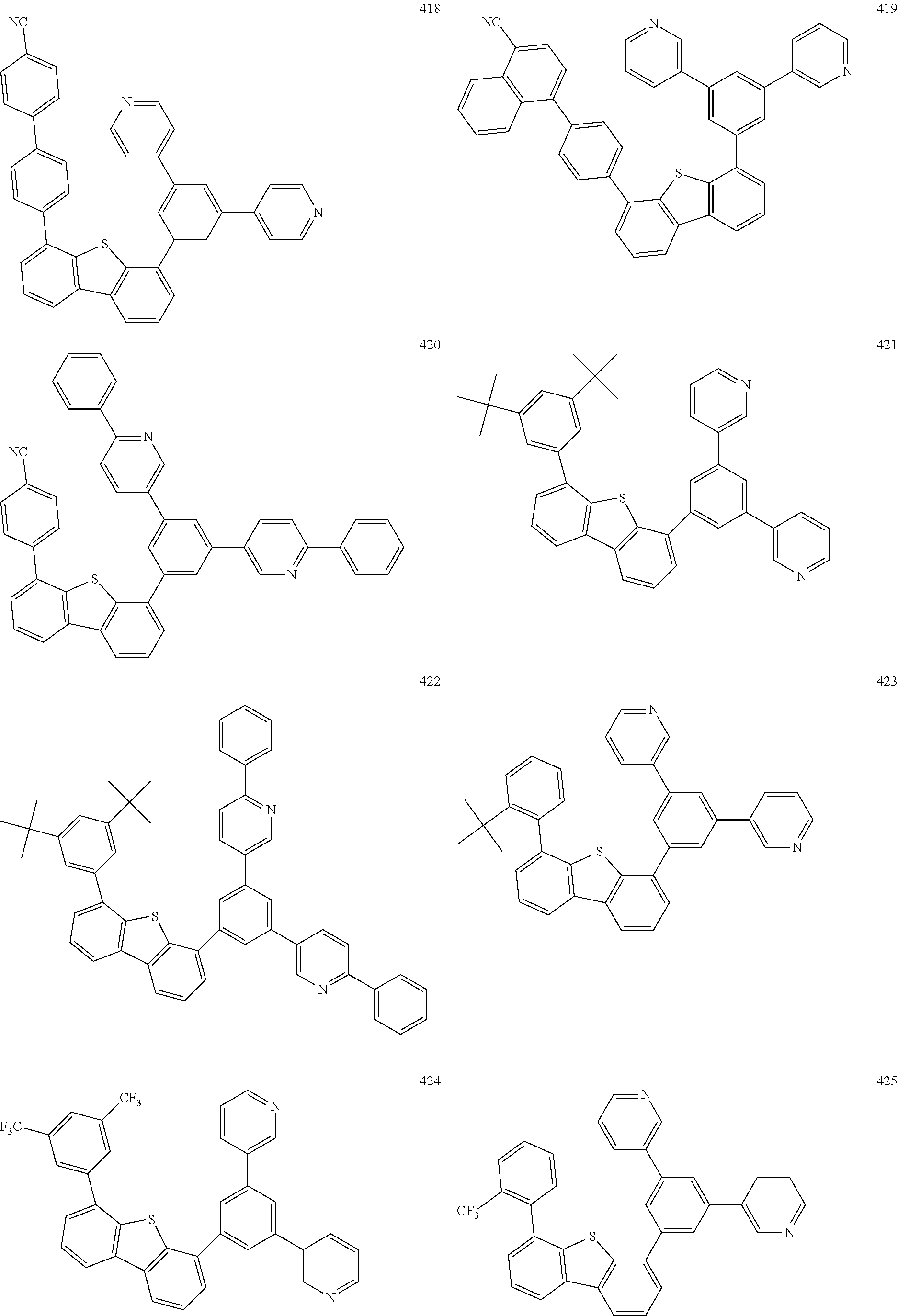

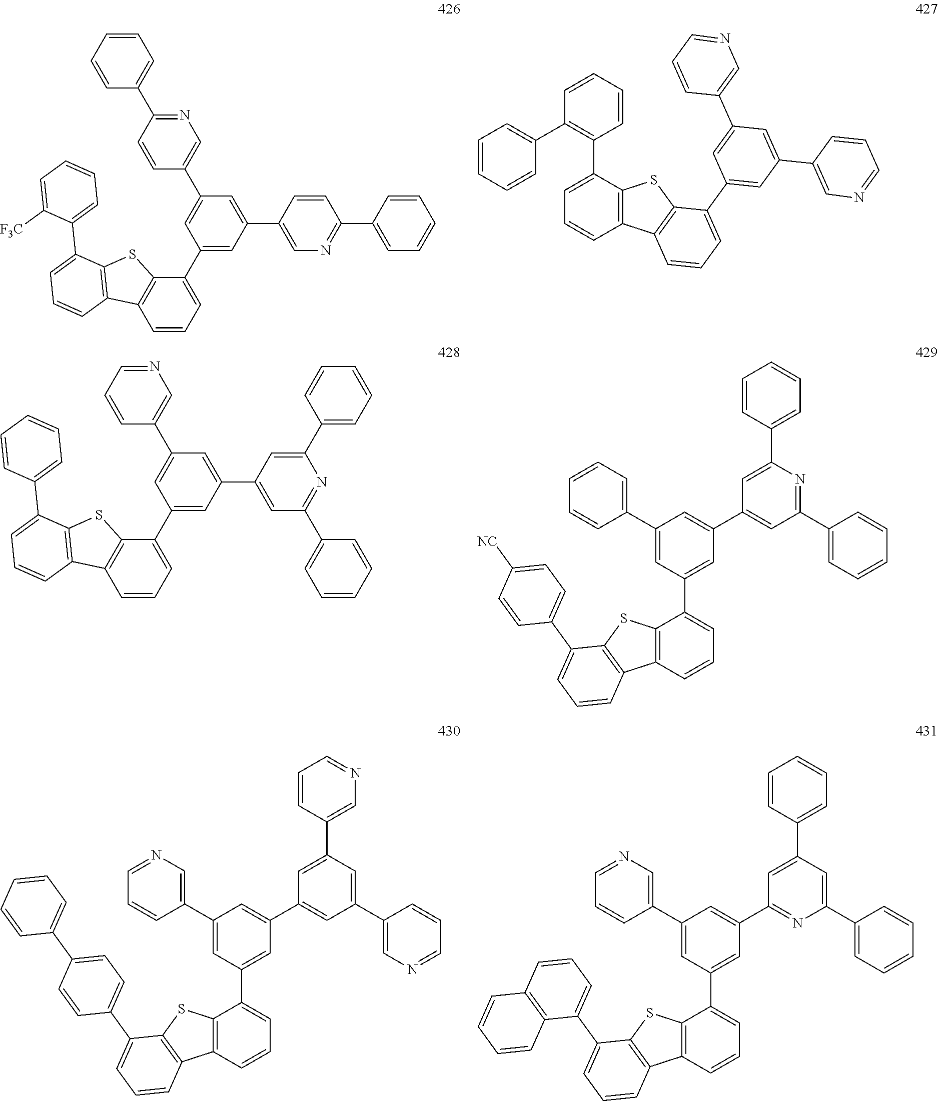

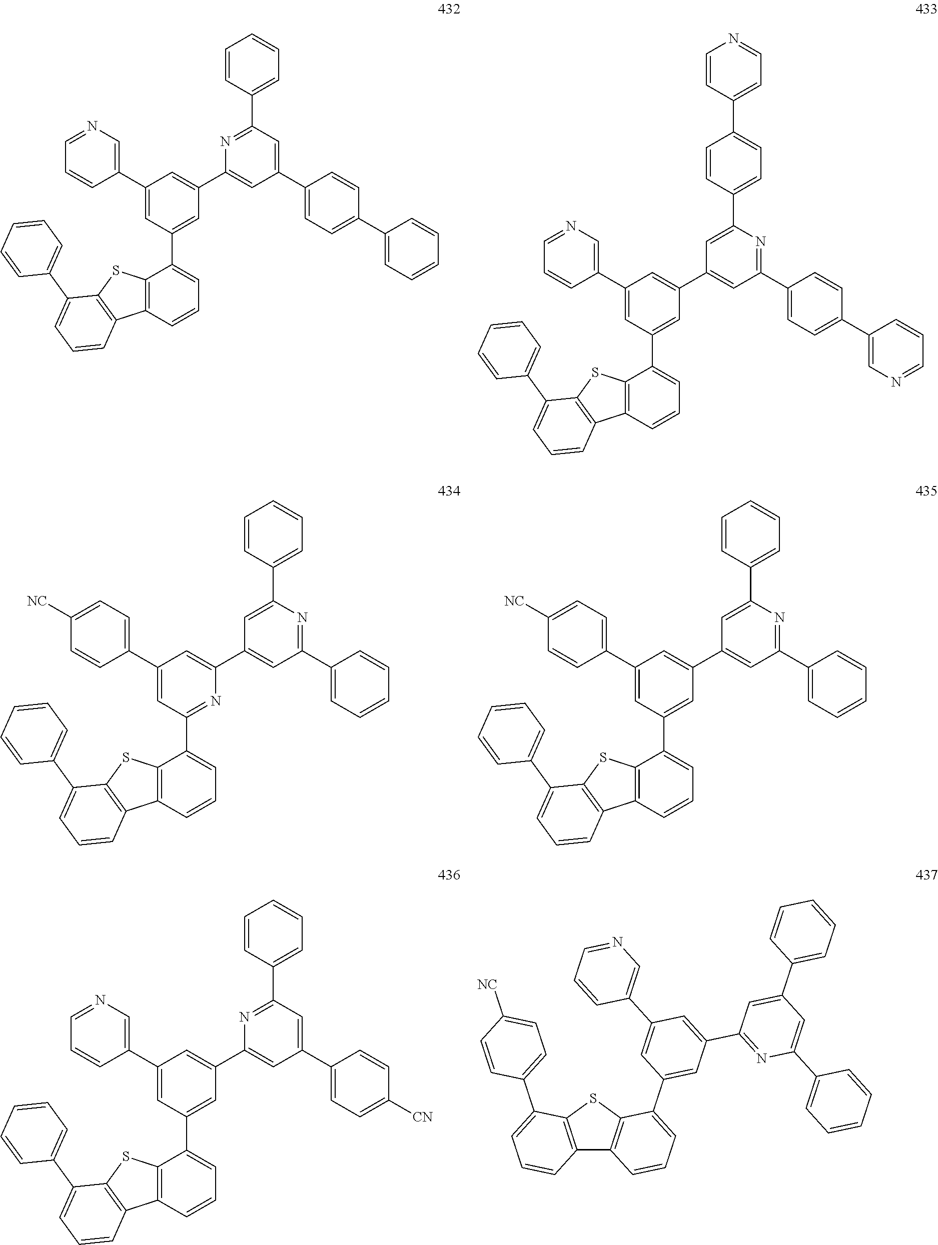

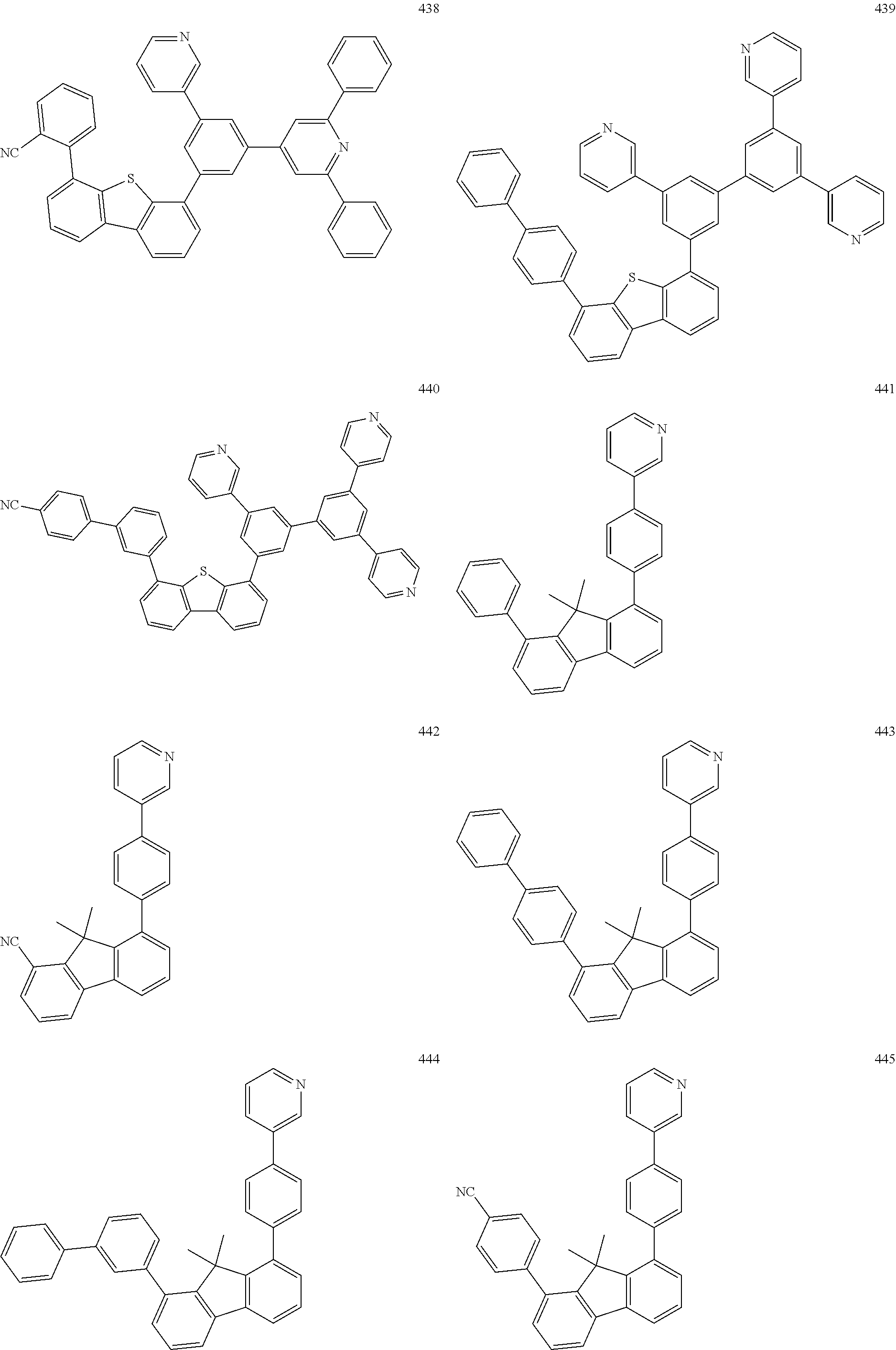

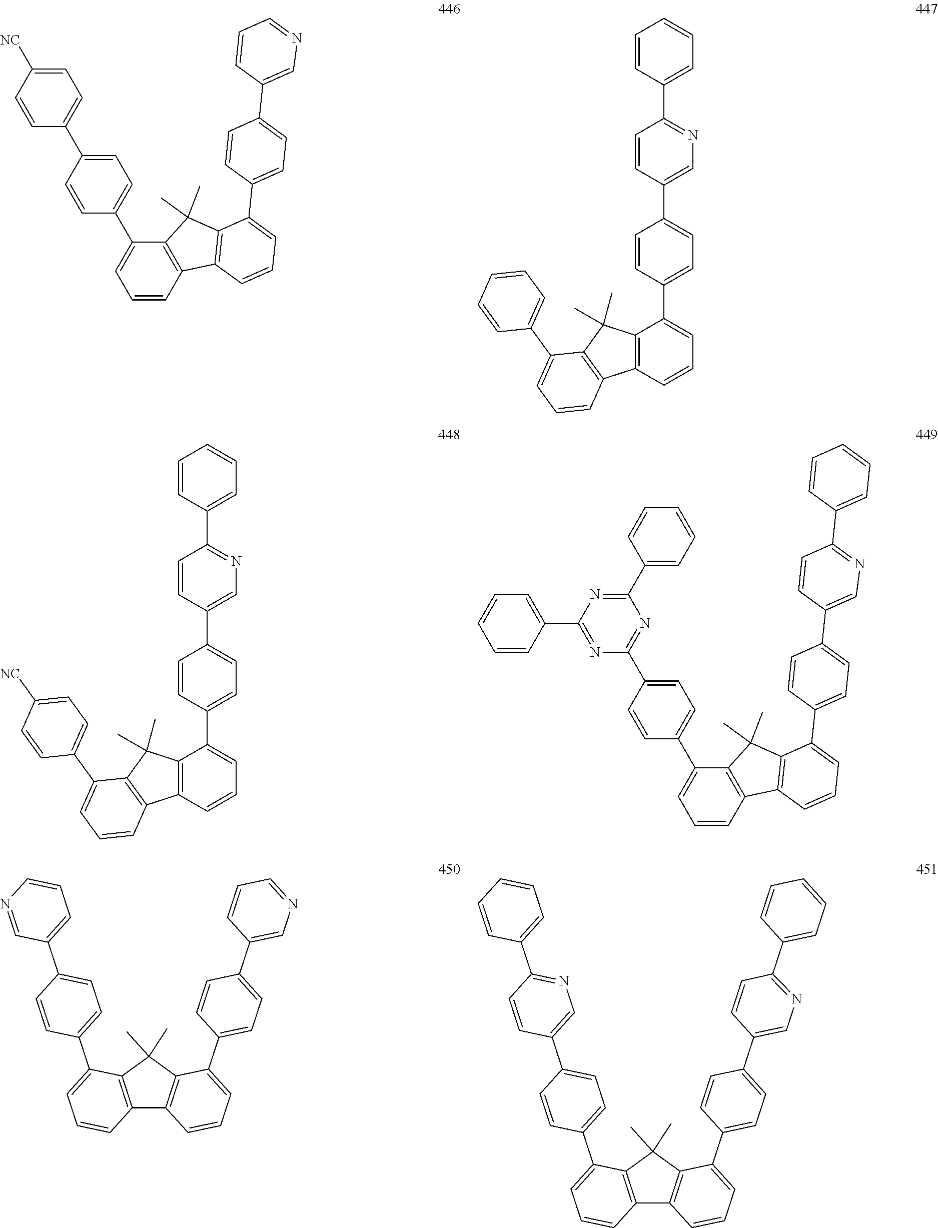 [SMILES: CC1(C)c2c(-c3ccc(-c4ccc(C#N)cc4)cc3)cccc2-c2cccc(-c3ccc(-c4cccnc4)cc3)c21 ; CC1(C)c2c(-c3ccccc3)cccc2-c2cccc(-c3ccc(-c4ccc(-c5ccccc5)nc4)cc3)c21 ; CC1(C)c2c(-c3ccc(C#N)cc3)cccc2-c2cccc(-c3ccc(-c4ccc(-c5ccccc5)nc4)cc3)c21 ; CC1(C)c2c(-c3ccc(-c4ccc(-c5ccccc5)nc4)cc3)cccc2-c2cccc(-c3ccc(-c4nc(-c5ccccc5)nc(-c5ccccc5)n4)cc3)c21 ; CC1(C)c2c(-c3ccc(-c4cccnc4)cc3)cccc2-c2cccc(-c3ccc(-c4cccnc4)cc3)c21 ; CC1(C)c2c(-c3ccc(-c4ccc(-c5ccccc5)nc4)cc3)cccc2-c2cccc(-c3ccc(-c4ccc(-c5ccccc5)nc4)cc3)c21]

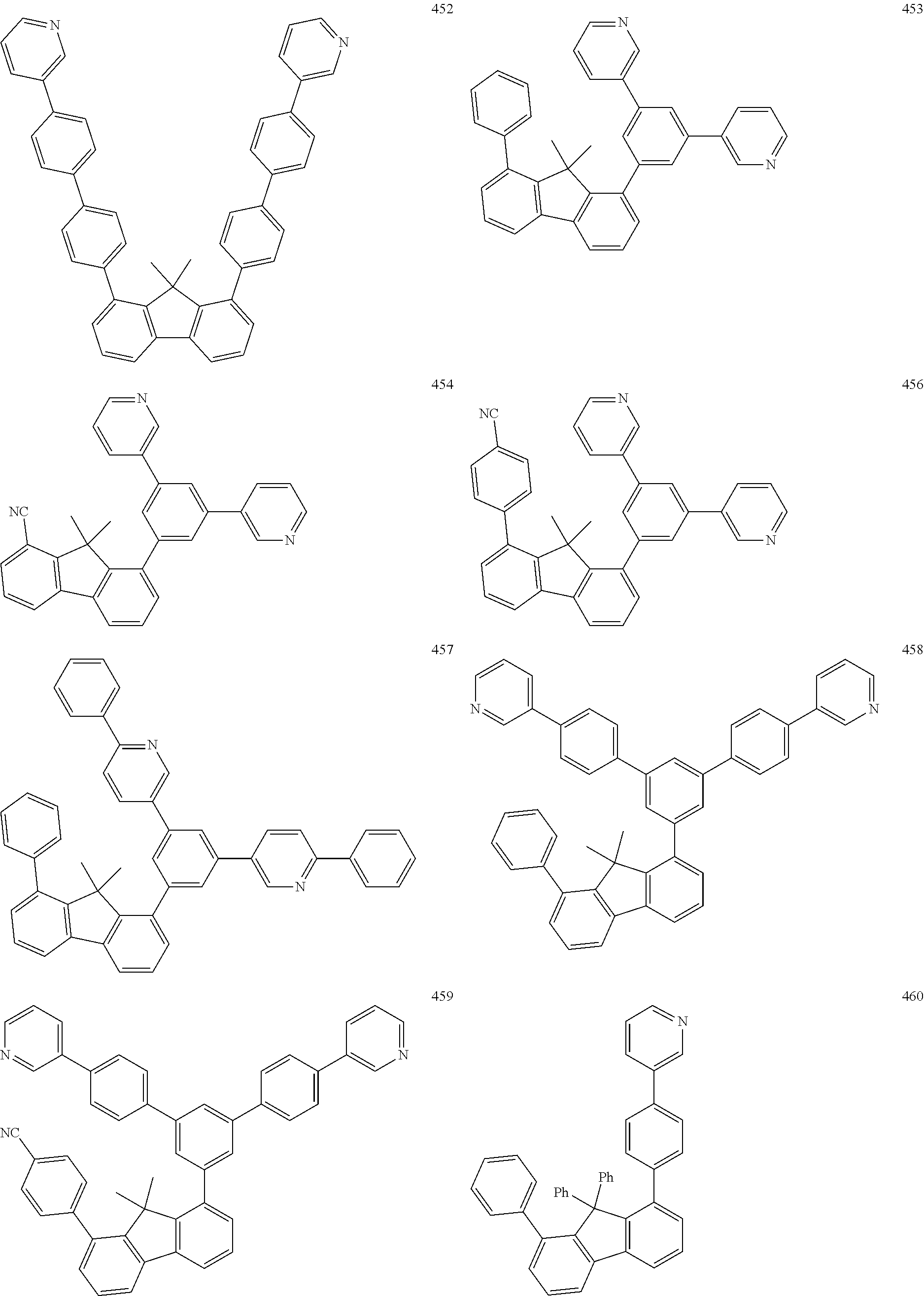

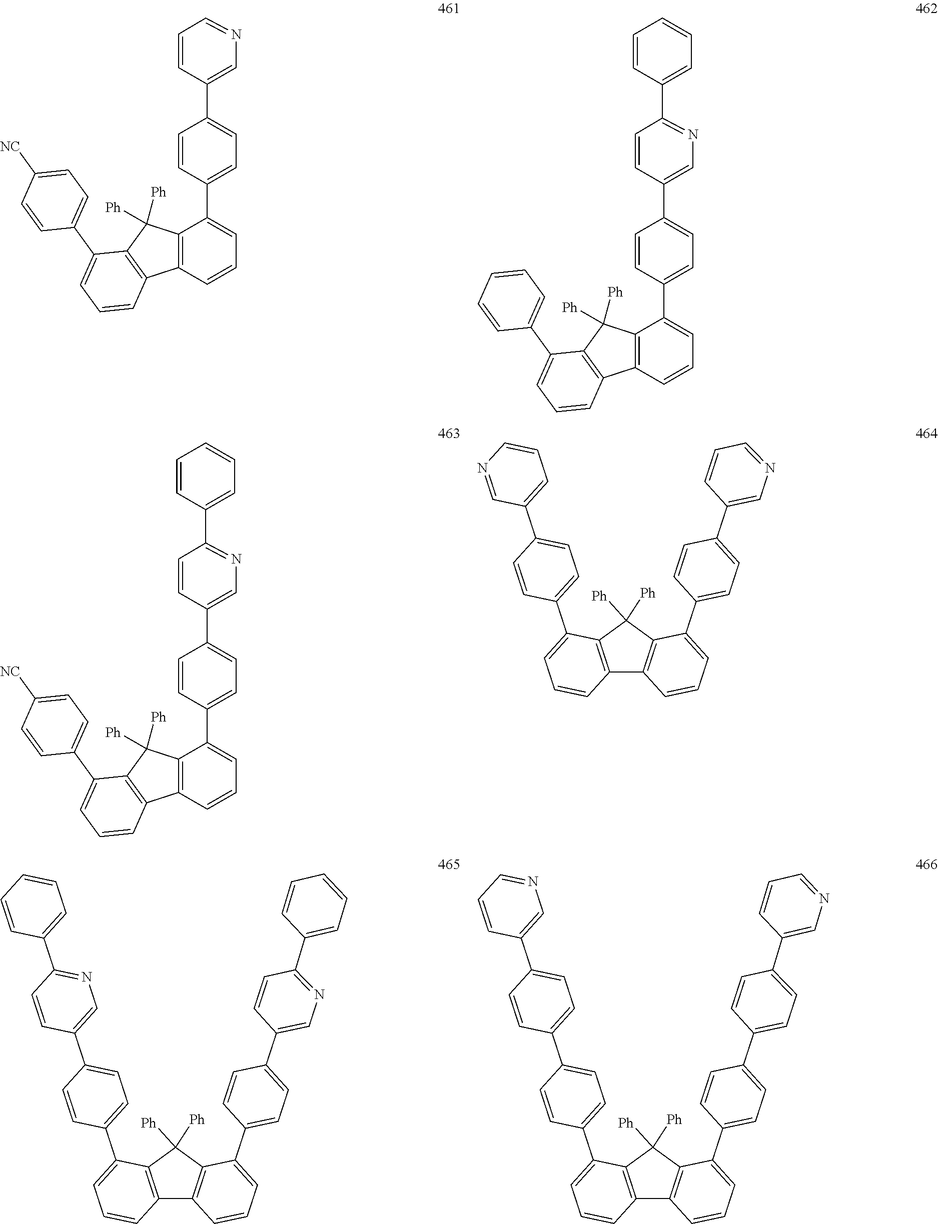

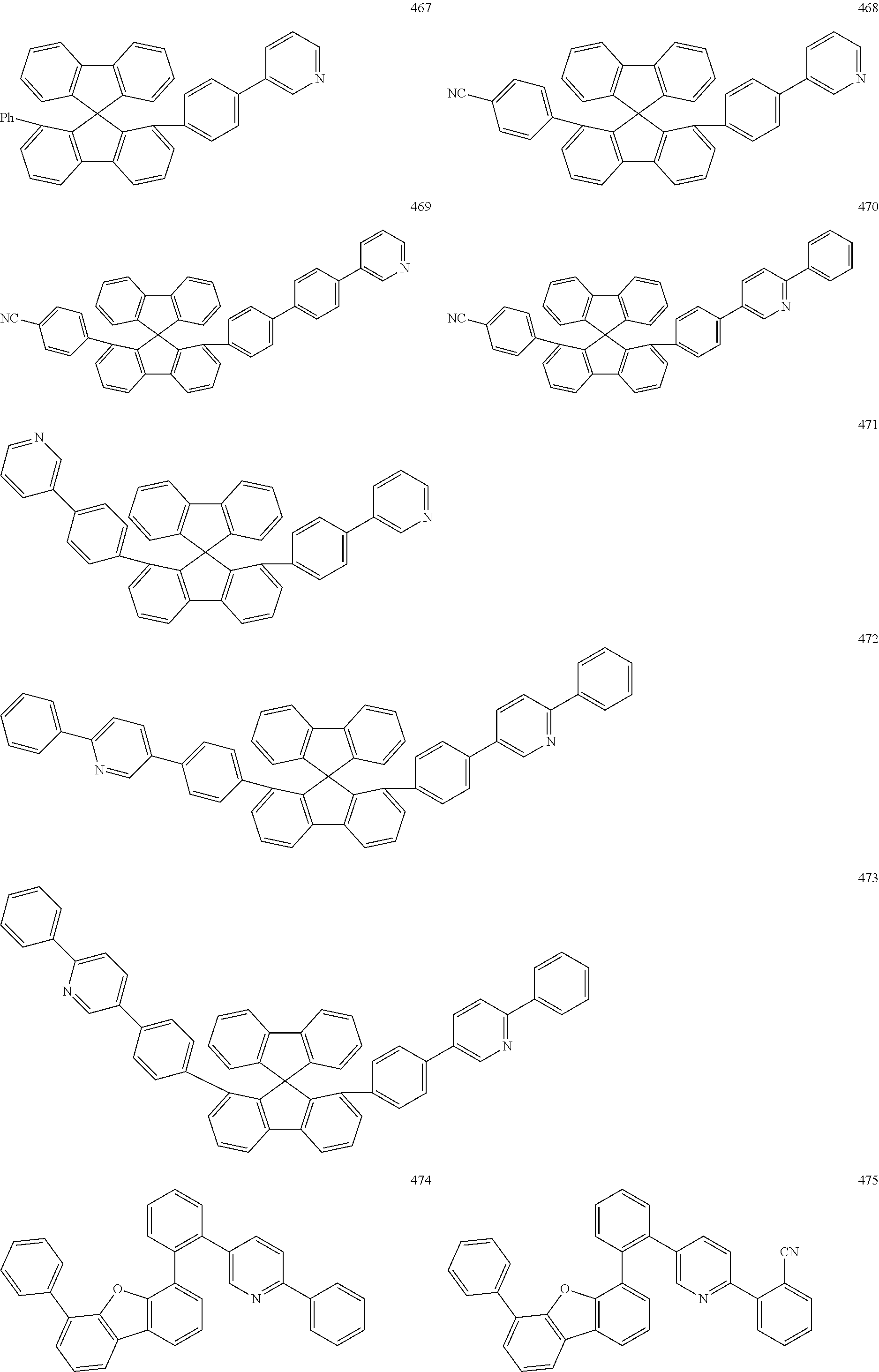

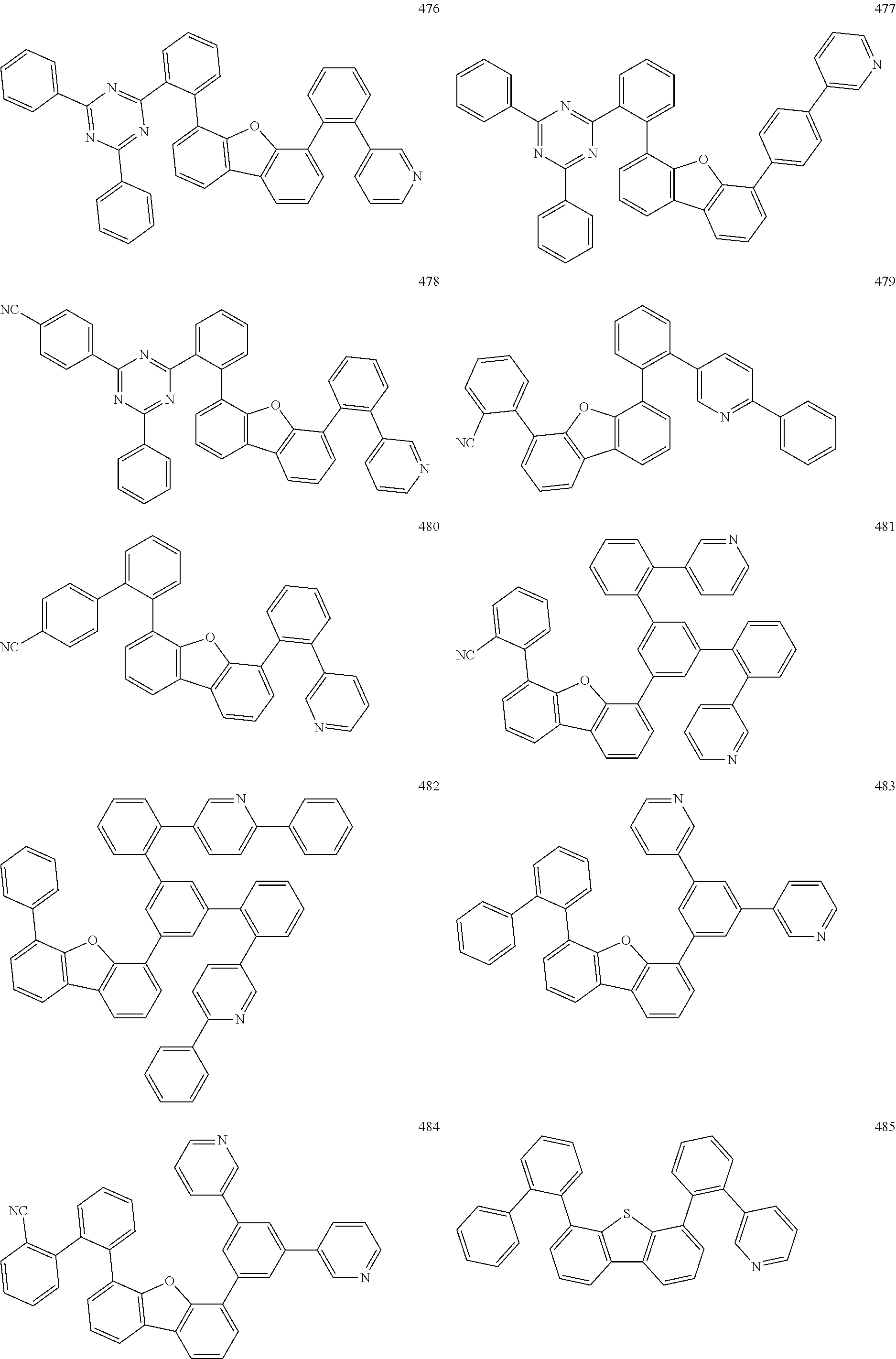

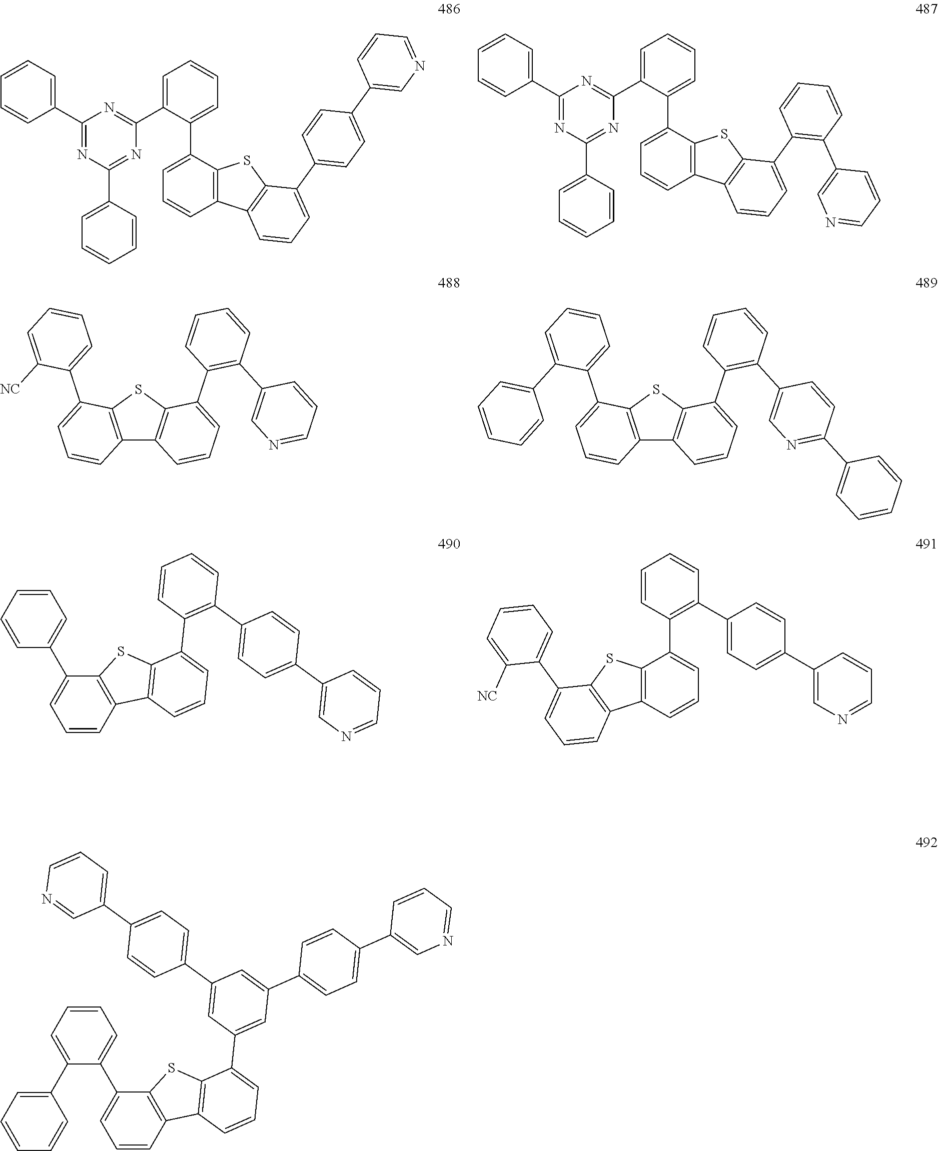

4. An organic light emitting device including a first electrode; a second electrode; and one or more organic layers disposed between the first electrode and the second electrode, wherein one or more the organic layers include an organic compound represented by Formula I according to claim 1.

5. The organic light emitting device of claim 4, wherein:
the organic layers include one or more layers selected from a hole injecting layer, a hole transport layer, a layer having functions of both hole injection and hole transport, an electron transport layer, an electron injecting layer, a layer having functions of both electron transport and electron injection, an electron blocking layer, a hole blocking layer, and a light emitting layer, and
the one or more layers include an organic compound represented by the Formula I

6. The organic light emitting device of claim 5, wherein any one of the electron transport layer, the electron injecting layer, and the layer having functions of both electron transport and electron injection includes an organic compound represented by the Formula I.
